SEQUENCE LISTING (192 sequences)

SEQ ID NO: 1 - DNA, 945 bp, Homo sapiens
attactcctgcaataatggcaaatctcacaatcgtgactgaatttatccttatggggttttctaccaataaaaatatgtgcattttgcattcgattctcttcttgttgatttatttgtgtgccctgatggggaatgtcctcattatcatgatcacaactttggaccatcatctccacacccccgtgtatttcttcttgaagaatctatctttcttggatctctgccttatttcagtcacggctcccaaatctatcgccaattctttgatacacaacaactccatttcattccttggctgtgtttcccaggtctttttgttgctttcttcagcatctgcagagctgctcctcctcacggtgatgtcctttgaccgctatactgctatatgtcaccctctgcactatgatgtcatcatggacaggagcacctgtgtccaaagagccactgtgtcttggctgtatgggggtctgattgctgtgatgcacacagctggcaccttctccttatcctactgtgggtccaacatggtccatcagttcttctgtgacattccccagttattagctatttcttgctcagaaaatttaataagagaaattgcactcatccttattaatgtagttttggatttctgctgttttattgtcatcatcattacctatgtccacgtcttctctacagtcaagaagatcccttccacagaaggccagtcaaaagcctactctatttgccttccacacttgctggttgtgttatttctttccactggattcattgcttatctgaagccagcttcagagtctccttctattttggatgctgtaatttctgtgttctacactatgctgcccccaacctttaatcccattatatacagtttgagaaacaaggccataaaggtggctctggggatgttgataaagggaaagctcaccaaaaagtaa

SEQ ID NO: 2 - DNA, 996 bp, Homo sapiens
atgagtcctgatgggaaccacagtagtgatccaacagagttcgtcctggcagggctcccaaatctcaacagcgcaagagtggaattattttctgtgtttcttcttgtctatctcctgaatctgacaggcaatgtgttgattgtgggggtggtaagggctgatactcgactacagacccctatgtacttctttctgggtaacctgtcctgcctagagatactgctcacttctgtcatcattccaaagatgctgagcaatttcctctcaaggcaacacactatttcctttgctgcatgtatcacccaattctatttctacttctttctcggggcctccgagttcttactgttggctgtcatgtctgcggatcgctacctggccatctgtcatcctctgcgctaccccttgctcatgagtggggctgtgtgctttcgtgtggccttggcctgctgggtggggggactcgtccctgtgcttggtcccacagtggctgtggccttgcttcctttctgtaagcagggtgctgtggtacagcacttcttctgcgacagtggcccactgctccgcctggcttgcaccaacaccaagaagctggaggagactgactttgtcctggcctccctcgtcattgtatcttccttgctgatcactgctgtgtcctacggcctcattgtgctggcagtcctgagcatcccctctgcttcaggccgtcagaaggccttctctacctgtacctcccacttgatagtggtgaccctcttctatggaagtgccatttttctctatgtgcggccatcgcagagtggttctgtggacactaactgggcagtgacagtaataacgacatttgtgacaccactgttgaatccattcatctatgccttacgtaatgagcaagtcaaggaagctttgaaggacatgtttaggaaggtagtggcaggcgttttagggaatcttttacttgataaatgtctcagtgagaaagcagtaaagtaa

SEQ ID NO: 3 - DNA, 954 bp, Homo sapiens
atgtcccaggtgactaacaccacacaagaaggcatctacttcatcctcacggacatccctggatttgaggcctcccacatctggatctccatccccgtctgctgtctctacaccatctccatcatgggcaataccaccatcctcactgtcattcgcacagagccatctgtccaccagcgcatgtatctgtttctctccatgctggccctgacggacctgggtctcaccctcaccaccctacccacagtcatgcagcttctctggttcaacgttcgtagaatcagctctgaggcctgttttgctcagtttttcttccttcatggattctcctttatggagtcttctgtcctcctggctatgtccgttgactgctatgtggccatctgctgtcccctccattatgcctccatcctcaccaatgaagtcattggtagaactgggttagccatcatttgctgctgtgttctggcggttcttccctcccttttcttactcaagcgactgcctttctgccactcccaccttctctctcgctcctattgcctccaccaggatatgatccgcctggtctgtgctgacatcaggctcaacagctggtatggatttgctcttgccttgctcattattatcgtggatcctctgctcattgtgatctcctatacacttattctgaaaaatatcttgggcacagccacctgggctgagcgactccgtgccctcaataactgcctgtcccacattctagctgtcctggtcctctacattcccatggttggtgtatctatgactcatcgctttgccaagcatgcctctccactggtccatgttatcatggccaatatctacctgctggcacccccggtgatgaaccccatcatttacagtgtaaagaacaagcagatccaatggggaatgttaaatttcctttccctcaaaaatatgcattcaagatga

SEQ ID NO: 4 - DNA, 975 bp, Homo sapiens
atgcctctatttaattcattatgctggtttccaacaattcatgtgactcctccatcttttattcttaatggaatacctggtctggaaagagtacatgtatggatctccctcccactctgcacaatgtacatcatcttccttgtggggaatcttggtcttgtgtacctcatttattatgaggagtccttacatcatccgatgtattttttttttggccatgctctctccctcattgacctccttacctgcaccaccactctacccaatgcactctgcatcttctggttcagtctcaaagaaattaacttcaatgcttgcttggcccagatgttctttgttcatgggttcacaggtgtggagtctggggtgctcatgctcatggctctagaccgctatatagccatttgctaccctttgcgttatgctaccacactcaccaaccctatcattgccaaggctgagcttgccaccttcctgaggggtgtattgctgatgattcctttcccattcttggttaagcgtttgcctttctgccaaagcaatattatctcccatacgtactgcgaccacatgtctgtagtaaagctatcttgtgccagcatcaaggtcaatgtaatctatggtctaatggttgctctcctgattggagtgtttgacatttgttgtatatctttgtcttacactttgatcctcaaggcagcgatcagcctctcttcatcagatgctcggcagaaggctttcagcacctgcactgcccatatatctgccatcatcatcacctatgttccagcattcttcactttctttgcccaccgttttgggggacacacaattcccccttctcttcacatcattgtggctaatctttatcttcttcttcccccaactctaaaccctattgtttatggagtaaagacaaaacagatacgcaagagtgtcataaagttcttccagggtgataagggtgcaggttga

SEQ ID NO: 5 - DNA, 971 bp, Homo sapiens
tttttttgtctgttcagacatgacaacttttatacccatcattttttccagtgtggtagtggttctatttgttattggaaattttgctaatggcttcatagcattggtaaattccattgagcgggtcaagagacaaaagatctcttttgctgaccagattctcactgctctggcggtctccagagttggtttgctctgggtattattattaaattggtattcaactgtgtttaatccagctttttatagtgtagaagtaagaactactgcttataatgtctgggcagtaaccggccatttcagcaactggcttgctactagcctcagcatattttatttgctcaagattgccaatttctccaaccttatttttcttcacttaaagaggagagttaagagtgtcattctggtgatgctgttggggcctttactatttttggcttgtcaactttttgtgataaacatgaaagagattgtacggacaaaagaatatgaaggaaacttgacttggaagatcaaattgaggagtgcagtgtacctttcagatgcgactgtaaccacgctaggaaacttagtgcccttcactctgaccctgctatgttttttgctgttaatctgttctctgtgtaaacatctcaagaagatgcagctccatggtaaaggatctcaagatcccagcaccaaggtccacataaaagctttgcaaactgtgatctttttcctcttgttatgtgccgtttactttctgtccataatgatatcagtttggagttttgggagtctggaaaacaaacctgtcttcatgttctgcaaagctattagattcagctatccttcaatccacccattcatcctgatttggggaaacaagaagctaaagcagacttttctttcagttttgcggcaagtgaggtactgggtgaaaggagagaagccttcatctccatagattcatgagaggggcattgtgt

SEQ ID NO: 6 - DNA, 2189 bp, Homo sapiens
gggtgtgggatgggagagcatccgcgtgcctgagtgagcgtgtgagtacaccgcatgcctgggtattgtgtgtctttgtgtgagcgtgggcgagtgcgtgtttgagtgtctgcaacatccttattcctgactgaagggcggcatgcaggaggggcttctcttagccagtctgtatcctcttcaaacgcactccagttaggggacagttcaccggcggccacccccagagtcgccacacacacgcacagggaggcacgcacccctctttccagagccagcaagctcagcttcccttgtcgaggaaaaccgacaacgctttagcatcctcgctgctccaacagccccaccccttcccccgttctttgccctaggtatagaatcctctatccggagatggactggaagcattggagaggggcaagagaggtggctgcgaggagagtggagaaaggcaaactgaggacacgctggaggagtgaggagccgctgcgggagaggcagatcctacccacaccacgcccctccctcggggttggagattggggatagcctgttttccactccggtcttccttcggggctcccaggtctaactgcatcttctcccctgaaagtggagccaagcgaggcggctgggaccccctcctcttccgcatccctcccaccccacacacactccgcttccaggcagccgctgattggctgcggggagcggcgtcccagccccccggctttgaggcgggagtggagcgggtccgaggtgggaggcgcacagacgggctccgggagcccctcccgaggccccgcgcagcgcgccccgcaccctgcgccccgcgccctgcgggagggctgagccaagactccaggcgggcaggtgcggagcgagcagaggggatcacggccaagggtaggagccagtcctgcggggagagaggcgctgctgctccagctgcctgctgcctccgcctgcgccaccaccgagccggcgaccagagtcggggctggcaggccgggcgcgaagcggcaaggggagcgaggggcgcgctcatggagcacacgcacgcccacctcgcagccaacagctcgctgtcttggtggtcccccggctcggcctgcggcttgggtttcgtgcccgtggtctactacagcctcttgctgtgcctcggtttaccagcaaatatcttgacagtgatcatcctctcccagctggtggcaagaagacagaagtcctcctacaactatctcttggcactcgctgctgccgacatcttggtcctctttttcatagtgtttgtggacttcctgttggaagatttcatcttgaacatgcagatgcctcaggtccccgacaagatcatagaagtgctggaattctcatccatccacacctccatatggattactgtaccgttaaccattgacaggtatatcgctgtctgccacccgctcaagtaccacacggtctcatacccagcccgcacccggaaagtcattgtaagtgtttacatcacctgcttcctgaccagcatcccctattactggtggcccaacatctggactgaagactacatcagcacctctgtgcatcacgtcctcatctggatccactgcttcaccgtctacctggtgccctgctccatcttcttcatcttgaactcaatcattgtgtacaagctcaggaggaagagcaattttcgtctccgtggctactccacggggaagaccaccgccatcttgttcaccattacctccatctttgccacactttgggccccccgcatcatcatgattctttaccacctctatggggcgcccatccagaaccgctggctggtacacatcatgtccgacattgccaacatgctagcccttctgaacacagccatcaacttcttcctctactgcttcatcagcaagcggttccgcaccatggcagccgccacgctcaaggctttcttcaagtgccagaagcaacctgtacagttctacaccaatcataacttttccataacaagtagcccctggatctcgccggcaaactcacactgcatcaagatgctggtgtaccagtatgacaaaaatggaaaaagtcgtaatgacagcaaaagctcctaccagtttgaagatgccattggagcttgtgtcatcatcctgtgaccagttaggacacaaagtagagaagtagtctgt

SEQ ID NO: 7 - DNA, 1997 bp, Homo sapiens
ccagctttcatttcagctcctgtaggcatctcctgaattaagcaacacagaaaagtcctctgaagtcactgaatcccagaaaggctctctacctttagcacaagggaggtcttcaccactggacaaagaaggaacgataaggggtcatcagactggtggtttctgagcatggattcaaccatcccagtcttgggtacagaactgacaccaatcaacggacgtgaggagactccttgctacaagcagaccctgagcttcacggggctgacgtgcatcgtttcccttgtcgcgctgacaggaaacgcggttgtgctctggctcctgggctgccgcatgcgcaggaacgctgtctccatctacatcctcaacctggtcgcggccgacttcctcttcctctgcttccagattataaattgcctggtgtacctcagtaacttcttctgttccatctccatcaatttccctagcttcttcaccactgtgatgacctgtgcctaccttgcaggcctgagcatgctgagcaccgtcagcaccgagcgctgcctgtccgtcctgtggcccatctggtatcgctgccgccgccccagacacctgtcagcggtcgtgtgtgtcctgctctgggccctgtccctactgctgagcatcttggaagggaagttctgtggcttcttatttagtgatggtgactctggttggtgtcagacatttgatttcatcactgcagcgtggctgatttttttattcatggttctctgtgggtccagtctggccctgctggtcaggatcctctgtggctccaggggtctgccactgaccaggctgtacctgaccatcctgctcacagtgctggtgttcctcctctgcggcctgccctttggcattcagtggttcctaatattatggatctggaaggattctgatgtcttattttgtcatattcatccagtttcagttgtcctgtcatctcttaacagcagtgccaaccccatcatttacttcttcgtgggctcttttaggaagcagtggcggctgcagcagccgatcctcaagctggctctccagagggctctgcaggacattgctgaggtggatgaaggtggagggtggcttcctcaggaaaccctggagctgtcgggaagcagattggagcagtgaggaagaacctctgccctgtcagacaggactttgagagcaatgctgccctgccacccttgacaattatatgcatttttcttagccttctgcctcagaaatgtctcagtggtccctcaaggtcttcgaatagatgtttatctaacctgacagttgcagttttcacccatggaaagcattagtctgacagtacaatgtttggattctccttgatattaccaatacattttccctgttatcttgcactgaatctttcctactgaacactttttctgcacttttcattgtaataaaaggagttgctgtccacaaccctaaaactcttctttatacttgtttcctacctgatagtatcaaaaaggaagattccttattaatctgtcagactatgttcccctgaaaatcatgttcccttttatgactggaggcattactgcagttggaagctcaattcttaataagtgagttctgctacctctaaattccattgaattctcagatataaagcaaaataatgaccttagagagagattctcccttcataaaaacagtcttagaaattggttttatgaatagccctctcctgtcatttgtccacagcatggtgacatgttggccttggtttctagtaaagacaatcgtggccccttccccttgagaactggtaagttcttatttagctcttcctggactaatgaactagtgaggagcctataaatatgtcccaccagtttcattttggccattggaaacctcaatattgattttaaagtggaaattatctttgtttttccatttattattcacggaaagaaaagaacagaatgtcaaagaaataagaaaaa

SEQ ID NO: 8 - DNA, 1458 bp, Homo sapiens
atgcccatcagcctggcccacggcatcatccgctcaaccgtgctggttatcttcctcgccgcctctttcgtcggcaacatagtgctggcgctagtgttgcagcgcaagccgcagctgctgcaggtgaccaaccgttttatctttaacctcctcgtcaccgacctgctgcagatttcgctcgtggccccctgggtggtggccacctctgtgcctctcttctggcccctcaacagccacttctgcacggccctggttagcctcacccacctgttcgccttcgccagcgtcaacaccattgtcgtggtgtcagtggatcgctacttgtccatcatccaccctctctcctacccgtccaagatgacccagcgccgcggttacctgctcctctatggcacctggattgtggccatcctgcagagcactcctccactctacggctggggccaggctgcctttgatgagcgcaatgctctctgctccatgatctggggggccagccccagctacactattctcagcgtggtgtccttcatcgtcattccactgattgtcatgattgcctgctactccgtggtgttctgtgcagcccggaggcagcatgctctgctgtacaatgtcaagagacacagcttggaagtgcgagtcaaggactgtgtggagaatgaggatgaagagggagcagagaagaaggaggagttccaggatgagagtgagtttcgccgccagcatgaaggtgaggtcaaggccaaggagggcagaatggaagccaaggacggcagcctgaaggccaaggaaggaagcacggggaccagtgagagtagtgtagaggccaggggcagcgaggaggtcagagagagcagcacggtggccagcgacggcagcatggagggtaaggaaggcagcaccaaagttgaggagaacagcatgaaggcagacaagggtcgcacagaggtcaaccagtgcagcattgacttgggtgaagatgacatggagtttggtgaagacgacatcaatttcagtgaggatgacgtcgaggcagtgaacatcccggagagcctcccacccagtcgtcgtaacagcaacagcaaccctcctctgcccaggtgctaccagtgcaaagctgctaaagtgatcttcatcatcattttctcctatgtgctatccctggggccctactgctttttagcagtcctggccgtgtgggtggatgtcgaaacccaggtaccccagtgggtgatcaccataatcatctggcttttcttcctgcagtgctgcatccacccctatgtctatggctacatgcacaagaccattaagaaggaaatccaggacatgctgaagaagttcttctgcaaggaaaagcccccgaaagaagatagccacccagacctgcccggaacagagggtgggactgaaggcaagattgtcccttcctacgattctgctacttttccttga

SEQ ID NO: 9 - DNA, 1001 bp, Homo sapiens
tacaatttttttcagagaggtcaggaatggttgaagaaaatcataccatgaaaaatgagtttatcctcacaggatttacagatcaccctgagctgaagactctgctgtttgtggtgttctttgccatctatctgatcaccgtggtggggaatattagtttggtggcactgatatttacacactgtcggcttcacacaccaatgtacatctttctgggaaatctggctcttgtggattcttgctgtgcctgtgctattacccccaaaatgttagagaacttcttttctgagggcaaaaggatttccctctatgaatgtgcagtacagttttattttctttgcactgtggaaactgcagactgctttcttctggcagcagtggcctatgaccgctatgtggccatctgcaacccactgcagtaccacatcatgatgtccaagaaactctgcattcagatgaccacaggcgccttcatagctggaaatctgcattccatgattcatgtagggcttgtatttaggttagttttctgtggattgaatcacatcaaccacttttactgtgatactcttcccttgtatagactctcctgtgttgaccctttcatcaatgaactggttctattcatcttctcaggttcagttcaagtctttaccataggtagtgtcttaatatcttatctctatattcttcttactattttcagaatgaaatccaaggagggaagggccaaagccttttctacttgtgcatcccacttttcatcagtttcattattctatggatctatttttttcctatacattagaccaaatttgcttgaagaaggaggtaatgatataccagctgctattttatttacaatagtagttcccttactaaatcctttcatttatagtctgagaaacaaggaagtaataagtgtcttaagaaaaattctgctgaaaataaaatctcaaggaagtgtgaacaaatgacatctatcctagcttaatggttta

SEQ ID NO: 10 - DNA, 961 bp, Homo sapiens
gaaactgactggaggagatgtcaggagaaaataattcctcagtgactgagttcattctggctgggctctcagaacagccagagctccagctgcccctcttcctcctgttcttaggaatctatgtggtcacagtggtgggcaacctgggcatgaccacactgatttggctcagttctcacctgcacacccctatgtactatttcctcagcagtctgtccttcattgacttctgccattccactgtcattacccctaagatgctggtgaactttgtgacagagaagaacatcatctcctaccctgaatgcatgactcagctctacttcttcctcgtttttgctattgcagagtgtcacatgttggctgcaatggcgtatgaccgttacatggccatctgtagccccttgctgtacagtgtcatcatatccaataaggcttgcttttctctgattttaggggtgtatataataggcctggtttgtgcatcagttcatacaggctgtatgtttagggttcaattctgcaaatttgatttgattaaccattatttctgtgatcttcttcccctcctaaagctctcttgctctagtatctatgtcaacaaactacttattctatgtgttggtgcatttaacatccttgtccccagcctgaccatcctttgctcttacatctttattattgccagcatcctccacattcgctccactgagggcaggtccaaagccttcagcacttgtagctcccacatgttggcggttgtaatcttttttggatctgcagcattcatgtacttgcagccatcttcaatcagctccatggaccaggggaaagtatcctctgtgttttatactattattgtgcccatgttgaaccctctgatttatagcctgaggaataaagatgtccatgtttccctgaagaaaatgctacagagaagaacattattgtaaacagtaat

SEQ ID NO: 11 - DNA, 1334 bp, Homo sapiens
atgtgtaaatgcttcagaagtggcaatagcactccagtcctgtgtcaccgaaactcagaagcatggcagcccaggaaggccccaagaacacagcaaactgacatgggttacaccaatttaaattccaagaaagagtgcatgtacattaaggaaaatttcaaaaagactgttgacaagatcgtggaccctggaaaccattcctcagtgactgagtccattctggctgggctctcagaacagccagagctccagctgcgcctcttcctcctgttcttaggaatctgtgtggtcacagtggtgggcaacttgggcatgatcacactgattgggctcagttctcacctgcacacacctatgtactatttcctcagcagtctgtccttcattgacttctgccattccactgtcattacccctaagatgctggtgaactttgcgacagagaagaacatcatctcctaccctgaatgcatggctcagctctatttattcagtatttttgctattgcagagtgtcacatgttggctgcaatggcgtatgactgttatgttgccatctgcagccccttgctgtacaatgtcatcatgtcctatcaccactgcttctggctcacagtgggagtttacattttaggcatccttggatctacaattcataccagttttatgttgagactctttttgtgcaagactaatgtgattaaccattatttttgtgatcttttccctctcttggggctctcctgctccagcacctacatcaatgaattactggttctggtcttgagtgcatttaacatcctgatgcctgccttaaccatccttgcttcttacatctttatcattgccagcatcctccgcattcactccactgagggcaggtccaaagccttcagcacttgcagctcccacatcttggctgttgctgttttctttggatctgcagcattcatgtacctgcagccatcatctgtcagctccatggaccagaggaaagtgtcgtctgtgttttatactactattgtgcccatgctgaaccccctgatctacagcctgaggaataaagatgtcaaacttgccgtgaagaaaattctgcatcagacagcatgttaatgaatagaatcaatgttatgttgttacatcaagataggtctttggtttgattagatatctaacttattggatttattgttgagatttatgaaaatttagtgatgctcttttatgtaacacctctccaaaatattcctccggtctgcttccatcgaacttatattccaatgagcatatgtaaagaaatacaaagaataaaatcaaaagacttttgaggttta

SEQ ID NO: 12 - DNA, 1015 bp, Homo sapiens
tgatatatatagtgtatatatacatacatgcacacatacactatatatatcaaggatttatatgataggattaattaagaaaaaaattagtggaataaaaataatgtttatgataattttggccatagaatatataatacagatgatgtgaagtacaaaatgttttttatacttcatattttgatgtacaaagtatgtttgtctttgtaattcagatgattactttgcacttgtgttcccatgaaaaatgcctttcatttctaagctggtattggcatctcagccaacacttttctccttcttttctgcgtcttctccttttctgctttttctggatctcaggccagagcgcacttacctaccagtctgtcatgtggccctcatccacatggtggtccttctcaccatggtgttcttgtctccacagctctttgaatcactgaattttcagaatgacttcaaatatgaggcatctttctacctgaggagggtgatcagggacctctccatttgtaccacctgcctcctgggcatgctgcaggtcgtcaacatcagccccagcatttcctggttggtgaggtttaaatggaaatccacaatttttaccttccatttgttctcatggtctctcagttttcctgttagtagtagcctgatcttttacactgtggcttcttccaatgtgacccagatcaatttgcatgtcagtaaatactgttcacttttcccaataaactccataatcagaggactgtttttcactctgtcattattcagagatgtttttcttaaacaaataatgctgttctcaagtgtctacatgatgactctcattcaggaactacaggagatcctggtaccttcacagccccagcctctacctaaggatctttgcagaggcaagagccatcagcacatcctgctgccggtgagtttctcggtgggcatgtacaagatggacttcatcatctcaacctcctcaacgttgccatgggcatatgaccgtggtgtctag

SEQ ID NO: 13 - DNA, 906 bp, Homo sapiens
caaattagaatgtactcaatgaatggaaggactcttttcaaaaagttgttccttggctttttctttgatttgtaaattactgatcatcattttttttttctatttgttggaacttgttcatgcagccagtctaggagtggcttcccaaattctagtgacaattgcagcctctgaccacgctcatggcgtatttgaatttagccctgagtcactctttgtcagtggaactgaaccagaagatgggtatagcactgttacattaaatgttataagacatcatggaactctgtctccagtgactttgcattggaacatagactctgatcctgatggtgatctcgccttcacctctggcaacatcacatttgagattgggcagacgagcgccaatatcactgtggagatattgcctgacgaagacccagaactggataaggcattctctgtgtcagtcctcagtgtttccagtggttctttgggagctcatattaatgccacgttaacagttttggctagtgatgatccatatgggatattcattttttctgagaaaaacagacctgttaaagttgaggaagcaacccagaacatcacactatcaataataaggttgaaaggcctcatgggaaaagtccttgtctcatatgcaacactagatgatatggaaaaaccaccttattttccacctaatttagcgagagcaactcaaggaagagactatataccagcttctggatttgctctttttggagctaatcagagtgaggcaacaatagctatttcaattttggatgatgatgagccagaaaggtccgaatctgtctttatcgaactactcaactctactttagtagcgaaagtacagagtcgttcaagtaagtatcccttagtgtgttattattatta

SEQ ID NO: 14 - protein, 309 aa, Homo sapiens
MANLTIVTEFILMGFSTNKNMCILHSILFLLIYLCALMGNVLIIMITTLDHHLHTPVYFFLKNLSFLDLCLISVTAPKSIANSLIHNNSISFLGCVSQVFLLLSSASAELLLLTVMSFDRYTAICHPLHYDVIMDRSTCVQRATVSWLYGGLIAVMHTAGTFSLSYCGSNMVHQFFCDIPQLLAISCSENLIREIALILINVVLDFCCFIVIIITYVHVFSTVKKIPSTEGQSKAYSICLPHLLVVLFLSTGFIAYLKPASESPSILDAVISVFYTMLPPTFNPIIYSLRNKAIKVALGMLIKGKLTKK

SEQ ID NO: 15 - protein, 331 aa, Homo sapiens
MSPDGNHSSDPTEFVLAGLPNLNSARVELFSVFLLVYLLNLTGNVLIVGVVRADTRLQTPMYFFLGNLSCLEILLTSVIIPKMLSNFLSRQHTISFAACITQFYFYFFLGASEFLLLAVMSADRYLAICHPLRYPLLMSGAVCFRVALACWVGGLVPVLGPTVAVALLPFCKQGAVVQHFFCDSGPLLRLACTNTKKLEETDFVLASLVIVSSLLITAVSYGLIVLAVLSIPSASGRQKAFSTCTSHLIVVTLFYGSAIFLYVRPSQSGSVDTNWAVTVITTFVTPLLNPFIYALRNEQVKEALKDMFRKVVAGVLGNLLLDKCLSEKAVK

SEQ ID NO: 16 - protein, 317 aa, Homo sapiens
MSQVTNTTQEGIYFILTDIPGFEASHIWISIPVCCLYTISIMGNTTILTVIRTEPSVHQRMYLFLSMLALTDLGLTLTTLPTVMQLLWFNVRRISSEACFAQFFFLHGFSFMESSVLLAMSVDCYVAICCPLHYASILTNEVIGRTGLAIICCCVLAVLPSLFLLKRLPFCHSHLLSRSYCLHQDMIRLVCADIRLNSWYGFALALLIIIVDPLLIVISYTLILKNILGTATWAERLRALNNCLSHILAVLVLYIPMVGVSMTHRFAKHASPLVHVIMANIYLLAPPVMNPIIYSVKNKQIQWGMLNFLSLKNMHSR

SEQ ID NO: 17 - protein, 324 aa, Homo sapiens
MPLFNSLCWFPTIHVTPPSFILNGIPGLERVHVWISLPLCTMYIIFLVGNLGLVYLIYYEESLHHPMYFFFGHALSLIDLLTCTTTLPNALCIFWFSLKEINFNACLAQMFFVHGFTGVESGVLMLMALDRYIAICYPLRYATTLTNPIIAKAELATFLRGVLLMIPFPFLVKRLPFCQSNIISHTYCDHMSVVKLSCASIKVNVIYGLMVALLIGVFDICCISLSYTLILKAAISLSSSDARQKAFSTCTAHISAIIITYVPAFFTFFAHRFGGHTIPPSLHIIVANLYLLLPPTLNPIVYGVKTKQIRKSVIKFFQGDKGAG

SEQ ID NO: 18 - protein, 309 aa, Homo sapiens
MTTFIPIIFSSVVVVLFVIGNFANGFIALVNSIERVKRQKISFADQILTALAVSRVGLLWVLLLNWYSTVFNPAFYSVEVRTTAYNVWAVTGHFSNWLATSLSIFYLLKIANFSNLIFLHLKRRVKSVILVMLLGPLLFLACQLFVINMKEIVRTKEYEGNLTWKIKLRSAVYLSDATVTTLGNLVPFTLTLLCFLLLICSLCKHLKKMQLHGKGSQDPSTKVHIKALQTVIFFLLLCAVYFLSIMISVWSFGSLENKPVFMFCKAIRFSYPSIHPFILIWGNKKLKQTFLSVLRQVRYWVKGEKPSSP

SEQ ID NO: 19 - protein, 372 aa, Homo sapiens
MEHTHAHLAANSSLSWWSPGSACGLGFVPVVYYSLLLCLGLPANILTVIILSQLVARRQKSSYNYLLALAAADILVLFFIVFVDFLLEDFILNMQMPQVPDKIIEVLEFSSIHTSIWITVPLTIDRYIAVCHPLKYHTVSYPARTRKVIVSVYITCFLTSIPYYWWPNIWTEDYISTSVHHVLIWIHCFTVYLVPCSIFFILNSIIVYKLRRKSNFRLRGYSTGKTTAILFTITSIFATLWAPRIIMILYHLYGAPIQNRWLVHIMSDIANMLALLNTAINFFLYCFISKRFRTMAAATLKAFFKCQKQPVQFYTNHNFSITSSPWISPANSHCIKMLVYQYDKNGKPIKSRNDSKSSYQFEDAIGACVIIL

SEQ ID NO: 20 - protein, 328 aa, Homo sapiens
MDSTIPVLGTELTPINGREETPCYKQTLSFTGLTCIVSLVALTGNAVVLWLLGCRMRRNAVSIYILNLVAADFLFLCFQIINCLVYLSNFFCSISINFPSFFTTVMTCAYLAGLSMLSTVSTERCLSVLWPIWYRCRRPRHLSAVVCVLLWALSLLLSILEGKFCGFLFSDGDSGWCQTFDFITAAWLIFLFMVLCGSSLALLVRILCGSRGLPLTRLYLTILLTVLVFLLCGLPFGIQWFLILWIWKDSDVLFCHIHPVSVVLSSLNSSANPIIYFFVGSFRKQWRLQQPILKLALQRALQDIAEVDEGGGWLPQETLELSGSRLEQ

SEQ ID NO: 21 - protein, 485 aa, Homo sapiens
MPISLAHGIIRSTVLVIFLAASFVGNIVLALVLQRKPQLLQVTNRFIFNLLVTDLLQISLVAPWVVATSVPLFWPLNSHFCTALVSLTHLFAFASVNTIVVVSVDRYLSIIHPLSYPSKMTQRRGYLLLYGTWIVAILQSTPPLYGWGQAAFDERNALCSMIWGASPSYTILSVVSFIVIPLIVMIACYSVVFCAARRQHALLYNVKRHSLEVRVKDCVENEDEEGAEKKEEFQDESEFRRQHEGEVKAKEGRMEAKDGSLKAKEGSTGTSESSVEARGSEEVRESSTVASDGSMEGKEGSTKVEENSMKADKGRTEVNQCSIDLGEDDMEFGEDDINFSEDDVEAVNIPESLPPSRRNSNSNPPLPRCYQCKAAKVIFIIIFSYVLSLGPYCFLAVLAVWVDVETQVPQWVITIIIWLFFLQCCIHPYVYGYMHKTIKKEIQDMLKKFFCKEKPPKEDSHPDLPGTEGGTEGKIVPSYDSATFP

SEQ ID NO: 22 - protein, 316 aa, Homo sapiens
MVEENHTMKNEFILTGFTDHPELKTLLFVVFFAIYLITVVGNISLVALIFTHCRLHTPMYIFLGNLALVDSCCACAITPKMLENFFSEGKRISLYECAVQFYFLCTVETADCFLLAAVAYDRYVAICNPLQYHIMMSKKLCIQMTTGAFIAGNLHSMIHVGLVFRLVFCGLNHINHFYCDTLPLYRLSCVDPFINELVLFIFSGSVQVFTIGSVLISYLYILLTIFRMKSKEGRAKAFSTCASHFSSVSLFYGSIFFLYIRPNLLEEGGNDIPAAILFTIVVPLLNPFIYSLRNKEVISVLRKILLKIKSQGSVNK

SEQ ID NO: 23 - protein, 311 aa, Homo sapiens
MSGENNSSVTEFILAGLSEQPELQLPLFLLFLGIYVVTVVGNLGMTTLIWLSSHLHTPMYYFLSSLSFIDFCHSTVITPKMLVNFVTEKNIISYPECMTQLYFFLVFAIAECHMLAAMAYDRYMAICSPLLYSVIISNKACFSLILGVYIIGLVCASVHTGCMFRVQFCKFDLINHYFCDLLPLLKLSCSSIYVNKLLILCVGAFNILVPSLTILCSYIFIIASILHIRSTEGRSKAFSTCSSHMLAVVIFFGSAAFMYLQPSSISSMDQGKVSSVFYTIIVPMLNPLIYSLRNKDVHVSLKKMLQRRTLL

SEQ ID NO: 24 - protein, 370 aa, Homo sapiens
MCKCFRSGNSTPVLCHRNSEAWQPRKAPRTQQTDMGYTNLNSKKECMYIKENFKKTVDKIVDPGNHSSVTESILAGLSEQPELQLRLFLLFLGICVVTVVGNLGMITLIGLSSHLHTPMYYFLSSLSFIDFCHSTVITPKMLVNFATEKNIISYPECMAQLYLFSIFAIAECHMLAAMAYDCYVAICSPLLYNVIMSYHHCFWLTVGVYILGILGSTIHTSFMLRLFLCKTNVINHYFCDLFPLLGLSCSSTYINELLVLVLSAFNILMPALTILASYIFIIASILRIHSTEGRSKAFSTCSSHILAVAVFFGSAAFMYLQPSSVSSMDQRKVSSVFYTTIVPMLNPLIYSLRNKDVKLAVKKILHQTAC

SEQ ID NO: 25 - protein, 255 aa, Homo sapiens [sequence omitted]

SEQ ID NO: 26 - protein, 295 aa, Homo sapiens
MEGLFSKSCSLAFSLICKLLIIIFFFYLLELVHAASLGVASQILVTIAASDHAHGVFEFSPESLFVSGTEPEDGYSTVTLNVIRHHGTLSPVTLHWNIDSDPDGDLAFTSGNITFEIGQTSANITVEILPDEDPELDKAFSVSVLSVSSGSLGAHINATLTVLASDDPYGIFIFSEKNRPVKVEEATQNITLSIIRLKGLMGKVLVSYATLDDMEKPPYFPPNLARATQGRDYIPASGFALFGANQSEATIAISILDDDEPERSESVFIELLNSTLVAKVQSRSSKYPLVCYYYN

SEQ ID NO: 27 - DNA, 20 bp, Homo sapiens
tcacggctcccaaatctatc

SEQ ID NO: 28 - DNA, 20 bp, Homo sapiens
tgtgcatcacagcaatcaga

SEQ ID NO: 29 - DNA, 20 bp, Homo sapiens
tctgtaagcagggtgctgtg

SEQ ID NO: 30 - DNA, 20 bp, Homo sapiens
acaatgaggccgtaggacac

SEQ ID NO: 31 - DNA, 20 bp, Homo sapiens
tctcaccctcaccaccctac

SEQ ID NO: 32 - DNA, 20 bp, Homo sapiens
cagcagcaaatgatggctaa

SEQ ID NO: 33 - DNA, 20 bp, Homo sapiens
acctgcaccaccactctacc

SEQ ID NO: 34 - DNA, 20 bp, Homo sapiens
gggaaaggaatcatcagcaa

SEQ ID NO: 35 - DNA, 20 bp, Homo sapiens
taaattccattgagcgggtc

SEQ ID NO: 36 - DNA, 20 bp, Homo sapiens
agcaagccagttgctgaaat

SEQ ID NO: 37 - DNA, 20 bp, Homo sapiens
gctggtacacatcatgtccg

SEQ ID NO: 38 - DNA, 20 bp, Homo sapiens
acaggttgcttctggcactt

SEQ ID NO: 39 - DNA, 20 bp, Homo sapiens
cctccatctttgccacactt

SEQ ID NO: 40 - DNA, 20 bp, Homo sapiens
ggcacttgaagaaagccttg

SEQ ID NO: 41 - DNA, 20 bp, Homo sapiens
cagctcctgtaggcatctcc

SEQ ID NO: 42 - DNA, 20 bp, Homo sapiens
caccagtctgatgacccctt

SEQ ID NO: 43 - DNA, 20 bp, Homo sapiens
tcagtgaggatgacgtcgag

SEQ ID NO: 44 - DNA, 20 bp, Homo sapiens
gcaggaagaaaagccagatg

SEQ ID NO: 45 - DNA, 20 bp, Homo sapiens
gtggcctatgaccgctatgt

SEQ ID NO: 46 - DNA, 20 bp, Homo sapiens
atggaatgcagatttccagc

SEQ ID NO: 47 - DNA, 20 bp, Homo sapiens
ataggcctggtttgtgcatc

SEQ ID NO: 48 - DNA, 20 bp, Homo sapiens
tgtgggagctacaagtgctg

SEQ ID NO: 49 - DNA, 20 bp, Homo sapiens
catgatcacactgattgggc

SEQ ID NO: 50 - DNA, 20 bp, Homo sapiens
ctctgtcgcaaagttcacca

SEQ ID NO: 51 - DNA, 20 bp, Homo sapiens
gccagagcgcacttacctac

SEQ ID NO: 52 - DNA, 20 bp, Homo sapiens
ctcaccaaccaggaaatgct

SEQ ID NO: 53 - DNA, 20 bp, Homo sapiens
gtgacaattgcagcctctga

SEQ ID NO: 54 - DNA, 20 bp, Homo sapiens
agtgatattggcgctcgtct

SEQ ID NO: 55 - DNA, 20 bp, Homo sapiens
cttcacctctggcaacatca

SEQ ID NO: 56 - DNA, 20 bp, Homo sapiens
acttttcccatgaggccttt

SEQ ID NO: 57 - DNA, 80 bp, Homo sapiens
tgtcaccctctgcactatgatgtcatcatggacaggagcacctgtgtccaaagagccactgtgtcttggctgtatggggg

SEQ ID NO: 58 - DNA, 80 bp, Homo sapiens
tccgcctggcttgcaccaacaccaagaagctggaggagactgactttgtcctggcctccctcgtcattgtatcttccttg

SEQ ID NO: 59 - DNA, 80 bp, Homo sapiens
tctgtcctcctggctatgtccgttgactgctatgtggccatctgctgtcccctccattatgcctccatcctcaccaatga

SEQ ID NO: 60 - DNA, 80 bp, Homo sapiens
ttgcttggcccagatgttctttgttcatgggttcacaggtgtggagtctggggtgctcatgctcatggctctagaccgct

SEQ ID NO: 61 - DNA, 80 bp, Homo sapiens
aagagacaaaagatctcttttgctgaccagattctcactgctctggcggtctccagagttggtttgctctgggtattatt

SEQ ID NO: 62 - DNA, 80 bp, Homo sapiens
ttctgaacacagccatcaacttcttcctctactgcttcatcagcaagcggttccgcaccatggcagccgccacgctcaag

SEQ ID NO: 63 - DNA, 80 bp, Homo sapiens
gcgcccatccagaaccgctggctggtacacatcatgtccgacattgccaacatgctagcccttctgaacacagccatcaa

SEQ ID NO: 64 - DNA, 80 bp, Homo sapiens
cctctgaagtcactgaatcccagaaaggctctctacctttagcacaagggaggtcttcaccactggacaaagaaggaacg

SEQ ID NO: 65 - DNA, 80 bp, Homo sapiens
cccacccagtcgtcgtaacagcaacagcaaccctcctctgcccaggtgctaccagtgcaaagctgctaaagtgatcttca

SEQ ID NO: 66 - DNA, 79 bp, Homo sapiens
ggccatctgcaacccactgcagtaccacatcatgatgtccaagaaactctgcattcagatgaccacaggcgccttcata

SEQ ID NO: 67 - DNA, 80 bp, Homo sapiens
ccccagcctgaccatcctttgctcttacatctttattattgccagcatcctccacattcgctccactgagggcaggtcca

SEQ ID NO: 68 - DNA, 80 bp, Homo sapiens
tctcacctgcacacacctatgtactatttcctcagcagtctgtccttcattgacttctgccattccactgtcattacccc

SEQ ID NO: 69 - DNA, 80 bp, Homo sapiens
cagtctgtcatgtggccctcatccacatggtggtccttctcaccatggtgttcttgtctccacagctctttgaatcactg

SEQ ID NO: 70 - DNA, 80 bp, Homo sapiens
tcatggaactctgtctccagtgactttgcattggaacatagactctgatcctgatggtgatctcgccttcacctctggca

SEQ ID NO: 71 - DNA, 80 bp, Homo sapiens
ttgggcagacgagcgccaatatcactgtggagatattgcctgacgaagacccagaactggataaggcattctctgtgtca

SEQ ID NO: 72 - protein, 321 aa, Homo sapiens
MVNLTSMSGFLLMGFSDERKLQILHALVFLVTYLLALTGNLLIITIITVDRRLHSPMYYFLKHLSLLDLCFISVTVPQSIANSLMGNGYISLVQCILQVFFFIALASSEVAILTVMSYDRYAAICQPLHYETIMDPRACRHAVIAVWIAGGLSGLMHAAINFSIPLCGKRVIHQFFCDVPQMLKLACSYEFINEIALAAFTTSAAFICLISIVLSYIRIFSTVLRIPSAEGRTKVFSTCLPHLFVATFFLSAAGFEFLRLPSDSSSTVDLVFSVFYTVIPPTLNPVIYSLRNDSMKAALRKMLSKEELPQRKMCLKAMFKL

SEQ ID NO: 73 - protein, 417 aa, Homo sapiens
MESSPIPQSSGNSSTLGRVPQTPGPSTASGVPEVGLRDVASESVALFFMLLLDLTAVAGNAAVMAVIAKTPALRKFVFVFHLCLVDLLAALTLMPLAMLSSPALFDHALFGEVACRLYLFLSVCFVSLAILSVSAINVERYYYVVHPMRYEVRMTLGLVASVLVGVWVKALAMASVPVLGRVSWEEGAPSVPPHCSLQWSHSAYCQLFVVVFAVLYFLLPLLLILLVYCSMFRVARVAAMPDGPLPTWMETPRQRSESLSSRSTMVTSSGAPQTTPHRTFGGGKAAVVLLAVGGQFLLCWLPYFSFHLYVALSAQPISTGQVESVVTWIGYFCFTSNPFFYGCLNRQIRGELSKQFVCFFKPAPEEELRLPSREGSIEENFLQFLQGTGCPSESWVSRPLPSPKQEPPAVDFRIQAR

SEQ ID NO: 74 - protein, 252 aa, Homo sapiens
VDLLAALTLMPLAMLSSSALFDHALFGEVACRLYLFLSVCFVSLAILSVSAINVERYYYVVHPMRYEVRMKLGLVASVLVGVWVKALAMASVPVLGRVSWEEGPPSVPPGCSLQWSHSAYCQLFVVVFAVLYFLLPLLLILVVYCSMFRVARVAAMQHGPLPTWMETPRQRSESLSSRSTMVTSSGAPQTTPHRTFGGGKAAVVLLAVGGQFLLCWLPYFSFHLYVALSAQPIAAGQVENVVTWIGYFCFTS

SEQ ID NO: 75 - protein, 319 aa, Mus musculus
MATSNSSTIVSSTFYLTGIPGYEEFHHWISIPFCFLYLVGITGNCMILHIVRTDPRLHEPMYYFLAMLSLTDMAMSLPTMMSLFRVLWSISREIQFNICVVQMFLIHTFSFTESSVLLAMALDRYVAICHPLRYATILTPKLIAKIGTAALLRSSILIIPLIARLAFFPFCGSHVLSHSYCLHQDMIRLACADIRFNVIYGLVLITLLWGMDSLGIFVSYVLILHSVLKIASREGRLKALNTCASHICAVLILYVPMIGLSIVHRFAKHSSPLIHIFMAHIYLLVPPVLNPIIYSVKTKQIREGILHLLCSPKISSITM

SEQ ID NO: 76 - protein, 326 aa, Mus musculus
MKVASSFHNDTNPQDVWYVLIGIPGLEDLHSWIAIPICSMYIVAVIGNVLLIFLIVTERSLHEPMYFFLSMLALADLLLSTATAPKMLAIFWFHSRGISFGSCVSQMFFIHFIFVAESAILLAMAFDRYVAICYPLRYTTILTSSVIGKIGTAAVVRSFLICFPFIFLVYRLLYCGKHIIPHSYCEHMGIARLACDNITVNIIYGLTMALLSTGLDILLIIISYTMILRTVFQIPSWAARYKALNTCGSHICVILLFYTPAFFSFFAHRFGGKTVPRHIHILVANLYVVVPPMLNPIIYGVKTKQIQDRVVFLFSSVSTCQHDSRC

SEQ ID NO: 77 - protein, 303 aa, Homo sapiens
MESALPSIFTLVIIAEFIIGNLSNGFIVLINCIDWVSKRELSSVDKLLIILAISRIGLIWEILVSWFLALHYLAIFVSGTGLRIMIFSWIVSNHFNLWLATIFSIFYLLKIASFSSPAFLYLKWRVNKVILMILLGTLVFLFLNLIQINMHIKDWLDRYERNTTWNFSMSDFETFSVSVKFTMTMFSLTPFTVAFISFLLLIFSLQKHLQKMQLNYKGHRDPRTKVHTNALKIVISFLLFYASFFLCVLISWISELYQNTVIYMLCETIGVFSPSSHSFLLILGNAKLRQAFLLVAAKVWAKR

SEQ ID NO: 78 - protein, 375 aa, Homo sapiens
ESPRNKDFTDKVPWNQREAGMETPNTTEDYDTTTEFDYGDATPCQKVNERAFGAQLLPPLYSLVFVIGLVGNILVVLVLVQYKRLKNMTSIYLLNLAISDLLFLFTLPFWIDYKLKDDWVFGDAMCKILSGFYYTGLYSEIFFIILLTIDRYLAIVHAVFALRARTVTFGVITSIIIWALAILASMPGLYFSKTQWEFTHHTCSLHFPHESLREWKLFQALKLNLFGLVLPLLVMIICYTGIIKILLRRPNEKKSKAVRLIFVIMIIFFLFWTPYNLTILISVFQDFLFTHECEQSRHLDLAVQVTEVIAYTHCCVNPVIYAFVGERFRKYLRQLFHRRVAVHLVKWLPFLSVDRLERVSSTSPSTGEHELSAGF

SEQ ID NO: 79 - protein, 314 aa, Homo sapiens
MDSTIPVLGTELTPINGREETPCYKQTLSFTGLTCIVSLVALTGNAVVLWLLGCRMRRNAVSIYILNLVAADFLFLSGHIICSPLRLINIRHPISKILSPVMTFPYFIGLSMLSAISTERCLSILWPIWYHCRRPRYLSSVMCVLLWALSLLRSILEWMFCDFLFSGADSVWCETSDFITIAWLVFLCVVLCGSSLVLLVRILCGSRKMPLTRLYVTILLTVLVFLLCGLPFGIQWALFSRIHLDWKVLFCHVHLVSIFLSALNSSANPIIYFFVGSFRQRQNRQNLQDTPEVDEGGGWLPQETLELSGSRLEQ

SEQ ID NO: 80 - protein, 407 aa, Homo sapiens
MSLNSSLSCRKELSNLTEEEGGEGGVIITQFIAIIVITIFVCLGNLVIVVTLYKKSYLLTLSNKFVFSLTLSNFLLSVLVLPFVVTSSIRREWIFGVVWCNFSALLYLLISSASMLTLGVIAIDRYYAVLYPMVYPMKITGNRAVMALVYIWLHSLIGCLPPLFGWSSVEFDEFKWMCVAAWHREPGYTAFWQIWCALFPFLVMLVCYGFIFRVARVKARKVHCGTVVIVEEDAQRTGRKNSSTSTSSSGSRRNAFQGVVYSANQCKALITILVVLGAFMVTWGPYMVVIASEALWGKSSVSPSLETWATWLSFASAVCHPLIYGLWNKTVRKELLGMCFGDRYYREPFVQRQRTSRLFSISNRITDLGLSPHLTALMAGGQPLGHSSSTGDTGFSCSQDSGNLRAL

SEQ ID NO: 81 - protein, 313 aa, Hylobates lar
MANENYTKVTEFIFTGLNYNPQLQVFLFLLFLTFYVISVTGNFGMIVLIRMDSRLHTPMYFFLSHLSFVDICFSSVVSPKMLTDFFVKRKAISFLGCALQQWFFGFFVAAECFLLASMAYDRYVAICNPLLYSVFMSQRLCIQLVVGPYVIGLMNTMTHTTNAFRLPFCGLNVINHFFCDMSPLLSLVCADTRLNKLAVFIMAGAVGVFSGLTILISYIYILMAILRIRSADGRCKTFSTCSSHLTAVFILYGTLFFIYVRPSASFPLDLNKVVSVFYTAVIPMLNPLIYSLRNKEVKDAIHRTVTQRKFCKA

SEQ ID NO: 82 - protein, 314 aa, Mus musculus
MNDMTSGNYCTVTEFFLAGLSEKPELQLPLFFLFIGIYMITVAGNLGMIILIGLSSHLHTPMYYFLSSLSFIDFCQSTVVTPKMLVNFVTEKNIISYPGCMTQLYFFLIFAIAECYILAAMAYDRYVAICNPLLYNVTMSYQIYIFLISGVYIIGVICASAHTGFMVRIRFCKLDVINHYFCDLLPLLKLACSNTYINEMLILFFGTLNIFVPILTIITSYIFIIASILRIRSTEGRSKAFSTCSSHILAVAVFFGSLAFMYLQPSSVSSMDQGKVSSVFYTIVVPMLNPLIYSLRNKDVAVALKKIIERKTFM

SEQ ID NO: 83 - protein, 307 aa, Mus musculus
MESGNLSMIIEFILTGFPTKPELQLPLFLLFLGIYLVTVLGNLGMIILIVLSSGLHTPMYFFLSSLSFIDLCHSTVITPKMLLNFLLEENIISYPECMTQLYFFSLFAIAECHMLAVMAYDRYVAICNPLLYKVVMSHHVCFWFTVGVYTLGILGSSVHTGLMLKLFFCKTNKINHYFCDLFPLLELSCSSIYINELLVLFLSALNILTPALTILMSYILIIVSILRIRSTEGRSKAFSTCSSHISAVALFYGSAAFTYLQPSSVSSMNQGKVSSVFYTTVVPMLNPLIYSLRNKDVKSSIKKILNR

SEQ ID NO: 84 - protein, 299 aa, Mus musculus
MFSLENALYIQAGLGVLANMCLLVFYIFMVLGHRPKPMDLISCQQTFIHIMLFFTAGDILHTDIFESMNIENDFKCKTTFYICRVMRGLSICTTCLLSVFQAVTISPNTSLLAKFKHKLKKYTINAFFYIWSFNLSFSSNLIFYVGAYTNVSETNQMKVTKYCSLFPMNYIIRGLILTVTTSRDVFLVGVMLITSTYMVIILFRHQRQCKHLHSIRHLRASPEKRATQTILLLVIFFVVMYWVDFIISSTSVLLWMYDPVILTVQKFVMNAYPTITPLVQISSDNRIINLLKNLQSKCH

SEQ ID NO: 85 - protein, 1967 aa, Homo sapiens
MQLCIFCCCCILFYFDLYDFGRGYDFTIQENGLQIDQPPEIGNISIVRIIIMKNDNAEGIIEFDPKYTAFEVEEDVGLIMIPVVRLHGTYGYVTADFISQSSSASPGGVDYILHGSTVTFQHGQNLSFINISIIDDNESEFEEPIEILLTGATGGAVLGRHLVSRIIIAKSDSPFGVIRFLNQSKISIANPNSTMILSLVLERTGGLLGEIQVNWETVGPNSQEALLPQNRDIADPVSGLFYFGEGEGGVRTIILTIYPHEEIEVEETFIIKLHLVKGEAKLDSRAKDVTLTIQEFGDPNGVVQFAPETLSKKTYSEPLALEGPLLITFFVRRVKGTFGEIMVYWELSSEFDITEDFLSTSGFFTIADGESEASFDVHLLPDEVPEIEEDYVIQLVSVEGGAELDLEKSITWFSVYANDDPHGVFALYSDRQSILIGQNLIRSIQINITRLAGTFGDVAVGLRISSDHKEQPIVTENAERQLVVKDGATYKVDVVPIKNQVFLSLGSNFTLQLVTVMLVGGRFYGMPTILQEAKSAVLPVSEKAANSQVGFESTAFQLMNITAGTSHVMISRRGTYGALSVAWTTGYAPGLEIPEFIVVGNMTPTLGSLSFSHGEQRKGVFLWTFPSPGWPEAFVLHLSGVQSSAPGGAQLRSGFIVAEIEPMGVFQFSTSSRNIIVSEDTQMIRLHVQRLFGFHSDLIKVSYQTTAGSAKPLEDFEPVQNGELFFQKFQTEVDFEITIINDQLSEIEEFFYINLTSVEIRGLQKFDVNWSPRLNLDFSVAVITILDNDDLAGMDISFPETTVAVAVDTTLIPVETESTTYLSTSKTTTILQPTNVVAIVTEATGVSAIPEKLVTLHGTPAVSEKPDVATVTANVSIHGTFSLGPSIVYIEEEMKNGTFNTAEVLIRRTGGFTGNVSITVKTFGERCAQMEPNALPFRGIYGISNLTWAVEEEDFEEQTLTLIFLDGERERKVSVQILDDDEPEGQEFFYVFLTNPQGGAQIVEGKDDTGFAAFAMVIITGSDLHNGIIGFSEESQSGLELREGAVMRRLHLIVTRQPNRAFEDVKVFWRVTLNKTVVVLQKDGVNLMEELQSVSGTTTCTMGQTKCFISIELKPEKVPQVEVYFFVELYEATAGAAINNSARFAQIKILESDESQSLVYFSVGSRLAVAHKKATLISLQVARDSGTGLMMSVNFSTQELRSAETIGRTIISPAISGKDFVITEGTLVFEPGQRSTVLDVILTPETGSLNSFPKRFQIVLFDPKGGARIDKVYGTANITLVSDADSQAIWGLADQLHQPVNDDILNRVLHTISMKVATENTDEQLSAMMHLIEKITTEGKIQAFSVASRTLFYEILCSLINPKRKDTRGFSHFAEVTENFAFSLLTNVTCGSPGEKSKTILDSCPYLSILALHWYPQQINGHKFEGKEGDYIRIPERLLDVQDAEIMAGKSTCKLVQFTEYSSQQWFISGNNLPTLKNKVLSLSVKGQSSQLLTNDNEVLYRIYAAEPRIIPQTSLCLLWNQAAASWLSDSQFCKVIEETADYVECACSHMSVYAVYARTDNLSSYNEAFFTSGFICISGLCLAVLSHIFCARYSMFAAKLLTHMMAASLGTQILFLASAYASPQLAEESCSAMAAVTHYLYLCQFSWMLIQSVNFWYVLVMNDEHTERRYLLFFLLSWGLPAFVVILLIVILKGIYHQSMSQIYGLIHGDLCFIPNVYAALFTAALVPLTCLVVVFVVFIHAYQVKPQWKAYDDVFRGRTNAAEIPLILYLFALISVTWLWGGLHMAYRHFWMLVLFVIFNSLQGLYVFMVYFILHNQMCCPMKASYTVEMNGHPGPSTAFFTPGSGMPPAGGEISKSTQNLIGAMEEVPPDWERASFQQGSQASPDLKPSPQNGATFPSSGGYGQGSLIADEESQEFDDLIFALKTGAGLSVSDNESGQGSQEGGTLTDSQIVELRRIPIADTHL

SEQ ID NO: 86 - protein, 259 aa, Homo sapiens
GNILVIWVICRHKRMRTPTNYFICNLAVADLLFCLTCPPWMLYYFHWGHHHWPFGRAMCKIWTYFFYMCCYASIFFLCCISIDRYWAICHPMRYRRRMTRPRHAWVMCLVIWVLAFLWSLPPLMFWWCYTHECPNHWNNCNHTWCFIDWPHESWHHWWTWWRYYYICSCIVGFYIPLLVMCFCYCRIYRTLWKAAKMLCVVVVVFFVCWLPYHIFMFMDTFCMHWWMIWTCELECVIPWAYQICVWLAYVNCCLNPIIY

SEQ ID NO: 87 - protein, 26 aa, Homo sapiens
LSFTGLTCIVSLVALTGNAVVLWLLG

SEQ ID NO: 88 - protein, 26 aa, Homo sapiens
IYILNLVAADFLFLCFQIINCLVYLS

SEQ ID NO: 89 - protein, 22 aa, Homo sapiens
FFTTVMTCAYLAGLSMLSTVST

SEQ ID NO: 90 - protein, 19 aa, Homo sapiens
LSAVVCVLLWALSLLLSIL

SEQ ID NO: 91 - protein, 23 aa, Homo sapiens
FITAAWLIFLFMVLCGSSLALLV

SEQ ID NO: 92 - protein, 21 aa, Homo sapiens
LYLTILLTVLVFLLCGLPFGI

SEQ ID NO: 93 - protein, 20 aa, Homo sapiens
VSVVLSSLNSSANPIIYFFV

SEQ ID NO: 94 - protein, 13 aa, Homo sapiens
LSTVSTERCLSVL

SEQ ID NO: 95 - protein, 13 aa, Homo sapiens
YFFVGSFRKQWRL

SEQ ID NO: 96 - protein, 14 aa, Homo sapiens
LSLLLSILEGKFCG

SEQ ID NO: 97 - protein, 14 aa, Homo sapiens
CGFLFSDGDSGWCQ

SEQ ID NO: 98 - protein, 10 aa, Homo sapiens
LELSGSRLEQ

SEQ ID NO: 99 - protein, 14 aa, Homo sapiens
VLSSLNSSANPIIY

SEQ ID NO: 100 - protein, 16 aa, Homo sapiens
FMVLCGSSLALLVRIL

SEQ ID NO: 101 - protein, 16 aa, Homo sapiens
LCGSRGLPLTRLYLTI

SEQ ID NO: 102 - protein, 16 aa, Homo sapiens
VFLLCGLPFGIQWFLI

SEQ ID NO: 103 - protein, 32 aa, Homo sapiens
CGSRGLPLTRLYLTILLTVLVFLLCGLPFGIQ

SEQ ID NO: 104 - protein, 27 aa, Homo sapiens
TCAYLAGLSMLSTVSTERCLSVLWPIW

SEQ ID NO: 105 - protein, 26 aa, Homo sapiens
LPLFLLFLGIYVVTVVGNLGMTTLIW

SEQ ID NO: 106 - protein, 23 aa, Homo sapiens
YFLSSLSFIDFCHSTVITPKMLV

SEQ ID NO: 107 - protein, 24 aa, Homo sapiens
CMTQLYFFLVFAIAECHMLAAMAY

SEQ ID NO: 108 - protein, 19 aa, Homo sapiens
ACFSLILGVYIIGLVCASV

SEQ ID NO: 109 - protein, 30 aa, Homo sapiens
LLILCVGAFNILVPSLTILCSYIFIIASIL

SEQ ID NO: 110 - protein, 22 aa, Homo sapiens
HMLAVVIFFGSAAFMYLQPSSI

SEQ ID NO: 111 - protein, 20 aa, Homo sapiens
VSSVFYTIIVPMLNPLIYSL

SEQ ID NO: 112 - protein, 13 aa, Homo sapiens
HSTVITPKMLVNF

SEQ ID NO: 113 - protein, 13 aa, Homo sapiens
LVNFVTEKNIISY

SEQ ID NO: 114 - protein, 13 aa, Homo sapiens
YSVIISNKACFSL

SEQ ID NO: 115 - protein, 13 aa, Homo sapiens
NPLIYSLRNKDVH

SEQ ID NO: 116 - protein, 13 aa, Homo sapiens
KDVHVSLKKMLQR

SEQ ID NO: 117 - protein, 14 aa, Homo sapiens
GENNSSVTEFILAG

SEQ ID NO: 118 - protein, 14 aa, Homo sapiens
FLSSLSFIDFCHST

SEQ ID NO: 119 - protein, 14 aa, Homo sapiens
EKNIISYPECMTQL

SEQ ID NO: 120 - protein, 14 aa, Homo sapiens
QPSSISSMDQGKVS

SEQ ID NO: 121 - protein, 13 aa, Homo sapiens
MSGENNSSVTEFI

SEQ ID NO: 122 - protein, 14 aa, Homo sapiens
MSGENNSSVTEFIL

SEQ ID NO: 123 - protein, 16 aa, Homo sapiens
GVYIIGLVCASVHTGC

SEQ ID NO: 124 - protein, 27 aa, Homo sapiens
LVFAIAECHMLAAMAYDRYMAICSPLL

SEQ ID NO: 125 - protein, 19 aa, Homo sapiens
LLIIIFFFYLLELVHAASL

SEQ ID NO: 126 - protein, 20 aa, Homo sapiens
FVSGTEPEDGYSTVTLNVIR

SEQ ID NO: 127 - protein, 21 aa, Homo sapiens
IDSDPDGDLAFTSGNITFEIG

SEQ ID NO: 128 - protein, 19 aa, Homo sapiens
AFSVSVLSVSSGSLGAHIN

SEQ ID NO: 129 - protein, 18 aa, Homo sapiens
KVEEATQNITLSIIRLKG

SEQ ID NO: 130 - protein, 25 aa, Homo sapiens
ATQGRDYIPASGFALFGANQSEATI

SEQ ID NO: 131 - protein, 19 aa, Homo sapiens
ESVFIELLNSTLVAKVQSR

SEQ ID NO: 132 - protein, 13 aa, Homo sapiens
GIFIFSEKNRPVK

SEQ ID NO: 133 - protein, 13 aa, Homo sapiens
KVQSRSSKYPLVC

SEQ ID NO: 134 - protein, 14 aa, Homo sapiens
ESLFVSGTEPEDGY

SEQ ID NO: 135 - protein, 14 aa, Homo sapiens
LFVSGTEPEDGYST

SEQ ID NO: 136 - protein, 14 aa, Homo sapiens
HWNIDSDPDGDLAF

SEQ ID NO: 137 - protein, 14 aa, Homo sapiens
LVSYATLDDMEKPP

SEQ ID NO: 138 - protein, 14 aa, Homo sapiens
ATIAISILDDDEPE

SEQ ID NO: 139 - protein, 14 aa, Homo sapiens
AFTSGNITFEIGQT

SEQ ID NO: 140 - protein, 14 aa, Homo sapiens
GQTSANITVEILPD

SEQ ID NO: 141 - protein, 14 aa, Homo sapiens
LGAHINATLTVLAS

SEQ ID NO: 142 - protein, 14 aa, Homo sapiens
EEATQNITLSIIRL

SEQ ID NO: 143 - protein, 14 aa, Homo sapiens
ALFGANQSEATIAI

SEQ ID NO: 144 - protein, 14 aa, Homo sapiens
FIELLNSTLVAKVQ

SEQ ID NO: 145 - protein, 16 aa, Homo sapiens
ITFEIGQTSANITVEI

SEQ ID NO: 146 - protein, 16 aa, Homo sapiens
LSVSSGSLGAHINATL

SEQ ID NO: 147 - protein, 16 aa, Homo sapiens
SSGSLGAHINATLTVL

SEQ ID NO: 148 - protein, 16 aa, Homo sapiens
GFALFGANQSEATIAI

SEQ ID NO: 149 - protein, 1615 aa, Homo sapiens
DAWADAWALYTCATLCLKEQACSAFSFFSASEGPQCFWMTSWISPAVNNSDFWTYRKNMTRVASLFSGQAVAGSDYEPVTRQWAIMQEGDEFANLTVSILPDDFPEMDESFLISLLEVHLMNISASLKNQPTIGQPNISTVVIALNGDAFGVFVIYSISPNTSEDGLFVEVQEQPQTLVELMIHRTGGSLGQVAVEWRVVGGTATEGLDFIGAGEILTFAEGETKKTVILTILDDSEPEDDESIIVSLVYTEGGSRILPSSDTVRVNILANDNVAGIVSFQTASRSVIGHEGEILQFHVIRTFPGRGNVTVNWKIIGQNLELNFANFSGQLFFPEGSLNTTLFVHLLDDNIPEEKEVYQVILYDVRTQGVPPAGIALLDAQGYAAVLTVEASDEPHGVLNFALSSRFVLLQEANITIQLFINREFGSLGAINVTYTTVPGMLSLKNQTVGNLAEPEVDFVPIIGFLILEEGETAAAINITILEDDVPELEEYFLVNLTYVGLTMAASTSFPPRLDSEGLTAQVIIDANDGARGVIEWQQSRFEVNETHGSLTLVAQRSREPLGHVSLFVYAQNLEAQVGLDYIFTPMILHFADGERYKNVNIMILDDDIPEGDEKFQLILTNPSPGLELGKNTIALIIVLANDDGPGVLSFNNSEHFFLREPTALYVQESVAVLYIVREPAQGLFGTVTVQFIVTEVNSSNESKDLTPSKGYIVLEEGVRFKALQISAILDTEPEMDEYFVCTLFNPTGGARLGVHVQTLITVLQNQAPLGLFSISAVENRATSIDIEEANRTVYLNVSRTNGIDLAVSVQWETVSETAFGMRGMDVVFSVFQSFLDESASGWCFFTLENLIYGIMLRKSSVTVYRWQGIFIPVEDLNIENPKTCEAFNIGFSPYFVITHEERNEEKPSLNSVFTFTSGFKLFLVQTIIILESSQVRYFTSDSQDYLIIASQRDDSELTQVFRWNGGSFVLHQKLPVRGVLTVALFNKGGSVFLAISQANARLNSLLFRWSGSGFINFQEVPVSGTTEVEALSSANDIYLIFAKNVFLGDQNSIDIFIWEMGQSSFRYFQSVDFAAVNRIHSFTPASGIAHILLIGQDMSALYCWNSERNQFSFVLEVPSAYDVASVTVKSLNSSKNLIALVGAHSHIYELAYISSHSDFIPSSGELIFEPGEREATIAVNILDDTVPEKEESFKVQLKNPKGGAEIGINDSVTITILSNDDAYGIVAFAQNSLYKQVEEMEQDSLVTLNVERLKGTYGRITIAWEADGSISDIFPTSGVILFTEGQVLSTITLTILADNIPELSEVVIVTLTRITTEGVEDSYKGATIDQDRSKSVITTLPNDSPFGLVGWRAASVFIRVAEPKENTTTLQLQIARDKGLLGDIAIHLRAQPNFLLHVDNQATENEDYVLQETIIIMKENIKEAHAEVSILPDDLPELEEGFIVTITEVNLVNSDFSTGQPSVRRPGMEIAEIMIEENDDPRGIFMFHVTRGAGEVITAYEVPPPLNVLQVPVVRLAGSFGAVNVYWKASPDSAGLEDFKPSHGILEFADKQVTAMIEITIIDDAEFELTETFNISLISVAGGGRLGDDVVVTVVIPQNDSPFGVFGFEEKTVS

SEQ ID NO: 150 - protein, 1577 aa, Homo sapiens
MTSWISPAVNNSDFWTYRKNMTRVASLFSGQAVAGSDYEPVTRQWAIMQEGDEFANLTVSILPDDFPEMDESFLISLLEVHLMNISASLKNQPTIGQPNISTVVIALNGDAFGVFVIYNISPNTSEDGLFVEVQEQPQTLVELMIHRTGGSLGQVAVEWRVVGGTATEGLDFIGAGEILTFAEGETKKTVILTILDDSEPEDDESIIVSLVYTEGGSRILPSSDTVRVNILANDNVAGIVSFQTASRSVIGHEGEILQFHVIRTFPGRGNVTVNWKIIGQNLELNFANFSGQLFFPEGSLNTTLFVHLLDDNIPEEKEVYQVILYDVRTQGVPPAGIALLDAQGYAAVLTVEASDEPHGVLNFALSSRFVLLQEANITIQLFINREFGSLGAINVTYTTVPGMLSLKNQTVGNLAEPEVDFVPIIGFLILEEGETAAAINITILEDDVPELEEYFLVNLTYVGLTMAASTSFPPRLDSEGLTAQVIIDANDGARGVIEWQQSRFEVNETHGSLTLVAQRSREPLGHVSLFVYAQNLEAQVGLDYIFTPMILHFADGERYKNVNIMILDDDIPEGDEKFQLILTNPSPGLELGKNTIALIIVLANDDGPGVLSFNNSEHFFLREPTALYVQESVAVLYIVREPAQGLFGTVTVQFIVTEVNSSNESKDLTPSKGYIVLEEGVRFKALQISAILDTEPEMDEYFVCTLFNPTGGARLGVHVQTLITVLQNQAPLGLFSISAVENRATSIDIEEANRTVYLNVSRTNGIDLAVSVQWETVSETAFGMRGMDVVFSVFQSFLDESASGWCFFTLENLIYGIMLRKSSVTVYRWQGIFIPVEDLNIENPKTCEAFNIGFSPYFVITHEERNEEKPSLNSVFTFTSGFKLFLVQTIIILESSQVRYFTSDSQDYLIIASQRDDSELTQVFRWNGGSFVLHQKLPVRGVLTVALFNKGGSVFLAISQANARLNSLLFRWSGSGFINFQEVPVSGTTEVEALSSANDIYLIFAENVFLGDQNSIDIFIWEMGQSSFRYFQSVDFAAVNRIHSFTPASGIAHILLIGQDMSALYCWNSERNQFSFVLEVPSAYDVASVTVKSLNSSKNLIALVGAHSHIYELAYISSHSDFIPSSGELIFEPGEREATIAVNILDDTVPEKEESFKVQLKNPKGGAEIGINDSVTITILSNDDAYGIVAFAQNSLYKQVEEMEQDSLVTLNVERLKGTYGRITIAWEADGSISDIFPTSGVILFTEGQVLSTITLTILADNIPELSEVVIVTLTRITTEGVEDSYKGATIDQDRSKSVITTLPNDSPFGLVGWRAASVFIRVAEPKENTTTLQLQIARDKGLLGDIAIHLRAQPNFLLHVDNQATENEDYVLQETIIIMKENIKEAHAEVSILPDDLPELEEGFIVTITEVNLVNSDFSTGQPSVRRPGMEIAEIMIEENDDPRGIFMFHVTRGAGEVITAYEVPPPLNVLQVPVVRLAGSFGAVNVYWKASPDSAGLEDFKPSHGILEFADKQVTAMIEITIIDDAEFELTETFNISLISVAGGGRLGDDVVVTVVIPQNDSPFGVFGFEEKTVS

SEQ ID NO: 151 - DNA, 20 bp, Homo sapiens
gtgacaattgcagcctctga

SEQ ID NO: 152 - DNA, 20 bp, Homo sapiens
agtgatattggcgctcgtct

SEQ ID NO: 153 - DNA, 20 bp, Homo sapiens
cttcacctctggcaacatca

SEQ ID NO: 154 - DNA, 20 bp, Homo sapiens
acttttcccatgaggccttt

SEQ ID NO: 155 - DNA, 80 bp, Homo sapiens; identical to SEQ ID NO: 70

SEQ ID NO: 156 - DNA, 80 bp, Homo sapiens; identical to SEQ ID NO: 71

SEQ ID NO: 157 - DNA, 18 bp, Bacteriophage SP6
atttaggtgacactatag

SEQ ID NO: 158 - DNA, 20 bp, Bacteriophage T7
taatacgactcactataggg

SEQ ID NO: 159 - DNA, 33 bp, Homo sapiens
cccaagcttatgcaggcgcttaacattaccccg

SEQ ID NO: 160 - DNA, 32 bp, Homo sapiens
cgggatccttaatgccactgtctaaaggaaga

SEQ ID NO: 161 - DNA, 68 bp, Homo sapiens
cgggatccttacttgtcgtcgtcgtccttgtagtccatatgcccactgtctaaaggagaattctcaac

SEQ ID NO: 162 - DNA, 39 bp, Homo sapiens
gcagcagcggccgcctcttttcaaaaagttgttccttgg

SEQ ID NO: 163 - DNA, 37 bp, Homo sapiens
gcagcagtcgacataataataacacactaagggatac

SEQ ID NO: 164 - DNA, 37 bp, Homo sapiens
gcagcagcggccgcatggaaggactcttttcaaaaag

SEQ ID NO: 165 - DNA, 36 bp, Homo sapiens
gcagcagtcgacgggatacttacttgaacgactctg

SEQ ID NO: 166 - DNA, 19 bp, Homo sapiens
gcagacgagcgccaatatc

SEQ ID NO: 167 - DNA, 25 bp, Homo sapiens
gacacagagaatgccttatccagtt

SEQ ID NO: 168 - DNA, 28 bp, Homo sapiens
tgggtcttcgtcaggcaatatctccaca

SEQ ID NO: 169 - protein, 17 aa, Homo sapiens
VSTERCLSVLWPIWYRC

SEQ ID NO: 170 - protein, 16 aa, Homo sapiens
HLSAVVCVLLWALSLL

SEQ ID NO: 171 - protein, 103 aa, Homo sapiens
ADFLFLCFQIINCLVYLSNFFCSISINFPSFFTTVMTCAYLAGLSMLSTVSTERCLSVLWPIWYRCRRPRHLSAVVCVLLWALSLLLSILEGKFCGFLFSDGD

SEQ ID NO: 172 - protein, 19 aa, Homo sapiens
TILLTVLVFLLCGLPFGIQ

SEQ ID NO: 173 - protein, 17 aa, Homo sapiens
LNSSANPIIYFFVGSFR

SEQ ID NO: 174 - protein, 76 aa, Homo sapiens
MDSTIPVLGTELTPINGREETPCYKQTLSFTGLTCIVSLVALTGNAVVLWLLGCRMRRNAVSIYILNLVAADFLFL

SEQ ID NO: 175 - protein, 49 aa, Homo sapiens
FDFITAAWLIFLFMVLCGSSLALLVRILCGSRGLPLTRLYLTILLTVLV

SEQ ID NO: 176 - protein, 58 aa, Homo sapiens
LLCGLPFGIQWFLILWIWKDSDVLFCHIHPVSVVLSSLNSSANPIIYFFVGSFRKQWR

SEQ ID NO: 177 - protein, 18 aa, Homo sapiens
PILKLALQRALQDIAEVD

SEQ ID NO: 178 - protein, 20 aa, Homo sapiens
LTPINGREETPCYKQTLSFT

SEQ ID NO: 179 - protein, 27 aa, Homo sapiens
LTGNAVVLWLLGCRMRRNAVSIYILNL

SEQ ID NO: 180 - protein, 12 aa, Mus musculus
INHYFCDLLPLL

SEQ ID NO: 181 - protein, 10 aa, Mus musculus
SKAFSTCSSH

SEQ ID NO: 182 - protein, 12 aa, Mus musculus
NPLIYSLRNKDV

SEQ ID NO: 183 - protein, 15 aa, Mus musculus
STEGRSKAFSTCSSH

SEQ ID NO: 184 - protein, 14 aa, Mus musculus
FFGSAAFMYLQPSS

SEQ ID NO: 185 - protein, 17 aa, Homo sapiens
IVPMLNPLIYSLRNKDV

SEQ ID NO: 186 - protein, 13 aa, Rattus norvegicus
SSMDQGKVSSVFY

SEQ ID NO: 187 - protein, 16 aa, Rattus norvegicus
VPMLNPLIYSLRNKDV

SEQ ID NO: 188 - protein, 20 aa, Homo sapiens
ITVEILPDEDPELDKAFSVS

SEQ ID NO: 189 - protein, 18 aa, Homo sapiens
LSVSSGSLGAHINATLTV

SEQ ID NO: 190 - protein, 77 aa, Homo sapiens
SDDPYGIFIFSEKNRPVKVEEATQNITLSIIRLKGLMGKVLVSYATLDDMEKPPYFPPNLARATQGRDYIPASGFAL

SEQ ID NO: 191 - protein, 13 aa, Homo sapiens
GANQSEATIAISI

SEQ ID NO: 192 - protein, 87 aa, Homo sapiens
KGLMGKVLVSYATLDDMEKPPYFPPNLARATQGRDYIPASGFALFGANQSEATIAISILDDDEPERSESVFIELLNSTLVAKVQSRS

(19) United States
(12) Patent Application Publication
(10) Pub. No.: US 2003/0096751 A1
(43) Pub. Date: May 22, 2003

(54) G-protein coupled receptor polynucleotides and methods of use thereof

(76) Inventors: Chandra S. Ramanathan, Wallingford, CT (US); Shuba Gopal, New York, NY (US); John N. Feder, Belle Mead, NJ (US); Gabriel A. Mintier, Hightstown, NJ (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/219,834

(22) Filed: Aug. 15, 2002

Related U.S. Application Data
(60) Provisional application No. 60/313,658, filed on Aug. 20, 2001.
(60) Provisional application No. 60/340,703, filed on Oct. 30, 2001.
(60) Provisional application No. 60/318,675, filed on Sep. 12, 2001.
(60) Provisional application No. 60/355,596, filed on Feb. 6, 2002.
(60) Provisional application No. 60/333,417, filed on Nov. 26, 2001.
(60) Provisional application No. 60/338,367, filed on Dec. 6, 2001.

(51) Int. Cl. A61K 38/17; C07H 21/04; C12P 21/02; C12N 5/06; C07K 14/705

(52) U.S. Cl. 514/12; 435/69.1; 435/320.1; 435/325; 530/350; 536/23.5

(57) ABSTRACT

The present invention describes human G-protein coupled receptors (GPCRs) and their encoding polynucleotides. Also described are expression vectors, host cells, antisense molecules, and antibodies associated with the GPCR polynucleotides and/or polypeptides of this invention. In addition, methods for treating, diagnosing, preventing, and screening for disorders or diseases associated with abnormal biological activity of GPCR are described, as are methods for screening for modulators, e.g., agonists or antagonists, of GPCR activity and/or function.

This application claims benefit to provisional application U.S. Serial No. 60/313,658 filed Aug. 20, 2001; to provisional application U.S. Serial No. 60/340,703, filed Oct. 30, 2001; to provisional application U.S. Serial No. 60/318,675, filed Sep. 12, 2001; to provisional application U.S. Serial No. 60/355,596, filed Feb. 6, 2002; to provisional application U.S. Serial No. 60/333,417, filed Nov. 26, 2001; and to provisional application U.S. Serial No. 60/338,367, filed Dec. 6, 2001. The entire teachings of the referenced applications are incorporated herein by reference.

### FIELD OF THE INVENTION

The present invention relates to novel G-protein coupled receptor (GPCR) nucleic acid or polynucleotide sequences (“genes”) which encode GPCR proteins. This invention further relates to fragments of novel GPCR nucleic acid sequences and their encoded amino acid sequences. Additionally, the invention relates to methods of using the GPCR polynucleotide sequences and encoded GPCR proteins for genetic screening and for the treatment of diseases, disorders, conditions, or syndromes associated with GPCRs.

### BACKGROUND OF THE INVENTION

Many medically significant biological processes that are mediated by proteins participating in signal transduction pathways involving G-proteins and/or second messengers, e.g., cAMP, have been established (Lefkowitz, Nature, 351:353-354 (1991)). These proteins are referred to herein as proteins participating in pathways with G-proteins or PPG proteins. Some examples of these proteins include the G protein-coupled receptors (GPCR), such as those for adrenergic agents and dopamine (Kobilka, B. K., et al., PNAS, 84:46-50 (1987); Kobilka, B. K., et al., Science, 238:650-656 (1987); Bunzow, J. R., et al., Nature, 336:783-787 (1988)), G-proteins themselves, effector proteins, e.g., phospholipase C, adenylate cyclase, and phosphodiesterase, and actuator proteins, e.g., protein kinase A and protein kinase C (Simon, M. I., et al., Science, 252:802-8 (1991)).

For example, in one form of signal transduction, the effect of hormone binding results in activation of the enzyme adenylate cyclase inside the cell. Enzyme activation by hormones is dependent on the presence of the nucleotide GTP, where GTP also influences hormone binding. A G-protein binds the hormone receptors to adenylate cyclase. The G-protein has further been shown to exchange GTP for bound GDP when activated by hormone receptors. The GTP-carrying form then binds to an activated adenylate cyclase. Hydrolysis of GTP to GDP, catalyzed by the G-protein itself, returns the G-protein to its basal, inactive form. Thus, the G-protein serves a dual role—as an intermediate that relays the signal from receptor to effector, and as a “clock” that controls the duration of the signal.

The membrane protein gene superfamily of G-protein coupled receptors (GPCRs) has been characterized as having seven putative transmembrane domains. The domains are believed to represent transmembrane α-helices connected by extracellular or cytoplasmic loops. GPCRs include a wide range of biologically active receptors, such as hormone, viral, growth factor, and neuronal receptors.

GPCRs are further characterized as having seven conserved hydrophobic stretches of about 20 to 30 amino acids, connecting at least eight divergent hydrophilic loops. The G-protein family of coupled receptors includes dopamine receptors, which bind to neuroleptic drugs, used for treating psychotic and neurological disorders. Other examples of members of this family of receptors include calcitonin, adrenergic, endothelin, cAMP, adenosine, muscarinic, acetylcholine, serotonin, histamine, thrombin, kinin, follicle stimulating hormone, opsins, endothelial differentiation gene-1 receptor, rhodopsins, odorant and cytomegalovirus receptors, etc.

Most GPCRs have single conserved cysteine residues in each of the first two extracellular loops which form disulfide bonds that are believed to stabilize functional protein structure. The 7 transmembrane regions are designated as TM1, TM2, TM3, TM4, TM5, TM6, and TM7. TM3 has been implicated in signal transduction. Phosphorylation and lipidation (palmitylation or farnesylation) of cysteine residues can influence signal transduction of some GPCRs. Most GPCRs contain potential phosphorylation sites within the third cytoplasmic loop and/or the carboxyl terminus.

For several GPCRs, such as the β-adrenoreceptor, phosphorylation by protein kinase A and/or specific receptor kinases mediates receptor desensitization. For some receptors, the ligand binding sites of GPCRs are believed to comprise a hydrophilic socket formed by the transmembrane domains of several GPCRs. This socket is surrounded by hydrophobic residues of the GPCRs. The hydrophilic side of each GPCR transmembrane helix is postulated to face inward and form the polar ligand-binding site. TM3 has been implicated in several GPCRs as having a ligand-binding site, which includes the TM3 aspartate residue. Additionally, TM5 serines, a TM6 asparagine and TM6 or TM7 phenylalanines or tyrosines are also implicated in ligand binding.

GPCRs can be intracellularly coupled by heterotrimeric G-proteins to various intracellular enzymes, ion channels and transporters (see, Johnson et al., Endoc., Rev., 10:317-331(1989)). Different G-protein β-subunits preferentially stimulate particular effectors to modulate various biological functions in a cell. Phosphorylation of cytoplasmic residues of GPCRs have been identified as an important mechanism for the regulation of G-protein coupling of some GPCRs. GPCRs are found in numerous sites within a mammalian host.

GPCRs are one of the largest receptor superfamilies known. These receptors are biologically important and malfunction of these receptors results in diseases such as Alzheimer's, Parkinson, diabetes, dwarfism, color blindness, retinal pigmentosa and asthma. GPCRs are also involved in depression, schizophrenia, sleeplessness, hypertension, anxiety, stress, renal failure and in several other cardiovascular, metabolic, neural, oncology and immune disorders (F. Horn and G. Vriend, J. Mol. Med., 76: 464-468 (1998)). They have also been shown to play a role in HIV infection (Y. Feng et al., Science, 272: 872-877 (1996)). The structure of GPCRs consists of seven transmembrane helices that are connected by loops. The N-terminus is always extracellular and C-terminus is intracellular. GPCRs are involved in signal transduction. The signal is received at the extracellular N-terminus side. The signal can be an endogenous ligand, a chemical moiety or light. This signal is then transduced through the membrane to the cytosolic side where a heterotrimeric protein G-protein is activated which in turn elicits a response (F. Horn et al., Recept. and Chann., 5: 305-314 (1998)). Ligands, agonists and antagonists, for these GPCRs are used for therapeutic purposes.

### SUMMARY OF THE INVENTION

The present invention provides GPCR polynucleotides, preferably full-length, and their encoded polypeptides. The GPCR polynucleotides and polypeptides, may be involved in a variety of diseases, disorders and conditions associated with GPCR activity. More specifically, the present invention is concerned with the modulation of these GPCR polynucleotides and encoded products, particularly in providing treatments and therapies for relevant diseases. Antagonizing or inhibiting the action of the GPCR polynucleotides and polypeptides is especially encompassed by the present invention.

It is an object of this invention to provide isolated GPCR polynucleotides as depicted in SEQ ID NOs:1-13. Another object of this invention is to provide GPCR polypeptides, encoded by the polynucleotide of SEQ ID NOs:1-13 and having the encoded amino acid sequences of SEQ ID NOs:14-26, or a functional or biologically active portion of these sequences.

It is yet another object of the invention to provide compositions comprising the GPCR polynucleotide sequences, or fragments thereof, or the encoded GPCR polypeptides, or fragments or portions thereof. In addition, this invention provides pharmaceutical compositions comprising at least one GPCR polypeptide, or functional portion thereof, wherein the compositions further comprise a pharmaceutically and physiologically acceptable carrier, excipient, or diluent.

A further embodiment of this invention presents polynucleotide sequences comprising the complement of SEQ ID NOs:1-13, or variants thereof. In addition, an object of the invention encompasses variations or modifications of the GPCR sequences which are a result of degeneracy of the genetic code, where the polynucleotide sequences can hybridize under moderate or high stringency conditions to the polynucleotide sequences of SEQ ID NOs:1-13.

It is another object of the invention to provide nucleic acid sequences encoding the novel GPCR polypeptides and antisense of the nucleic acid sequences, as well as oligonucleotides, fragments, or portions of the nucleic acid molecules or antisense molecules. Also provided are expression vectors and host cells comprising polynucleotides that encode the GPCR polypeptides.

A further object of the present invention encompasses amino acid sequences encoded by the novel GPCR nucleic acid sequences. The amino acid sequences of SEQ ID NOs:14-26 are encoded by the nucleic acid sequences SEQ ID NOs:1-13, respectively. More specifically, these GPCR polypeptides are of several types, namely, sensory GPCRs, orphan GPCRs, chemokine GPCRs, or very large GPCRs.

GPCRs have been described in relation to dopamine receptors, rhodopsin receptors, kinin receptors, N-formyl peptide receptors, opioid receptors, calcitonin receptors, adrenergic receptors, endothelin receptors, cAMP receptors, adenosine receptors, muscarinic receptors, acetylcholine receptors, serotonin receptors, histamine receptors, thrombin receptors, follicle stimulating hormone receptors, opsin receptors, endothelial differentiation gene-I receptors, odorant receptors, and cytomegalovirus receptors.

In yet another object, the present invention provides pharmaceutical compositions comprising the GPCR polynucleotide sequences, or fragments thereof, or the encoded GPCR polypeptide sequences, or fragments or portions thereof. Also provided are pharmaceutical compositions comprising GPCR polypeptide sequences, homologues, or one or more functional portions thereof, wherein the compositions further comprise a pharmaceutically- and/or physiologically-acceptable carrier, excipient, or diluent. All fragments or portions of the GPCR polynucleotides and polypeptides are preferably functional or active.

Another object of the invention is to provide methods for producing a polypeptide comprising the amino acid sequences of SEQ ID NOs:14-26, or a fragment thereof, preferably, a functional fragment or portion thereof, comprising the steps of a) cultivating a host cell containing an expression vector containing at least a functional fragment of the polynucleotide sequence encoding the GPCR proteins according to this invention under conditions suitable for the expression of the polypeptide; and b) recovering the polypeptide from the host cell.

Another object of this invention is to provide a substantially purified modulator, preferably an antagonist or inhibitor, of one or more of the GPCR polypeptides having SEQ ID NOs:14-26. In this regard, and by way of example, a purified antibody, or antigenic epitope thereof that binds to a polypeptide comprising the amino acid sequence of SEQ ID NOs:14-26, or homologue encoded by a polynucleotide having a nucleic acid sequence, or degenerate thereof, as set forth in any one of SEQ ID NOs:1-13 is provided.

It is yet another object of the present invention to provide GPCR nucleic acid sequences, polypeptides, peptides and antibodies for use in the diagnosis and/or screening of disorders or diseases associated with expression of one or more of the GPCR polynucleotides and their encoded polypeptide products as described herein. Another object of this invention is to provide diagnostic probes or primers for detecting GPCR-related diseases and/or for monitoring a patient's response to therapy. The probe or primer sequences comprise nucleic acid or amino acid sequences of the GPCRs described herein.

It is another object of the present invention to provide a method for detecting a polynucleotide that encodes a described GPCR polypeptide in a biological sample comprising the steps of: a) hybridizing the complement of the polynucleotide sequence encoding SEQ ID NOs:1-13 to the nucleic acid material of a biological sample, thereby forming a hybridization complex; and b) detecting the hybridization complex, wherein the presence of the complex correlates with the presence of a polynucleotide encoding a GPCR polypeptide in the biological sample. The nucleic acid material may be further amplified by the polymerase chain reaction prior to hybridization.

Another object of this invention is to provide methods for screening for agents which modulate GPCR polypeptides, e.g., agonists and antagonists, particularly those that are obtained from the screening methods as described. As yet a further object, the invention provides methods for detecting genetic predisposition, susceptibility and response to therapy of various GPCR-related diseases, disorders, or conditions.

It is another object of the present invention to provide methods for the treatment or prevention of several GPCR-associated diseases or disorders including, but not limited to, cancers, and/or cardiovascular, immune, or neurological diseases or disorders. The methods involve administering to an individual in need of such treatment or prevention an effective amount of a purified antagonist of one or more of GPCR polypeptide.

It is yet another object of this invention to provide diagnostic kits for the determination of the nucleotide sequences of human GPCR alleles. The kits can comprise reagents and instructions for amplification-based assays, nucleic acid probe assays, protein nucleic acid probe assays, antibody assays or any combination thereof. Such kits are suitable for screening and the diagnosis of disorders associated with aberrant or uncontrolled cellular development and with the expression of one or more GPCR polynucleotide and encoded GPCR polypeptide as described herein. Further objects, features, and advantages of the present invention will be better understood upon a reading of the detailed description of the invention when considered in connection with the accompanying figures or drawings.

The invention further relates to a polynucleotide encoding a polypeptide fragment of SEQ ID NO:20, 23, and/or 26, or a polypeptide fragment encoded by the cDNA sequence included in the deposited clone, which is hybridizable to SEQ ID NO:7, 10, and/or 13.

The invention further relates to a polynucleotide encoding a polypeptide domain of SEQ ID NO:20, 23, and/or 26 or a polypeptide domain encoded by the cDNA sequence included in the deposited clone, which is hybridizable to SEQ ID NO:7, 10, and/or 13.

The invention further relates to a polynucleotide encoding a polypeptide epitope of SEQ ID NO:20, 23, and/or 26 or a polypeptide epitope encoded by the cDNA sequence included in the deposited clone, which is hybridizable to SEQ ID NO:7, 10, and/or 13.

The invention further relates to a polynucleotide encoding a polypeptide of SEQ ID NO:20, 23, and/or 26 or the cDNA sequence included in the deposited clone, which is hybridizable to SEQ ID NO:7, 10, and/or 13, having biological activity.

The invention further relates to a polynucleotide which is a variant of SEQ ID NO:7, 10, and/or 13.

The invention further relates to a polynucleotide which is an allelic variant of SEQ ID NO:7, 10, and/or 13.

The invention further relates to a polynucleotide which encodes a species homologue of the SEQ ID NO:20, 23, and/or 26.

The invention further relates to a polynucleotide which represents the complimentary sequence (antisense) of SEQ ID NO:7, 10, and/or 13.

The invention further relates to a polynucleotide capable of hybridizing under stringent conditions to any one of the polynucleotides specified herein, wherein said polynucleotide does not hybridize under stringent conditions to a nucleic acid molecule having a nucleotide sequence of only A residues or of only T residues.

The invention further relates to an isolated nucleic acid molecule of SEQ ID NO:20, 23, and/or 26, wherein the polynucleotide fragment comprises a nucleotide sequence encoding a GPCR protein.

The invention further relates to an isolated nucleic acid molecule of SEQ ID NO:7, 10, and/or 13, wherein the polynucleotide fragment comprises a nucleotide sequence encoding the sequence identified as SEQ ID NO:20, 23, and/or 26 or the polypeptide encoded by the cDNA sequence included in the deposited clone, which is hybridizable to SEQ ID NO:7, 10, and/or 13.

The invention further relates to an isolated nucleic acid molecule of of SEQ ID NO:7, 10, and/or 13, wherein the polynucleotide fragment comprises the entire nucleotide sequence of SEQ ID NO:7, 10, and/or 13 or the cDNA sequence included in the deposited clone, which is hybridizable to SEQ ID NO:7, 10, and/or 13.

The invention further relates to an isolated nucleic acid molecule of SEQ ID NO:7, 10, and/or 13, wherein the nucleotide sequence comprises sequential nucleotide deletions from either the C-terminus or the N-terminus.

The invention further relates to an isolated polypeptide comprising an amino acid sequence that comprises a polypeptide fragment of SEQ ID NO:20, 23, and/or 26 or the encoded sequence included in the deposited clone.

The invention further relates to a polypeptide fragment of SEQ ID NO:20, 23, and/or 26 or the encoded sequence included in the deposited clone, having biological activity.

The invention further relates to a polypeptide domain of SEQ ID NO:20, 23, and/or 26 or the encoded sequence included in the deposited clone.

The invention further relates to a polypeptide epitope of SEQ ID NO:20, 23, and/or 26 or the encoded sequence included in the deposited clone.

The invention further relates to a full length protein of SEQ ID NO:20, 23, and/or 26 or the encoded sequence included in the deposited clone.

The invention further relates to a variant of SEQ ID NO:20, 23, and/or 26.

The invention further relates to an allelic variant of SEQ ID NO:20, 23, and/or 26. The invention further relates to a species homologue of SEQ ID NO:20, 23, and/or 26.

The invention further relates to the isolated polypeptide of of SEQ ID NO:20, 23, and/or 26, wherein the full length protein comprises sequential amino acid deletions from either the C-terminus or the N-terminus.

The invention further relates to an isolated antibody that binds specifically to the isolated polypeptide of SEQ ID NO:20, 23, and/or 26.

The invention further relates to a method for preventing, treating, or ameliorating a medical condition, comprising administering to a mammalian subject a therapeutically effective amount of the polypeptide of SEQ ID NO:20, 23, and/or 26 or the polynucleotide of SEQ ID NO:7, 10, and/or 13.

The invention further relates to a method of diagnosing a pathological condition or a susceptibility to a pathological condition in a subject comprising the steps of (a) determining the presence or absence of a mutation in the polynucleotide of SEQ ID NO:7, 10, and/or 13; and (b) diagnosing a pathological condition or a susceptibility to a pathological condition based on the presence or absence of said mutation.

The invention further relates to a method of diagnosing a pathological condition or a susceptibility to a pathological condition in a subject comprising the steps of (a) determining the presence or amount of expression of the polypeptide of SEQ ID NO:20, 23, and/or 26 in a biological sample; and diagnosing a pathological condition or a susceptibility to a pathological condition based on the presence or amount of expression of the polypeptide.

The invention further relates to a method for identifying a binding partner to the polypeptide of SEQ ID NO:20, 23, and/or 26 comprising the steps of (a) contacting the polypeptide of SEQ ID NO:20, 23, and/or 26 with a binding partner; and (b) determining whether the binding partner effects an activity of the polypeptide.

The invention further relates to a gene corresponding to the cDNA sequence of SEQ ID NO:7, 10, and/or 13. The invention further relates to a method of identifying an activity in a biological assay, wherein the method comprises the steps of expressing SEQ ID NO:7, 10, and/or 13 in a cell, (b) isolating the supernatant; (c) detecting an activity in a biological assay; and (d) identifying the protein in the supernatant having the activity.

The invention further relates to a process for making polynucleotide sequences encoding gene products having altered SEQ ID NO:20, 23, and/or 26 activity comprising the steps of (a) shuffling a nucleotide sequence of SEQ ID NO:7, 10, and/or 13, (b) expressing the resulting shuffled nucleotide sequences and, (c) selecting for altered activity as compared to the activity of the gene product of said unmodified nucleotide sequence.

The invention further relates to a shuffled polynucleotide sequence produced by a shuffling process, wherein said shuffled DNA molecule encodes a gene product having enhanced tolerance to an inhibitor of SEQ ID NO:20, 23, and/or 26 activity.

The invention further relates to a method of identifying a compound that modulates the biological activity of Gene 7, 10, and/or 13, comprising the steps of, (a) combining a candidate modulator compound with Gene 7, 10, and/or 13 having the sequence set forth in one or more of SEQ ID NO:20, 23, and/or 26; and measuring an effect of the candidate modulator compound on the activity of Gene 7, 10, and/or 13.

The invention further relates to a method of identifying a compound that modulates the biological activity of a GPCR, comprising the steps of, (a) combining a candidate modulator compound with a host cell expressing Gene 7, 10, and/or 13 having the sequence as set forth in SEQ ID NO:20, 23, and/or 26; and, (b) measuring an effect of the candidate modulator compound on the activity of the expressed Gene 7, 10, and/or 13.

The invention further relates to a method of identifying a compound that modulates the biological activity of Gene 7, 10, and/or 13, comprising the steps of, (a) combining a candidate modulator compound with a host cell containing a vector described herein, wherein Gene 7, 10, and/or 13 is expressed by the cell; and, (b) measuring an effect of the candidate modulator compound on the activity of the expressed Gene 7, 10, and/or 13.

The invention further relates to a method of screening for a compound that is capable of modulating the biological activity of Gene 7, 10, and/or 13, comprising the steps of: (a) providing a host cell described herein; (b) determining the biological activity of Gene 7, 10, and/or 13 in the absence of a modulator compound; (c) contacting the cell with the modulator compound; and (d) determining the biological activity of Gene 7, 10, and/or 13 in the presence of the modulator compound; wherein a difference between the activity of Gene 7, 10,, and/or 13 in the presence of the modulator compound and in the absence of the modulator compound indicates a modulating effect of the compound.

The invention further relates to a compound that modulates the biological activity of human Gene 7, 10, and/or 13 as identified by the methods described herein.

The invention further relates to a method for preventing, treating, or ameliorating a medical condition with the polypeptide provided as SEQ ID NO:26, in addition to, its encoding nucleic acid, wherein the medical condition is a neural disorder.

The invention further relates to a method for preventing, treating, or ameliorating a medical condition with the polypeptide provided as SEQ ID NO:26, in addition to, its encoding nucleic acid, wherein the medical condition is an endocrine disorder.

The invention further relates to a method for preventing, treating, or ameliorating a medical condition with the polypeptide provided as SEQ ID NO:26, in addition to, its encoding nucleic acid, wherein the medical condition is a sleep disorder.

The invention further relates to a method for preventing, treating, or ameliorating a medical condition with the polypeptide provided as SEQ ID NO:26, in addition to, its encoding nucleic acid, wherein the medical condition is a member of the group consisting of disorders that affect the nucleus accumbens, disorders that affect the brains ‘reward center’ function, neurotransmitter release disorders, disorders affecting the release of dopamine, disorders affecting the release of opioid peptides, disorders affecting the release of serotonin, disorders affecting the release of GABA, pineal gland disorders, disorders affecting the establishment of circadian rhythms, disorders affecting the maintenance of circadian rhythms, disorders affecting the control of the sleep/wake cycle.

The invention further relates to a method for preventing, treating, or ameliorating a medical condition with the polypeptide provided as SEQ ID NO:26, in addition to, its encoding nucleic acid, wherein the medical condition is a member of the group consisting of melatonin secretion disorders, pituitary hormone secretion disorders, oxytocin secretion disorders, disorders affecting neuroendocrine response to stressful stimuli, disorders affecting oxytocin secretion during neuroendocrine response to stressful stimuli, disorders affecting nocturnal patterns of hormone secretion, disorders affecting the nocturnal hormone secretion of prolactin, disorders affecting the nocturnal hormone secretion of cortisol, and/or disorders affecting the nocturnal hormone secretion of growth hormone.

The invention further relates to a method for preventing, treating, or ameliorating a medical condition with the polypeptide provided as SEQ ID NO:26, in addition to, its encoding nucleic acid, wherein the medical condition is a member of the group consisting of neuro-pathologies, including responses to stress, and propensity to develop addictive behaviors, as well as a vast number of neuroendocrine abnormalities including sleep disorders.

The present invention also relates to an isolated polynucleotide consisting of a portion of the human Gene 7 gene consisting of at least 8 bases, specifically excluding the polynucleotide sequence provided in Genbank Accession Nos. BG198766; BI828553; BG210740; BE439409; AF003828; and/or AL705589.

The present invention also relates to an isolated polynucleotide consisting of a nucleotide sequence encoding a fragment of the human Gene 7 protein, wherein said fragment displays one or more functional activities specifically excluding the polynucleotide sequence provided in Genbank Accession Nos. BGI98766; BI828553; BG210740; BE439409; AF003828; and/or AL705589.

The present invention also relates to the polynucleotide of SEQ ID NO:7 consisting of at least 10 to 50 bases, wherein said at least 10 to 50 bases specifically exclude the polynucleotide sequence of Genbank Accession Nos. BG198766; BI828553; BG210740; BE439409; AF003828; and/or AL705589.

The present invention also relates to the polynucleotide of SEQ ID NO:7 consisting of at least 15 to 100 bases, wherein said at least 15 to 100 bases specifically exclude the polynucleotide sequence of Genbank Accession Nos. BG198766; BI828553; BG210740; BE439409; AF003828; and/or AL705589.

The present invention also relates to the polynucleotide of SEQ ID NO:7 consisting of at least 100 to 1000 bases, wherein said at least 100 to 1000 bases specifically exclude the polynucleotide sequence of Genbank Accession Nos. BG198766; BI828553; BG210740; BE439409; AF003828; and/or AL705589.

The present invention also relates to an isolated polypeptide fragment of the human Gene 7 protein, wherein said polypeptide fragment does not consist of the polypeptide encoded by the polynucleotide sequence of Genbank Accession Nos. BG198766; BI828553; BG210740; BE439409; AF003828; and/or AL705589.

The present invention also relates to an isolated polynucleotide consisting of a portion of the human Gene 10 gene consisting of at least 8 bases, specifically excluding the polynucleotide sequence provided in Genbank Accession Nos. BB201968; BB206141); AI962273; and/or BI274717.

The present invention also relates to an isolated polynucleotide consisting of a nucleotide sequence encoding a fragment of the human Gene 10 protein, wherein said fragment displays one or more functional activities specifically excluding the polynucleotide sequence provided in Genbank Accession Nos. BB201968; BB206141); AI962273; and/or BI274717.

The present invention also relates to the polynucleotide of SEQ ID NO:10 consisting of at least 10 to 50 bases, wherein said at least 10 to 50 bases specifically exclude the polynucleotide sequence of Genbank Accession Nos. BB201968; BB206141); AI962273; and/or BI274717.

The present invention also relates to the polynucleotide of SEQ ID NO:10 consisting of at least 15 to 100 bases, wherein said at least 15 to 100 bases specifically exclude the polynucleotide sequence of Genbank Accession Nos. BB201968; BB206141); AI962273; and/or BI274717.

The present invention also relates to the polynucleotide of SEQ ID NO:10 consisting of at least 100 to 1000 bases, wherein said at least 100 to 1000 bases specifically exclude the polynucleotide sequence of Genbank Accession Nos. BB201968; BB206141); AI962273; and/or BI274717.

The present invention also relates to an isolated polypeptide fragment of the human Gene 10 protein, wherein said polypeptide fragment does not consist of the polypeptide encoded by the polynucleotide sequence of Genbank Accession Nos. BB201968; BB206141); AI962273; and/or BI274717.

The present invention also relates to an isolated polynucleotide consisting of a nucleotide sequence encoding a fragment of the human Gene 13 protein, wherein said fragment displays one or more functional activities specifically excluding the polynucleotide sequence provided in Genbank Accession Nos. BQ339434; and/or BG003773.

The present invention also relates to the polynucleotide of SEQ ID NO:13 consisting of at least 10 to 50 bases, wherein said at least 10 to 50 bases specifically exclude the polynucleotide sequence of Genbank Accession Nos. BQ339434; and/or BG003773.

The present invention also relates to the polynucleotide of SEQ ID NO:13 consisting of at least 15 to 100 bases, wherein said at least 15 to 100 bases specifically exclude the polynucleotide sequence of Genbank Accession Nos. BQ339434; and/or BG003773.

The present invention also relates to the polynucleotide of SEQ ID NO:13 consisting of at least 100 to 1000 bases, wherein said at least 100 to 1000 bases specifically exclude the polynucleotide sequence of Genbank Accession Nos. BQ339434; and/or BG003773.

The present invention also relates to an isolated polypeptide fragment of the human Gene 13 protein, wherein said polypeptide fragment does not consist of the polypeptide encoded by the polynucleotide sequence of Genbank Accession Nos. BQ339434; and/or BG003773.

### BRIEF DESCRIPTION OF THE FIGURES

FIG. 1 presents the nucleic acid sequence (SEQ ID NO:1) of a novel human sensory GPCR, called Gene 1 herein.

FIG. 2 presents the amino acid sequence (SEQ ID NO:14) encoded by the nucleic acid sequence (SEQ ID NO:1) of Gene 1.

FIG. 3 presents the nucleic acid sequence (SEQ ID NO:2) of a novel human sensory GPCR, called Gene 2 herein.

FIG. 4 presents the amino acid sequence (SEQ ID NO:15) encoded by the nucleic acid sequence (SEQ ID NO:2) of Gene 2.

FIG. 5 presents the nucleic acid sequence (SEQ ID NO:3) of a novel human sensory GPCR, called Gene 3 herein.

FIG. 6 presents the amino acid sequence (SEQ ID NO:16) encoded by the nucleic acid sequence (SEQ ID NO:3) of Gene 3.

FIG. 7 shows the nucleic acid sequence (SEQ ID NO:4) of a novel human sensory GPCR, called Gene 4 herein.

FIG. 8 shows the amino acid sequence (SEQ ID NO:17) encoded by the nucleic acid sequence (SEQ ID NO:4) of Gene 4.

FIG. 9 shows the nucleic acid sequence (SEQ ID NO:5) of a novel human sensory GPCR, called Gene 5 herein.

FIG. 10 shows the amino acid sequence (SEQ ID NO:18) encoded by the nucleic acid sequence (SEQ ID NO:5) of Gene 5.

FIG. 11 shows the nucleic acid sequence (SEQ ID NO:6) of a novel human chemokine GPCR, called Gene 6 herein.

FIG. 12 shows the amino acid sequence (SEQ ID NO:19) encoded by the nucleic acid sequence (SEQ ID NO:6) of Gene 6.

FIGS. 13A, 13B, and 13C present the nucleic acid sequence (SEQ ID NO:7) of a novel human orphan GPCR, called Gene 7 herein.

FIG. 14 presents the amino acid sequence (SEQ ID NO:20) encoded by the nucleic acid sequence (SEQ ID NO:7) of Gene 7.

FIGS. 15A and 15B present the nucleic acid sequence (SEQ ID NO:8) of a novel human orphan GPCR, called Gene 8 herein.

FIG. 16 presents the amino acid sequence (SEQ ID NO:21) encoded by the nucleic acid sequence (SEQ ID NO:8) of Gene 8.

FIG. 17 presents the nucleic acid sequence (SEQ ID NO:9) of a novel human sensory GPCR, called Gene 9 herein.

FIG. 18 presents the amino acid sequence (SEQ ID NO:22) encoded by the nucleic acid sequence (SEQ ID NO:9) of Gene 9.

FIGS. 19A-B shows the nucleic acid sequence (SEQ ID NO:10) of a novel human sensory GPCR, called Gene 10 herein.

FIG. 20 shows the amino acid sequence (SEQ ID NO:23) encoded by the nucleic acid sequence (SEQ ID NO:10) of Gene 10.

FIGS. 21A and 21B show the nucleic acid sequence (SEQ ID NO:11) of a novel human sensory GPCR, called Gene 11 herein.

FIG. 22 shows the amino acid sequence (SEQ ID NO:24) encoded by the nucleic acid sequence (SEQ ID NO:11) of Gene 11.

FIG. 23 presents the nucleic acid sequence (SEQ ID NO:12) of a novel human sensory GPCR, called Gene 12 herein.

FIG. 24 presents the amino acid sequence (SEQ ID NO:25) encoded by the nucleic acid sequence (SEQ ID NO:12) of Gene 12.

FIGS. 25A-B presents the nucleic acid sequence (SEQ ID NO:13) of a novel human very large GPCR, called Gene 13 herein.

FIG. 26 presents the amino acid sequence (SEQ ID NO:26) encoded by the nucleic acid sequence (SEQ ID NO:13) of Gene 13.

FIG. 27A illustrates an alignment of the novel human sensory GPCR Gene 1 with the top hit protein human olfactory receptor 5U1 (Genbank Accession No: gi|14423824; SEQ ID NO:72) a transmembrane receptor) using the protein sequence database and BLAST analysis as known and as described herein. FIG. 27B illustrates the domain prediction for the GPCR encoded by Gene 1. (“T” denotes “target”, and represents a portion of the amino acid sequence of Gene 1 provided as SEQ ID NO:14). Domain predictions are valuable for suggesting possible functional domains in the predicted protein. These predictions are based on comparisons of the given protein sequence (the “query”, or “Q” represents the Pfam PF00007 Rhodopsin model sequence provided as SEQ ID NO:86) against a collection of statistical models known as Hidden Markov Models (HMMs) (the targets, or T). HMMs represent consensus patterns for known functional domains and this method of comparison allows for the prediction of functional domains in novel protein sequences. HMMs are built from the Pfam alignments. The Pfam is a database of multiple alignments of protein domains or conserved protein regions. The alignments represent some evolutionary conserved structure, which has implications for the protein's function. Such alignment analysis can be very useful for automatically recognizing that a new protein belongs to an existing protein family, even if the homology is weak (See, A. Bateman, E. Birney, R. Durbin, S. R. Eddy, K. L. Howe, and E. L. L. Sonnhammer. The Pfam Protein Families Database. Nucleic Acids Research, 28:263-266, 2000).

In FIG. 27A, the query (or “Q”) sequence is that of Gene 1 (SEQ ID NO: 14), while the subject (“sbjct”) sequence is that of the sequence having the highest percent identity (50%), i.e., human olfactory receptor 5U1, for this GPCR sequence (Genbank Accession No: gi|14423824; SEQ ID NO:72).

FIGS. 28A and 28B illustrate an alignment of the novel human sensory GPCR Gene 2 with the top hit proteins from the protein sequence database and BLAST analysis as known and also as described herein. FIG. 28A shows that the GPCR Gene 2 amino acid sequence is highly similar to human G protein coupled receptor 61 protein (Genbank Accession No: gi|13994320; SEQ ID NO:73). FIG. 27B shows that the Gene 2 amino acid sequence is also highly similar to rabbit G protein coupled receptor protein (Genbank Accession No: gi|AAR91232; SEQ ID NO:74). In FIGS. 28A, 28B, and 28C, the query (or “Q”) sequence is that of Gene 2 (SEQ ID NO:15); in FIG. 28A, the subject (“sbjct”) sequence(s) is/are the amino acid sequence(s) having the highest percent identity (98%) to that of Gene 2. FIG. 28C illustrates the predicted domains in the GPCR encoded by Gene 2. (“T” denotes “target” and represents the Pfam PF00007 Rhodopsin model sequence provided as SEQ ID NO:86).

FIG. 29A illustrates an alignment of the novel human sensory GPCR Gene 3 with the top hit protein, i.e., MOR 3′Beta4 protein of mouse (Genbank Accession No: gi|11908220; SEQ ID NO:75), from the protein sequence database and BLAST analysis as known and as described herein. FIG. 29B illustrates the domain prediction in the GPCR encoded by Gene 3. (“T” denotes “target” and represents the Pfam PF00007 Rhodopsin model sequence provided as SEQ ID NO:86). In FIGS. 29A and 29B, the query (or “Q”) sequence is that of Gene 3, while the subject (“sbjct”) sequence is the amino acid sequence having the highest percent identity (48%) to that of Gene 3 (Genbank Accession No: gi|11908220; SEQ ID NO:75).

FIG. 30A illustrates an alignment of the novel human sensory GPCR Gene 4 with the top hit protein, i.e., MOR 3′Beta1 protein (olfactory receptor 67) of mouse (Genbank Accession No: gi|4761597; SEQ ID NO:76), from the protein sequence database and BLAST analysis as known and as described herein. FIG. 30B illustrates the domain prediction in the GPCR encoded by Gene 4. (“T” denotes “target” and represents the Pfam PF00007 Rhodopsin model sequence provided as SEQ ID NO:86). In FIGS. 30A and 30B, the query (or “Q”) sequence is that of Gene 4, while the subject (“sbjct”) sequence is the amino acid sequence having the highest percent identity (48%) to that of Gene 4 (Genbank Accession No: gi|4761597; SEQ ID NO:76).

FIG. 31 illustrates an alignment of the novel human sensory GPCR Gene 5 with the top hit protein, i.e., human taste receptor protein (Genbank Accession No: gi|7262621; SEQ ID NO:77) from the protein sequence database and BLAST analysis as known and as described herein. In FIG. 31, the query (or “Q”) sequence is that of Gene 5, while the subject (“sbjct”) sequence is the amino acid sequence having the highest percent identity (44%) to that of Gene 5 (Genbank Accession No: gi|7262621; SEQ ID NO:77).

FIG. 32 illustrates an alignment of the novel human chemokine GPCR Gene 6 with the top hit protein, i.e., human chemokine receptor 1 (Genbank Accession No: gi|12729981; SEQ ID NO:78) from the protein sequence database and BLAST analysis as known and as described herein. In FIG. 32, the query (or “Q”) sequence is that of Gene 6, while the subject (“sbjct”) sequence is the amino acid sequence having the highest percent identity (26%) to that of Gene 6 (Genbank Accession No: gi|12729981; SEQ ID NO:78).

FIG. 33 illustrates an alignment of the novel human orphan GPCR Gene 7 with the top hit protein, i.e., human G-protein coupled receptor hHI7T213 (Genbank Accession No: gi|AAY90761; SEQ ID NO:79) from the protein sequence database and BLAST analysis as known and as described herein. In FIG. 33, the query (or “Q”) sequence is that of Gene 7, while the subject (“sbjct”) sequence is the amino acid sequence having the highest percent identity (77%) to that of Gene 7 (Genbank Accession No: gi|AAY90761; SEQ ID NO:79).

FIG. 34 illustrates an alignment of the novel human orphan GPCR Gene 8 with the top hit protein, i.e., human G-protein coupled receptor RE2 (Genbank Accession No: gi|13637713; SEQ ID NO:80) from the protein sequence database and BLAST analysis as known and as described herein. In FIG. 34, the query (or “Q”) sequence is that of Gene 8, while the subject (“sbjct”) sequence is the amino acid sequence having the highest percent identity (32%) to that of Gene 8 (Genbank Accession No: gi|13637713; SEQ ID NO:80).

FIG. 35 illustrates an alignment of the novel human sensory GPCR Gene 9 with the top hit protein, i.e., human sensory GPCR receptor (Genbank Accession No: gi|3746448; SEQ ID NO:81) from the protein sequence database and BLAST analysis as known and as described herein. In FIG. 35, the query (or “Q”) sequence is that of Gene 9, while the subject (“sbjct”) sequence is the amino acid sequence having the highest percent identity (47%) to that of Gene 9 (Genbank Accession No: gi|3746448; SEQ ID NO:81).

FIG. 36 illustrates an alignment of the novel human sensory GPCR Gene 10 with the top hit protein, i.e., odorant receptor K11 of mouse (Genbank Accession No: gi|11692519; SEQ ID NO:82), from the protein sequence database and BLAST analysis as known and as described herein. In FIG. 36, the query (or “Q”) sequence is that of Gene 10, while the subject (“sbjct”) sequence is the amino acid sequence having the highest percent identity (76%) to that of Gene 10 (Genbank Accession No: gi|11692519; SEQ ID NO:82).

FIG. 37 illustrates an alignment of the novel human sensory GPCR Gene 11 with the top hit protein, i.e., odorant receptor K4h11 of mouse (Genbank Accession No: gi|11692563; SEQ ID NO:83), from the protein sequence database and BLAST analysis as known and as described herein. In FIG. 37, the query (or “Q”) sequence is that of Gene 11, while the subject (“sbjct”) sequence is the amino acid sequence having the highest percent identity (78%) to that of Gene 11 (Genbank Accession No: gi|11692563; SEQ ID NO:83).

FIG. 38 illustrates an alignment of the novel human sensory GPCR Gene 12 with the top hit protein, i.e., vomeronasal receptor V1RC3 of mouse (Genbank Accession No: gi|11967419; SEQ ID NO:84), from the protein sequence database and BLAST analysis as known and as described herein. In FIG. 38, the query (or “Q”) sequence is that of Gene 12, while the subject (“sbjct”) sequence is the amino acid sequence having the highest percent identity (43%) to that of Gene 12 (Genbank Accession No: gi|11967419; SEQ ID NO:84).

FIG. 39 illustrates an alignment of the novel the human very large GPCR Gene 13 with the top hit protein, i.e., human very large G-protein coupled receptor-1 (Genbank Accession No: gi|5902966; SEQ ID NO:85), from the protein sequence database and BLAST analysis as known and as described herein. In FIG. 39, the query (or “Q”) sequence is that of Gene 13, while the subject (“sbjct”) sequence is the amino acid sequence having the highest percent identity (30%) to that of Gene 13 (Genbank Accession No: gi|5902966; SEQ ID NO:85).

FIGS. 40A-40E illustrate a multiple sequence alignment of the amino acid sequence of GPCR, Gene 13, (SEQ ID NO:26) with the amino acid sequences of other human GPCR proteins, namely, human_hypothetical 1 (SEQ ID NO:149) and human_hypothetical 2 (SEQ ID NO:150). The GCG pileup program was used to generate the alignment. The blackened areas represent identical amino acids in more than half of the listed sequences and the gray highlighted areas represent similar amino acids. Dashes represent no comparison and dots represent gaps in the alignment.

FIG. 41 presents the tissue expression profile of the novel human GPCR, Gene 13. A PCR primer was designed from SEQ ID NO:13 and was used to measure the steady state levels of mRNA by quantitative PCR. Transcripts corresponding to the GPCR, Gene 13, is highly expressed in brain tissues and the pituitary.

FIG. 42 presents the brain sub-region expression profile of the novel human GPCR, Gene 13. A PCR primer was designed from SEQ ID NO:13 and was used to measure the steady state levels of mRNA by quantitative PCR. Transcripts corresponding to the GPCR, HGPRBMY 37, is highly expressed in the following brain sub-regions: amygdala, cerebellum, corpus callosum, caudate nucleus, hippocampus, subtantia nigra and thalamus.

FIG. 43 presents a schematic of the cell-based reporter assay system based on Fluorescence Resonance Energy Transfer (FRET) to detect Gene 13 functional coupling as described in Example 8. Gene 13 is transfected into the Cho/NFAT-CRE reporter cell line and changes in real-time gene expression, as a consequence of constitutive G-protein coupling of Gene 13 GPCR, is examined by analyzing the fluorescence emission of the transformed cells at 447 nm and 518 nm

FIG. 44 shows an expanded expression profile of the novel G-protein coupled receptor, Gene 13. The figure illustrates the relative expression level of Gene 13 amongst various mRNA tissue sources. As shown, the Gene 13 polypeptide was expressed predominately in the nervous system, with lesser amounts found in the respiratory and endocrine systems. Specifically, Gene 13 was expressed at the highest steady state levels in the nucleus accumbens, followed by the pineal and pituitary gland. Expression of Gene 13 was also significantly expressed at near equal levels across the cortex, hippocampus, amygdala, and choroid plexus. Expression of Gene 13 was also significantly expressed to a lesser extent in in the caudate, the cerebellum and the hypothalamus. Expression data was obtained by measuring the steady state Gene 13 mRNA levels by quantitative PCR using the PCR primer pair provided as SEQ ID NO:166 and 167, and Taqman probe (SEQ ID NO:168) as described in Example 10 herein.

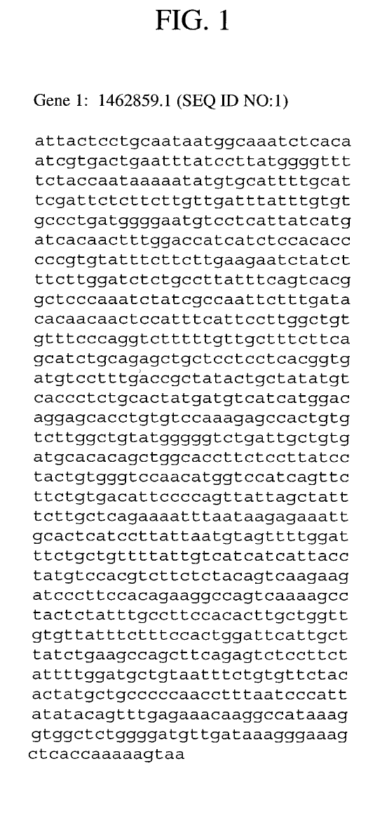

### DETAILED DESCRIPTION OF THE INVENTION

The present invention provides novel human GPCR (GPCR) genes (i.e., polynucleotide or nucleic acid sequences) which encode GPCR proteins (polypeptides), preferably full-length GPCR polypeptides. The invention further relates to fragments and portions of novel GPCR nucleic acid sequences and their encoded amino acid sequences (peptides). Preferably, the fragments and portions of the GPCR polypeptides are functional or active. The invention also provides methods of using the novel GPCR polynucleotide sequences and the encoded GPCR polypeptides for genetic screening and for the treatment of diseases, disorders, conditions, or syndromes associated with GPCRs and GPCR activity and function.

Definitions

The following definitions are provided to more fully describe the present invention in its various aspects. The definitions are intended to be useful for guidance and elucidation, and are not intended to limit the disclosed invention or its embodiments.

“Amino acid sequence” as used herein can refer to an oligopeptide, peptide, polypeptide, or protein sequence, and fragments or portions thereof, as well as to naturally occurring or synthetic molecules, preferably isolated polypeptides of the GPCR. Amino acid sequence fragments are typically from about 4 to about 30, preferably from about 5 to about 15, more preferably from about 5 to about 15 amino acids in length and preferably retain the biological activity or function of a GPCR polypeptide. GPCR amino acid sequences of this invention are set forth in SEQ ID NOs:14-26 of Table 1 and in description of the Figures. The terms GPCR polypeptide and GPCR protein are used interchangeably herein to refer to the encoded products of the GPCR nucleic acid sequences according to the present invention.

As will be appreciated by the skilled practitioner, should the amino acid fragment comprise an antigenic epitope, for example, biological function per se need not be maintained. The terms Gene 7, 10, and/or 13 polypeptide and Gene 7, 10, and/or 13 protein are used interchangeably herein to refer to the encoded product of the Gene 7, 10, and/or 13 nucleic acid sequence according to the present invention.

Isolated GPCR polypeptide refers to the amino acid sequence of substantially purified GPCR, which may be obtained from any species, preferably mammalian, and more preferably, human, and from a variety of sources, including natural, synthetic, semi-synthetic, or recombinant. More particularly, the GPCR polypeptides of this invention are identified in SEQ ID NOs:14-26. Functional fragments of the GPCR polypeptides are also embraced by the present invention.

“Similar” amino acids are those which have the same or similar physical properties and in many cases, the function is conserved with similar residues. For example, amino acids lysine and arginine are similar; while residues such as proline and cysteine do not share any physical property and are not considered to be similar. The term “consensus” refers to a sequence that reflects the most common choice of base or amino acid at each position among a series of related DNA, RNA or protein sequences. Areas of particularly good agreement often represent conserved functional domains.

A “variant” of a GPCR polypeptide refers to an amino acid sequence that is altered by one or more amino acids. The variant may have “conservative” changes, in which a substituted amino acid has similar structural or chemical properties, e.g., replacement of leucine with isoleucine. More rarely, a variant may have “non-conservative” changes, for example, replacement of a glycine with a tryptophan. The encoded protein may also contain deletions, insertions, or substitutions of amino acid residues, which produce a silent change and result in a functionally equivalent GPCR protein. Deliberate amino acid substitutions may be made on the basis of similarity in polarity, charge, solubility, hydrophobicity, hydrophilicity, and/or the amphipathic nature of the residues, as long as the biological activity of GPCR protein is retained.

For example, negatively charged amino acids may include aspartic acid and glutamic acid; positively charged amino acids may include lysine and arginine; and amino acids with uncharged polar head groups having similar hydrophilicity values may include leucine, isoleucine, and valine; glycine and alanine; asparagine and glutamine; serine and threonine; and phenylalanine and tyrosine. Guidance in determining which amino acid residues may be substituted, inserted, or deleted without abolishing functional biological or immunological activity may be found using computer programs well known in the art, for example, DNASTAR, Inc. software (Madison, Wis.).

The term “mimetic”, as used herein, refers to a molecule, having a structure which is developed from knowledge of the structure of a GPCR protein, or portions thereof, and as such, is able to affect some or all of the actions of the GPCR protein. A mimetic may comprise of a synthetic peptide or an organic molecule.

“Nucleic acid or polynucleotide sequence”, as used herein, refers to an isolated oligonucleotide (“oligo”), nucleotide, or polynucleotide, and fragments thereof, and to DNA or RNA of genomic or synthetic origin which may be single- or double-stranded, and represent the sense or anti-sense strand, preferably of the GPCR. By way of non-limiting examples, fragments include nucleic acid sequences that are greater than 20-60 nucleotides in length, and preferably include fragments that are at least 70-100 nucleotides, or which are at least 1000 nucleotides or greater in length. GPCR nucleic acid sequences of this invention are specifically identified in SEQ ID NOs:1-13 of Table 1 and as illustrated in the Figures.

An “allele” or “allelic sequence” is an alternative form of a GPCR nucleic acid sequence. Alleles may result from at least one mutation in a GPCR nucleic acid sequence and may yield altered mRNAs or polypeptides whose structure or function may or may not be altered. Any given gene, whether natural or recombinant, may have none, one, or many allelic forms. Common mutational changes, which give rise to alleles, are generally ascribed to natural deletions, additions, or substitutions of nucleotides. Each of these types of changes may occur alone, or in combination with the others, one or more times in a given sequence.

“Peptide nucleic acid” (PNA) refers to an antisense molecule or anti-gene agent which comprises an oligonucleotide (“oligo”) linked via an amide bond, similar to the peptide backbone of amino acid residues. PNAs typically comprise oligos of at least 5 nucleotides linked via amide bonds. PNAs may or may not terminate in positively charged amino acid residues to enhance binding affinities to DNA. Such amino acids include, for example, lysine and arginine, among others. These small molecules stop transcript elongation by binding to their complementary strand of nucleic acid (P. E. Nielsen et al., 1993, Anticancer Drug Des., 8:53-63). PNA may be pegylated to extend their lifespan in the cell where they preferentially bind to complementary single stranded DNA and RNA.

“Oligonucleotides” or “oligomers”, as defined herein, refer to a GPCR nucleic acid sequence comprising contiguous nucleotides, of at least about 5 nucleotides to about 60 nucleotides, preferably at least about 8 to 10 nucleotides in length, more preferably at least about 12 nucleotides in length, for example, about 15 to 35 nucleotides, or about 15 to 25 nucleotides, or about 20 to 35 nucleotides, which can be typically used in PCR amplification assays, hybridization assays, or in microarrays. It will be understood that the term oligonucleotide is substantially equivalent to the terms primer, probe, or amplimer, as commonly defined in the art. GPCR primers of this invention, (i.e., left, right, and internal primers), are set forth SEQ ID NOs:27-71 in Tables 2 and 3 herein.

The term “antisense” refers to nucleotide sequences, and compositions containing nucleic acid sequences, which are complementary to a specific DNA or RNA sequence. The term “antisense strand” is used in reference to a nucleic acid strand that is complementary to the “sense” strand. Antisense (i.e., complementary) nucleic acid molecules include PNAs and may be produced by any method, including synthesis or transcription. Once introduced into a cell, the complementary nucleotides combine with natural sequences produced by the cell to form duplexes, which block either transcription or translation. The designation “negative” is sometimes used in reference to the antisense strand, and “positive” is sometimes used in reference to the sense strand.

“Altered” nucleic acid sequences encoding a GPCR polypeptide include nucleic acid sequences containing deletions, insertions and/or substitutions of different nucleotides resulting in a polynucleotide that encodes the same or a functionally equivalent GPCR polypeptide. Altered nucleic acid sequences may further include polymorphisms of the polynucleotide encoding a GPCR polypeptide; such polymorphisms may or may not be readily detectable using a particular oligonucleotide probe.

The terms “Expressed Sequence Tag” or “EST” refers to the partial sequence of a cDNA insert which has been made by reverse transcription of mRNA extracted from a tissue, followed by insertion into a vector as known in the art (Adams, M. D., et al. Science (1991) 252:1651-1656; Adams, M. D. et al., Nature, (1992) 355:632-634; Adams, M. D., et al., Nature (1995) 377 Supp:3-174).

The term “biologically active”, i.e., functional, refers to a protein or polypeptide or fragment thereof, having structural, regulatory, or biochemical functions of a naturally occurring molecule. Likewise, “immunologically active” refers to the capability of a natural, recombinant, or synthetic GPCR, or an oligopeptide thereof, to induce a specific immune response in appropriate animals or cells, for example, to generate antibodies, to bind with specific antibodies, and/or to elicit a cellular immune response.

An “agonist” refers to a molecule which, when bound to, or associated with, a GPCR polypeptide, or a functional fragment thereof, increases or prolongs the duration of the effect of the GPCR polypeptide. Agonists may include proteins, nucleic acids, carbohydrates, or any other molecules that bind to and modulate the effect of GPCR polypeptide. Agonists typically enhance, increase, or augment the function or activity of a GPCR molecule.

An “antagonist” refers to a molecule which, when bound to, or associated with, a GPCR polypeptide, or a functional fragment thereof, decreases the amount or duration of the biological or immunological activity of GPCR polypeptide. Antagonists may include proteins, nucleic acids, carbohydrates, antibodies, or any other molecules that decrease or reduce the effect of a GPCR polypeptide. Antagonists typically, diminish, inhibit, or reduce the function or activity of a GPCR molecule.

The terms “complementary” or “complementarity” refer to the natural binding of polynucleotides under permissive salt and temperature conditions by base pairing. For example, the sequence “A-G-T” binds to the complementary sequence “T-C-A”. Complementarity between two single-stranded molecules may be “partial”, in which only some of the nucleic acids bind, or it may be “complete” when total complementarity exists between single stranded molecules. The degree of complementarity between nucleic acid strands has significant effects on the efficiency and strength of hybridization between nucleic acid strands. This is of particular importance in amplification reactions, which depend upon binding between nucleic acids strands, as well as in the design and use of PNA molecules.

The term “homology” refers to a degree of complementarity. There may be partial homology or complete homology, wherein complete homology is equivalent to identity. A partially complementary sequence that at least partially inhibits an identical sequence from hybridizing to a target nucleic acid is referred to as the functional term “substantially homologous”. The inhibition of hybridization of the completely complementary sequence to the target sequence may be examined using a hybridization assay (for example, Southern or Northern blot, solution hybridization, and the like) under conditions of low stringency. A substantially homologous sequence or probe will compete for and inhibit the binding (i.e., the hybridization) of a completely homologous sequence or probe to the target sequence under conditions of low stringency. Nonetheless, conditions of low stringency do not permit non-specific binding; low stringency conditions require that the binding of two sequences to one another be a specific (i.e., selective) interaction. The absence of non-specific binding may be tested by the use of a second target sequence which lacks even a partial degree of complementarity (for example, less than about 30% identity). In the absence of non-specific binding, the probe will not hybridize to the second non-complementary target sequence.

As used herein the terms “modulate” or “modulates” refer to an increase or decrease in the amount, quality or effect of a particular activity, DNA, RNA, or protein. The definition of “modulate” or “modulates” as used herein is meant to encompass agonists and/or antagonists of a particular activity, DNA, RNA, or protein.

It is another aspect of the present invention to provide modulators of the Gene 1, 2, 3, 4, 5, 6, 7, 8, 9, 10, 11, 12 and/or 13 protein and Gene 1, 2, 3, 4, 5, 6, 7, 8, 9, 10, 11, 12 and/or 13 peptide targets which can affect the function or activity of Gene 1, 2, 3, 4, 5, 6, 7, 8, 9, 10, 11, 12 and/or 13in a cell in which Gene 1, 2, 3, 4, 5, 6, 7, 8, 9, 10, 11, 12 and/or 13 function or activity is to be modulated or affected. In addition, modulators of Gene 1, 2, 3, 4, 5, 6, 7, 8, 9, 10, 11, 12 and/or 13 can affect downstream systems and molecules that are regulated by, or which interact with, Gene 1, 2, 3, 4, 5, 6, 7, 8, 9, 10, 11, 12 and/or 13 in the cell. Modulators of Gene 1, 2, 3, 4, 5, 6, 7, 8, 9, 10, 11, 12 and/or 13 include compounds, materials, agents, drugs, and the like, that antagonize, inhibit, reduce, block, suppress, diminish, decrease, or eliminate Gene 1, 2, 3, 4, 5, 6, 7, 8, 9, 10, 11, 12 and/or 13 function and/or activity. Such compounds, materials, agents, drugs and the like can be collectively termed “antagonists”. Alternatively, modulators of Gene 1, 2, 3, 4, 5, 6, 7, 8, 9, 10, 11, 12 and/or 13 include compounds, materials, agents, drugs, and the like, that agonize, enhance, increase, augment, or amplify Gene 1, 2, 3, 4, 5, 6, 7, 8, 9, 10, 11, 12 and/or 13 function in a cell. Such compounds, materials, agents, drugs and the like can be collectively termed “agonists”.

As a practical matter, whether any particular nucleic acid molecule or polypeptide is at least about 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to a nucleotide sequence of the present invention can be determined conventionally using known computer programs. A preferred method for determining the best overall match between a query sequence (a sequence of the present invention) and a subject sequence, also referred to as a global sequence alignment, can be determined using the CLUSTALW computer program (Thompson, J. D., et al., Nucleic Acids Research, 2(22):4673-4680, (1994)), which is based on the algorithm of Higgins, D. G., et al., Computer Applications in the Biosciences (CABIOS), 8(2):189-191, (1992). In a sequence alignment the query and subject sequences are both DNA sequences. An RNA sequence can be compared by converting U's to T's. However, the CLUSTALW algorithm automatically converts U's to T's when comparing RNA sequences to DNA sequences. The result of said global sequence alignment is in percent identity. Preferred parameters used in a CLUSTALW alignment of DNA sequences to calculate percent identity via pairwise alignments are: Matrix=IUB, k-tuple=1, Number of Top Diagonals=5, Gap Penalty=3, Gap Open Penalty 10, Gap Extension Penalty=0.1, Scoring Method=Percent, Window Size=5 or the length of the subject nucleotide sequence, whichever is shorter. For multiple alignments, the following CLUSTALW parameters are preferred: Gap Opening Penalty=10; Gap Extension Parameter=0.05; Gap Separation Penalty Range=8; End Gap Separation Penalty=Off; % Identity for Alignment Delay=40%; Residue Specific Gaps:Off; Hydrophilic Residue Gap=Off; and Transition Weighting=0. The pairwise and multple alignment parameters provided for CLUSTALW above represent the default parameters as provided with the AlignX software program (Vector NTI suite of programs, version 6.0).

The present invention encompasses the application of a manual correction to the percent identity results, in the instance where the subject sequence is shorter than the query sequence because of 5′ or 3′ deletions, not because of internal deletions. If only the local pairwise percent identity is required, no manual correction is needed. However, a manual correction may be applied to determine the global percent identity from a global polynucleotide alignment. Percent identity calculations based upon global polynucleotide alignments are often preferred since they reflect the percent identity between the polynucleotide molecules as a whole (i.e., including any polynucleotide overhangs, not just overlapping regions), as opposed to, only local matching polynucleotides. Manual corrections for global percent identity determinations are required since the CLUSTALW program does not account for 5′ and 3′ truncations of the subject sequence when calculating percent identity. For subject sequences truncated at the 5′ or 3′ ends, relative to the query sequence, the percent identity is corrected by calculating the number of bases of the query sequence that are 5′ and 3′ of the subject sequence, which are not matched/aligned, as a percent of the total bases of the query sequence. Whether a nucleotide is matched/aligned is determined by results of the CLUSTALW sequence alignment. This percentage is then subtracted from the percent identity, calculated by the above CLUSTALW program using the specified parameters, to arrive at a final percent identity score. This corrected score may be used for the purposes of the present invention. Only bases outside the 5′ and 3′ bases of the subject sequence, as displayed by the CLUSTALW alignment, which are not matched/aligned with the query sequence, are calculated for the purposes of manually adjusting the percent identity score.

For example, a 90 base subject sequence is aligned to a 100 base query sequence to determine percent identity. The deletions occur at the 5′ end of the subject sequence and therefore, the CLUSTALW alignment does not show a matched/alignment of the first 10 bases at 5′ end. The 10 unpaired bases represent 10% of the sequence (number of bases at the 5′ and 3′ ends not matched/total number of bases in the query sequence) so 10% is subtracted from the percent identity score calculated by the CLUSTALW program. If the remaining 90 bases were perfectly matched the final percent identity would be 90%. In another example, a 90 base subject sequence is compared with a 100 base query sequence. This time the deletions are internal deletions so that there are no bases on the 5′ or 3′ of the subject sequence which are not matched/aligned with the query. In this case the percent identity calculated by CLUSTALW is not manually corrected. Once again, only bases 5′ and 3′ of the subject sequence which are not matched/aligned with the query sequence are manually corrected for. No other manual corrections are required for the purposes of the present invention.

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, or substituted with another amino acid. These alterations of the reference sequence may occur at the amino- or carboxy-terminal positions of the reference amino acid sequence or anywhere between those terminal positions, interspersed either individually among residues in the reference sequence or in one or more contiguous groups within the reference sequence.

As a practical matter, whether any particular polypeptide is at least about 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to, for instance, an amino acid sequence referenced in Table 1 (SEQ ID NO:14, 15, 16, 17, 18, 19, 20, 21, 22, 23, 24, 25, or 26) or to the amino acid sequence encoded by cDNA contained in a deposited clone, can be determined conventionally using known computer programs. A preferred method for determining the best overall match between a query sequence (a sequence of the present invention) and a subject sequence, also referred to as a global sequence alignment, can be determined using the CLUSTALW computer program (Thompson, J. D., et al., Nucleic Acids Research, 2(22):4673-4680, (1994)), which is based on the algorithm of Higgins, D. G., et al., Computer Applications in the Biosciences (CABIOS), 8(2):189-191, (1992). In a sequence alignment the query and subject sequences are both amino acid sequences. The result of said global sequence alignment is in percent identity. Preferred parameters used in a CLUSTALW alignment of DNA sequences to calculate percent identity via pairwise alignments are: Matrix=BLOSUM, k-tuple=1, Number of Top Diagonals=5, Gap Penalty=3, Gap Open Penalty 10, Gap Extension Penalty=0.1, Scoring Method=Percent, Window Size=5 or the length of the subject nucleotide sequence, whichever is shorter. For multiple alignments, the following CLUSTALW parameters are preferred: Gap Opening Penalty=10; Gap Extension Parameter=0.05; Gap Separation Penalty Range=8; End Gap Separation Penalty=Off; % Identity for Alignment Delay=40%;

Residue Specific Gaps:Off; Hydrophilic Residue Gap=Off; and Transition Weighting=0. The pairwise and multple alignment parameters provided for CLUSTALW above represent the default parameters as provided with the AlignX software program (Vector NTI suite of programs, version 6.0).

The present invention encompasses the application of a manual correction to the percent identity results, in the instance where the subject sequence is shorter than the query sequence because of N- or C-terminal deletions, not because of internal deletions. If only the local pairwise percent identity is required, no manual correction is needed. However, a manual correction may be applied to determine the global percent identity from a global polypeptide alignment. Percent identity calculations based upon global polypeptide alignments are often preferred since they reflect the percent identity between the polypeptide molecules as a whole (i.e., including any polypeptide overhangs, not just overlapping regions), as opposed to, only local matching polypeptides. Manual corrections for global percent identity determinations are required since the CLUSTALW program does not account for N- and C-terminal truncations of the subject sequence when calculating percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the CLUSTALW sequence alignment. This percentage is then subtracted from the percent identity, calculated by the above CLUSTALW program using the specified parameters, to arrive at a final percent identity score. This final percent identity score is what may be used for the purposes of the present invention. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence.

For example, a 90 amino acid residue subject sequence is aligned with a 100 residue query sequence to determine percent identity. The deletion occurs at the N-terminus of the subject sequence and therefore, the CLUSTALW alignment does not show a matching/alignment of the first 10 residues at the N-terminus. The 10 unpaired residues represent 10% of the sequence (number of residues at the N- and C-termini not matched/total number of residues in the query sequence) so 10% is subtracted from the percent identity score calculated by the CLUSTALW program. If the remaining 90 residues were perfectly matched the final percent identity would be 90%. In another example, a 90 residue subject sequence is compared with a 100 residue query sequence. This time the deletions are internal deletions so there are no residues at the N- or C-termini of the subject sequence, which are not matched/aligned with the query. In this case the percent identity calculated by CLUSTALW is not manually corrected. Once again, only residue positions outside the N- and C-terminal ends of the subject sequence, as displayed in the CLUSTALW alignment, which are not matched/aligned with the query sequence are manually corrected for. No other manual corrections are required for the purposes of the present invention.

In addition to the above method of aligning two or more polynucleotide or polypeptide sequences to arrive at a percent identity value for the aligned sequences, it may be desirable in some circumstances to use a modified version of the CLUSTALW algorithm which takes into account known structural features of the sequences to be aligned, such as for example, the SWISS-PROT designations for each sequence. The result of such a modifed CLUSTALW algorithm may provide a more accurate value of the percent identity for two polynucleotide or polypeptide sequences. Support for such a modified version of CLUSTALW is provided within the CLUSTALW algorithm and would be readily appreciated to one of skill in the art of bioinformatics.

Also available to those having skill in this art are the BLAST and BLAST 2.0 algorithms (Altschul et al., 1977, Nuc. Acids Res., 25:3389-3402 and Altschul et al., 1990, J. Mol. Biol., 215:403-410). The BLASTN program for nucleic acid sequences uses as defaults a wordlength (W) of 11, an expectation (E) of 10, M=5, N=4, and a comparison of both strands. For amino acid sequences, the BLASTP program uses as defaults a wordlength (W) of 3, and an expectation (E) of 10. The BLOSUM62 scoring matrix (Henikoff & Henikoff, 1989, Proc. Natl. Acad. Sci., USA, 89:10915) uses alignments (B) of 50, expectation (E) of 10, M=5, N=4, and a comparison of both strands.

The invention encompasses polypeptides having a lower degree of identity but having sufficient similarity so as to perform one or more of the same functions performed by the polypeptide of the present invention. Similarity is determined by conserved amino acid substitution. Such substitutions are those that substitute a given amino acid in a polypeptide by another amino acid of like characteristics (e.g., chemical properties). According to Cunningham et al above, such conservative substitutions are likely to be phenotypically silent. Additional guidance concerning which amino acid changes are likely to be phenotypically silent are found in Bowie et al., Science 247:1306-1310 (1990).

Tolerated conservative amino acid substitutions of the present invention involve replacement of the aliphatic or hydrophobic amino acids Ala, Val, Leu and Ile; replacement of the hydroxyl residues Ser and Thr; replacement of the acidic residues Asp and Glu; replacement of the amide residues Asn and Gln, replacement of the basic residues Lys, Arg, and His; replacement of the aromatic residues Phe, Tyr, and Trp, and replacement of the small-sized amino acids Ala, Ser, Thr, Met, and Gly.

In addition, the present invention also encompasses the conservative substitutions provided in Table 4 below.

TABLE 4

| For Amino Acid | Code | Replace with any of: |
|---|---|---|
| Alanine | A | D-Ala, Gly, beta-Ala, L-Cys, D-Cys |
| Arginine | R | D-Arg, Lys, D-Lys, homo-Arg, D-homo-Arg, |
| | | Met, Ile, D-Met, D-Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S-Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, β-Ala, Acp |
| Isoleucine | I | D-Ile, Val, D-Val, Leu, D-Leu, Met, D-Met |
| Leucine | L | D-Leu, Val, D-Val, Met, D-Met |
| Lysine | K | D-Lys, Arg, D-Arg, homo-Arg, D-homo-Arg, |
| | | Met, D-Met, Ile, D-Ile, Orn, D-Orn |
| Methionine | M | D-Met, S-Me-Cys, Ile, D-Ile, Leu, D-Leu, |
| | | Val, D-Val |
| Phenylalanine | F | D-Phe, Tyr, D-Thr, L-Dopa, His, D-His, Trp, |
| | | D-Trp, Trans-3,4, or 5-phenylproline, cis-3,4, |
| | | or 5-phenylproline |
| Proline | P | D-Pro, L-1-thioazolidine-4-carboxylic acid, |
| | | D- or L-1-oxazolidine-4-carboxylic acid |
| Serine | S | D-Ser, Thr, D-Thr, allo-Thr, Met, D-Met, |
| | | Met(O), D-Met(O), L-Cys, D-Cys |
| Threonine | T | D-Thr, Ser, D-Ser, allo-Thr, Met, D-Met, |
| | | Met(O), D-Met(O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met |

Aside from the uses described above, such amino acid substitutions may also increase protein or peptide stability. The invention encompasses amino acid substitutions that contain, for example, one or more non-peptide bonds (which replace the peptide bonds) in the protein or peptide sequence. Also included are substitutions that include amino acid residues other than naturally occurring L-amino acids, e.g., D-amino acids or non-naturally occurring or synthetic amino acids, e.g., β or γ amino acids.

Both identity and similarity can be readily calculated by reference to the following publications: Computational Molecular Biology, Lesk, A. M., ed., Oxford University Press, New York, 1988; Biocomputing: Informatics and Genome Projects, Smith, D. W., ed., Academic Press, New York, 1993; Informatics Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991.

In addition, the present invention also encompasses substitution of amino acids based upon the probability of an amino acid substitution resulting in conservation of function. Such probabilities are determined by aligning multiple genes with related function and assessing the relative penalty of each substitution to proper gene function. Such probabilities are often described in a matrix and are used by some algorithms (e.g., BLAST, CLUSTALW, GAP, etc.) in calculating percent similarity wherein similarity refers to the degree by which one amino acid may substitute for another amino acid without lose of function. An example of such a matrix is the PAM250 or BLOSUM62 matrix.

Aside from the canonical chemically conservative substitutions referenced above, the invention also encompasses substitutions which are typically not classified as conservative, but that may be chemically conservative under certain circumstances. Analysis of enzymatic catalysis for proteases, for example, has shown that certain amino acids within the active site of some enzymes may have highly perturbed pKa's due to the unique microenvironment of the active site. Such perturbed pKa's could enable some amino acids to substitute for other amino acids while conserving enzymatic structure and function. Examples of amino acids that are known to have amino acids with perturbed pKa's are the Glu-35 residue of Lysozyme, the Ile-16 residue of Chymotrypsin, the His-159 residue of Papain, etc. The conservation of function relates to either anomalous protonation or anomalous deprotonation of such amino acids, relative to their canonical, non-perturbed pKa. The pKa perturbation may enable these amino acids to actively participate in general acid-base catalysis due to the unique ionization environment within the enzyme active site. Thus, substituting an amino acid capable of serving as either a general acid or general base within the microenvironment of an enzyme active site or cavity, as may be the case, in the same or similar capacity as the wild-type amino acid, would effectively serve as a conservative amino substitution.

The present invention is directed to polynucleotide fragments of the polynucleotides of the invention, in addition to polypeptides encoded therein by said polynucleotides and/or fragments.

In the present invention, a “polynucleotide fragment” refers to a short polynucleotide having a nucleic acid sequence which: is a portion of that contained in a deposited clone, or encoding the polypeptide encoded by the cDNA in a deposited clone; is a portion of that shown in SEQ ID NO:1, 2, 3, 4, 5, 6, 7, 8, 9, 10, 11, 12, or 13 or the complementary strand thereto, or is a portion of a polynucleotide sequence encoding the polypeptide of SEQ ID NO:14, 15, 16, 17, 18, 19, 20, 21, 22, 23, 24, 25, or 26. The nucleotide fragments of the invention are preferably at least about 15 nt, and more preferably at least about 20 nt, still more preferably at least about 30 nt, and even more preferably, at least about 40 nt, at least about 50 nt, at least about 75 nt, or at least about 150 nt in length. A fragment “at least 20 nt in length,” for example, is intended to include 20 or more contiguous bases from the cDNA sequence contained in a deposited clone or the nucleotide sequence shown in SEQ ID NO:1, 2, 3, 4, 5, 6, 7, 8, 9, 10, 11, 12, or 13. In this context “about” includes the particularly recited value, a value larger or smaller by several (5, 4, 3, 2, or 1) nucleotides, at either terminus, or at both termini. These nucleotide fragments have uses that include, but are not limited to, as diagnostic probes and primers as discussed herein. Of course, larger fragments (e.g., 50, 150, 500, 600, 2000 nucleotides) are preferred.

Moreover, representative examples of polynucleotide fragments of the invention, include, for example, fragments comprising, or alternatively consisting of, a sequence from about nucleotide number 1-50, 51-100, 101-150, 151-200, 201-250, 251-300, 301-350, 351-400, 401-450, 451-500, 501-550, 551-600, 651-700, 701-750, 751-800, 800-850, 851-900, 901-950, 951-1000, 1001-1050, 1051-1100, 1101-1150, 1151-1200, 1201-1250, 1251-1300, 1301-1350, 1351-1400, 1401-1450, 1451-1500, 1501-1550, 1551-1600, 1601-1650, 1651-1700, 1701-1750, 1751-1800, 1801-1850, 1851-1900, 1901-1950, 1951-2000, or 2001 to the end of SEQ ID NO:1, 2, 3, 4, 5, 6, 7, 8, 9, 10, 11, 12, or 13, or the complementary strand thereto, or the cDNA contained in a deposited clone. In this context “about” includes the particularly recited ranges, and ranges larger or smaller by several (5, 4, 3, 2, or 1) nucleotides, at either terminus or at both termini. Preferably, these fragments encode a polypeptide which has biological activity. More preferably, these polynucleotides can be used as probes or primers as discussed herein. Also encompassed by the present invention are polynucleotides which hybridize to these nucleic acid molecules under stringent hybridization conditions or lower stringency conditions, as are the polypeptides encoded by these polynucleotides.

In the present invention, a “polypeptide fragment” refers to an amino acid sequence which is a portion of that contained in SEQ ID NO:14, 15, 16, 17, 18, 19, 20, 21, 22, 23, 24, 25, or 26 or encoded by the cDNA contained in a deposited clone. Protein (polypeptide) fragments may be “free-standing,” or comprised within a larger polypeptide of which the fragment forms a part or region, most preferably as a single continuous region. Representative examples of polypeptide fragments of the invention, include, for example, fragments comprising, or alternatively consisting of, from about amino acid number 1-20, 21-40, 41-60, 61-80, 81-100, 102-120, 121-140, 141-160, or 161 to the end of the coding region. Moreover, polypeptide fragments can be about 20, 30, 40, 50, 60, 70, 80, 90, 100, 110, 120, 130, 140, or 150 amino acids in length. In this context “about” includes the particularly recited ranges or values, and ranges or values larger or smaller by several (5, 4, 3, 2, or 1) amino acids, at either extreme or at both extremes. Polynucleotides encoding these polypeptides are also encompassed by the invention.

Preferred polypeptide fragments include the full-length protein. Further preferred polypeptide fragments include the full-length protein having a continuous series of deleted residues from the amino or the carboxy terminus, or both. For example, any number of amino acids, ranging from 1-60, can be deleted from the amino terminus of the full-length polypeptide. Similarly, any number of amino acids, ranging from 1-30, can be deleted from the carboxy terminus of the full-length protein. Furthermore, any combination of the above amino and carboxy terminus deletions are preferred. Similarly, polynucleotides encoding these polypeptide fragments are also preferred.

Also preferred are polypeptide and polynucleotide fragments characterized by structural or functional domains, such as fragments that comprise alpha-helix and alpha-helix forming regions, beta-sheet and beta-sheet-forming regions, turn and turn-forming regions, coil and coil-forming regions, hydrophilic regions, hydrophobic regions, alpha amphipathic regions, beta amphipathic regions, flexible regions, surface-forming regions, substrate binding region, and high antigenic index regions. Polypeptide fragments of SEQ ID NO:14, 15, 16, 17, 18, 19, 20, 21, 22, 23, 24, 25, or 26 falling within conserved domains are specifically contemplated by the present invention. Moreover, polynucleotides encoding these domains are also contemplated.

Other preferred polypeptide fragments are biologically active fragments. Biologically active fragments are those exhibiting activity similar, but not necessarily identical, to an activity of the polypeptide of the present invention. The biological activity of the fragments may include an improved desired activity, or a decreased undesirable activity. Polynucleotides encoding these polypeptide fragments are also encompassed by the invention.

In a preferred embodiment, the functional activity displayed by a polypeptide encoded by a polynucleotide fragment of the invention may be one or more biological activities typically associated with the full-length polypeptide of the invention. Illustrative of these biological activities includes the fragments ability to bind to at least one of the same antibodies which bind to the full-length protein, the fragments ability to interact with at lease one of the same proteins which bind to the full-length, the fragments ability to elicit at least one of the same immune responses as the full-length protein (i.e., to cause the immune system to create antibodies specific to the same epitope, etc.), the fragments ability to bind to at least one of the same polynucleotides as the full-length protein, the fragments ability to bind to a receptor of the full-length protein, the fragments ability to bind to a ligand of the full-length protein, and the fragments ability to multimerize with the full-length protein. However, the skilled artisan would appreciate that some fragments may have biological activities which are desirable and directly inapposite to the biological activity of the full-length protein. The functional activity of polypeptides of the invention, including fragments, variants, derivatives, and analogs thereof can be determined by numerous methods available to the skilled artisan, some of which are described elsewhere herein.

The term “hybridization” refers to any process by which a strand of nucleic acids binds with a complementary strand through base pairing. The term “hybridization complex” refers to a complex formed between two nucleic acid sequences by virtue of the formation of hydrogen bonds between complementary G and C bases and between complementary A and T bases. The hydrogen bonds may be further stabilized by base stacking interactions. The two complementary nucleic acid sequences hydrogen bond in an anti-parallel configuration. A hybridization complex may be formed in solution (for example, Cₒt or Rₒt analysis), or between one nucleic acid sequence present in solution and another nucleic acid sequence immobilized on a solid phase or support (for example, membranes, filters, chips, pins, or glass slides, or any other appropriate substrate to which cells or their nucleic acids have been affixed).

The terms “stringency” or “stringent conditions” refer to the conditions for hybridization as defined by nucleic acid composition, salt, and temperature. These conditions are well known in the art and may be altered to identify and/or detect identical or related polynucleotide sequences in a sample. A variety of equivalent conditions comprising either low, moderate, or high stringency depend on factors such as the length and nature of the sequence (DNA, RNA, base composition), reaction milieu (in solution or immobilized on a solid substrate), nature of the target nucleic acid (DNA, RNA, base composition), concentration of salts and the presence or absence of other reaction components (for example, formamide, dextran sulfate and/or polyethylene glycol) and reaction temperature (within a range of from about 5° C. below the melting temperature of the probe to about 20° C. to 25° C. below the melting temperature). One or more factors may be varied to generate conditions, either low or high stringency that is different from but equivalent to the aforementioned conditions.

As will be understood by those of skill in the art, the stringency of hybridization may be altered in order to identify or detect identical or related polynucleotide sequences. As will be further appreciated by the skilled practitioner, the melting temperature, Tm, can be approximated by the formulas as well known in the art, depending on a number of parameters, such as the length of the hybrid or probe in number of nucleotides, or hybridization buffer ingredients and conditions (see, for example, T. Maniatis et al., Molecular Cloning: A Laboratory Manual, Cold Spring Harbor Laboratory, Cold Spring Harbor, N.Y., 1982 and J. Sambrook et al., Molecular Cloning: A Laboratory Manual, Cold Spring Harbor Laboratory, Cold Spring Harbor, N.Y., 1989; Current Protocols in Molecular Biology, Eds. F. M. Ausubel et al., Vol. 1, “Preparation and Analysis of DNA”, John Wiley and Sons, Inc., 1994-1995, Suppls. 26, 29, 35 and 42;

pp. 2.10.7-2.10.16; G. M. Wahl and S. L. Berger (1987; Methods Enzymol. 152:399-407); and A. R. Kimmel, 1987; Methods of Enzymol. 152:507-511).

As a general guide, Tₘ decreases approximately 1° C.-1.5° C. with every 1% decrease in sequence homology. Also, in general, the stability of a hybrid is a function of sodium ion concentration and temperature. Typically, the hybridization reaction is initially performed under conditions of low stringency, followed by washes of varying, but higher stringency. Reference to hybridization stringency, for example, high, moderate, or low stringency, typically relates to such washing conditions. It is to be understood that the low, moderate and high stringency hybridization or washing conditions can be varied using a variety of ingredients, buffers and temperatures well known to and practiced by the skilled artisan.

A “composition”, as defined herein, refers broadly to any composition containing a GPCR polynucleotide, polypeptide, derivative, or mimetic thereof, or antibodies thereto. The composition may comprise a dry formulation or an aqueous solution. Compositions comprising GPCR polynucleotide sequences (SEQ ID NOs:1-13) encoding GPCR polypeptides (SEQ ID NOs:14-26), or fragments thereof, may be employed as hybridization probes. The probes may be stored in a freeze-dried form and may be in association with a stabilizing agent such as a carbohydrate. In hybridizations, the probe may be employed in an aqueous solution containing salts (for example, NaCl), detergents or surfactants (for example, SDS) and other components (for example, Denhardt's solution, dry milk, salmon sperm DNA, and the like).

The term “substantially purified” refers to nucleic acid sequences or amino acid sequences that are removed from their natural environment, isolated or separated, and are at least 60% free, preferably 75% to 85% free, and most preferably 90% to 95%, or greater, free from other components with which they are naturally associated.

The term “sample”, or “biological sample”, is meant to be interpreted in its broadest sense. A non-limiting example of a biological sample suspected of containing a GPCR nucleic acid encoding GPCR protein, or fragments thereof, or a GPCR protein itself, may comprise, but is not limited to, a body fluid, an extract from cells or tissue, chromosomes isolated from a cell (for example, a spread of metaphase chromosomes), organelle, or membrane isolated from a cell, a cell, nucleic acid such as genomic GPCR DNA (in solution or bound to a solid support such as, for example, for Southern analysis), GPCR RNA (in solution or bound to a solid support such as for Northern analysis), GPCR cDNA (in solution or bound to a solid support), a tissue, a tissue print, and the like.

“Transformation” or transfection refers to a process by which exogenous DNA, preferably GPCR, enters and changes a recipient cell. It may occur under natural or artificial conditions using various methods well known in the art. Transformation may rely on any known method for the insertion of foreign nucleic acid sequences into a prokaryotic or eukaryotic host cell. The method is selected based on the type of host cell being transformed and may include, but is not limited to, viral infection, electroporation, heat shock, lipofection, and partial bombardment. Such “transformed” cells include stably transformed cells in which the inserted DNA is capable of replication either as an autonomously replicating plasmid or as part of the host chromosome. Transformed cells also include those cells, which transiently express the inserted DNA or RNA for limited periods of time.

The term “correlates with expression of a polynucleotide” indicates that the detection of the presence of ribonucleic acid that is similar to the nucleic acid sequence of GPCRs by Northern analysis is indicative of the presence of mRNA encoding GPCR polypeptides (SEQ ID NOs:14-26) in a sample and thereby correlates with expression of the transcript from the polynucleotide encoding the protein.

An alteration in the polynucleotide of SEQ ID NOs:1-13 comprises any alteration in the sequence of the polynucleotides encoding GPCR polypeptides, including deletions, insertions, and point mutations that may be detected using hybridization assays. Included within this definition is the detection of alterations to the genomic DNA sequence which encodes GPCR polypeptides (e.g., by alterations in the pattern of restriction fragment length polymorphisms capable of hybridizing to nucleic acid sequences SEQ ID NOs:1-13), the inability of a selected fragment of SEQ ID NOs:1-13 to hybridize to a sample of genomic DNA (e.g., using allele-specific oligonucleotide probes), and improper or unexpected hybridization, such as hybridization to a locus other than the normal chromosomal locus for the polynucleotide sequence encoding GPCR polypeptide (e.g., using fluorescent in situ hybridization (FISH) to metaphase chromosome spreads).

The term “antibody” refers to intact molecules as well as fragments thereof, such as Fab, F(ab′)₂, Fv, which are capable of binding an epitopic or antigenic determinant. Antibodies that bind to GPCR polypeptides can be prepared using intact polypeptides or fragments containing small peptides of interest or prepared recombinantly for use as the immunizing antigen. The polypeptide or oligopeptide used to immunize an animal can be derived from the transition of RNA or synthesized chemically, and can be conjugated to a carrier protein, if desired. Commonly used carriers that are chemically coupled to peptides include, but are not limited to, bovine serum albumin (BSA), keyhole limpet hemocyanin (KLH), and thyroglobulin. The coupled peptide is then used to immunize the animal (for example, a mouse, a rat, or a rabbit).

The term “humanized” antibody refers to antibody molecules in which amino acids have been replaced in the non-antigen binding regions (i.e., framework regions) of the immunoglobulin in order to more closely resemble a human antibody, while still retaining the original binding capability, for example, as described in U.S. Pat. No. 5,585,089 to C. L. Queen et al. In the present instance, humanized antibodies are preferably anti-GPCR specific antibodies.

The term “antigenic determinant” refers to that portion of a molecule that makes contact with a particular antibody (i.e., an epitope). When a protein or fragment of a protein, preferably a GPCR protein, is used to immunize a host animal, numerous regions of the protein may induce the production of antibodies which bind specifically to a given region or three-dimensional structure on the protein; these regions or structures are referred to an antigenic determinants. An antigenic determinant may compete with the intact antigen (i.e., the immunogen used to elicit the immune response) for binding to an antibody.

The terms “specific binding” or “specifically binding” refer to the interaction between a protein or peptide, preferably a GPCR protein, and a binding molecule, such as an agonist, an antagonist, or an antibody. The interaction is dependent upon the presence of a particular structure (i.e., an antigenic determinant or epitope) of the protein that is recognized by the binding molecule.

The present invention provides novel GPCR polynucleotides and encoded GPCR polypeptides. The GPCRs according to this invention are preferably full-length molecules. More specifically, the GPCRs according to the invention are sensory GPCRs, chemokine GPCRs, orphan GPCRs, and very large GPCRs.

GPCRs can also include dopamine receptors, rhodopsin receptors, kinin receptors, N-formyl peptide receptors, opioid receptors, calcitonin receptors, adrenergic receptors, endothelin receptors, cAMP receptors, adenosine receptors, muscarinic receptors, acetylcholine receptors, serotonin receptors, histamine receptors, thrombin receptors, follicle stimulating hormone receptors, opsin receptors, endothelial differentiation gene-1 receptors, odorant receptors, or cytomegalovirus receptors.

Features of the Polypeptide Encoded by Gene No:7

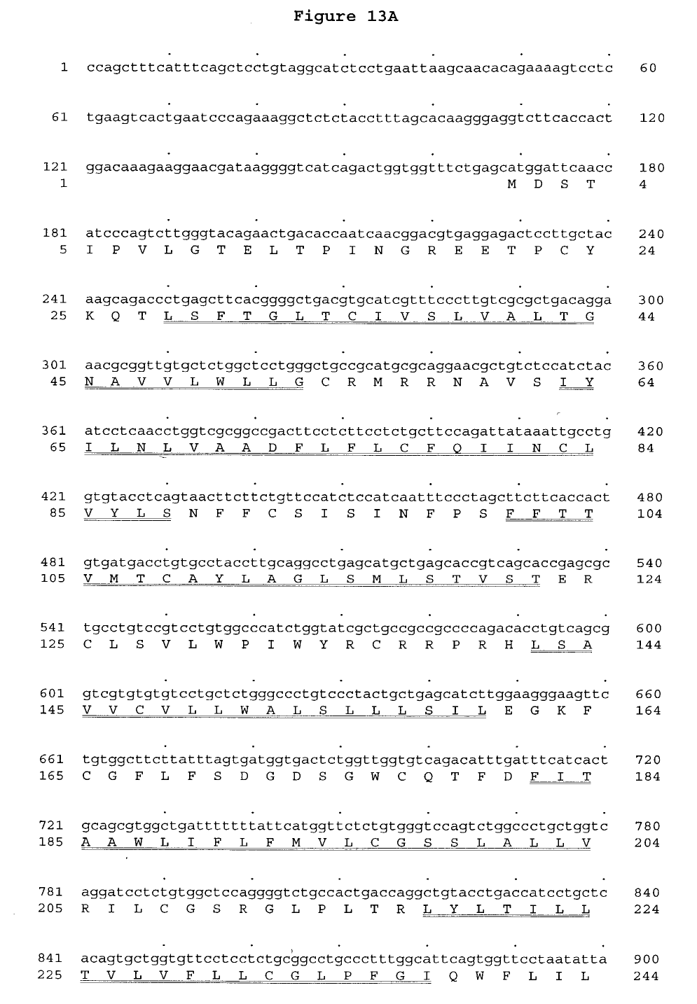

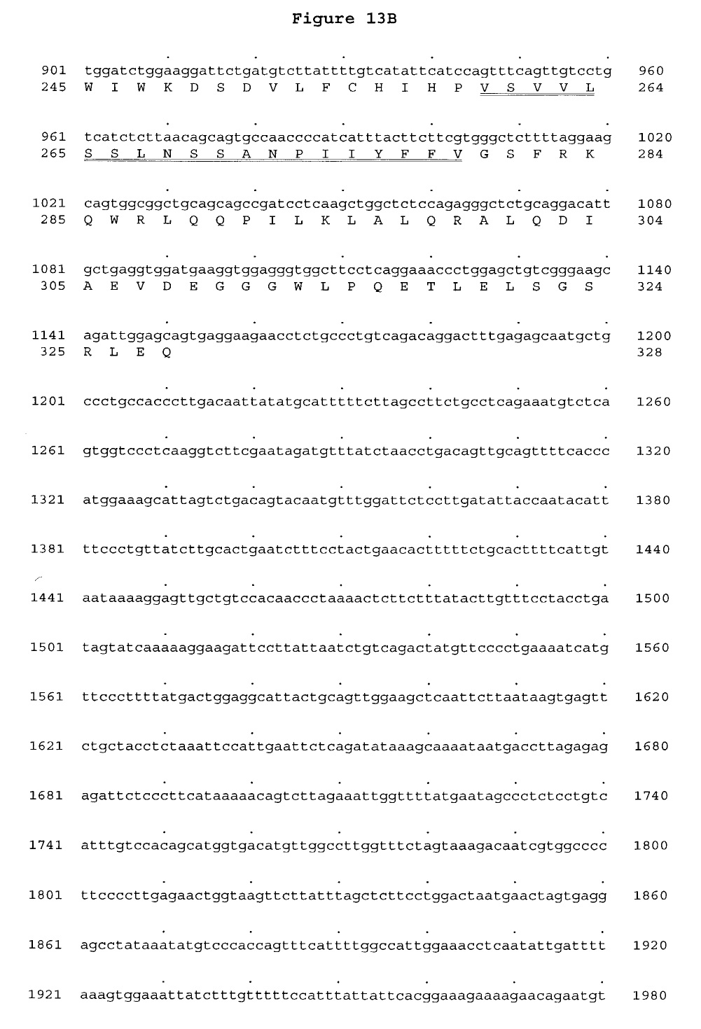

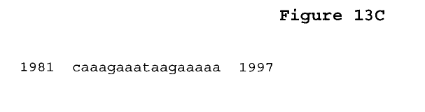

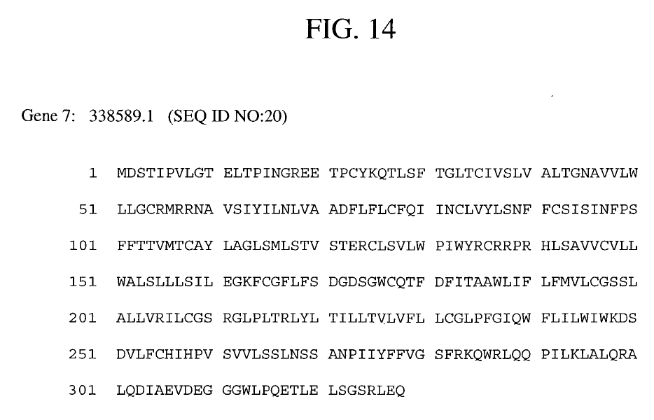

The determined nucleotide sequence of the Gene 7 cDNA in FIGS. 13A-C (SEQ ID NO:7) contains an open reading frame encoding a protein of about 328 amino acid residues, with a deduced molecular weight of about 36.9 kDa. The amino acid sequence of the predicted Gene 7 polypeptide is shown in FIGS. 13A-C and in FIG. 14 (SEQ ID NO:20).

The Gene 7 polypeptide was predicted to comprise seven transmembrane domains (TM1 to TM7) located from about amino acid 28 to about amino acid 53 (TM1; SEQ ID NO:87); from about amino acid 63 to about amino acid 88 (TM2; SEQ ID NO:88); from about amino acid 101 to about amino acid 122 (TM3; SEQ ID NO:89); from about amino acid 142 to about amino acid 160 (TM4; SEQ ID NO:90); from about amino acid 182 to about amino acid 204 (TM5; SEQ ID NO:91); from about amino acid 218 to about amino acid 238 (TM6; SEQ ID NO:92); and/or from about amino acid 260 to about amino acid 279 (TM7; SEQ ID NO:93) of SEQ ID NO:20 (FIGS. 13A-C and FIG. 14). In this context, the term “about” may be construed to mean 1, 2, 3, 4, 5, 6, 7, 8, 9, or 10 amino acids beyond the N-Terminus and/or C-terminus of the above referenced transmembrane domain polypeptides.

In preferred embodiments, the following transmembrane domain polypeptides are encompassed by the present invention: LSFTGLTCIVSLVALTGNAVVLWLLG (SEQ ID NO:87), IYILNLVAADFLFLCFQIINCLVYLS (SEQ ID NO:88), FFTTVMTCAYLAGLSMLSTVST (SEQ ID NO:89), LSAVVCVLLWALSLLLSIL (SEQ ID NO:90), FITAAWLIFLFMVLCGSSLALLV (SEQ ID NO:91), LYLTILLTVLVFLLCGLPFGI (SEQ ID NO:92), and/or VSVVLSSLNSSANPIIYFFV (SEQ ID NO:93). Polynucleotides encoding these polypeptides are also provided. The present invention also encompasses the use of these Gene 7 transmembrane domain polypeptides as immunogenic and/or antigenic epitopes as described elsewhere herein.

In preferred embodiments, the present invention encompasses the use of N-terminal deletions, C-terminal deletions, or any combination of N-terminal and C-terminal deletions of any one or more of the Gene 7 TM1 thru TM7 transmembrane domain polypeptides as antigenic and/or immunogenic epitopes.

In preferred embodiments, the present invention also encompasses the use of N-terminal deletions, C-terminal deletions, or any combination of N-terminal and C-terminal deletions of any one or more of the amino acids intervening (i.e., GPCR extracellular or intracellular loops) any pair of Gene 7 TM1 thru TM7 transmembrane domain polypeptides, and/or the amino acids intervening any pair of the Gene 7 TM1 thru TM7 transmembrane domain polypeptides themselves, as antigenic and/or immunogenic epitopes.

The coding region of the Gene 7 polynucleotide is predicted to be from nucleotide 169 to nucleotide 1152 of SEQ ID NO:7 as shown in FIGS. 13A-C, and the polypeptide corresponding to amino acids 1 thru 328 of SEQ ID NO:20. The present invention encompasses the polynucleotide encompassing the entire coding region of Gene 7.

Alternatively, the present invention encompasses a polynucleotide lacking the initiating start codon, in addition to, the resulting encoded polypeptide of Gene 7. Specifically, the present invention encompasses the polynucleotide corresponding to nucleotides 172 thru 1152 of SEQ ID NO:7, and the polypeptide corresponding to amino acids 2 thru 328 of SEQ ID NO:20. Also encompassed are recombinant vectors comprising said encoding sequence, and host cells comprising said vector.

In preferred embodiments, the following N-terminal Gene 7 deletion polypeptides are encompassed by the present invention: M1-Q328, D2-Q328, S3-Q328, T4-Q328, I5-Q328, P6-Q328, V7-Q328, L8-Q328, G9-Q328, T10-Q328, E11-Q328, L12-Q328, T13-Q328, P14-Q328, I15-Q328, N16-Q328, G17-Q328, R18-Q328, E19-Q328, E20-Q328, T21-Q328, P22-Q328, C23-Q328, Y24-Q328, K25-Q328, Q26-Q328, T27-Q328, L28-Q328, S29-Q328, F30-Q328, T31-Q328, G32-Q328, L33-Q328, T34-Q328, C35-Q328, 136-Q328, V37-Q328, S38-Q328, L39-Q328, V40-Q328, A41-Q328, L42-Q328, T43-Q328, G44-Q328, N45-Q328, A46-Q328, V47-Q328, V48-Q328, L49-Q328, W50-Q328, L51-Q328, L52-Q328, G53-Q328, C54-Q328, R55-Q328, M56-Q328, R57-Q328, R58-Q328, N59-Q328, A60-Q328, V61-Q328, S62-Q328, I63-Q328, Y64-Q328, I65-Q328, L66-Q328, N67-Q328, L68-Q328, V69-Q328, A70-Q328, A71-Q328, D72-Q328, F73-Q328, L74-Q328, F75-Q328, L76-Q328, C77-Q328, F78-Q328, Q79-Q328, I80-Q328, I81-Q328, N82-Q328, C83-Q328, L84-Q328, V85-Q328, Y86-Q328, L87-Q328, S88-Q328, N89-Q328, F90-Q328, F91-Q328, C92-Q328, S93-Q328, I94-Q328, S95-Q328, I96-Q328, N97-Q328, F98-Q328, P99-Q328, S100-Q328, F101-Q328, F102-Q328, T103-Q328, T104-Q328, V105-Q328, M106-Q328, T107-Q328, C108-Q328, A109-Q328, Y100-Q328, L111-Q328, A112-Q328, G113-Q328, L114-Q328, S115-Q328, M116-Q328, L117-Q328, S118-Q328, T119-Q328, V120-Q328, S121-Q328, T122-Q328, E123-Q328, R124-Q328, C125-Q328, L126-Q328, S127-Q328, V128-Q328, L129-Q328, W130-Q328, P131-Q328, I132-Q328, W133-Q328, Y134-Q328, R135-Q328, C136-Q328, R137-Q328, R138-Q328, P139-Q328, R140-Q328, H141-Q328, L142-Q328, S143-Q328, A144-Q328, V145-Q328, V146-Q328, C147-Q328, V148-Q328, L149-Q328, L150-Q328, W151-Q328, A152-Q328, L153-Q328, S154-Q328, L155-Q328, L156-Q328, L157-Q328, S158-Q328, 1159-Q328, L160-Q328, E161-Q328, G162-Q328, K163-Q328, F164-Q328, C165-Q328, G166-Q328, F167-Q328, L168-Q328, F169-Q328, S170-Q328, D171-Q328, G172-Q328, D173-Q328, S174-Q328, G175-Q328, W176-Q328, C177-Q328, Q178-Q328, T179-Q328, F180-Q328, D181-Q328, F182-Q328, I183-Q328, T184-Q328, A185-Q328, A186-Q328, W187-Q328, L188-Q328, I189-Q328, F190-Q328, L191-Q328, F192-Q328, M193-Q328, V194-Q328, L195-Q328, C196-Q328, G197-Q328, S198-Q328, S199-Q328, L200-Q328, A201-Q328, L202-Q328, L203-Q328, V204-Q328, R205-Q328, I206-Q328, L207-Q328, C208-Q328, G209-Q328, S210-Q328, R211-Q328, G212-Q328, L213-Q328, P214-Q328, L215-Q328, T216-Q328, R217-Q328, L218-Q328, Y219-Q328, L220-Q328, T221-Q328, I222-Q328, L223-Q328, L224-Q328, T225-Q328, V226-Q328, L227-Q328, V228-Q328, F229-Q328, L230-Q328, L231-Q328, C232-Q328, G233-Q328, L234-Q328, P235-Q328, F236-Q328, G237-Q328, I238-Q328, Q239-Q328, W240-Q328, F241-Q328, L242-Q328, I243-Q328, L244-Q328, W245-Q328, I246-Q328, W247-Q328, K248-Q328, D249-Q328, S250-Q328, D251-Q328, V252-Q328, L253-Q328, F254-Q328, C255-Q328, H256-Q328, I257-Q328, H258-Q328, P259-Q328, V260-Q328, S261-Q328, V262-Q328, V263-Q328, L264-Q328, S265-Q328, S266-Q328, L267-Q328, N268-Q328, S269-Q328, S270-Q328, A271-Q328, N272-Q328, P273-Q328, I274-Q328, 1275-Q328, Y276-Q328, F277-Q328, F278-Q328, V279-Q328, G280-Q328, S281-Q328, F282-Q328, R283-Q328, K284-Q328, Q285-Q328, W286-Q328, R287-Q328, L288-Q328, Q289-Q328, Q290-Q328, P291-Q328, I292-Q328, L293-Q328, K294-Q328, L295-Q328, A296-Q328, L297-Q328, Q298-Q328, R299-Q328, A300-Q328, L301-Q328, Q302-Q328, D303-Q328, I304-Q328, A305-Q328, E306-Q328, V307-Q328, D308-Q328, E309-Q328, G310-Q328, G311-Q328, G312-Q328, W313-Q328, L314-Q328, P315-Q328, Q316-Q328, E317-Q328, T318-Q328, L319-Q328, E320-Q328, L321-Q328, and/or S322-Q328 of SEQ ID NO:20. Polynucleotide sequences encoding these polypeptides are also provided. The present invention also encompasses the use of these N-terminal Gene 7 deletion polypeptides as immunogenic and/or antigenic epitopes as described elsewhere herein.

In preferred embodiments, the following C-terminal Gene 7 deletion polypeptides are encompassed by the present invention: M1-Q328, M1-E327, M1-L326, M1-R325, M1-S324, M1-G323, M1-S322, M1-L321, M1-E320, M1-L319, M1-T318, M1-E317, M1-Q316, M1-P315, M1-L314, M1-W313, M1-G312, M1-G311, M1-G310, M1-E309, M1-D308, M1-V307, M1-E306, M1-A305, M1-I304, M1-D303, M1-Q302, M1-L301, M1-A300, M1-R299, M1-Q298, M1-L297, M1-A296, M1-L295, M1-K294, M1-L293, M1-I292, M1-P291, M1-Q290, M1-Q289, M1-L288, M1-R287, M1-W286, M1-Q285, M1-K284, M1-R283, M1-F282, M1-S281, M1-G280, M1-V279, M1-F278, M1-F277, M1-Y276, M1-I275, M1-I274, M1-P273, M1-N272, M1-A271, M1-S270, M1-S269, M1-N268, M1-L267, M1-S266, M1-S265, M1-L264, M1-V263, M1-V262, M1-S261, M1-V260, M1-P259, M1-H258, M1-I257, M1-H256, M1-C255, M1-F254, M1-L253, M1-V252, M1-D251, M1-S250, M1-D249, M1-K248, M1-W247, M1-I246, M1-W245, M1-L244, M1-I243, M1-L242, M1-F241, M1-W240, M1-Q239, M1-I238, M1-G237, M1-F236, M1-P235, M1-L234, M1-G233, M1-C232, M1-L231, M1-L230, M1-F229, M1-V228, M1-L227, M1-V226, M1-T225, M1-L224, M1-L223, M1-I222, M1-T221, M1-L220, M1-Y219, M1-L218, M1-R217, M1-T216, M1-L215, M1-P214, M1-L213, M1-G212, M1-R211, M1-S210, M1-G209, M1-C208, M1-L207, M1-I206, M1-R205, M1-V204, M1-L203, M1-L202, M1-A201, M1-L200, M1-S199, M1-S198, M1-G197, M1-C196, M1-L195, M1-V194, M1-M193, M1-F192, M1-L191, M1-F190, M1-I189, M1-L188, M1-W187, M1-A186, M1-A185, M1-T184, M1-I183, M1-F182, M1-D181, M1-F180, M1-T179, M1-Q178, M1-C₁₇₇, M1-W176, M1-G175, M1-S174, M1-D173, M1-G172, M1-D171, M1-S170, M1-F169, M1-L168, M1-F167, M1-G166, M1-C165, M1-F164, M1-K163, M1-G162, M1-E161, M1-L160, M1-I159, M1-S158, M1-L157, M1-L156, M1-L155, M1-S154, M1-L153, M1-A152, M1-W151, M1-L150, M1-L149, M1-V148, M1-C₁₄₇, M1-V146, M1-V145, M1-A144, M1-S143, M1-L142, M1-H141, M1-R140, M1-P139, M1-R138, M1-R137, M1-C136, M1-R135, M1-Y134, M1-W133, M1-I132, M1-P131, M1-W130, M1-L129, M1-V128, M1-S127, M1-L126, M1-C125, M1-R124, M1-E123, M1-T122, M1-S121, M1-V120, M1-T119, M1-S118, M1-L117, M1-M116, M1-S115, M1-L114, M1-G113, M1-A112, M1-L111, M1-Y110, M1-A109, M1-C108, M1-T107, M1-M106, M1-V105, M1-T104, M1-T103, M1-F102, M1-F110, M1-S100, M1-P99, M1-F98, M1-N97, M1-I96, M1-S95, M1-I94, M1-S93, M1-C92, M1-F91, M1-F90, M1-N89, M1-S88, M1-L87, M1-Y86, M1-V85, M1-L84, M1-C83, M1-N82, M1-I81, M1-I80, M1-Q79, M1-F78, M1-C77, M1-L76, M1-F75, M1-L74, M1-F73, M1-D72, M1-A71, M1-A70, M1-V69, M1-L68, M1-N67, M1-L66, M1-I65, M1-Y64, M1-I63, M1-S62, M1-V61, M1-A60, M1-N59, M1-R58, M1-R57, M1-M56, M1-R55, M1-C54, M1-G53, M1-L52, M1-L51, M1-W50, M1-L49, M1-V48, M1-V47, M1-A46, M1-N45, M1-G44, M1-T43, M1-L42, M1-A41, M1-V40, M1-L39, M1-S38, M1-V37, M1-I36, M1-C35, M1-T34, M1-L33, M1-G32, M1-T31, M1-F30, M1-S29, M1-L28, M1-T27, M1-Q26, M1-K25, M1-Y24, M1-C23, M1-P22, M1-T21, M1-E20, M1-E19, M1-R18, M1-G17, M1-N16, M1-I15, M1-P14, M1-T13, M1-L12, M1-E11, M1-T10, M1-G9, M1-L8, and/or M1-V7 of SEQ ID NO:20. Polynucleotide sequences encoding these polypeptides are also provided. The present invention also encompasses the use of these C-terminal Gene 7 deletion polypeptides as immunogenic and/or antigenic epitopes as described elsewhere herein.

The Gene 7 polypeptides of the present invention were determined to comprise several phosphorylation sites based upon the Motif algorithm (Genetics Computer Group, Inc.). The phosphorylation of such sites may regulate some biological activity of the Gene 7 polypeptide. For example, phosphorylation at specific sites may be involved in regulating the proteins ability to associate or bind to other molecules (e.g., proteins, ligands, substrates, DNA, etc.). In the present case, phosphorylation may modulate the ability of the Gene 7 polypeptide to associate with other polypeptides, particularly cognate ligand for Gene 7, or its ability to modulate certain cellular signal pathways.

The Gene 7 polypeptide was predicted to comprise two PKC phosphorylation sites using the Motif algorithm (Genetics Computer Group, Inc.). In vivo, protein kinase C exhibits a preference for the phosphorylation of serine or threonine residues. The PKC phosphorylation sites have the following consensus pattern: [ST]-x-[RK], where S or T represents the site of phosphorylation and ‘x’ an intervening amino acid residue. Additional information regarding PKC phosphorylation sites can be found in Woodget J. R., Gould K. L., Hunter T., Eur. J. Biochem. 161:177-184(1986), and Kishimoto A., Nishiyama K., Nakanishi H., Uratsuji Y., Nomura H., Takeyama Y., Nishizuka Y., J. Biol. Chem. 260:12492-12499(1985); which are hereby incorporated by reference herein.

In preferred embodiments, the following PKC phosphorylation site polypeptides are encompassed by the present invention: LSTVSTERCLSVL (SEQ ID NO:94), and/or YFFVGSFRKQWRL (SEQ ID NO:95). Polynucleotides encoding these polypeptides are also provided. The present invention also encompasses the use of the Gene 1, 2, 3, 4, 5, 6, 7, 8, 9, 10, 11, 12, or 13 PKC phosphorylation site polypeptides as immunogenic and/or antigenic epitopes as described elsewhere herein. The Gene 7 polypeptide was predicted to comprise three casein kinase II phosphorylation sites using the Motif algorithm (Genetics Computer Group, Inc.). Casein kinase II (CK-2) is a protein serine/threonine kinase whose activity is independent of cyclic nucleotides and calcium. CK-2 phosphorylates many different proteins. The substrate specificity [1] of this enzyme can be summarized as follows: (1) Under comparable conditions Ser is favored over Thr.; (2) An acidic residue (either Asp or Glu) must be present three residues from the C-terminal of the phosphate acceptor site; (3) Additional acidic residues in positions +1, +2, +4, and +5 increase the phosphorylation rate. Most physiological substrates have at least one acidic residue in these positions; (4) Asp is preferred to Glu as the provider of acidic determinants; and (5) A basic residue at the N-terminal of the acceptor site decreases the phosphorylation rate, while an acidic one will increase it.

A consensus pattern for casein kinase II phosphorylations site is as follows: [ST]-x(2)-[DE], wherein ‘x’ represents any amino acid, and S or T is the phosphorylation site.

Additional information specific to casein kinase II phosphorylation sites may be found in reference to the following publication: Pinna L. A., Biochim. Biophys. Acta 1054:267-284(1990); which is hereby incorporated herein in its entirety.

In preferred embodiments, the following casein kinase II phosphorylation site polypeptide is encompassed by the present invention: LSLLLSILEGKFCG (SEQ ID NO:96), CGFLFSDGDSGWCQ (SEQ ID NO:97), and/or LELSGSRLEQ (SEQ ID NO:98). Polynucleotides encoding these polypeptides are also provided. The present invention also encompasses the use of this casein kinase II phosphorylation site polypeptide as an immunogenic and/or antigenic epitope as described elsewhere herein.

The Gene 7 polypeptide has been shown to comprise one glycosylation site according to the Motif algorithm (Genetics Computer Group, Inc.). As discussed more specifically herein, protein glycosylation is thought to serve a variety of functions including: augmentation of protein folding, inhibition of protein aggregation, regulation of intracellular trafficking to organelles, increasing resistance to proteolysis, modulation of protein antigenicity, and mediation of intercellular adhesion.

Asparagine glycosylation sites have the following consensus pattern, N-{P}-[ST]-{P}, wherein N represents the glycosylation site. However, it is well known that that potential N-glycosylation sites are specific to the consensus sequence Asn-Xaa-Ser/Thr. However, the presence of the consensus tripeptide is not sufficient to conclude that an asparagine residue is glycosylated, due to the fact that the folding of the protein plays an important role in the regulation of N-glycosylation. It has been shown that the presence of proline between Asn and Ser/Thr will inhibit N-glycosylation; this has been confirmed by a recent statistical analysis of glycosylation sites, which also shows that about 50% of the sites that have a proline C-terminal to Ser/Thr are not glycosylated. Additional information relating to asparagine glycosylation may be found in reference to the following publications, which are hereby incorporated by reference herein: Marshall R. D., Annu. Rev. Biochem. 41:673-702(1972); Pless D. D., Lennarz W. J., Proc. Natl. Acad. Sci. U.S.A. 74:134-138(1977); Bause E., Biochem. J. 209:331-336(1983); Gavel Y., von Heijne G., Protein Eng. 3:433-442(1990); and Miletich J. P., Broze G. J. Jr., J. Biol. Chem. 265:11397-11404(1990).

In preferred embodiments, the following asparagine glycosylation site polypeptides are encompassed by the present invention: VLSSLNSSANPIIY (SEQ ID NO:99). Polynucleotides encoding these polypeptides are also provided. The present invention also encompasses the use of this Gene 7 asparagine glycosylation site polypeptide as an immunogenic and/or antigenic epitope as described elsewhere herein.

The Gene 7 polypeptide was predicted to comprise three N-myristoylation sites using the Motif algorithm (Genetics Computer Group, Inc.). An appreciable number of eukaryotic proteins are acylated by the covalent addition of myristate (a C14-saturated fatty acid) to their N-terminal residue via an amide linkage. The sequence specificity of the enzyme responsible for this modification, myristoyl CoA:protein N-myristoyl transferase (NMT), has been derived from the sequence of known N-myristoylated proteins and from studies using synthetic peptides. The specificity seems to be the following: i.) The N-terminal residue must be glycine; ii.) In position 2, uncharged residues are allowed; iii.) Charged residues, proline and large hydrophobic residues are not allowed; iv.) In positions 3 and 4, most, if not all, residues are allowed; v.) In position 5, small uncharged residues are allowed (Ala, Ser, Thr, Cys, Asn and Gly). Serine is favored; and vi.) In position 6, proline is not allowed.

A consensus pattern for N-myristoylation is as follows: G-{EDRKHPFYW}-x(2)-[STAGCN]-{P}, wherein ‘x’ represents any amino acid, and G is the N-myristoylation site.

Additional information specific to N-myristoylation sites may be found in reference to the following publication: Towler D. A., Gordon J. I., Adams S. P., Glaser L., Annu. Rev. Biochem. 57:69-99(1988); and Grand R. J. A., Biochem. J. 258:625-638(1989); which is hereby incorporated herein in its entirety.

In preferred embodiments, the following N-myristoylation site polypeptides are encompassed by the present invention: FMVLCGSSLALLVRIL (SEQ ID NO:100), LCGSRGLPLTRLYLTI (SEQ ID NO:101), and/or VFLLCGLPFGIQWFLI (SEQ ID NO:102). Polynucleotides encoding these polypeptides are also provided. The present invention also encompasses the use of these N-myristoylation site polypeptides as immunogenic and/or antigenic epitopes as described elsewhere herein.

The Gene 7 polypeptide has been shown to comprise one leucine zipper site according to the Motif algorithm (Genetics Computer Group, Inc.). Leucine zipper sites have been proposed to explain how some eukaryotic gene regulatory proteins work. The leucine zipper consists of a periodic repetition of leucine residues at every seventh position over a distance covering eight helical turns. The segments containing these periodic arrays of leucine residues seem to exist in an alpha-helical conformation. The leucine side chains extending from one alpha-helix interact with those from a similar alpha helix of a second polypeptide, facilitating dimerization; the structure formed by cooperation of these two regions forms a coiled coil. The leucine zipper pattern is present in many gene regulatory proteins, such as i.) the CCATT-box and enhancer binding protein (C/EBP); ii) the cAMP response element (CRE) binding proteins (CREB, CRE-BP1, ATFs); the Jun/AP1 family of transcription factors; iv.) the yeast general control protein GCN4; v.) the fos oncogene, and the fos-related proteins fra-1 and fos B; vi.) the C-myc, L-myc and N-myc oncogenes; and vii.) the octamer-binding transcription factor 2 (Oct-2/OTF-2). Leucine zipper motifs have the following concensus pattern: L-x(6)-L-x(6)-L-x(6)-L, wherein ‘x’ represents any amino acid. Additional information relating to leucine zipper motifs may be found in reference to the following publications, which are hereby incorporated by reference herein: Landschulz W. H., Johnson P. F., McKnight S. L., Science 240:1759-1764(1988); Busch S. J., Sassone-Corsi P., Trends Genet. 6:36-40(1990); and/or O'Shea E. K., Rutkowski R., Kim P. S., Science 243:538-542(1989).

In preferred embodiments, the following leucine zipper site polypeptide is encompassed by the present invention: CGSRGLPLTRLYLTILLTVLVFLLCGLPFGIQ (SEQ ID NO:103). Polynucleotides encoding these polypeptides are also provided. The present invention also encompasses the use of this Gene 7 leucine zipper site polypeptide as an immunogenic and/or antigenic epitope as described elsewhere herein.

Moreover, in confirmation of Gene 7 representing a novel GPCR, the Gene 7 polypeptide was predicted to comprise a G-protein coupled receptor motif using the Motif algorithm (Genetics Computer Group, Inc.). G-protein coupled receptors (also called R7G) are an extensive group of hormones, neurotransmitters, odorants and light receptors which transduce extracellular signals by interaction with guanine nucleotide-binding (G) proteins. Some examples of receptors that belong to this family are provided as follows: 5-hydroxytryptamine (serotonin) 1A to 1F, 2A to 2C, 4, SA, 5B, 6 and 7, Acetylcholine, muscarinic-type, M1 to M5, Adenosine A1, A2A, A2B and A3, Adrenergic alpha-1A to -1C; alpha-2A to -2D; beta-1 to -3, Angiotensin II types I and II, Bombesin subtypes 3 and 4, Bradykinin B1 and B2, c3a and C5a anaphylatoxin, Cannabinoid CB1 and CB2, Chemokines C—C CC-CKR-1 to CC-CKR-8, Chemokines C-X-C CXC-CKR-1 to CXC-CKR-4, Cholecystokinin-A and cholecystokinin-B/gastrin, Dopamine D1 to D5, Endothelin ET-a and ET-b, fMet-Leu-Phe (fMLP) (N-formyl peptide), Follicle stimulating hormone (FSH-R), Galanin, Gastrin-releasing peptide (GRP-R), Gonadotropin-releasing hormone (GNRH-R), Histamine H1 and H2 (gastric receptor I), Lutropin-choriogonadotropic hormone (LSH-R), Melanocortin MC1R to MC5R, Melatonin, Neuromedin B (NMB-R), Neuromedin K (NK-3R), Neuropeptide Y types 1 to 6, Neurotensin (NT-R), Octopamine (tyramine) from insects, Odorants, Opioids delta-, kappa- and mu-types, Oxytocin (OT-R), Platelet activating factor (PAF-R), Prostacyclin, Prostaglandin D2, Prostaglandin E2, EP1 to EP4 subtypes, Prostaglandin F2, Purinoreceptors (ATP), Somatostatin types 1 to 5, Substance-K (NK-2R), Substance-P (NK-1R), Thrombin, Thromboxane A2, Thyrotropin (TSH-R), Thyrotropin releasing factor (TRH-R), Vasopressin V1a, V1b and V2, Visual pigments (opsins and rhodopsin), Proto-oncogene mas, Caenorhabditis elegans putative receptors C06G4.5, C38C10.1, C43C3.2, T27D1.3 and ZC84.4, Three putative receptors encoded in the genome of cytomegalovirus: US27, US28, and UL33., ECRF3, a putative receptor encoded in the genome of herpesvirus saimiri.

The structure of all GPCRs are thought to be identical. They have seven hydrophobic regions, each of which most probably spans the membrane. The N-terminus is located on the extracellular side of the membrane and is often glycosylated, while the C-terminus is cytoplasmic and generally phosphorylated. Three extracellular loops alternate with three intracellular loops to link the seven transmembrane regions. Most, but not all of these receptors, lack a signal peptide. The most conserved parts of these proteins are the transmembrane regions and the first two cytoplasmic loops. A conserved acidic-Arg-aromatic triplet is present in the N-terminal extremity of the second cytoplasmic loop and could be implicated in the interaction with G proteins.

The putative consensus sequence for GPCRs comprises the conserved triplet and also spans the major part of the third transmembrane helix, and is as follows: [GSTALIVMFYWC]-[GSTANCPDE]-{EDPKRH}-x(2)-[LIVMNQGA]-x(2)-[LIVMFT]-[GSTANC]-[LIVMFYWSTAC]-[DENH]-R-[FYWCSH]-x(2)-[LIVM], where “X” represents any amino acid.

Additional information relating to G-protein coupled receptors may be found in reference to the following publications: Strosberg A. D., Eur. J. Biochem. 196:1-10(1991); Kerlavage A. R., Curr. Opin. Struct. Biol. 1:394-401(1991); Probst W. C., Snyder L. A., Schuster D. I., Brosius J., Sealfon S. C., DNA Cell Biol. 11:1-20(1992); Savarese T. M., Fraser C. M., Biochem. J. 283:1-9(1992); Branchek T., Curr. Biol. 3:315-317(1993); Stiles G. L., J. Biol. Chem. 267:6451-6454(1992); Friell T., Kobilka B. K., Lefkowitz R. J., Caron M. G., Trends Neurosci. 11:321-324(1988); Stevens C. F., Curr. Biol. 1:20-22(1991); Sakurai T., Yanagisawa M., Masaki T., Trends Pharmacol. Sci. 13:103-107(1992); Salesse R., Remy J. J., Levin J. M., Jallal B., Garnier J., Biochimie 73:109-120(1991); Lancet D., Ben-Arie N., Curr. Biol. 3:668-674(1993); Uhl G. R., Childers S., Pasternak G., Trends Neurosci. 17:89-93(1994); Barnard E. A., Burnstock G., Webb T. E., Trends Pharmacol. Sci. 15:67-70(1994); Applebury M. L., Hargrave P. A., Vision Res. 26:1881-1895(1986); Attwood T. K., Eliopoulos E. E., Findlay J. B. C., Gene 98:153-159(1991); http://www.gcrdb.uthscsa.edu/; and http://swift.embl-heidelberg.de/7m/.

In preferred embodiments, the following G-protein coupled receptors signature polypeptide is encompassed by the present invention: TCAYLAGLSMLSTVSTERCLSVLWPIW (SEQ ID NO:104). Polynucleotides encoding this polypeptide are also provided. The present invention also encompasses the use of the Gene 7 G-protein coupled receptors signature polypeptide as immunogenic and/or antigenic epitopes as described elsewhere herein.

Many polynucleotide sequences, such as EST sequences, are publicly available and accessible through sequence databases. Some of these sequences are related to SEQ ID NO:7 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides consisting of a nucleotide sequence described by the general formula of a-b, where a is any integer between 1 to 1983 of SEQ ID NO:7, b is an integer between 15 to 1997, where both a and b correspond to the positions of nucleotide residues shown in SEQ ID NO:7, and where b is greater than or equal to a+14.

In one embodiment, a Gene 7 polypeptide comprises a portion of the amino sequence depicted in FIGS. 13A-C and/or FIG. 14. In another embodiment, a Gene 7 polypeptide comprises at least 4, 5, 6, 7, 8, 9, 10, 11, 12, 13, 14, 15, 16, 17, 18, 19 or 20 amino acids of the amino sequence depicted in FIGS. 13A-C and/or FIG. 14. In further embodiments, the following Gene 7 polypeptide fragments are specifically excluded from the present invention: VSTERCLSVLWPIWYRC (SEQ ID NO:169); HLSAVVCVLLWALSLL (SEQ ID NO:170); ADFLFLCFQIINCLVYLSNFFCSISINFPSFFTTVMTCAYLAGLSMLSTVSTERC LSVLWPIWYRCRRPRHLSAVVCVLLWALSLLLSILEGKFCGFLFSDGD (SEQ ID NO:171); TILLTVLVFLLCGLPFGIQ (SEQ ID NO:172); LNSSANPIIYFFVGSFR (SEQ ID NO:173); MDSTIPVLGTELTPINGREETPCYKQTLSFTGLTCIVSLVALTGNAVVLWLLGC RMRRNAVSIYILNLVAADFLFL (SEQ ID NO:174); FDFITAAWLIFLFMVLCGSSLALLVRILCGSRGLPLTRLYLTILLTVLV (SEQ ID NO:175); LLCGLPFGIQWFLILWIWKDSDVLFCHIHPVSVVLSSLNSSANPIIYFFVGSFRK QWR (SEQ ID NO:176); PILKLALQRALQDIAEVD (SEQ ID NO:177); LTPINGREETPCYKQTLSFT (SEQ ID NO:178); and/or LTGNAVVLWLLGCRMRRNAVSIYILNL (SEQ ID NO:179).

Features of the Polypeptide Encoded by Gene No:10

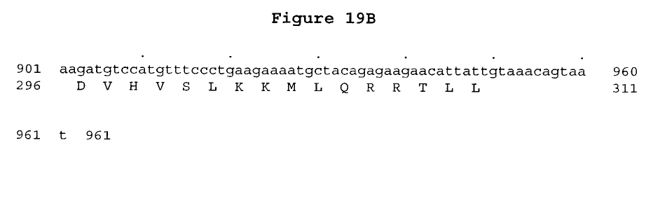

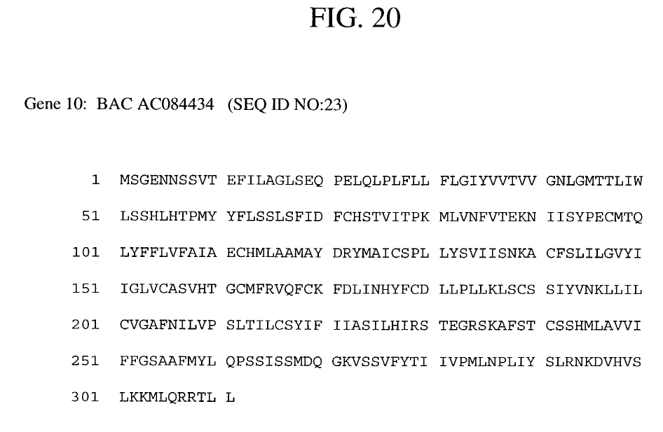

The determined nucleotide sequence of the Gene 10 cDNA in FIGS. 19A-B (SEQ ID NO:10) contains an open reading frame encoding a protein of about 311 amino acid residues, with a deduced molecular weight of about 34.9 kDa. The amino acid sequence of the predicted Gene 10 polypeptide is shown in FIGS. 19A-B and in FIG. 20 (SEQ ID NO:23).

The Gene 10 polypeptide was predicted to comprise seven transmembrane domains (TM1 to TM7) located from about amino acid 25 to about amino acid 50 (TM1; SEQ ID NO:105); from about amino acid 61 to about amino acid 83 (TM2;

SEQ ID NO:106); from about amino acid 97 to about amino acid 120 (TM3; SEQ ID NO: 107); from about amino acid 140 to about amino acid 158 (TM4; SEQ ID NO: 108); from about amino acid 197 to about amino acid 226 (TM5; SEQ ID NO: 109); from about amino acid 244 to about amino acid 265 (TM6; SEQ ID NO:110); and/or from about amino acid 273 to about amino acid 292 (TM7; SEQ ID NO:11I) of SEQ ID NO:23 (FIGS. 19A-B and FIG. 20). In this context, the term “about” may be construed to mean 1, 2, 3, 4, 5, 6, 7, 8, 9, or 10 amino acids beyond the N-Terminus and/or C-terminus of the above referenced transmembrane domain polypeptides.

In preferred embodiments, the following transmembrane domain polypeptides are encompassed by the present invention: LPLFLLFLGIYVVTVVGNLGMTTLIW (SEQ ID NO:105), YFLSSLSFIDFCHSTVITPKMLV (SEQ ID NO:106), CMTQLYFFLVFAIAECHMLAAMAY (SEQ ID NO:107), ACFSLILGVYIIGLVCASV (SEQ ID NO:108), LLILCVGAFNILVPSLTILCSYIFIIASIL (SEQ ID NO:109), HMLAVVIFFGSAAFMYLQPSSI (SEQ ID NO:110), and/or VSSVFYTIIVPMLNPLIYSL (SEQ ID NO:111). Polynucleotides encoding these polypeptides are also provided. The present invention also encompasses the use of these Gene 10 transmembrane domain polypeptides as immunogenic and/or antigenic epitopes as described elsewhere herein.

In preferred embodiments, the present invention encompasses the use of N-terminal deletions, C-terminal deletions, or any combination of N-terminal and C-terminal deletions of any one or more of the Gene 10 TM1 thru TM7 transmembrane domain polypeptides as antigenic and/or immunogenic epitopes.

In preferred embodiments, the present invention also encompasses the use of N-terminal deletions, C-terminal deletions, or any combination of N-terminal and C-terminal deletions of any one or more of the amino acids intervening (i.e., GPCR extracellular or intracellular loops) any pair of Gene 10 TM1 thru TM7 transmembrane domain polypeptides, and/or the amino acids intervening any pair of the Gene 10 TM1 thru TM7 transmembrane domain polypeptides themselves, as antigenic and/or immunogenic epitopes.

The coding region of the Gene 10 polynucleotide is predicted to be from nucleotide 18 to nucleotide 950 of SEQ ID NO:10 as shown in FIGS. 19A-B, and the polypeptide corresponding to amino acids 1 thru 311 of SEQ ID NO:23. The present invention encompasses the polynucleotide encompassing the entire coding region of Gene 10.

Alternatively, the present invention encompasses a polynucleotide lacking the initiating start codon, in addition to, the resulting encoded polypeptide of Gene 10. Specifically, the present invention encompasses the polynucleotide corresponding to nucleotides 21 thru 950 of SEQ ID NO:10, and the polypeptide corresponding to amino acids 2 thru 311 of SEQ ID NO:23. Also encompassed are recombinant vectors comprising said encoding sequence, and host cells comprising said vector.

In preferred embodiments, the following N-terminal Gene 10 deletion polypeptides are encompassed by the present invention: M1-L311, S2-L311, G3-L311, E4-L311, N5-L311, N6-L311, S7-L311, S8-L311, V9-L311, T1O-L311, E11-L311, F12-L311, 113-L311, L14-L311, A15-L311, G16-L311, L17-L311, S18-L311, E19-L311, Q20-L311, P21-L311, E22-L311, L23-L311, Q24-L311, L25-L311, P26-L311, L27-L311, F28-L311, L29-L311, L30-L311, F3i-L311, L32-L311, G33-L311, I34-L311, Y35-L311, V36-L311, V37-L311, T38-L311, V39-L311, V40-L311, G41-L311, N42-L311, L43-L311, G44-L311, M45-L311, T46-L311, T47-L311, L48-L311, I49-L311, W50-L311, L51-L311, S52-L311, S53-L311, H54-L311, L55-L311, H56-L311, T57-L311, P58-L311, M59-L311, Y60-L311, Y61-L311, F62-L311, L63-L311, S64-L311, S65-L311, L66-L311, S67-L311, F68-L311, 169-L311, D70-L311, F71-L311, C72-L311, H73-L311, S74-L311, T75-L311, V76-L311, 177-L311, T78-L311, P79-L311, K80-L311, M81-L311, L82-L311, V83-L311, N84-L311, F85-L311, V86-L311, T87-L311, E88-L311, K89-L311, N90-L311, 191-L311, 192-L311, S93-L311, Y94-L311, P95-L311, E96-L311, C97-L311, M98-L311, T99-L311, Q100-L311, L101-L311, Y102-L311, F103-L311, F104-L311, L105-L311, V106-L311, F107-L311, A108-L311, I109-L311, A110-L311, E111-L311, C112-L311, H113-L311, M114-L311, L115-L311, A116-L311, A117-L311, M118-L311, A119-L311, Y120-L311, D121-L311, R122-L311, Y123-L311, M124-L311, A125-L311, I126-L311, C127-L311, S128-L311, P129-L311, L130-L311, L131-L311, Y132-L311, S133-L311, V134-L311, I135-L311, I136-L311, S137-L311, N138-L311, K139-L311, A140-L311, C141-L311, F142-L311, S143-L311, L144-L311, I145-L311, L146-L311, G147-L311, V148-L311, Y149-L311, I150-L311, I151-L311, G152-L311, L153-L311, V154-L311, C155-L311, A156-L311, S157-L311, V158-L311, H159-L311, T160-L311, G161-L311, C162-L311, M163-L311, F164-L311, R165-L311, V166-L311, Q167-L311, F168-L311, C169-L311, K170-L311, F171-L311, D172-L311, L173-L311, I174-L311, N175-L311, H176-L311, Y177-L311, F178-L311, C179-L311, D180-L311, L181-L311, L182-L311, P183-L311, L184-L311, L185-L311, K186-L311, L187-L311, S188-L311, C189-L311, S190-L311, S191-L311, I192-L311, Y193-L311, V194-L311, N195-L311, K196-L311, L197-L311, L198-L311, I199-L311, L200-L311, C201-L311, V202-L311, G203-L311, A204-L311, F205-L311, N206-L311, I207-L311, L208-L311, V209-L311, P210-L311, S211-L311, L212-L311, T213-L311, I214-L311, L215-L311, C216-L311, S217-L311, Y218-L311, I219-L311, F220-L311, I221-L311, I222-L311, A223-L311, S224-L311, I225-L311, L226-L311, H227-L311, I228-L311, R229-L311, S230-L311, T231-L311, E232-L311, G233-L311, R234-L311, S235-L311, K236-L311, A237-L311, F238-L311, S239-L311, T240-L311, C241-L311, S242-L311, S243-L311, H244-L311, M245-L311, L246-L311, A247-L311, V248-L311, V249-L311, I250-L311, F251-L311, F252-L311, G253-L311, S254-L311, A255-L311, A256-L311, F257-L311, M258-L311, Y259-L311, L260-L311, Q261-L311, P262-L311, S263-L311, S264-L311, I265-L311, S266-L311, S267-L311, M268-L311, D269-L311, Q270-L311, G271-L311, K272-L311, V273-L311, S274-L311, S275-L311, V276-L311, F277-L311, Y278-L311, T279-L311, I280-L311, I281-L311, V282-L311, P283-L311, M284-L311, L285-L311, N286-L311, P287-L311, L288-L311, I289-L311, Y290-L311, S291-L311, L292-L311, R293-L311, N294-L311, K295-L311, D296-L311, V297-L311, H298-L311, V299-L311, S300-L311, L301-L311, K302-L311, K303-L311, M304-L311, and/or L305-L311 of SEQ ID NO:23. Polynucleotide sequences encoding these polypeptides are also provided. The present invention also encompasses the use of these N-terminal Gene 10 deletion polypeptides as immunogenic and/or antigenic epitopes as described elsewhere herein.

In preferred embodiments, the following C-terminal Gene 10 deletion polypeptides are encompassed by the present invention: M1-L311, M1-L310, M1-T309, M1-R308, M1-R307, M1-Q306, M1-L305, M1-M304, M1-K303, M1-K302, M1-L301, M1-S300, M1-V299, M1-H298, M1-V297, M1-D296, M1-K295, M1-N294, M1-R293, M1-L292, M1-S291, M1-Y290, M1-I289, M1-L288, M1-P287, M1-N286, M1-L285, M1-M284, M1-P283, M1-V282, M1-I281, M1-I280, M1-T279, M1-Y278, M1-F277, M1-V276, M1-S275, M1-S274, M1-V273, M1-K272, M1-G271, M1-Q270, M1-D269, M1-M268, M1-S267, M1-S266, M1-1265, M1-S264, M1-S263, M1-P262, M1-Q261, M1-L260, M1-Y259, M1-M258, M1-F257, M1-A256, M1-A255, M1-S254, M1-G253, M1-F252, M1-F251, M1-I250, M1-V249, M1-V248, M1-A247, M1-L246, M1-M245, M1-H244, M1-S243, M1-S242, M1-C241, M1-T240, M1-S239, M1-F238, M1-A237, M1-K236, M1-S235, M1-R234, M1-G233, M1-E232, M1-T231, M1-S230, M1-R229, M1-I228, M1-H227, M1-L226, M1-I225, M1-S224, M1-A223, M1-I222, M1-I221, M1-F220, M1-1219, M1-Y218, M1-S217, M1-C216, M1-L215, M1-I214, M1-T213, M1-L212, M1-S211, M1-P210, M1-V209, M1-L208, M1-I207, M1-N206, M1-F205, M1-A204, M1-G203, M1-V202, M1-C201, M1-L200, M1-I199, M1-L198, M1-L197, M1-K196, M1-N195, M1-V194, M1-Y193, M1-I192, M1-S191, M1-S190, M1-C189, M1-S188, M1-L187, M1-K186, M1-L185, M1-L184, M1-P183, M1-L182, M1-L181, M1-D180, M1-C179, M1-F178, M1-Y177, M1-H176, M1-N175, M1-1174, M1-L173, M1-D172, M1-F171, M1-K170, M1-C169, M1-F168, M1-Q167, M1-V166, M1-R165, M1-F164, M1-M163, M1-C162, M1-G161, M1-T160, M1-H159, M1-V158, M1-S157, M1-A156, M1-C155, M1-V154, M1-L153, M1-G152, M1-I151, M1-I150, M1-Y149, M1-V148, M1-G147, M1-L146, M1-1145, M1-L144, M1-S143, M1-F142, M1-C141, M1-A140, M1-K139, M1-N138, M1-S137, M1-I136, M1-I135, M1-V134, M1-S133, M1-Y132, M1-L131, M1-L130, M1-P129, M1-S128, M1-C127, M1-I126, M1-A125, M1-M124, M1-Y123, M1-R122, M1-D121, M1-Y120, M1-A119, M1-M118, M1-A117, M1-A116, M1-L115, M1-M114, M1-H113, M1-C112, M1-E111, M1-A110, M1-I109, M1-A108, M1-F107, M1-V106, M1-L105, M1-F104, M1-F103, M1-Y102, M1-L101, M1-Q100, M1-T99, M1-M98, M1-C97, M1-E96, M1-P95, M1-Y94, M1-S93, M1-192, M1-191, M1-N90, M1-K89, M1-E88, M1-T87, M1-V86, M1-F85, M1-N84, M1-V83, M1-L82, M1-M81, M1-K80, M1-P79, M1-T78, M1-I77, M1-V76, M1-T75, M1-S74, M1-H73, M1-C72, M1-F71, M1-D70, M1-I69, M1-F68, M1-S67, M1-L66, M1-S65, M1-S64, M1-L63, M1-F62, M1-Y61, M1-Y60, M1-M59, M1-P58, M1-T57, M1-H56, M1-L55, M1-H54, M1-S53, M1-S52, M1-L51, M1-W50, M1-I49, M1-L48, M1-T47, M1-T46, M1-M45, M1-G44, M1-L43, M1-N42, M1-G41, M1-V40, M1-V39, M1-T38, M1-V37, M1-V36, M1-Y35, M1-I34, M1-G33, M1-L32, M1-F31, M1-L30, M1-L29, M1-F28, M1-L27, M1-P26, M1-L25, M1-Q24, M1-L23, M1-E22, M1-P21, M1-Q20, M1-E19, M1-S18, M1-L17, M1-G16, M1-A15, M1-L14, M1-I13, M1-F12, M1-E11, M1-T10, M1-V9, M1-S8, and/or M1-S7 of SEQ ID NO:23. Polynucleotide sequences encoding these polypeptides are also provided. The present invention also encompasses the use of these C-terminal Gene 10 deletion polypeptides as immunogenic and/or antigenic epitopes as described elsewhere herein.

The Gene 10 polypeptides of the present invention were determined to comprise several phosphorylation sites based upon the Motif algorithm (Genetics Computer Group, Inc.). The phosphorylation of such sites may regulate some biological activity of the Gene 10 polypeptide. For example, phosphorylation at specific sites may be involved in regulating the proteins ability to associate or bind to other molecules (e.g., proteins, ligands, substrates, DNA, etc.). In the present case, phosphorylation may modulate the ability of the Gene 10 polypeptide to associate with other polypeptides, particularly cognate ligand for Gene 10, or its ability to modulate certain cellular signal pathways.

The Gene 10 polypeptide was predicted to comprise five PKC phosphorylation sites using the Motif algorithm (Genetics Computer Group, Inc.). In vivo, protein kinase C exhibits a preference for the phosphorylation of serine or threonine residues. The PKC phosphorylation sites have the following consensus pattern: [ST]-x-[RK], where S or T represents the site of phosphorylation and ‘x’ an intervening amino acid residue. Additional information regarding PKC phosphorylation sites can be found in Woodget J. R., Gould K. L., Hunter T., Eur. J. Biochem. 161:177-184(1986), and Kishimoto A., Nishiyama K., Nakanishi H., Uratsuji Y., Nomura H., Takeyama Y., Nishizuka Y., J. Biol. Chem. 260:12492-12499(1985); which are hereby incorporated by reference herein.

In preferred embodiments, the following PKC phosphorylation site polypeptides are encompassed by the present invention: HSTVITPKMLVNF (SEQ ID NO:112), LVNFVTEKNIISY (SEQ ID NO:113), YSVIISNKACFSL (SEQ ID NO:114), NPLIYSLRNKDVH (SEQ ID NO:115), and/or KDVHVSLKKMLQR (SEQ ID NO:116). Polynucleotides encoding these polypeptides are also provided. The present invention also encompasses the use of the Gene 1, 2, 3, 4, 5, 6, 7, 8, 9, 10, 11, 12, or 13 PKC phosphorylation site polypeptides as immunogenic and/or antigenic epitopes as described elsewhere herein.

The Gene 10 polypeptide was predicted to comprise four casein kinase II phosphorylation sites using the Motif algorithm (Genetics Computer Group, Inc.). Casein kinase II (CK-2) is a protein serine/threonine kinase whose activity is independent of cyclic nucleotides and calcium. CK-2 phosphorylates many different proteins. The substrate specificity [1] of this enzyme can be summarized as follows: (1) Under comparable conditions Ser is favored over Thr.; (2) An acidic residue (either Asp or Glu) must be present three residues from the C-terminal of the phosphate acceptor site; (3) Additional acidic residues in positions +1, +2, +4, and +5 increase the phosphorylation rate. Most physiological substrates have at least one acidic residue in these positions; (4) Asp is preferred to Glu as the provider of acidic determinants; and (5) A basic residue at the N-terminal of the acceptor site decreases the phosphorylation rate, while an acidic one will increase it.

A consensus pattern for casein kinase II phosphorylations site is as follows: [ST]-x(2)-[DE], wherein ‘x’ represents any amino acid, and S or T is the phosphorylation site.

Additional information specific to casein kinase II phosphorylation sites may be found in reference to the following publication: Pinna L. A., Biochim. Biophys. Acta 1054:267-284(1990); which is hereby incorporated herein in its entirety.

In preferred embodiments, the following casein kinase II phosphorylation site polypeptide is encompassed by the present invention: GENNSSVTEFILAG (SEQ ID NO:117), FLSSLSFIDFCHST (SEQ ID NO:118), EKNIISYPECMTQL (SEQ ID NO: 119), and/or QPSSISSMDQGKVS (SEQ ID NO:120). Polynucleotides encoding these polypeptides are also provided. The present invention also encompasses the use of this casein kinase II phosphorylation site polypeptide as an immunogenic and/or antigenic epitope as described elsewhere herein.

The Gene 10 polypeptide has been shown to comprise two glycosylation sites according to the Motif algorithm (Genetics Computer Group, Inc.). As discussed more specifically herein, protein glycosylation is thought to serve a variety of functions including: augmentation of protein folding, inhibition of protein aggregation, regulation of intracellular trafficking to organelles, increasing resistance to proteolysis, modulation of protein antigenicity, and mediation of intercellular adhesion.

Asparagine glycosylation sites have the following consensus pattern, N-{P}-[ST]-{P}, wherein N represents the glycosylation site. However, it is well known that that potential N-glycosylation sites are specific to the consensus sequence Asn-Xaa-Ser/Thr. However, the presence of the consensus tripeptide is not sufficient to conclude that an asparagine residue is glycosylated, due to the fact that the folding of the protein plays an important role in the regulation of N-glycosylation. It has been shown that the presence of proline between Asn and Ser/Thr will inhibit N-glycosylation; this has been confirmed by a recent statistical analysis of glycosylation sites, which also shows that about 50% of the sites that have a proline C-terminal to Ser/Thr are not glycosylated. Additional information relating to asparagine glycosylation may be found in reference to the following publications, which are hereby incorporated by reference herein: Marshall R. D., Annu. Rev. Biochem. 41:673-702(1972); Pless D. D., Lennarz W. J., Proc. Natl. Acad. Sci. U.S.A. 74:134-138(1977); Bause E., Biochem. J. 209:331-336(1983); Gavel Y., von Heijne G., Protein Eng. 3:433-442(1990); and Miletich J. P., Broze G. J. Jr., J. Biol. Chem. 265:11397-11404(1990).

In preferred embodiments, the following asparagine glycosylation site polypeptides are encompassed by the present invention: MSGENNSSVTEFI (SEQ ID NO: 121), and/or MSGENNSSVTEFIL (SEQ ID NO:122). Polynucleotides encoding these polypeptides are also provided. The present invention also encompasses the use of these Gene 10 asparagine glycosylation site polypeptide as immunogenic and/or antigenic epitopes as described elsewhere herein.

The Gene 10 polypeptide was predicted to comprise one N-myristoylation sites using the Motif algorithm (Genetics Computer Group, Inc.). An appreciable number of eukaryotic proteins are acylated by the covalent addition of myristate (a C14-saturated fatty acid) to their N-terminal residue via an amide linkage. The sequence specificity of the enzyme responsible for this modification, myristoyl CoA:protein N-myristoyl transferase (NMT), has been derived from the sequence of known N-myristoylated proteins and from studies using synthetic peptides. The specificity seems to be the following: i.) The N-terminal residue must be glycine; ii.) In position 2, uncharged residues are allowed; iii.) Charged residues, proline and large hydrophobic residues are not allowed; iv.) In positions 3 and 4, most, if not all, residues are allowed; v.) In position 5, small uncharged residues are allowed (Ala, Ser, Thr, Cys, Asn and Gly). Serine is favored; and vi.) In position 6, proline is not allowed.

A consensus pattern for N-myristoylation is as follows: G-{EDRKHPFYW}-x(2)-[STAGCN]-{P}, wherein ‘x’ represents any amino acid, and G is the N-myristoylation site.

Additional information specific to N-myristoylation sites may be found in reference to the following publication: Towler D. A., Gordon J. I., Adams S. P., Glaser L., Annu. Rev. Biochem. 57:69-99(1988); and Grand R. J. A., Biochem. J. 258:625-638(1989); which is hereby incorporated herein in its entirety.

In preferred embodiments, the following N-myristoylation site polypeptides are encompassed by the present invention: GVYIIGLVCASVHTGC (SEQ ID NO: 123). Polynucleotides encoding these polypeptides are also provided. The present invention also encompasses the use of these N-myristoylation site polypeptides as immunogenic and/or antigenic epitopes as described elsewhere herein.

Moreover, in confirmation of Gene 10 representing a novel GPCR, the Gene 10 polypeptide was predicted to comprise a G-protein coupled receptor motif using the Motif algorithm (Genetics Computer Group, Inc.). G-protein coupled receptors (also called R7G) are an extensive group of hormones, neurotransmitters, odorants and light receptors which transduce extracellular signals by interaction with guanine nucleotide-binding (G) proteins. Some examples of receptors that belong to this family are provided as follows: 5-hydroxytryptamine (serotonin) 1A to 1F, 2A to 2C, 4, 5A, 5B, 6 and 7, Acetylcholine, muscarinic-type, M1 to M5, Adenosine A1, A2A, A2B and A3, Adrenergic alpha-1A to —IC; alpha-2A to −2D; beta-1 to -3, Angiotensin II types I and II, Bombesin subtypes 3 and 4, Bradykinin B 1 and B2, c3a and C5a anaphylatoxin, Cannabinoid CB1 and CB2, Chemokines C-C CC-CKR-1 to CC-CKR-8, Chemokines C-X-C CXC-CKR-1 to CXC-CKR-4, Cholecystokinin-A and cholecystokinin-B/gastrin, Dopamine D1 to D5, Endothelin ET-a and ET-b, fMet-Leu-Phe (fMLP) (N-formyl peptide), Follicle stimulating hormone (FSH-R), Galanin, Gastrin-releasing peptide (GRP-R), Gonadotropin-releasing hormone (GNRH-R), Histamine H1 and H2 (gastric receptor I), Lutropin-choriogonadotropic hormone (LSH-R), Melanocortin MC1R to MC5R, Melatonin, Neuromedin B (NMB-R), Neuromedin K (NK-3R), Neuropeptide Y types 1 to 6, Neurotensin (NT-R), Octopamine (tyramine) from insects, Odorants, Opioids delta-, kappa- and mu-types, Oxytocin (OT-R), Platelet activating factor (PAF-R), Prostacyclin, Prostaglandin D2, Prostaglandin E2, EP1 to EP4 subtypes, Prostaglandin F2, Purinoreceptors (ATP), Somatostatin types 1 to 5, Substance-K (NK-2R), Substance-P (NK-1R), Thrombin, Thromboxane A2, Thyrotropin (TSH-R), Thyrotropin releasing factor (TRH-R), Vasopressin V1a, V1b and V2, Visual pigments (opsins and rhodopsin), Proto-oncogene mas, Caenorhabditis elegans putative receptors C06G4.5, C38C10. 1, C43C3.2, T27D1.3 and ZC84.4, Three putative receptors encoded in the genome of cytomegalovirus: US27, US28, and UL33., ECRF3, a putative receptor encoded in the genome of herpesvirus saimiri.

The structure of all GPCRs are thought to be identical. They have seven hydrophobic regions, each of which most probably spans the membrane. The N-terminus is located on the extracellular side of the membrane and is often glycosylated, while the C-terminus is cytoplasmic and generally phosphorylated. Three extracellular loops alternate with three intracellular loops to link the seven transmembrane regions. Most, but not all of these receptors, lack a signal peptide. The most conserved parts of these proteins are the transmembrane regions and the first two cytoplasmic loops. A conserved acidic-Arg-aromatic triplet is present in the N-terminal extremity of the second cytoplasmic loop and could be implicated in the interaction with G proteins.

The putative consensus sequence for GPCRs comprises the conserved triplet and also spans the major part of the third transmembrane helix, and is as follows: [GSTALIVMFYWC]-[GSTANCPDE]-{EDPKRH}-x(2)-[LIVMNQGA]-x(2)-[LIVMFT]-[GSTANC]-[LIVMFYWSTAC]-[DENH]-R-[FYWCSH]-x(2)-[LIVM], where “X” represents any amino acid.

Additional information relating to G-protein coupled receptors may be found in reference to the following publications: Strosberg A. D., Eur. J. Biochem. 196:1-10(1991); Kerlavage A. R., Curr. Opin. Struct. Biol. 1:394-401(1991); Probst W. C., Snyder L. A., Schuster D. I., Brosius J., Sealfon S. C., DNA Cell Biol. 11:1-20(1992); Savarese T. M., Fraser C. M., Biochem. J. 283:1-9(1992); Branchek T., Curr. Biol. 3:315-317(1993); Stiles G. L., J. Biol. Chem. 267:6451-6454(1992); Friell T., Kobilka B. K., Lefkowitz R. J., Caron M. G., Trends Neurosci. 11:321-324(1988); Stevens C. F., Curr. Biol. 1:20-22(1991); Sakurai T., Yanagisawa M., Masaki T., Trends Pharmacol. Sci. 13:103-107(1992); Salesse R., Remy J. J., Levin J. M., Jallal B., Garnier J., Biochimie 73:109-120(1991); Lancet D., Ben-Arie N., Curr. Biol. 3:668-674(1993); Uhl G. R., Childers S., Pasternak G., Trends Neurosci. 17:89-93(1994); Barnard E. A., Burnstock G., Webb T. E., Trends Pharmacol. Sci. 15:67-70(1994); Applebury M. L., Hargrave P. A., Vision Res. 26:1881-1895(1986); Attwood T. K., Eliopoulos E. E., Findlay J. B. C., Gene 98:153-159(1991); http://www.gcrdb.uthscsa.edu/; and http://swift.embl-heidelberg.de/7tm/.

In preferred embodiments, the following G-protein coupled receptors signature polypeptide is encompassed by the present invention: LVFAIAECHMLAAMAYDRYMAICSPLL (SEQ ID NO:123). Polynucleotides encoding this polypeptide are also provided. The present invention also encompasses the use of the Gene 10 G-protein coupled receptors signature polypeptide as immunogenic and/or antigenic epitopes as described elsewhere herein.

Many polynucleotide sequences, such as EST sequences, are publicly available and accessible through sequence databases. Some of these sequences are related to SEQ ID NO:10 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides consisting of a nucleotide sequence described by the general formula of a-b, where a is any integer between 1 to 947 of SEQ ID NO:10, b is an integer between 15 to 961, where both a and b correspond to the positions of nucleotide residues shown in SEQ ID NO:10, and where b is greater than or equal to a+14.

In one embodiment, a Gene 10 polypeptide comprises a portion of the amino sequence depicted in FIGS. 19A-B and/or FIG. 20. In another embodiment, a Gene 10 polypeptide comprises at least 4, 5, 6, 7, 8, 9, 10, 11, 12, 13, 14, 15, 16, 17, 18, 19 or 20 amino acids of the amino sequence depicted in FIGS. 19A-B and/or FIG. 20. In further embodiments, the following Gene 10 polypeptide fragments are specifically excluded from the present invention: INHYFCDLLPLL (SEQ ID NO:180); SKAFSTCSSH (SEQ ID NO:181); NPLIYSLRNKDV (SEQ ID NO:182); STEGRSKAFSTCSSH (SEQ ID NO:183); FFGSAAFMYLQPSS (SEQ ID NO:184); IVPMLNPLIYSLRNKDV (SEQ ID NO:185); SSMDQGKVSSVFY (SEQ ID NO:186); and/or VPMLNPLIYSLRNKDV (SEQ ID NO:187).

Features of the Polypeptide Encoded by Gene No:13

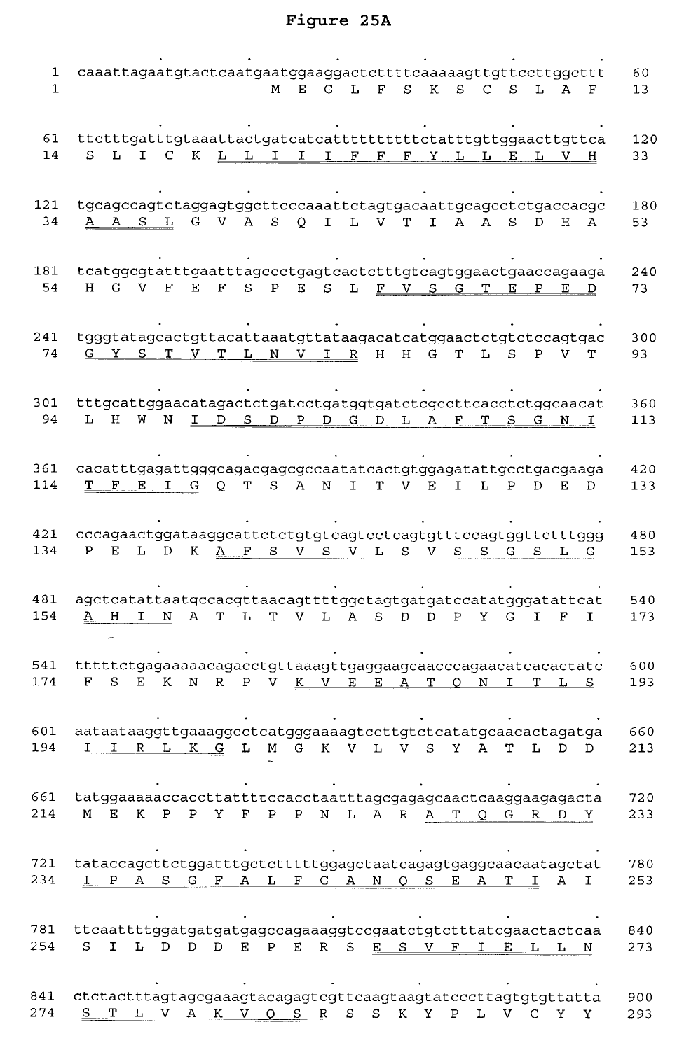

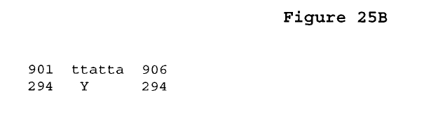

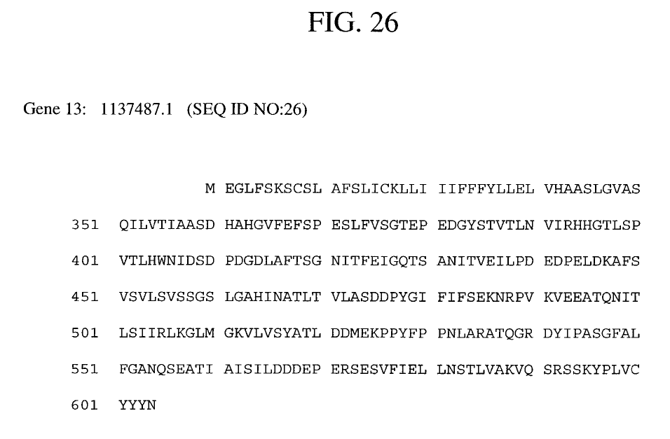

The determined nucleotide sequence of the Gene 13 (also referred to as Gene 13; GPCR-P20; and GPCR-P151) cDNA in FIGS. 25A-B (SEQ ID NO:13) contains an open reading frame encoding a protein of about 295 amino acid residues, with a deduced molecular weight of about 32.1 kDa. The amino acid sequence of the predicted Gene 13 polypeptide is shown in FIGS. 25A-B and in FIG. 26 (SEQ ID NO:26).

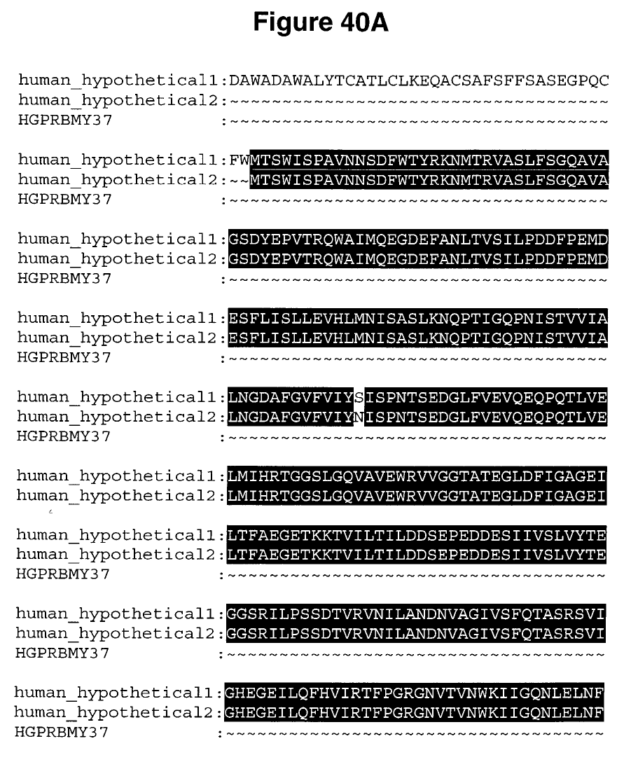

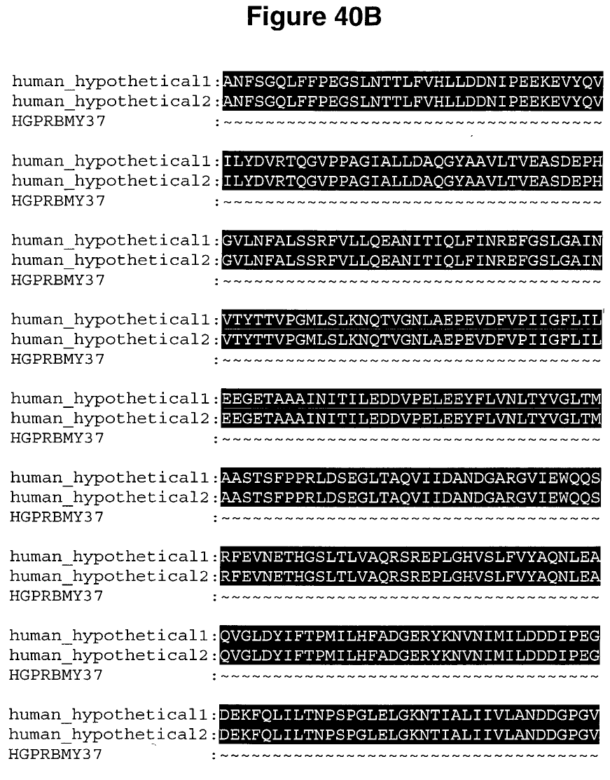

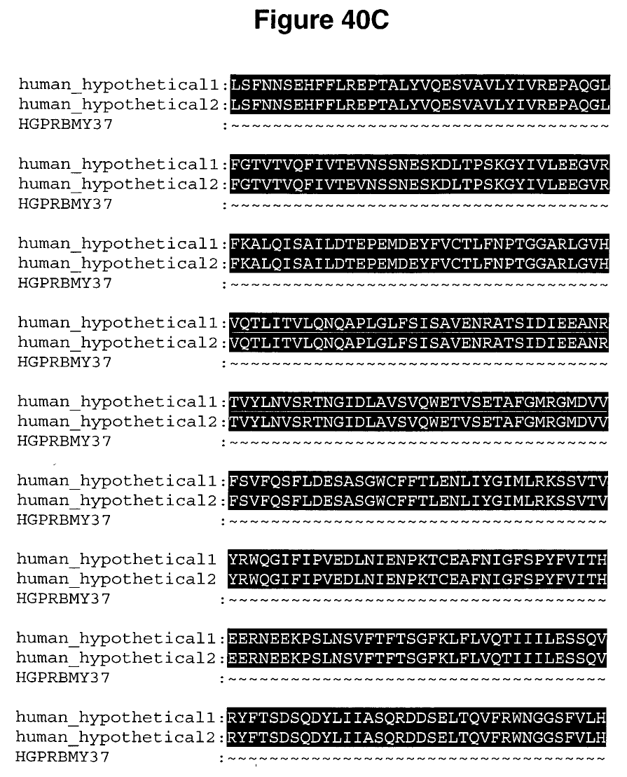

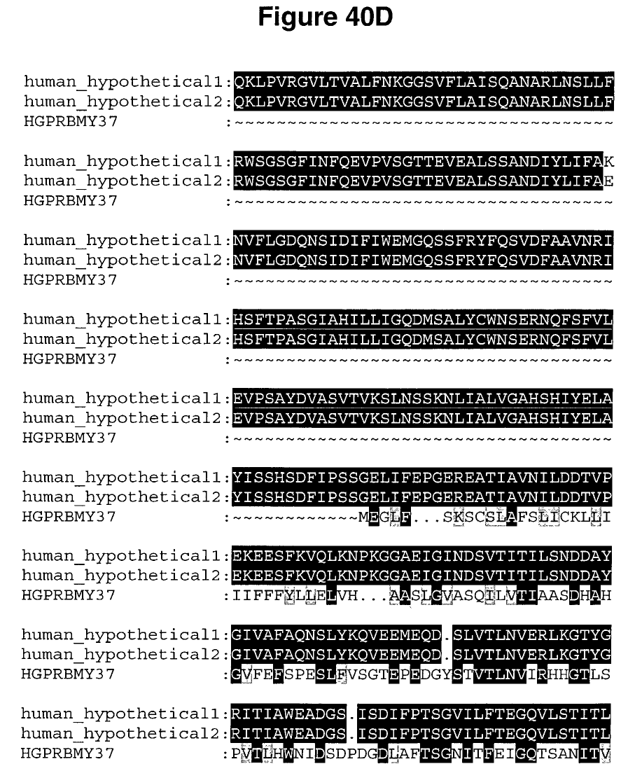

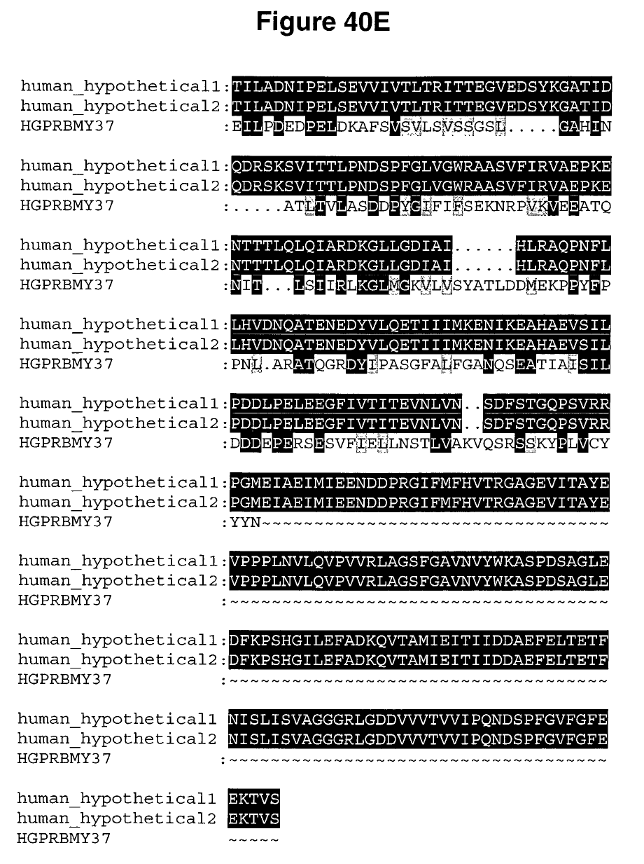

The Gene 13 polypeptide was determined to share 29.7% amino acid sequence identity and 41.6% amino acid sequence similarity with the human GPCR protein human_hypothetical 1 (SEQ ID NO:149); and to share 29.7% amino acid sequence identity and 41.6% amino acid sequence similarity with the human GPCR protein human_hypothetical 2 (SEQ ID NO:150) as shown in FIGS. 40A-40E.

The Gene 13 polypeptide was predicted to comprise seven transmembrane domains (TM1 to TM7) located from about amino acid 19 to about amino acid 37 (TM1; SEQ ID NO:125); from about amino acid 65 to about amino acid 84 (TM2; SEQ ID NO:126); from about amino acid 98 to about amino acid 118 (TM3; SEQ ID NO:127); from about amino acid 139 to about amino acid 157 (TM4; SEQ ID NO:128); from about amino acid 182 to about amino acid 199 (TM5; SEQ ID NO:129); from about amino acid 227 to about amino acid 251 (TM6; SEQ ID NO:130); and/or from about amino 265 to about amino acid 283 (TM7; SEQ ID NO: 131) of SEQ ID NO:26 (FIGS. 25A-B and FIG. 26). In this context, the term “about” may be construed to mean 1, 2, 3, 4, 5, 6, 7, 8, 9, or 10 amino acids beyond the N-Terminus and/or C-terminus of the above referenced transmembrane domain polypeptides.

In preferred embodiments, the following transmembrane domain polypeptides are encompassed by the present invention: LLIIIFFFYLLELVHAASL (SEQ ID NO: 125), FVSGTEPEDGYSTVTLNVIR (SEQ ID NO:126), IDSDPDGDLAFTSGNITFEIG (SEQ ID NO:127), AFSVSVLSVSSGSLGAHIN (SEQ ID NO:128), KVEEATQNITLSIIRLKG (SEQ ID NO:129), ATQGRDYIPASGFALFGANQSEATI (SEQ ID NO:130), and/or ESVFIELLNSTLVAKVQSR (SEQ ID NO:131). Polynucleotides encoding these polypeptides are also provided. The present invention also encompasses the use of these Gene 13 transmembrane domain polypeptides as immunogenic and/or antigenic epitopes as described elsewhere herein.

In preferred embodiments, the present invention encompasses the use of N-terminal deletions, C-terminal deletions, or any combination of N-terminal and C-terminal deletions of any one or more of the Gene 13 TM1 thru TM7 transmembrane domain polypeptides as antigenic and/or immunogenic epitopes.

In preferred embodiments, the present invention also encompasses the use of N-terminal deletions, C-terminal deletions, or any combination of N-terminal and C-terminal deletions of any one or more of the amino acids intervening (i.e., GPCR extracellular or intracellular loops) any pair of Gene 13 TM1 thru TM7 transmembrane domain polypeptides, and/or the amino acids intervening any pair of the Gene 13 TM1 thru TM7 transmembrane domain polypeptides themselves, as antigenic and/or immunogenic epitopes.

The coding region of the Gene 13 polynucleotide is predicted to be from nucleotide 23 to nucleotide 906 of SEQ ID NO:13 as shown in FIGS. 25A-B, and the polypeptide corresponding to amino acids 1 thru 295 of SEQ ID NO:26. The present invention encompasses the polynucleotide encompassing the entire coding region of Gene 13.

Alternatively, the present invention encompasses a polynucleotide lacking the initiating start codon, in addition to, the resulting encoded polypeptide of Gene 13. Specifically, the present invention encompasses the polynucleotide corresponding to nucleotides 26 thru 906 of SEQ ID NO:13, and the polypeptide corresponding to amino acids 2 thru 295 of SEQ ID NO:26. Also encompassed are recombinant vectors comprising said encoding sequence, and host cells comprising said vector.

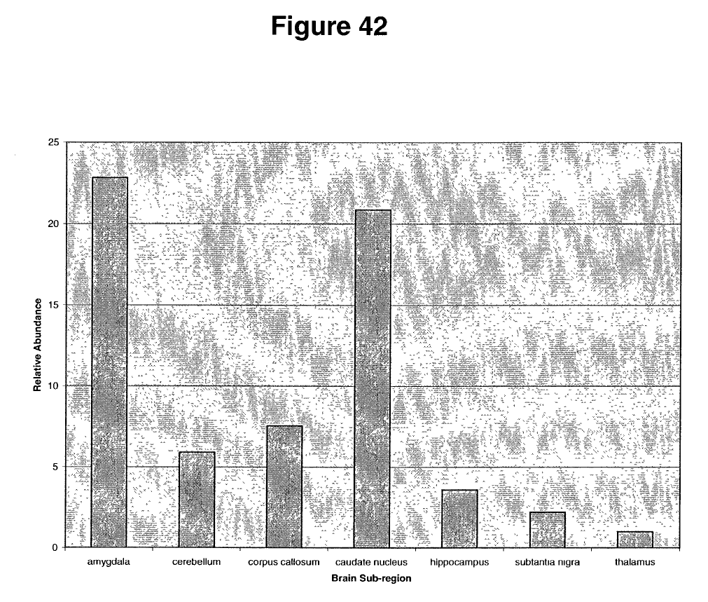

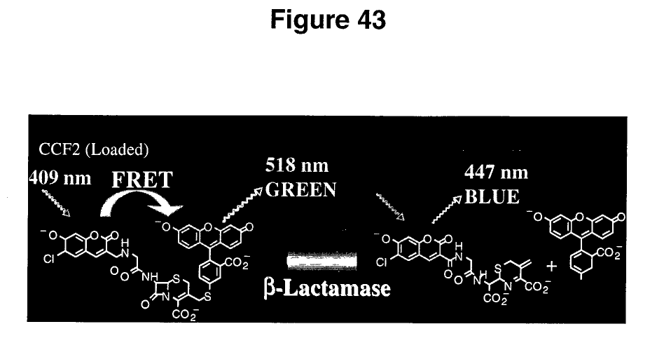

Expression profiling of Gene 13 indicates it is highly expressed in brain and pituitary (FIG. 42). In particular, Gene 13 was expressed in the following brain sub-regions: amygdala, caudate nucleus, corpus callosum, cerebellum, hippocampus, thalamus and subtantia nigra (FIG. 43). Gene 13 has also been found to be expressed in the heart, kidney, lung, pancreas, prostate, small intestine, spinal cord, testis and thymus.

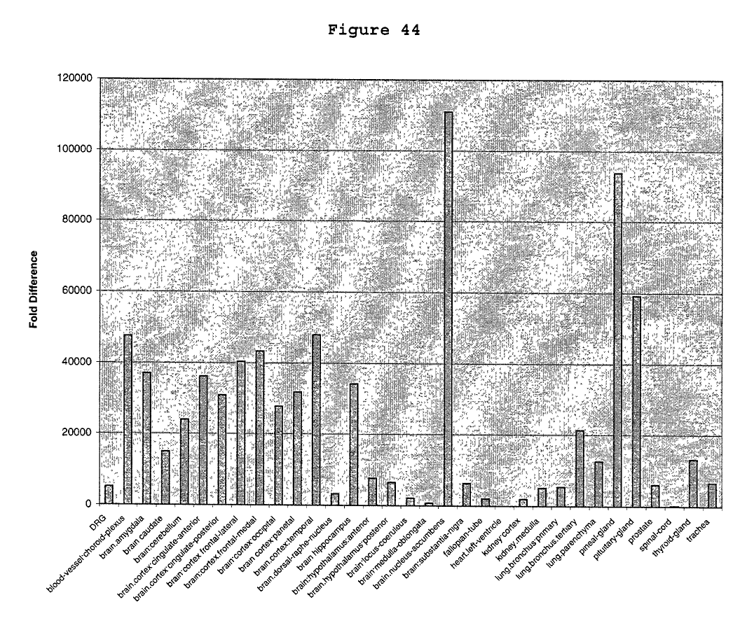

Expanded analysis of Gene 13 expression levels by TaqMan™ quantitative PCR (see FIG. 44) confirmed that the Gene 13 polypeptide is expressed in brain, and pituitary (FIG. 42). Gene 13 mRNA was expressed predominately in the in the nervous system, with lesser amounts found in the respiratory and endocrine systems. Specifically, Gene 13 was expressed at the highest steady state levels in the nucleus accumbens, followed by the pineal and pituitary gland. Expression of Gene 13 was also significantly expressed at near equal levels across the cortex, hippocampus, amygdala, and choroid plexus. Expression of Gene 13 was also significantly expressed to a lesser extent in in the caudate, the cerebellum and the hypothalamus.

Collectively the expression data suggests a role for HGRPBMY37 in neural processes that connect, either directly or indirectly, the nucleus accumbens and its ‘reward center’ functions which include, for example, the release of neurotransmitters such as dopamine, opioid peptides, serotonin, GABA and the pineal gland, best known for its involvement in the establishment and maintenance of circadian rhythms and the control of the sleep/wake cycle.

Polnucleotides and polypeptide of Gene 13, in addition to modulators thereof, are useful for detecting, treating, and/or ameliorating neural disorders, particularly those disorders that affect the nucleus accumbens, disorders that affect the brains ‘reward center’ function, neurotransmitter release disorders, disorders affecting the release of dopamine, disorders affecting the release of opioid peptides, disorders affecting the release of serotonin, disorders affecting the release of GABA, pineal gland disorders, disorders affecting the establishment of circadian rhythms, disorders affecting the maintenance of circadian rhythms, disorders affecting the control of the sleep/wake cycle.

The pituitary gland expression may suggest a link between Gene 13 and melatonin and pituitary hormone secretions, particularly those involved in oxytocin secretion during the neuroendocrine response to stressful stimuli. Gene 13 may also have a role in how the melatonin rhythm may affect other neuroendocrine functions such as the nocturnal pattern of hormone secretion, prolactin and cortisol release.

Polnucleotides and polypeptide of Gene 13, in addition to modulators thereof, are useful for detecting, treating, and/or ameliorating melatonin secretion disorders, pituitary hormone secretion disorders, oxytocin secretion disorders, disorders affecting neuroendocrine response to stressful stimuli, disorders affecting oxytocin secretion during neuroendocrine response to stressful stimuli, disorders affecting nocturnal patterns of hormone secretion, disorders affecting the nocturnal hormone secretion of prolactin, disorders affecting the nocturnal hormone secretion of cortisol, and/or disorders affecting the nocturnal hormone secretion of growth hormone.

Gene 13 expression throughout the cortex suggests involvement in the execution of functions concerned with the organization of behavior, memory and cognitive reasoning. These data suggest modulators of Gene 13 function may have utility in a variety of neuro-pathologies, including responses to stress, and propensity to develop addictive behaviors, as well as a vast number of neuroendocrine abnormalities including sleep disorders.

The Gene 13 polynucleotide and polypeptide, may be involved in a variety of diseases, disorders and conditions associated with Gene 13 activity, which include, but are not limited to, immune-related disorders, acute heart failure, hypotension, hypertension, endocrinal diseases, growth disorders, neuropathic pain, obesity, anorexia, HIV infections, cancers, bulimia, asthma, Parkinson's disease, osteoporosis, angina pectoris, myocardial infarction, psychotic, metabolic, cardiovascular and neurological disorders. More specifically, the present invention is concerned with modulation of the Gene 13 polynucleotide and encoded products, particularly in providing treatments and therapies for relevant diseases. Antagonizing or inhibiting the action of the Gene 13 polynucleotide and polypeptide is especially encompassed by the present invention.

The invention further relates to fragments and portions of the novel Gene 13 nucleic acid sequence and its encoded amino acid sequence (peptides and polypeptides). Preferably, the fragments and portions of the Gene 13 polypeptide are functional or active. The invention also provides methods of using the novel Gene 13 polynucleotide sequence and the encoded Gene 13 polypeptide for diagnosis, genetic screening and treatment of diseases, disorders, conditions, or syndromes associated with Gene 13 and Gene 13 activity and function. The Gene 13 polynucleotide and polypeptide, may be involved in a variety of diseases, disorders and conditions associated with Gene 13 activity, which include, but are not limited to, immune-related disorders, acute heart failure, hypotension, hypertension, endocrinal diseases, growth disorders, neuropathic pain, obesity, anorexia, HIV infections, cancers, bulimia, asthma, Parkinson's disease, osteoporosis, angina pectoris, myocardial infarction, psychotic, metabolic, cardiovascular and neurological disorders. Neurological or nervous system-related conditions are particularly relevant.

The Gene 13 polynucleotide and/or polypeptide of this invention are useful for diagnosing diseases related to over- or under-expression of the Gene 13 protein. For example, such Gene 13-associated diseases can be assessed by identifying mutations in the Gene 13 gene using Gene 13 probes or primers, or by determining Gene 13 protein or mRNA expression levels. A Gene 13 polypeptide is also useful for screening compounds which affect activity of the protein. The invention further encompasses the polynucleotide encoding the Gene 13 polypeptide and the use of the Gene 13 polynucleotide or polypeptide, or compositions thereof, in the screening, diagnosis, treatment, or prevention of disorders associated with aberrant or uncontrolled cellular growth and/or function, such as neoplastic diseases (for example, cancers and tumors). Gene 13 probes or primers can be used, for example, to screen for diseases associated with Gene 13.

The Gene 13 protein according to this invention may play a role in cell signaling, in cell cycle regulation, and/or in neurological disorders. The Gene 13 protein may further be involved in neoplastic, cardiovascular, and immunological disorders.

In one embodiment in accordance with the present invention, the novel Gene 13 protein may play a role in neoplastic disorders. An antagonist or inhibitor of the Gene 13 protein may be administered to an individual to prevent or treat a neoplastic disorder. Such disorders may include, but are not limited to, adenocarcinoma, leukemia, lymphoma, melanoma, myeloma, sarcoma, and teratocarcinoma, and particularly, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus. In a related aspect, an antibody which specifically binds to Gene 13 may be used directly as an antagonist or indirectly as a targeting or delivery mechanism for bringing a pharmaceutical agent to cells or tissue which express the Gene 13 polypeptide.

In yet another embodiment of the present invention, an antagonist or inhibitory agent of the Gene 13 polypeptide may be administered therapeutically to an individual to prevent or treat an immunological disorder. Such disorders may include, but are not limited to, AIDS, HIV infection, Addison's disease, adult respiratory distress syndrome, allergies, anemia, asthma, atherosclerosis, bronchitis, cholecystitis, Crohn's disease, ulcerative colitis, atopic dermatitis, dermatomyositis, diabetes mellitus, emphysema, erythema nodosum, atrophic gastritis, glomerulonephritis, gout, Graves' disease, hypereosinophilia, irritable bowel syndrome, lupus erythematosus, multiple sclerosis, myasthenia gravis, myocardial or pericardial inflammation, osteoarthritis, osteoporosis, pancreatitis, polymyositis, rheumatoid arthritis, scleroderma, Sjogren's syndrome, and autoimmune thyroiditis; complications of cancer, hemodialysis, extracorporeal circulation; viral, bacterial, fungal, parasitic, protozoal, and helminthic infections, trauma, and neurological disorders including, but not limited to, akathesia, Alzheimer's disease, amnesia, amyotrophic lateral sclerosis, bipolar disorder, catatonia, cerebral neoplasms, dementia, depression, Down's syndrome, tardive dyskinesia, dystonias, epilepsy, Huntington's disease, multiple sclerosis, Parkinson's disease, paranoid psychoses, schizophrenia, and Tourette's disorder.

A preferred method of treating a Gene 13 associated disease, disorder, syndrome, or condition in a mammal comprises administration of a modulator, preferably an inhibitor or antagonist, of a Gene 13 polypeptide or homologue of the invention, in an amount effective to treat, reduce, and/or ameliorate the symptoms incurred by the Gene 13-associated disease, disorder, syndrome, or condition. In some instances, an agonist or enhancer of a Gene 13 polypeptide or homologue of the invention is administered in an amount effective to treat and/or ameliorate the symptoms incurred by a Gene 13-related disease, disorder, syndrome, or condition. In other instances, the administration of a novel Gene 13 polypeptide or homologue thereof pursuant to the present invention is envisioned for administration to treat a Gene 13 associated disease.

In a preferred embodiment, polynucleotides and polypeptides of Gene 13, in addition to modulators thereof, are useful for treating, preventing, and/or ameliorating the following diseases or disorders, brain development disorders, peripheral nervous system disorders, audiogenic epilepsy, epilepsy, embryonal neurogenesis disorders, eye development disorders, and neuronal excitability disorders.

Nervous system diseases, disorders, and/or conditions, which can be treated, prevented, and/or diagnosed with the compositions of the invention (e.g., polypeptides, polynucleotides, and/or agonists or antagonists), include, but are not limited to, nervous system injuries, and diseases, disorders, and/or conditions which result in either a disconnection of axons, a diminution or degeneration of neurons, or demyelination. Nervous system lesions which may be treated, prevented, and/or diagnosed in a patient (including human and non-human mammalian patients) according to the invention, include but are not limited to, the following lesions of either the central (including spinal cord, brain) or peripheral nervous systems: (1) ischemic lesions, in which a lack of oxygen in a portion of the nervous system results in neuronal injury or death, including cerebral infarction or ischemia, or spinal cord infarction or ischemia; (2) traumatic lesions, including lesions caused by physical injury or associated with surgery, for example, lesions which sever a portion of the nervous system, or compression injuries; (3) malignant lesions, in which a portion of the nervous system is destroyed or injured by malignant tissue which is either a nervous system associated malignancy or a malignancy derived from non-nervous system tissue; (4) infectious lesions, in which a portion of the nervous system is destroyed or injured as a result of infection, for example, by an abscess or associated with infection by human immunodeficiency virus, herpes zoster, or herpes simplex virus or with Lyme disease, tuberculosis, syphilis; (5) degenerative lesions, in which a portion of the nervous system is destroyed or injured as a result of a degenerative process including but not limited to degeneration associated with Parkinson's disease, Alzheimer's disease, Huntington's chorea, or amyotrophic lateral sclerosis (ALS); (6) lesions associated with nutritional diseases, disorders, and/or conditions, in which a portion of the nervous system is destroyed or injured by a nutritional disorder or disorder of metabolism including but not limited to, vitamin B12 deficiency, folic acid deficiency, Wernicke disease, tobacco-alcohol amblyopia, Marchiafava-Bignami disease (primary degeneration of the corpus callosum), and alcoholic cerebellar degeneration; (7) neurological lesions associated with systemic diseases including, but not limited to, diabetes (diabetic neuropathy, Bell's palsy), systemic lupus erythematosus, carcinoma, or sarcoidosis; (8) lesions caused by toxic substances including alcohol, lead, or particular neurotoxins; and (9) demyelinated lesions in which a portion of the nervous system is destroyed or injured by a demyelinating disease including, but not limited to, multiple sclerosis, human immunodeficiency virus-associated myelopathy, transverse myelopathy or various etiologies, progressive multifocal leukoencephalopathy, and central pontine myclinolysis.

In a preferred embodiment, the polypeptides, polynucleotides, or agonists or antagonists of the invention are used to protect neural cells from the damaging effects of cerebral hypoxia. According to this embodiment, the compositions of the invention are used to treat, prevent, and/or diagnose neural cell injury associated with cerebral hypoxia. In one aspect of this embodiment, the polypeptides, polynucleotides, or agonists or antagonists of the invention are used to treat, prevent, and/or diagnose neural cell injury associated with cerebral ischemia. In another aspect of this embodiment, the polypeptides, polynucleotides, or agonists or antagonists of the invention are used to treat, prevent, and/or diagnose neural cell injury associated with cerebral infarction. In another aspect of this embodiment, the polypeptides, polynucleotides, or agonists or antagonists of the invention are used to treat, prevent, and/or diagnose or prevent neural cell injury associated with a stroke. In a further aspect of this embodiment, the polypeptides, polynucleotides, or agonists or antagonists of the invention are used to treat, prevent, and/or diagnose neural cell injury associated with a heart attack.

The compositions of the invention which are useful for treating or preventing a nervous system disorder may be selected by testing for biological activity in promoting the survival or differentiation of neurons. For example, and not by way of limitation, compositions of the invention which elicit any of the following effects may be useful according to the invention: (1) increased survival time of neurons in culture; (2) increased sprouting of neurons in culture or in vivo; (3) increased production of a neuron-associated molecule in culture or in vivo, e.g., choline acetyltransferase or acetylcholinesterase with respect to motor neurons; or (4) decreased symptoms of neuron dysfunction in vivo. Such effects may be measured by any method known in the art. In preferred, non-limiting embodiments, increased survival of neurons may routinely be measured using a method set forth herein or otherwise known in the art, such as, for example, the method set forth in Arakawa et al. (J. Neurosci. 10:3507-3515 (1990)); increased sprouting of neurons may be detected by methods known in the art, such as, for example, the methods set forth in Pestronk et al. (Exp. Neurol. 70:65-82 (1980)) or Brown et al. (Ann. Rev. Neurosci. 4:17-42 (1981)); increased production of neuron-associated molecules may be measured by bioassay, enzymatic assay, antibody binding, Northern blot assay, etc., using techniques known in the art and depending on the molecule to be measured; and motor neuron dysfunction may be measured by assessing the physical manifestation of motor neuron disorder, e.g., weakness, motor neuron conduction velocity, or functional disability.

In specific embodiments, motor neuron diseases, disorders, and/or conditions that may be treated, prevented, and/or diagnosed according to the invention include, but are not limited to, diseases, disorders, and/or conditions such as infarction, infection, exposure to toxin, trauma, surgical damage, degenerative disease or malignancy that may affect motor neurons as well as other components of the nervous system, as well as diseases, disorders, and/or conditions that selectively affect neurons such as amyotrophic lateral sclerosis, and including, but not limited to, progressive spinal muscular atrophy, progressive bulbar palsy, primary lateral sclerosis, infantile and juvenile muscular atrophy, progressive bulbar paralysis of childhood (Fazio-Londe syndrome), poliomyelitis and the post polio syndrome, and Hereditary Motorsensory Neuropathy (Charcot-Marie-Tooth Disease).

In preferred embodiments, the following N-terminal Gene 13 deletion polypeptides are encompassed by the present invention: M1-N295, E2-N295, G3-N295, L4-N295, F5-N295, S6-N295, K7-N295, S8-N295, C9-N295, S10-N295, L11-N295, A12-N295, F13-N295, S14-N295, L15-N295, I16-N295, C17-N295, K18-N295, L19-N295, L20-N295, I21-N295, I22-N295, I23-N295, F24-N295, F25-N295, F26-N295, Y27-N295, L28-N295, L29-N295, E30-N295, L31-N295, V32-N295, H33-N295, A34-N295, A35-N295, S36-N295, L37-N295, G38-N295, V39-N295, A40-N295, S41-N295, Q42-N295, I43-N295, L44-N295, V45-N295, T46-N295, I47-N295, A48-N295, A49-N295, S50-N295, D51-N295, H52-N295, A53-N295, H54-N295, G55-N295, V56-N295, F57-N295, E58-N295, F59-N295, S60-N295, P61-N295, E62-N295, S63-N295, L64-N295, F65-N295, V66-N295, S67-N295, G68-N295, T69-N295, E70-N295, P71-N295, E72-N295, D73-N295, G74-N295, Y75-N295, S76-N295, T77-N295, V78-N295, T79-N295, L80-N295, N81-N295, V82-N295, I83-N295, R84-N295, H85-N295, H86-N295, G87-N295, T88-N295, L89-N295, S90-N295, P91-N295, V92-N295, T93-N295, L94-N295, H95-N295, W96-N295, N97-N295, I98-N295, D99-N295, S100-N295, D101-N295, P102-N295, D103-N295, G104-N295, D105-N295, L106-N295, A107-N295, F108-N295, T109-N295, S110-N295, G111-N295, N112-N295, I113-N295, T114-N295, F115-N295, E116-N295, I117-N295, G118-N295, Q119-N295, T120-N295, S121-N295, A122-N295, N123-N295, I124-N295, T125-N295, V126-N295, E127-N295L, I128-N295, L129-N295, P130-N295, D131-N295, E132-N295, D133-N295, P134-N295, E135-N295, L136-N295, D137-N295, K138-N295, A139-N295, F140-N295, S141-N295, V142-N295, S143-N295, V144-N295, L145-N295, S146-N295, V147-N295, S148-N295, S149-N295, G150-N295, S151-N295, L152-N295, G153-N295, A154-N295, H155-N295, I156-N295, N157-N295, A158-N295, T159-N295, L160-N295, T161-N295, V162-N295, L163-N295, A164-N295, S165-N295, D166-N295, D167-N295, P168-N295, Y169-N295, G170-N295, I171-N295, F172-N295, I173-N295, F174-N295, S175-N295, E176-N295, K177-N295, N178-N295, R179-N295, P180-N295, V181-N295, K182-N295, V183-N295, E184-N295, E185-N295, A186-N295, T187-N295, Q188-N295, N189-N295, I190-N295, T191-N295, L192-N295, S193-N295, I194-N295, I195-N295, R196-N295, L197-N295, K198-N295, G199-N295, L200-N295, M201-N295, G202-N295, K203-N295, V204-N295, L205-N295, V206-N295, S207-N295, Y208-N295, A209-N295, T210-N295, L211-N295, D212-N295, D213-N295, M214-N295, E215-N295, K216-N295, P217-N295, P218-N295, Y219-N295, F220-N295, P221-N295, P222-N295, N223-N295, L224-N295, A225-N295, R226-N295, A227-N295, T228-N295, Q229-N295, G230-N295, R231-N295, D232-N295, Y233-N295, I234-N295, P235-N295, A236-N295, S237-N295, G238-N295, F239-N295, A240-N295, L241-N295, F242-N295, G243-N295, A244-N295, N245-N295, Q246-N295, S247-N295, E248-N295, A249-N295, T250-N295, I251-N295, A252-N295, I253-N295, S254-N295, I255-N295, L256-N295, D257-N295, D258-N295, D259-N295, E260-N295, P261-N295, E262-N295, R263-N295, S264-N295, E265-N295, S266-N295, V267-N295, F268-N295, I269-N295, E270-N295, L271-N295, L272-N295, N273-N295, S274-N295, T275-N295, L276-N295, V277-N295, A278-N295, K279-N295, V280-N295, Q281-N295, S282-N295, R283-N295, S284-N295, S285-N295, K286-N295, Y287-N295, P288-N295, and/or L289-N295 of SEQ ID NO:26. Polynucleotide sequences encoding these polypeptides are also provided. The present invention also encompasses the use of these N-terminal Gene 13 deletion polypeptides as immunogenic and/or antigenic epitopes as described elsewhere herein.

In preferred embodiments, the following C-terminal Gene 13 deletion polypeptides are encompassed by the present invention: M1-N295, M1-Y294, M1-Y293, M1-Y292, M1-C291, M1-V290, M1-L289, M1-P288, M1-Y287, M1-K286, M1-S285, M1-S284, M1-R283, M1-S282, M1-Q281, M1-V280, M1-K279, M1-A278, M1-V277, M1-L276, M1-T275, M1-S274, M1-N273, M1-L272, M1-L271, M1-E270, M1-I269, M1-F268, M1-V267, M1-S266, M1-E265, M1-S264, M1-R263, M1-E262, M1-P261, M1-E260, M1-D259, M1-D258, M1-D257, M1-L256, M1-I255, M1-S254, M1-I253, M1-A252, M1-I251, M1-T250, M1-A249, M1-E248, M1-S247, M1-Q246, M1-N245, M1-A244, M1-G243, M1-F242, M1-L241, M1-A240, M1-F239, M1-G238, M1-S237, M1-A236, M1-P235, M1-1234, M1-Y233, M1-D232, M1-R231, M1-G230, M1-Q229, M1-T228, M1-A227, M1-R226, M1-A225, M1-L224, M1-N223, M1-P222, M1-P221, M1-F220, M1-Y219, M1-P218, M1-P217, M1-K216, M1-E215, M1-M214, M1-D213, M1-D212, M1-L211, M1-T210, M1-A209, M1-Y208, M1-S207, M1-V206, M1-L205, M1-V204, M1-K203, M1-G202, M1-M201, M1-L200, M1-G199, M1-K198, M1-L197, M1-R196, M1-I195, M1-I194, M1-S193, M1-L192, M1-T191, M1-I190, M1-N189, M1-Q188, M1-T187, M1-A186, M1-E185, M1-E184, M1-V183, M1-K182, M1-V181, M1-P180, M1-R179, M1-N178, M1-K177, M1-E176, M1-S175, M1-F174, M1-I173, M1-F172, M1-I171, M1-G170, M1-Y169, M1-P168, M1-D167, M1-D166, M1-S165, M1-A164, M1-L163, M1-V162, M1-T161, M1-L160, M1-T159, M1-A158, M1-N157, M1-I156, M1-H155, M1-A154, M1-G153, M1-L152, M1-S151, M1-G150, M1-S149, M1-S148, M1-V147, M1-S146, M1-L145, M1-V144, M1-S143, M1-V142, M1-S141, M1-F140, M1-A139, M1-K138, M1-D137, M1-L136, M1-E135, M1-P134, M1-D133, M1-E132, M1-D131, M1-P130, M1-L129, M1-I128, M1-E127, M1-V126, M1-T125, M1I124, M1-N123, M1-A122, M1-S121, M1-T120, M1-Q119, M1-G118, M1-I117, M1-E116, M1-F115, M1-T114, M1-I113, M1-N112, M1-G111, M1-S110, M1-T109, M1-F108, M1-A107, M1-L106, M1-D105, M1-G104, M1-D103, M1-P102, M1-D101, M1-S100, M1-D99, M1-198, M1-N97, M1-W96, M1-H95, M1-L94, M1-T93, M1V92, M1-P91, M1-S90, M1-L89, M1-T88, M1-G87, M1-H86, M1-H85, M1-R84, M1-I83, M1-V82, M1-N81, M1-L80, M1-T79, M1-V78, M1-T77, M1-S76, M1-Y75, M1-G74, M1-D73, M1-E72, M1-P71, M1-E70, M1-T69, M1-G68, M1-S67, M1-V66, M1-F65, M1-L64, M1-S63, M1-E62, M1-P61, M1-S60, M1-F59, M1-E58, M1-F57, M1-V56, M1-G55, M1-H54, M1-A53, M1-H52, M1-D51, M1-S50, M1-A49, M1-A48, M1-I47, M1-T46, M1-V45, M1-L44, M1-I43, M1-Q42, M1-S41, M1-A40, M1-V39, M1-G38, M1-L37, M1-S36, M1-A35, M1-A34, M1-H33, M1-V32, M1-L31, M1-E30, M1-L29, M1-L28, M1-Y27, M1-F26, M1-F25, M1-F24, M1-I23, M1-I22, M1-121, M1-L20, M1-L19, M1-K18, M1-C17, M1-I16, M1-L15, M1-S14, M1-F13, M1-A12, M1-L11, M1-S10, M1-C9, M1-S8, and/or M1-K7 of SEQ ID NO:26. Polynucleotide sequences encoding these polypeptides are also provided. The present invention also encompasses the use of these C-terminal Gene 13 deletion polypeptides as immunogenic and/or antigenic epitopes as described elsewhere herein.

The Gene 13 polypeptides of the present invention were determined to comprise several phosphorylation sites based upon the Motif algorithm (Genetics Computer Group, Inc.). The phosphorylation of such sites may regulate some biological activity of the Gene 13 polypeptide. For example, phosphorylation at specific sites may be involved in regulating the proteins ability to associate or bind to other molecules (e.g., proteins, ligands, substrates, DNA, etc.). In the present case, phosphorylation may modulate the ability of the Gene 13 polypeptide to associate with other polypeptides, particularly cognate ligand for Gene 13, or its ability to modulate certain cellular signal pathways.

The Gene 13 polypeptide was predicted to comprise two PKC phosphorylation sites using the Motif algorithm (Genetics Computer Group, Inc.). In vivo, protein kinase C exhibits a preference for the phosphorylation of serine or threonine residues. The PKC phosphorylation sites have the following consensus pattern: [ST]-x-[RK], where S or T represents the site of phosphorylation and ‘x’ an intervening amino acid residue. Additional information regarding PKC phosphorylation sites can be found in Woodget J. R., Gould K. L., Hunter T., Eur. J. Biochem. 161:177-184(1986), and Kishimoto A., Nishiyama K., Nakanishi H., Uratsuji Y., Nomura H., Takeyama Y., Nishizuka Y., J. Biol. Chem. 260:12492-12499(1985); which are hereby incorporated by reference herein.

In preferred embodiments, the following PKC phosphorylation site polypeptides are encompassed by the present invention: GIFIFSEKNRPVK (SEQ ID NO:132), and/or KVQSRSSKYPLVC (SEQ ID NO:133). Polynucleotides encoding these polypeptides are also provided. The present invention also encompasses the use of the Gene 1, 2, 3, 4, 5, 6, 7, 8, 9, 10, 11, 12, or 13 PKC phosphorylation site polypeptides as immunogenic and/or antigenic epitopes as described elsewhere herein.

The Gene 13 polypeptide was predicted to comprise five casein kinase II phosphorylation sites using the Motif algorithm (Genetics Computer Group, Inc.). Casein kinase II (CK-2) is a protein serine/threonine kinase whose activity is independent of cyclic nucleotides and calcium. CK-2 phosphorylates many different proteins. The substrate specificity [1] of this enzyme can be summarized as follows: (1) Under comparable conditions Ser is favored over Thr.; (2) An acidic residue (either Asp or Glu) must be present three residues from the C-terminal of the phosphate acceptor site; (3) Additional acidic residues in positions +1, +2, +4, and +5 increase the phosphorylation rate. Most physiological substrates have at least one acidic residue in these positions; (4) Asp is preferred to Glu as the provider of acidic determinants; and (5) A basic residue at the N-terminal of the acceptor site decreases the phosphorylation rate, while an acidic one will increase it.

A consensus pattern for casein kinase II phosphorylations site is as follows: [ST]-x(2)-[DE], wherein ‘x’ represents any amino acid, and S or T is the phosphorylation site.

Additional information specific to casein kinase II phosphorylation sites may be found in reference to the following publication: Pinna L. A., Biochim. Biophys. Acta 1054:267-284(1990); which is hereby incorporated herein in its entirety.

In preferred embodiments, the following casein kinase II phosphorylation site polypeptide is encompassed by the present invention: ESLFVSGTEPEDGY (SEQ ID NO:134), LFVSGTEPEDGYST (SEQ ID NO:135), HWNIDSDPDGDLAF (SEQ ID NO:136), LVSYATLDDMEKPP (SEQ ID NO:137), and/or ATIAISILDDDEPE (SEQ ID NO:138). Polynucleotides encoding these polypeptides are also provided. The present invention also encompasses the use of this casein kinase II phosphorylation site polypeptide as an immunogenic and/or antigenic epitope as described elsewhere herein.

The Gene 13 polypeptide has been shown to comprise six glycosylation sites according to the Motif algorithm (Genetics Computer Group, Inc.). As discussed more specifically herein, protein glycosylation is thought to serve a variety of functions including: augmentation of protein folding, inhibition of protein aggregation, regulation of intracellular trafficking to organelles, increasing resistance to proteolysis, modulation of protein antigenicity, and mediation of intercellular adhesion.

Asparagine glycosylation sites have the following consensus pattern, N-{P}-[ST]-{P}, wherein N represents the glycosylation site. However, it is well known that that potential N-glycosylation sites are specific to the consensus sequence Asn-Xaa-Ser/Thr. However, the presence of the consensus tripeptide is not sufficient to conclude that an asparagine residue is glycosylated, due to the fact that the folding of the protein plays an important role in the regulation of N-glycosylation. It has been shown that the presence of proline between Asn and Ser/Thr will inhibit N-glycosylation; this has been confirmed by a recent statistical analysis of glycosylation sites, which also shows that about 50% of the sites that have a proline C-terminal to Ser/Thr are not glycosylated. Additional information relating to asparagine glycosylation may be found in reference to the following publications, which are hereby incorporated by reference herein: Marshall R. D., Annu. Rev. Biochem. 41:673-702(1972); Pless D. D., Lennarz W. J., Proc. Natl. Acad. Sci. U.S.A. 74:134-138(1977); Bause E., Biochem. J. 209:331-336(1983); Gavel Y., von Heijne G., Protein Eng. 3:433-442(1990); and Miletich J.P., Broze G. J. Jr., J. Biol. Chem. 265:11397-11404(1990).

In preferred embodiments, the following asparagine glycosylation site polypeptides are encompassed by the present invention: AFTSGNITFEIGQT (SEQ ID NO:139), GQTSANITVEILPD (SEQ ID NO:140), LGAHINATLTVLAS (SEQ ID NO:141), EEATQNITLSIIRL (SEQ ID NO:142), ALFGANQSEATIAI (SEQ ID NO:143), and/or FIELLNSTLVAKVQ (SEQ ID NO:144). Polynucleotides encoding these polypeptides are also provided. The present invention also encompasses the use of these Gene 13 asparagine glycosylation site polypeptide as immunogenic and/or antigenic epitopes as described elsewhere herein.

The Gene 13 polypeptide was predicted to comprise four N-myristoylation sites using the Motif algorithm (Genetics Computer Group, Inc.). An appreciable number of eukaryotic proteins are acylated by the covalent addition of myristate (a C14-saturated fatty acid) to their N-terminal residue via an amide linkage. The sequence specificity of the enzyme responsible for this modification, myristoyl CoA:protein N-myristoyl transferase (NMT), has been derived from the sequence of known N-myristoylated proteins and from studies using synthetic peptides. The specificity seems to be the following: i.) The N-terminal residue must be glycine; ii.) In position 2, uncharged residues are allowed; iii.) Charged residues, proline and large hydrophobic residues are not allowed; iv.) In positions 3 and 4, most, if not all, residues are allowed; v.) In position 5, small uncharged residues are allowed (Ala, Ser, Thr, Cys, Asn and Gly). Serine is favored; and vi.) In position 6, proline is not allowed.

A consensus pattern for N-myristoylation is as follows: G-{EDRKHPFYW}-x(2)-[STAGCN]-{P}, wherein ‘x’ represents any amino acid, and G is the N-myristoylation site.

Additional information specific to N-myristoylation sites may be found in reference to the following publication: Towler D. A., Gordon J. I., Adams S. P., Glaser L., Annu. Rev. Biochem. 57:69-99(1988); and Grand R. J. A., Biochem. J. 258:625-638(1989); which is hereby incorporated herein in its entirety.

In preferred embodiments, the following N-myristoylation site polypeptides are encompassed by the present invention: ITFEIGQTSANITVEI (SEQ ID NO:145), LSVSSGSLGAHINATL (SEQ ID NO:146), SSGSLGAHINATLTVL (SEQ ID NO:147), and/or GFALFGANQSEATIAI (SEQ ID NO:148). Polynucleotides encoding these polypeptides are also provided. The present invention also encompasses the use of these N-myristoylation site polypeptides as immunogenic and/or antigenic epitopes as described elsewhere herein.

Many polynucleotide sequences, such as EST sequences, are publicly available and accessible through sequence databases. Some of these sequences are related to SEQ ID NO:13 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides consisting of a nucleotide sequence described by the general formula of a-b, where a is any integer between 1 to 892 of SEQ ID NO:13, b is an integer between 15 to 906, where both a and b correspond to the positions of nucleotide residues shown in SEQ ID NO:13, and where b is greater than or equal to a+14.

In one embodiment, a Gene 13 polypeptide comprises a portion of the amino sequence depicted in FIGS. 25A-B and/or FIG. 26. In another embodiment, a Gene 13 polypeptide comprises at least 4, 5, 6, 7, 8, 9, 10, 11, 12, 13, 14, 15, 16, 17, 18, 19 or 20 amino acids of the amino sequence depicted in FIGS. 25A-B and/or FIG. 26. In further embodiments, the following Gene 13 polypeptide fragments are specifically excluded from the present invention: ITVEILPDEDPELDKAFSVS (SEQ ID NO:188); LSVSSGSLGAHINATLTV (SEQ ID NO:189); SDDPYGIFIFSEKNRPVKVEEATQNITLSIIRLKGLMGKVLVSYATLDDMEKPP YFPPNLARATQGRDYIPASGFAL (SEQ ID NO:190); GANQSEATIAISI (SEQ ID NO:191); or KGLMGKVLVSYATLDDMEKPPYFPPNLARATQGRDYIPASGFALFGANQSEA TIAISILDDDEPERSESVFIELLNSTLVAKVQSRS (SEQ ID NO:192).

GPCR polynucleotides and/or polypeptides are useful for diagnosing diseases related to over- or under-expression of GPCR proteins. For example, such GPCR-associated diseases can be assessed by identifying mutations in a GPCR gene using GPCR probes or primers, or by determining GPCR protein or mRNA expression levels. GPCR polypeptides are also useful for screening compounds which affect activity of the protein. The invention further encompasses the polynucleotides encoding the GPCR polypeptides and the use of the GPCR polynucleotides or polypeptides, or compositions thereof, in the screening, diagnosis, treatment, or prevention of disorders associated with aberrant or uncontrolled cellular growth and/or function, such as neoplastic diseases (for example, cancers and tumors).

GPCR probes or primers can be used, for example, to screen for diseases associated with GPCRs. Table 2 lists the predicted left and right primers, i.e., SEQ ID NOs:27, 29, 31, 33, 35, 37, 39, 41, 43, 45, 47, 49, 51, 53, 55 and SEQ ID NOs:28, 30, 32, 34, 36, 38, 40, 42, 44, 46, 48, 50, 52, 54 56, respectively, as determined from the disclosed GPCR nucleic acid sequences. Table 3 lists the predicted internal primers for the GPCR polynucleotides of the present invention (SEQ ID NOs:57-71).

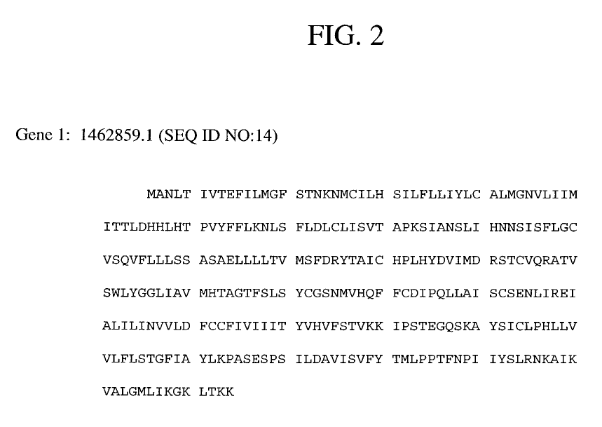

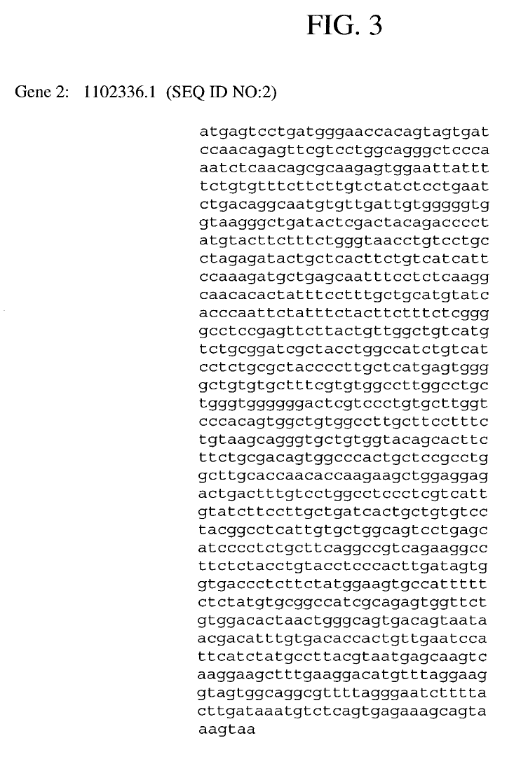

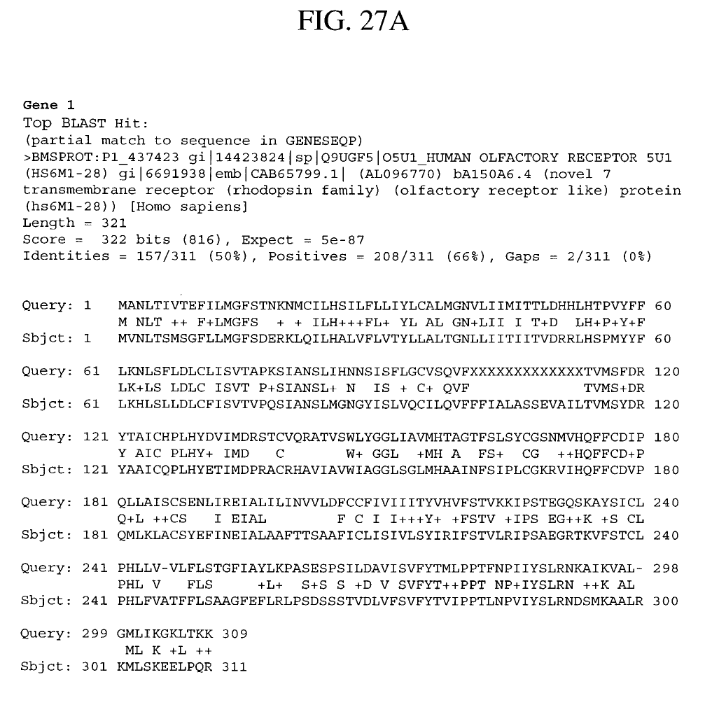

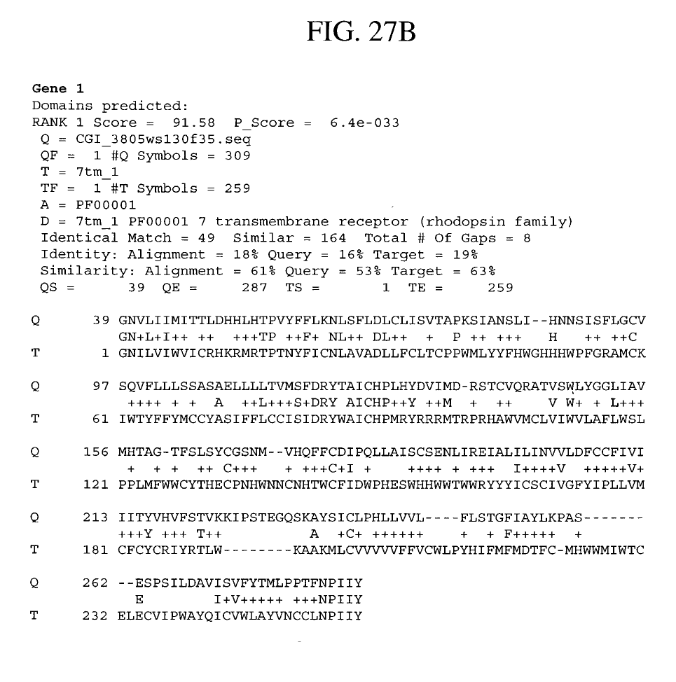

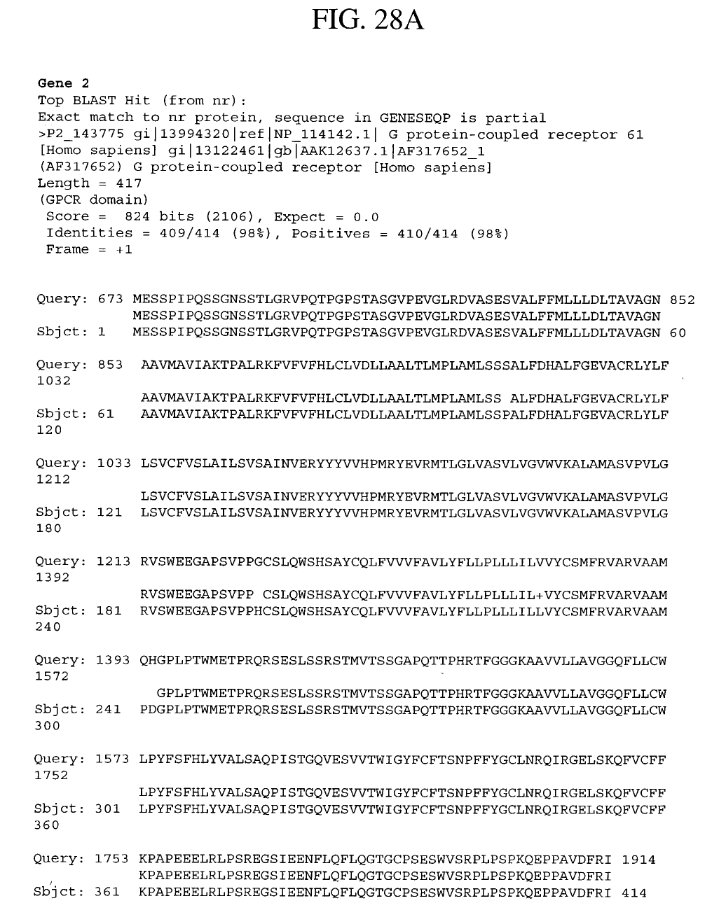

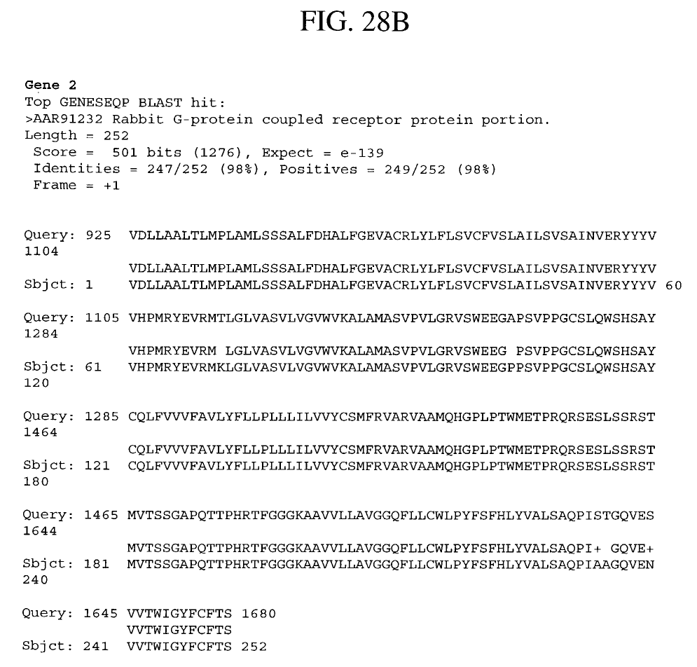

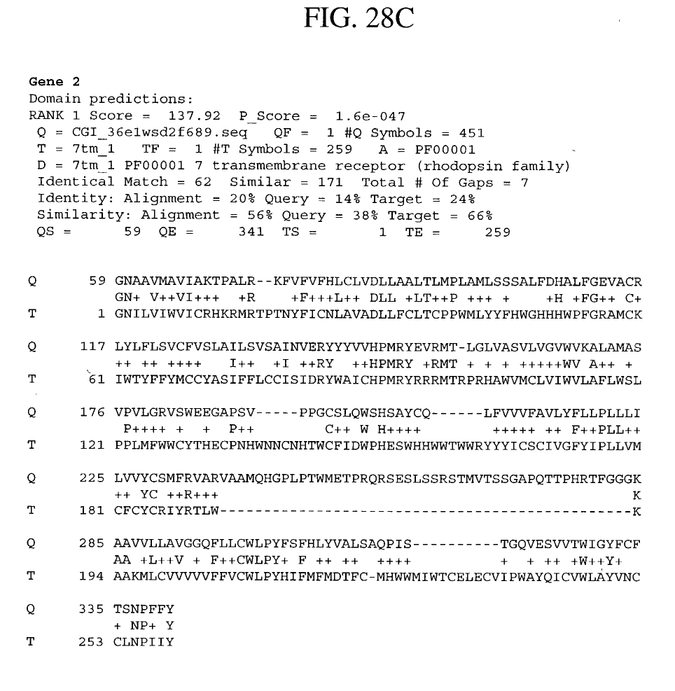

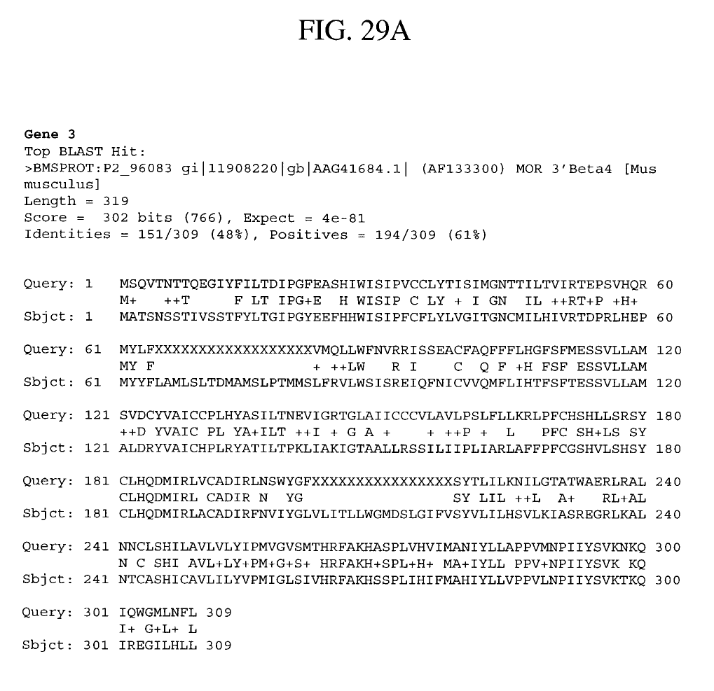

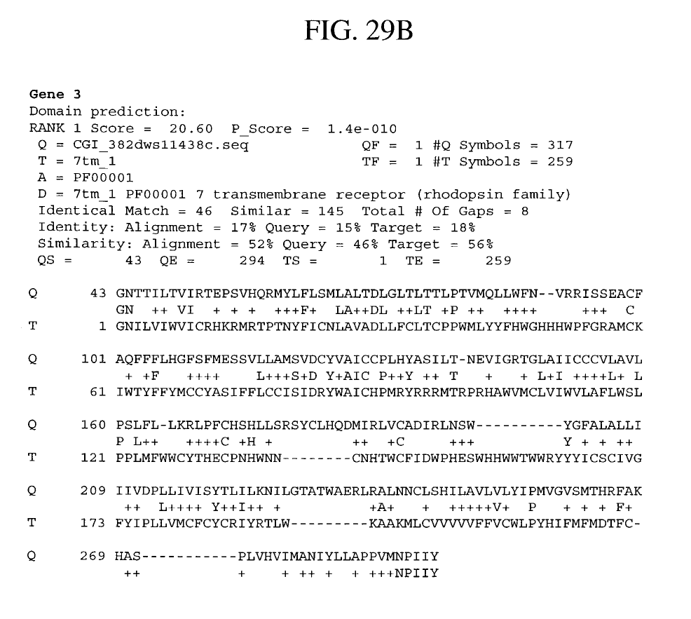

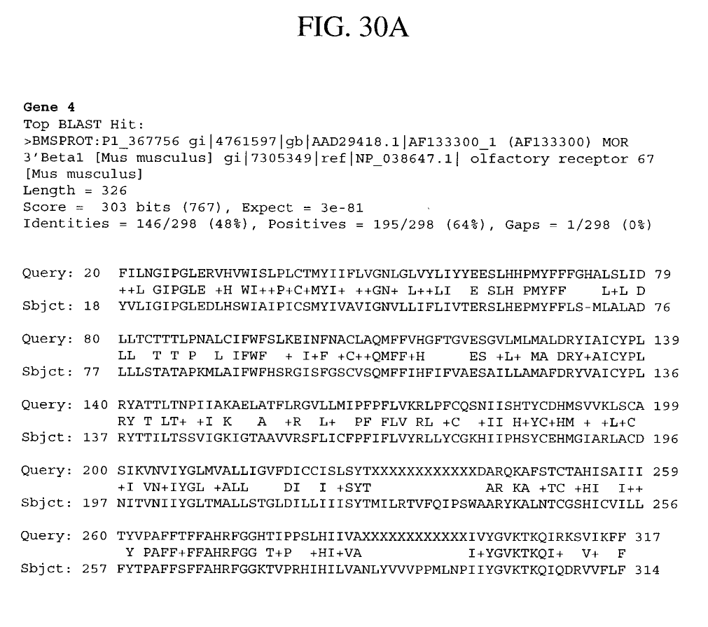

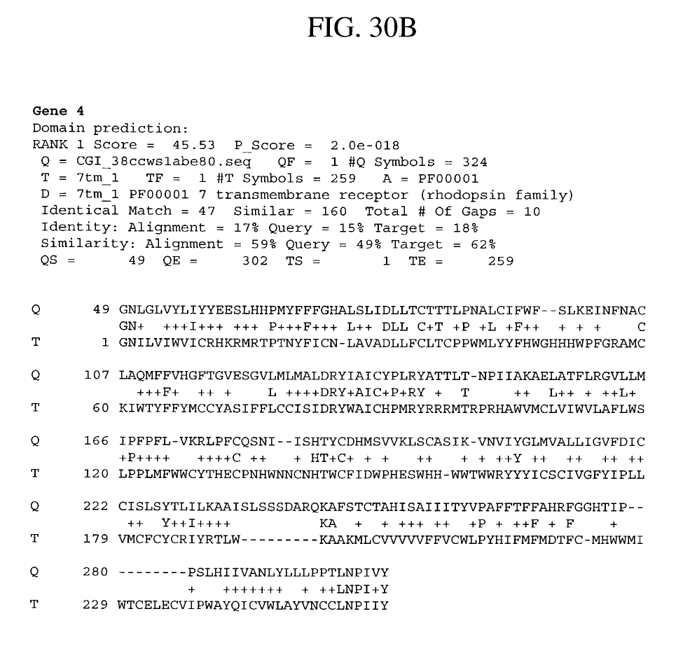

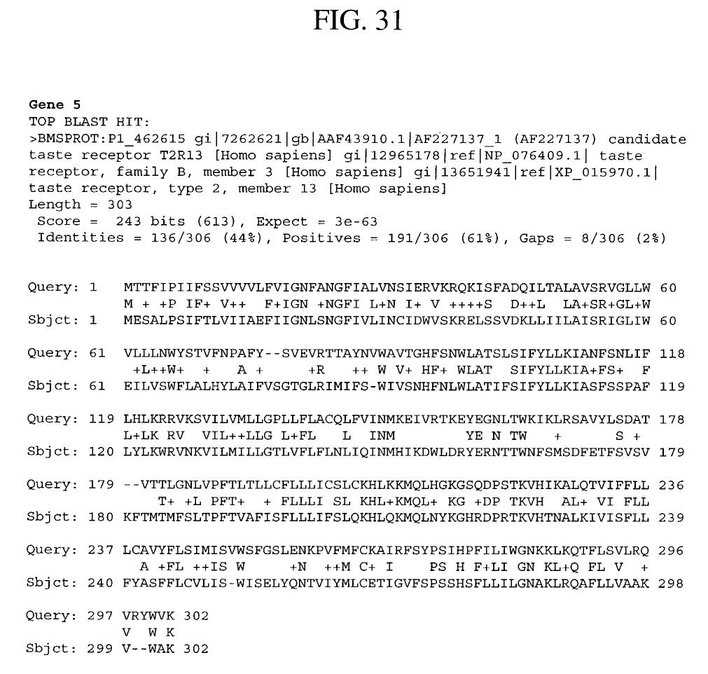

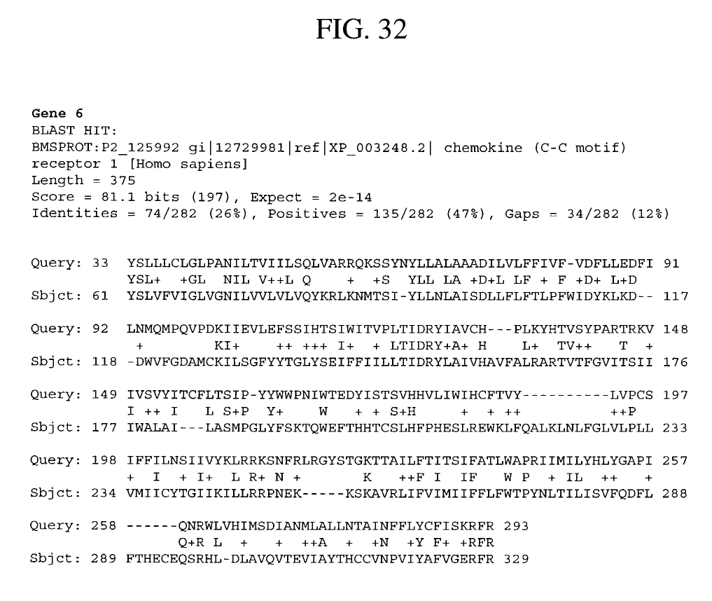

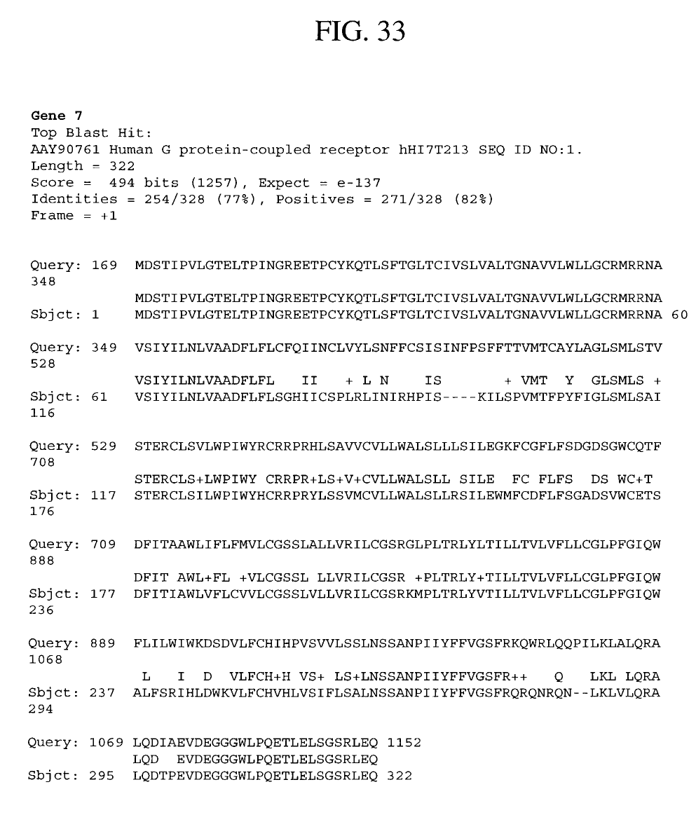

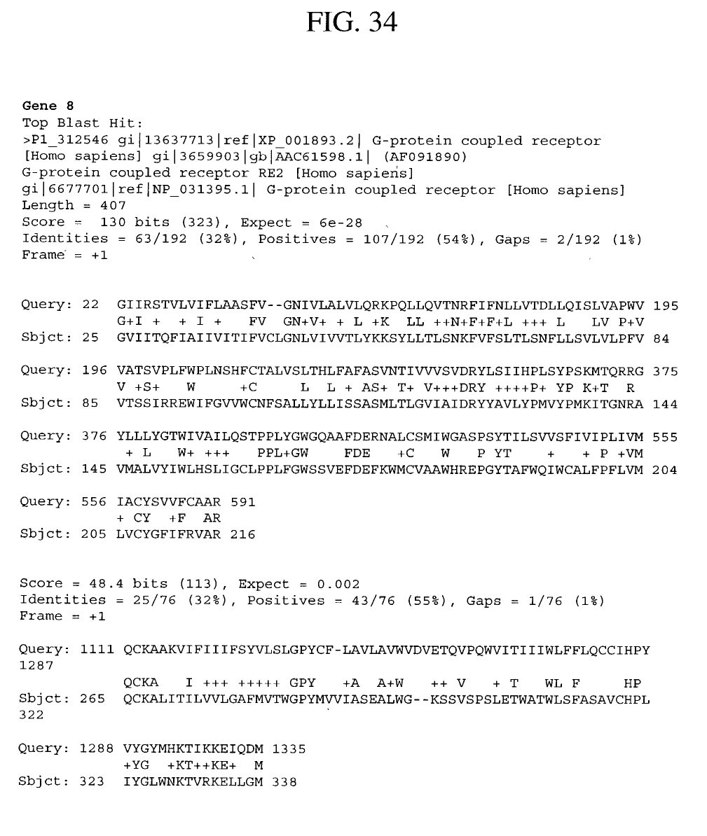

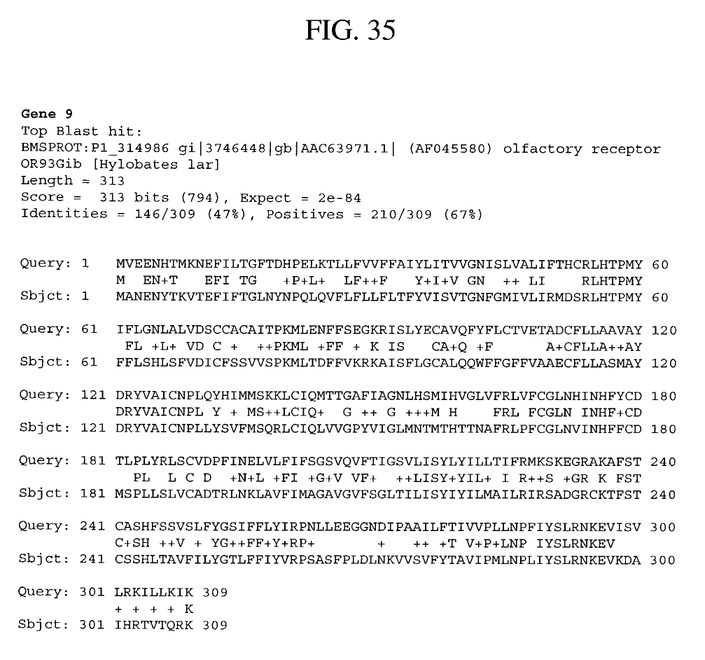

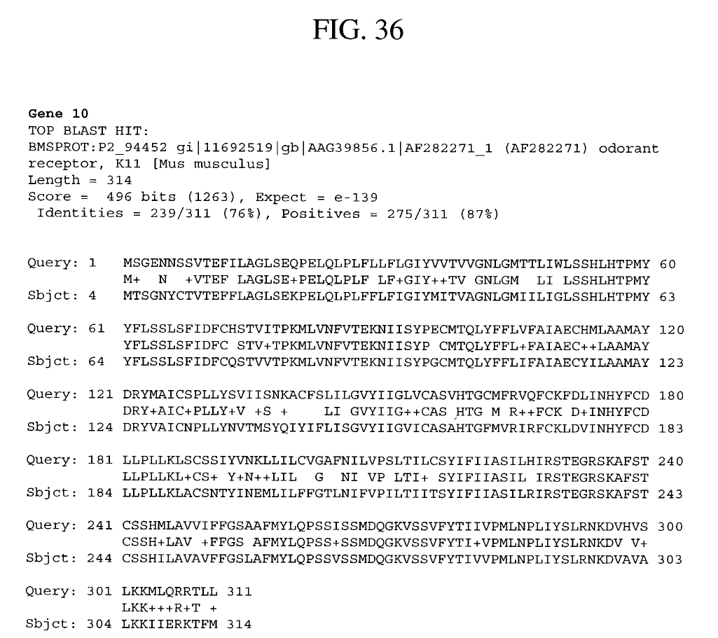

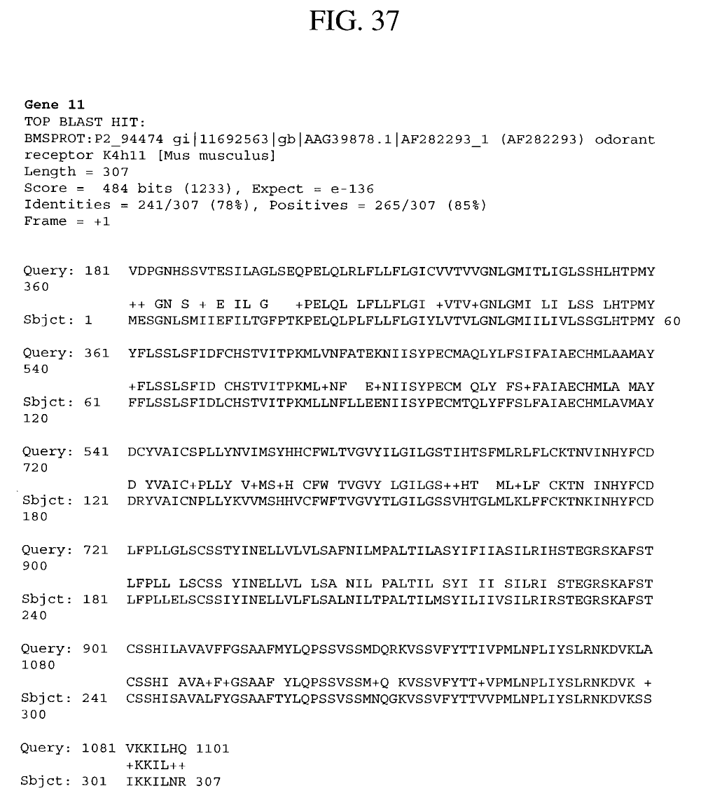

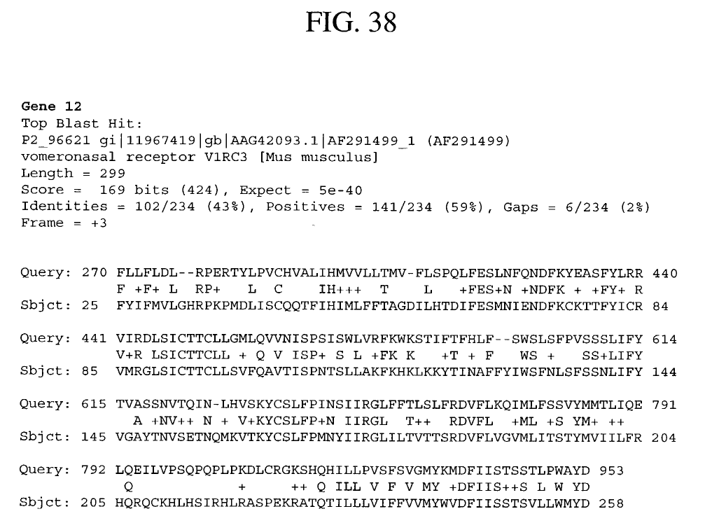

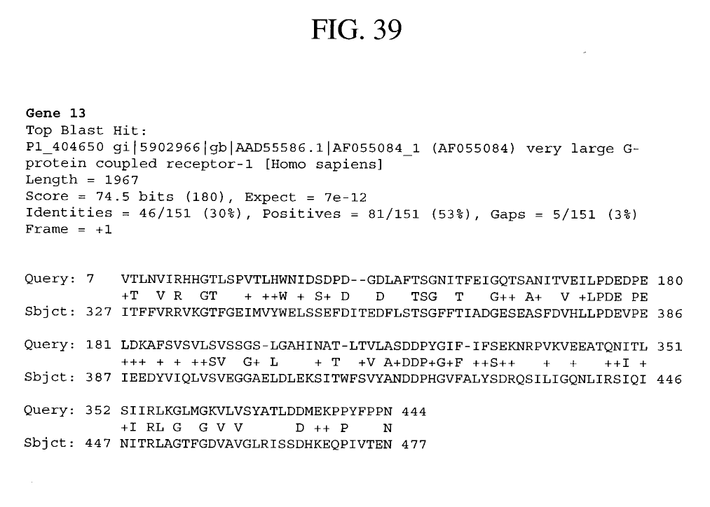

One embodiment of the present invention encompasses novel GPCR polypeptides comprising the amino acid sequences of SEQ ID NOs:14-26 as shown in FIGS. 2, 4, 6, 8, 10, 12, 14, 16, 18, 20, 22, 24 and 26, respectively. More specifically, the sensory GPCR polypeptide of SEQ ID NO:14 is 309 amino acids in length and has 50% amino acid sequence identity with the human olfactory receptor 5U1 (SEQ ID NO:72), (FIG. 27A). The sensory GPCR polypeptide of SEQ ID NO:15 comprises 331 amino acids and has 98% sequence identity with human G protein-coupled receptor 61 (SEQ ID NO:73), (FIG. 28A), and 98% sequence identity with a portion of rabbit G protein-coupled receptor (SEQ ID NO:74), (FIG. 28B). The sensory GPCR polypeptide of SEQ ID NO:16 comprises 317 amino acids and has 48% sequence identity with olfactory receptor 3′Beta4 of mouse (SEQ ID NO:75), (FIG. 29A). The sensory GPCR polypeptide of SEQ ID NO:17 comprises 324 amino acids and has 48% sequence identity with the olfactory receptor 3′Beta1 of mouse (SEQ ID NO:76), (FIG. 30A). The polynucleotide encoding the GPCR of SEQ ID NO:17 has been localized to lung on the basis of tissue expression analysis. The sensory GPCR polypeptide of SEQ ID NO:18 comprises 309 amino acids and has 44% sequence identity with human taste receptor T2R13, family B (SEQ ID NO:77), (FIG. 31). The polynucleotide encoding the GPCR of SEQ ID NO:18 has been localized to uterus on the basis of tissue expression analysis. The chemokine GPCR polypeptide of SEQ ID NO:19 comprises 372 amino acids and has 26% sequence identity with human chemokine receptor 1 (SEQ ID NO:78), (FIG. 32). The human orphan GPCR polypeptide of SEQ ID NO:20 comprises 328 amino acids and has 77% sequence identity with human G protein coupled receptor hHI7T213 (SEQ ID NO:79), (FIG. 33). The-polynucleotide encoding the GPCR of SEQ ID NO:20 has been found in skull tumor on the basis of tissue expression analysis. The human orphan GPCR polypeptide of SEQ ID NO:21 comprises 485 amino acids and has 32% sequence identity with human G protein coupled receptor RE2 (SEQ ID NO:80), (FIG. 34). The human sensory GPCR polypeptide of SEQ ID NO:22 comprises 316 amino acids and has 47% sequence identity with olfactory receptor OR993Gib (SEQ ID NO:81), (FIG. 35). The polynucleotide encoding the GPCR of SEQ ID NO:22 has been found in cartilage on the basis of tissue expression analysis. The human sensory GPCR polypeptide of SEQ ID NO:23 comprises 311 amino acids and has 76% sequence identity with odorant receptor K11 of mouse (SEQ ID NO:82), (FIG. 36). The human sensory GPCR polypeptide of SEQ ID NO:24 comprises 370 amino acids and has 78% sequence identity with odorant receptor K4h11 of mouse (SEQ ID NO:83), (FIG. 37). The human sensory GPCR polypeptide of SEQ ID NO:25 comprises 255 amino acids and has 43% sequence identity with vomeronasal receptor V1RC3 of mouse (SEQ ID NO:84), (FIG. 38). The very large human GPCR polypeptide of SEQ ID NO:26 comprises 295 amino acids and has 30% sequence identity with human very large G-protein coupled receptor-1 (SEQ ID NO:85), (FIG. 39). The polynucleotide encoding the GPCR of SEQ ID NO:26 has been found in brain on the basis of tissue expression analysis

Variants of GPCR polypeptides are also encompassed by the present invention. Preferably, a GPCR variant has at least 75 to 80%, more preferably at least 85 to 90%, and even more preferably at least 90% amino acid sequence identity to a GPCR amino acid sequence disclosed herein, and more preferably, retains at least one biological, immunological, or other functional characteristic or activity of the non-variant GPCR polypeptide. Most preferred are GPCR variants or substantially purified fragments thereof having at least 95% amino acid sequence identity to those of SEQ ID NOs:14-26. Variants of GPCR polypeptides or substantially purified fragments of the polypeptides can also include amino acid sequences that differ from any one of the SEQ ID NOs:14-26 amino acid sequences only by conservative substitutions. The invention also encompasses polypeptide homologues of any one of amino acid sequences as set forth in SEQ ID NOs:14-26.

In another embodiment, the present invention encompasses polynucleotides which encode GPCR polypeptides. Accordingly, any nucleic acid sequence that encodes the amino acid sequence of a GPCR polypeptide of the invention can be used to produce recombinant molecules that express a GPCR protein. More particularly, the invention encompasses the GPCR polynucleotides comprising the nucleic acid sequences of SEQ ID NOs:1-13. The present invention also provides GPCR cDNA clones, specifically clones corresponding to Gene 12, deposited at the American Type Culture Collection (ATCC), 10801 University Boulevard, Manassas, Va. 20110-2209 on Dec. 22, 2001 and under ATCC Accession No(s). PTA-3949 according to the terms of the Budapest Treaty.

As will be appreciated by the skilled practitioner in the art, the degeneracy of the genetic code results in many nucleotide sequences that can encode the described GPCR polypeptides. Some of the sequences bear minimal or no homology to the nucleotide sequences of any known and naturally occurring gene. Accordingly, the present invention contemplates each and every possible variation of nucleotide sequence that could be made by selecting combinations based on possible codon choices. These combinations are made in accordance with the standard triplet genetic code as applied to the nucleotide sequence of naturally occurring GPCR, and all such variations are to be considered as being specifically disclosed and able to be understood by the skilled practitioner.

Although nucleic acid sequences which encode the GPCR polypeptides and variants thereof are preferably capable of hybridizing to the nucleotide sequence of the naturally occurring GPCR polypeptide under appropriately selected conditions of stringency, it may be advantageous to produce nucleotide sequences encoding GPCR polypeptides, or derivatives thereof, which possess a substantially different codon usage. For example, codons may be selected to increase the rate at which expression of the peptide/polypeptide occurs in a particular prokaryotic or eukaryotic host in accordance with the frequency with which particular codons are utilized by the host. Another reason for substantially altering the nucleotide sequence encoding a GPCR polypeptide, or its derivatives, without altering the encoded amino acid sequences, includes the production of RNA transcripts having more desirable properties, such as a greater half-life, than transcripts produced from the naturally occurring sequence.

The present invention also encompasses production of DNA sequences, or portions thereof, which encode the GPCR polypeptides, or derivatives thereof, entirely by synthetic chemistry. After production, the synthetic sequence may be inserted into any of the many available expression vectors and cell systems using reagents that are well known and practiced by those in the art. Moreover, synthetic chemistry may be used to introduce mutations into a sequence encoding a GPCR polypeptide, or any fragment thereof.

In an embodiment of the present invention, a gene delivery vector containing the polynucleotide, or functional fragment thereof is provided. Preferably, the gene delivery vector contains the polynucleotide, or functional fragment thereof comprising an isolated and purified polynucleotide encoding a human GPCR having the sequence as set forth in any one of SEQ ID NOs:1-13.

It will also be appreciated by those skilled in the pertinent art that in addition to the primers disclosed in Tables 2 and 3 herein, a longer oligonucleotide probe, or mixtures of probes, for example, degenerate probes, can be used to detect longer, or more complex, nucleic acid sequences, such as, for example, genomic or full length DNA. In such cases, the probe may comprise at least 20-300 nucleotides, preferably, at least 30-100 nucleotides, and more preferably, 50-100 nucleotides.

The present invention also provides methods of obtaining the full length sequence of the GPCR polypeptides as described herein. In one instance, the method of multiplex cloning was devised as a means of extending large numbers of bioinformatic gene predictions into full length sequences by multiplexing probes and cDNA libraries in an effort to minimize the overall effort typically required for cDNA cloning. The method relies on the conversion of plasmid-based, directionally cloned cDNA libraries into a population of pure, covalently-closed, circular, single-stranded molecules and long biotinylated DNA oligonucleotide probes designed from predicted gene sequences.

Probes and libraries were subjected to solution hybridization in a formamide buffer which has been found to be superior to aqueous buffers typically used in other biotin/streptavidin cDNA capture methods (i.e., GeneTrapper). The hybridization was performed without prior knowledge of the clones represented in the libraries. Hybridization was performed two times. After the first selection, the isolated sequences were screened with PCR primers specific for the targeted clones. The second hybridization was carried out with only those oligo probes whose gene-specific PCR assays gave positive results.

The secondary hybridization serves to ‘normalize’ the selected library, thereby decreasing the amount of screening needed to identify particular clones. The method is robust and sensitive. Typically, dozens of cDNAs are isolated for any one particular gene, thereby increasing the chances of obtaining a full length cDNA. The entire complexity of any cDNA library is screened in the solution hybridization process, which is advantageous for finding rare sequences. The procedure is scaleable, with 50 oligonucleotide probes per experiment currently being used, although this is not to be considered a limiting number.

Using bioinformatic predicted gene sequence, the following types of PCR primers and cloning oligos can be designed: A) PCR primer pairs that reside within a single predicted exon; B) PCR primer pairs that cross putative exon/intron boundaries; and C) 80mer antisense and sense oligos containing a biotin moiety on the 5′ end. The primer pairs of the A type above are optimized on human genomic DNA; the B type primer pairs are optimized on a mixture of first strand cDNAs made with and without reverse transcriptase. Primers will be optimized using mRNA derived from appropriate tissues sources, for example, brain, lung, uterus, cartilage, and testis poly A+ RNA.

The information obtained with the B type primers is used to assess those putative expressed sequences which can be experimentally observed to have reverse transcriptase-dependent expression. The primer pairs of the A type are less stringent in terms of identifying expressed sequences. However, because they amplify genomic DNA as well as cDNA, their ability to amplify genomic DNA provides for the necessary positive control for the primer pair. Negative results with the B type are subject to the caveat that the sequence(s) may not be expressed in the tissue first strand that is under examination.

The biotinylated 80-mer oligonucleotides are added en mass to pools of single strand cDNA libraries. Up to 50 probes have been successfully used on pools for 15 different libraries. After the primary selection is performed, all of the captured DNA is repaired to double strand form using the T7 primer for the commercial libraries in pCMVSPORT, and the Sp6 primer for other constructed libraries in pSPORT. The resulting DNA is electroporated into E. coli DH12S and plated onto 150 mm plates with nylon filters. The cells are scraped and a frozen stock is made, thereby comprising the primary selected library.

One-fifth of the library is generally converted into single strand form and the DNA is assayed with gene specific primer pairs (GSPs). The next round of solution hybridization capture is carried out with 80 mer oligos for only those sequences that are positive with the gene-specific-primers. After the second round, the captured single strand DNAs are repaired with a pool of GSPs, where only the primer complementary to polarity of the single-stranded circular DNA is used (i.e., the antisense primer for pCMVSPORT and pSPORT1 and the sense primer for pSPORT2).

The resulting colonies are screened by PCR using the GSPs. Typically, greater than 80% of the clones are positive for any given GSP. The entire 96 well block of clones is subjected to “mini-prep”, as known in the art, and each of clones is sized by either PCR or restriction enzyme digestion. A selection of different sized clones for each targeted sequence is chosen for transposon-hopping and DNA sequencing.

Preferably, as for established cDNA cloning methods used by the skilled practitioner, the libraries employed are of high quality. High complexity and large average insert size are optimal. High Pressure Liquid Chromatography (HPLC) may be employed as a means of fractionating cDNA for the purpose of constructing libraries.

Another embodiment of the present invention provides a method of identifying full-length genes encoding the disclosed polypeptides. The GPCR polynucleotides of the present invention, the polynucleotides encoding the GPCR polypeptides of the present invention, or the polypeptides encoded by the deposited clone(s) preferably represent the complete coding region (i.e., full-length gene).

Several methods are known in the art for the identification of the 5′ or 3′ non-coding and/or coding portions of a given gene. The methods described herein are exemplary and should not be construed as limiting the scope of the invention. These methods include, but are not limited to, filter probing, clone enrichment using specific probes, and protocols similar or identical to 5′ and 3′ “RACE” protocols that are well known in the art. For instance, a method similar to 5′ RACE is available for generating the missing 5′ end of a desired full-length transcript. (Fromont-Racine et al., Nucleic Acids Res. 21(7):1683-1684 (1993)).

Briefly, in the RACE method, a specific RNA oligonucleotide is ligated to the 5′ ends of a population of RNA presumably containing full-length gene RNA transcripts. A primer set containing a primer specific to the ligated RNA oligonucleotide and a primer specific to a known sequence of the gene of interest is used to PCR amplify the 5′ portion of the desired full-length gene. This amplified product may then be sequenced and used to generate the full-length gene.

The above method utilizes total RNA isolated from the desired source, although poly-A+ RNA can be used. The RNA preparation is treated with phosphatase, if necessary, to eliminate 5′ phosphate groups on degraded or damaged RNA that may interfere with the later RNA ligase step. The phosphatase is preferably inactivated and the RNA is treated with tobacco acid pyrophosphatase in order to remove the cap structure present at the 5′ ends of messenger RNAs. This reaction leaves a 5′ phosphate group at the 5′ end of the cap cleaved RNA which can then be ligated to an RNA oligonucleotide using T4 RNA ligase.

The above-described modified RNA preparation is used as a template for first strand cDNA synthesis employing a gene specific oligonucleotide. The first strand synthesis reaction is used as a template for PCR amplification of the desired 5′ end using a primer specific to the ligated RNA oligonucleotide and a primer specific to the known sequence of the gene of interest. The resultant product is then sequenced and analyzed to confirm that the 5′ end sequence belongs to the desired gene. It may also be advantageous to optimize the RACE protocol to increase the probability of isolating additional 5′ or 3′ coding or non-coding sequences. Various methods of optimizing a RACE protocol are known in the art; for example, a detailed description summarizing these methods can be found in B. C. Schaefer, Anal. Biochem., 227:255-273, (1995).

An alternative method for carrying out 5′ or 3′ RACE for the identification of coding or non-coding nucleic acid sequences is provided by Frohman, M. A., et al., Proc. Nat'l. Acad. Sci. USA, 85:8998-9002 (1988). Briefly, a cDNA clone missing either the 5′ or 3′ end can be reconstructed to include the absent base pairs extending to the translational start or stop codon, respectively. In some cases, cDNAs are missing the start of translation for an encoded product. A brief description of a modification of the original 5′ RACE procedure is as follows. Poly A+ or total RNA is reverse transcribed with Superscript II (Gibco/BRL) and an antisense or an I complementary primer specific to any one of the cDNA sequences provided as SEQ ID NOS:1-13. The primer is removed from the reaction with a Microcon Concentrator (Amicon). The first-strand cDNA is then tailed with dATP and terminal deoxynucleotide transferase (Gibco/BRL). Thus, an anchor sequence is produced which is needed for PCR amplification. The second strand is synthesized from the dA-tail in PCR buffer, Taq DNA polymerase (Perkin-Elmer Cetus), an oligo-dT primer containing three adjacent restriction sites (XhoIJ Sail and ClaI) at the 5′ end and a primer containing just these restriction sites. This double-stranded cDNA is PCR amplified for 40 cycles with the same primers, as well as a nested cDNA-specific antisense primer. The PCR products are size-separated on an ethidium bromide-agarose gel and the region of gel containing cDNA products having the predicted size of missing protein-coding DNA is removed.

cDNA is purified from the agarose with the Magic PCR Prep kit (Promega), restriction digested with XhoI or SalI, and ligated to a plasmid such as pBluescript SKII (Stratagene) at XhoI and EcoRV sites. This DNA is transformed into bacteria and the plasmid clones sequenced to identify the correct protein-coding inserts. Correct 5′ ends are confirmed by comparing this sequence with the putatively identified homologue and overlap with the partial cDNA clone. Similar methods known in the art and/or commercial kits are used to amplify and recover 3′ ends.

Several quality-controlled kits are commercially available for purchase. Similar reagents and methods to those above are supplied in kit form from Gibco/BRL for both 5′ and 3′ RACE for recovery of full length genes. A second kit is available from Clontech which is a modification of a related technique, called single-stranded ligation to single-stranded cDNA, (SLIC), developed by Dumas et al., Nucleic Acids Res., 19:5227-32(1991). The major difference in the latter procedure is that the RNA is alkaline hydrolyzed after reverse transcription and RNA ligase is used to join a restriction site-containing anchor primer to the first-strand cDNA. This obviates the necessity for the dA-tailing reaction which results in a polyT stretch that can impede sequencing.

An alternative to generating 5′ or 3′ cDNA from RNA is to use cDNA library double-stranded DNA. An asymmetric PCR-amplified antisense cDNA strand is synthesized with an antisense cDNA-specific primer and a plasmid-anchored primer. These primers are removed and a symmetric PCR reaction is performed with a nested cDNA-specific antisense primer and the plasmid-anchored primer.

Also encompassed by the present invention are polynucleotide sequences that are capable of hybridizing to the novel GPCR nucleic acid sequences, as set forth in SEQ ID NOs:1-13, under various conditions of stringency. Hybridization conditions are typically based on the melting temperature (Tₘ) of the nucleic acid binding complex or probe (see, G. M. Wahl and S. L. Berger, 1987; Methods Enzymol., 152:399-407 and A. R. Kimmel, 1987; Methods of Enzymol., 152:507-511), and may be used at a defined stringency. For example, included in the present invention are sequences capable of hybridizing under moderately stringent conditions to the GPCR sequences of SEQ ID NOs:1-13 and other sequences which are degenerate to those which encode the novel GPCR polypeptides. For example, a non-limiting example of moderate stringency conditions include prewashing solution of 2×SSC, 0.5% SDS, 1.0 mM EDTA, pH 8.0, and hybridization conditions of 50° C., 5×SSC, overnight.

The nucleic acid sequence encoding the GPCR proteins of the present invention may be extended by utilizing a partial nucleotide sequence and employing various methods known in the art to detect upstream sequences such as promoters and regulatory elements. For example, one method that can be employed is restriction-site PCR, which utilizes universal primers to retrieve unknown sequence adjacent to a known locus (See, e.g., G. Sarkar, 1993, PCR Methods Applic., 2:318-322). In particular, genomic DNA is first amplified in the presence of a primer to a linker sequence and a primer specific to the known region. The amplified sequences are then subjected to a second round of PCR with the same linker primer and another specific primer internal to the first one. Products of each round of PCR are transcribed with an appropriate RNA polymerase and sequenced using reverse transcriptase.

Inverse PCR may also be used to amplify or extend sequences using divergent primers based on a known region or sequence (T. Triglia et al., 1988, Nucleic Acids Res., 16:8186). The primers may be designed using OLIGO 4.06 Primer Analysis software (National Biosciences, Inc., Plymouth, Minn.), or another appropriate program, to be 22-30 nucleotides in length, to have a GC content of 50% or more, and to anneal to the target sequence at temperatures about 68° C.-72° C. The method uses several restriction enzymes to generate a suitable fragment in the known region of a gene. The fragment is then circularized by intramolecular ligation and used as a PCR template.

Another method which may be used to amplify or extend sequences is capture PCR which involves PCR amplification of DNA fragments adjacent to a known sequence in human and yeast artificial chromosome (YAC) DNA (M. Lagerstrom et al., 1991, PCR Methods Applic., 1:111-119). In this method, multiple restriction enzyme digestions and ligations may also be used to place an engineered double-stranded sequence into an unknown portion of the DNA molecule before performing PCR. J. D. Parker et al. (1991; Nucleic Acids Res., 19:3055-3060) provide another method which may be used to retrieve unknown sequences. Bacterial artificial chromosomes (BACs) are also used for such applications. In addition, PCR, nested primers, and PROMOTERFINDER libraries can be used to “walk” genomic DNA (Clontech, Palo Alto, Calif.). This process avoids the need to screen libraries and is useful in finding intron/exon junctions.

When screening for full-length cDNAs, it is preferable to use libraries that have been size-selected to include larger cDNAs. Also, random-primed libraries are also preferable, since such libraries will contain more sequences that comprise the 5′ regions of genes. The use of a randomly primed library may be especially preferable for situations in which an oligo d(T) library does not yield a full-length cDNA. Genomic libraries may be useful for extension of sequence into the 5′ and 3′ non-transcribed regulatory regions.

The embodiments of the present invention can be practiced using methods for DNA sequencing which are well known and generally available in the art. The methods may employ such enzymes as the Klenow fragment of DNA polymerase I, SEQUENASE (US Biochemical Corp. Cleveland, Ohio), Taq polymerase (PE Biosystems), thermostable T7 polymerase (Amersham Pharmacia Biotech, Piscataway, N.J.), or combinations of recombinant polymerases and proofreading exonucleases such as the ELONGASE Amplification System marketed by Life Technologies (Gaithersburg, Md.). Preferably, the process is automated with machines such as the Hamilton Micro Lab 2200 (Hamilton, Reno, Nev.), Peltier Thermal Cycler (PTC200; MJ Research, Watertown, Mass.) and the ABI Catalyst and 373 and 377 DNA sequencers (PE Biosystems). Commercially available capillary electrophoresis systems may be used to analyze the size or confirm the nucleotide sequence of sequencing or PCR products. Capillary electrophoresis is especially preferable for the sequencing of small pieces of DNA, which might be present in limited amounts in a particular sample.

In another embodiment of the present invention, polynucleotide sequences or portions thereof which encode GPCR polypeptides or peptides can comprise recombinant DNA molecules to direct the expression of GPCR polypeptide products, peptide fragments, or functional equivalents thereof, in appropriate host cells. Because of the inherent degeneracy of the genetic code, other DNA sequences, which encode substantially the same or a functionally equivalent amino acid sequence, may be produced and these sequences may be used to clone and express the GPCR proteins as described.

As will be appreciated by those having skill in the art, it may be advantageous to produce GPCR polypeptide-encoding nucleotide sequences possessing non-naturally occurring codons. For example, codons preferred by a particular prokaryotic or eukaryotic host can be selected to increase the rate of protein expression or to produce a recombinant RNA transcript having desirable properties, such as a half-life which is longer than that of a transcript generated from the naturally occurring sequence.

The nucleotide sequences of the present invention can be engineered using methods generally known in the art in order to alter the GPCR polypeptide-encoding sequences for a variety of reasons, including, but not limited to, alterations which modify the cloning, processing, and/or expression of the gene product. DNA shuffling by random fragmentation, PCR reassembly of gene fragments and synthetic oligonucleotides may be used to engineer the nucleotide sequences. For example, site-directed mutagenesis may be used to insert new restriction sites, alter glycosylation patterns, change codon preference, produce splice variants, or introduce mutations, and the like.

In another embodiment of the present invention, natural, modified, or recombinant nucleic acid sequences encoding the GPCR polypeptides may be ligated to a heterologous sequence to encode a fusion (or chimeric or hybrid) protein. For example, a fusion protein can comprise any one of the amino acid sequences as set forth in SEQ ID NOs:14-26 and an amino acid sequence of an Fc portion (or constant region) of a human immunoglobulin protein. The fusion protein may further comprise an amino acid sequence that differs from any one of SEQ ID NOs:14-26 only by conservative substitutions. As another example, to screen peptide libraries for inhibitors of GPCR activity, it may be useful to encode a chimeric GPCR protein that can be recognized by a commercially available antibody. A fusion protein may also be engineered to contain a cleavage site located between the GPCR protein-encoding sequence and the heterologous protein sequence, so that the GPCR protein may be cleaved and purified away from the heterologous moiety.

In a further embodiment, sequences encoding the GPCR polypeptides may be synthesized in whole, or in part, using chemical methods well known in the art (see, for example, M. H. Caruthers et al., 1980, Nucl. Acids Res. Symp. Ser., 215-223 and T. Horn et al., 1980, Nucl. Acids Res. Symp. Ser., 225-232). Alternatively, the GPCR protein itself, or a fragment or portion thereof, may be produced using chemical methods to synthesize the amino acid sequence of the GPCR polypeptide, or a fragment or portion thereof. For example, peptide synthesis can be performed using various solid-phase techniques (J. Y. Roberge et al., 1995, Science, 269:202-204) and automated synthesis can be achieved, for example, using the ABI 431A Peptide Synthesizer (PE Biosystems).

The newly synthesized GPCR polypeptide or peptide can be substantially purified by preparative high performance liquid chromatography (e.g., T. Creighton, 1983, Proteins, Structures and Molecular Principles, W. H. Freeman and Co., New York, N.Y.), by reverse-phase high performance liquid chromatography (HPLC), or other purification methods as known and practiced in the art. The composition of the synthetic peptides may be confirmed by amino acid analysis or sequencing (e.g., the Edman degradation procedure; Creighton, supra). In addition, the amino acid sequence of a GPCR polypeptide, or any portion thereof, can be altered during direct synthesis and/or combined using chemical methods with sequences from other proteins, or any part thereof, to produce a variant polypeptide.

To express a biologically active GPCR polypeptide or peptide, the nucleotide sequences encoding the GPCR polypeptide, or functional equivalents, may be inserted into an appropriate expression vector, i.e., a vector, which contains the necessary elements for the transcription and translation of the inserted coding sequence.

In one embodiment of the present invention, an expression vector contains an isolated and purified polynucleotide sequence as set forth in any one of SEQ ID NOs:1-13, encoding a human GPCR, or a functional fragment thereof, in which the human GPCR comprises the amino acid sequence as set forth in any one of SEQ ID NOs:14-26. Alternatively, an expression vector can contain the complement of the aforementioned GPCR nucleic acid sequences.

Expression vectors derived from retroviruses, adenovirus, herpes or vaccinia viruses, or from various bacterial plasmids can be used for the delivery of nucleotide sequences to a target organ, tissue or cell population. Methods, which are well known to those skilled in the art, may be used to construct expression vectors containing sequences encoding one or more GPCR polypeptide along with appropriate transcriptional and translational control elements. These methods include in vitro recombinant DNA techniques, synthetic techniques, and in vivo genetic recombination. Such techniques are described in J. Sambrook et al., 1989, Molecular Cloning, A Laboratory Manual, Cold Spring Harbor Press, Plainview, N.Y. and in F. M. Ausubel et al., 1989, Current Protocols in Molecular Biology, John Wiley & Sons, New York, N.Y.

A variety of expression vector/host systems may be utilized to contain and express sequences encoding the GPCR polypeptides or peptides. Such expression vector/host systems include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus (CaMV) and tobacco mosaic virus (TMV)), or with bacterial expression vectors (e.g., Ti or pBR322 plasmids); or animal cell systems, including mammalian cell systems. The host cell employed is not limiting to the present invention. Preferably, the host cell of the invention contains an expression vector comprising an isolated and purified polynucleotide having a nucleic acid sequence selected from any one of SEQ ID NOs:1-13 and encoding a human GPCR of this invention, or a functional fragment thereof, comprising an amino acid sequence as set forth in any one of SEQ ID NOs:14-26.

Bacterial artificial chromosomes (BACs) may be used to deliver larger fragments of DNA than can be contained and expressed in a plasmid vector. BACs are vectors used to clone DNA sequences of 100-300 kb, on average 150 kb, in size in E. coli cells. BACs are constructed and delivered via conventional delivery methods (e.g., liposomes, polycationic amino polymers, or vesicles) for therapeutic purposes.

“Control elements” or “regulatory sequences” are those non-translated regions of the vector, e.g., enhancers, promoters, 5′ and 3′ untranslated regions, which interact with host cellular proteins to carry out transcription and translation. Such elements may vary in their strength and specificity. Depending on the vector system and host utilized, any number of suitable transcription and translation elements, including constitutive and inducible promoters, may be used. Specific initiation signals may also be used to achieve more efficient translation of sequences encoding a GPCR polypeptide. Such signals include the ATG initiation codon and adjacent sequences. In cases where sequences encoding a GPCR polypeptide, its initiation codon, and upstream sequences are inserted into the appropriate expression vector, no additional transcriptional or translational control signals may be needed. However, in cases where only a GPCR coding sequence, or a fragment thereof, is inserted, exogenous translational control signals, including the ATG initiation codon, are optimally provided. Furthermore, the initiation codon should be in the correct reading frame to insure translation of the entire insert. Exogenous translational elements and initiation codons can be of various origins, both natural and synthetic. The efficiency of expression can be enhanced by the inclusion of enhancers which are appropriate for the particular cell system that is used, such as those described in the literature (see, e.g., D. Scharf et al., 1994, Results Probl. Cell Differ., 20:125-162).

In bacterial systems, a number of expression vectors may be selected, depending upon the use intended for the expressed GPCR product. For example, when large quantities of expressed protein are needed for the generation of antibodies, vectors that direct high level expression of fusion proteins that can be readily purified may be used. Such vectors include, but are not limited to, the multifunctional E. coli cloning and expression vectors such as BLUESCRIPT (Stratagene), in which the sequence encoding the GPCR polypeptide can be ligated into the vector in-frame with sequences for the amino-terminal Met and the subsequent 7 residues of β-galactosidase, so that a hybrid protein is produced; pIN vectors (see, G. Van Heeke and S. M. Schuster, 1989, J. Biol. Chem., 264:5503-5509); and the like. pGEX vectors (Promega, Madison, Wis.) can also be used to express foreign polypeptides as fusion proteins with glutathione S-transferase (GST). In general, such fusion proteins are soluble and can be easily purified from lysed cells by adsorption to glutathione-agarose beads followed by elution in the presence of free glutathione. Proteins made in such systems can be designed to include heparin, thrombin, or factor XA protease cleavage sites so that the cloned polypeptide of interest can be released from the GST moiety at will.

In mammalian host cells, a number of viral-based expression systems can be utilized. In cases where an adenovirus is used as an expression vector, sequences encoding the GPCR polypeptide may be ligated into an adenovirus transcription/translation complex containing the late promoter and tripartite leader sequence. Insertion into a non-essential E1 or E3 region of the viral genome may be used to obtain a viable virus which is capable of expressing GPCR polypeptide in infected host cells (J. Logan and T. Shenk, 1984, Proc. Natl. Acad. Sci., 81:3655-3659). In addition, transcription enhancers, such as the Rous sarcoma virus (RSV) enhancer, may be used to increase expression in mammalian host cells. Other expression systems can also be used, such as, but not limited to yeast, plant, and insect vectors.

Moreover, a host cell strain may be chosen for its ability to modulate the expression of the inserted sequences or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” form of the protein may also be used to facilitate correct insertion, folding and/or function. Different host cells having specific cellular machinery and characteristic mechanisms for such post-translational activities (e.g., CHO, HeLa, MDCK, HEK293, and W138) are available from the American Type Culture Collection (ATCC), 10801 University Boulevard, Manassas, Va. 20110-2209, and may be chosen to ensure the correct modification and processing of the foreign protein.

Host cells transformed with nucleotide sequences encoding a GPCR protein, or fragments thereof, may be cultured under conditions suitable for the expression and recovery of the protein from cell culture. The protein produced by a recombinant cell may be secreted or contained intracellularly depending on the sequence and/or the vector used. As will be understood by those having skill in the art, expression vectors containing polynucleotides which encode a GPCR protein can be designed to contain signal sequences which direct secretion of the GPCR protein through a prokaryotic or eukaryotic cell membrane. Other constructions can be used to join nucleic acid sequences encoding a GPCR protein to a nucleotide sequence encoding a polypeptide domain, which will facilitate purification of soluble proteins. Such purification facilitating domains include, but are not limited to, metal chelating peptides such as histidine-tryptophan modules that allow purification on immobilized metals; protein A domains that allow purification on immobilized immunoglobulin; and the domain utilized in the FLAGS extension/affinity purification system (Immunex Corp., Seattle, Wash.). The inclusion of cleavable linker sequences such as those specific for Factor XA or enterokinase (Invitrogen, San Diego, Calif.) between the purification domain and GPCR protein may be used to facilitate purification. One such expression vector provides for expression of a fusion protein containing GPCR and a nucleic acid encoding 6 histidine residues preceding a thioredoxin or an enterokinase cleavage site. The histidine residues facilitate purification on IMAC (immobilized metal ion affinity chromatography) as described by J. Porath et al., 1992, Prot. Exp. Purif., 3:263-281, while the enterokinase cleavage site provides a means for purifying from the fusion protein. For a discussion of suitable vectors for fusion protein production, see D. J. Kroll et al., 1993; DNA Cell Biol., 12:441-453.

Any number of selection systems may be used to recover transformed cell lines. These include, but are not limited to, the Herpes Simplex Virus thymidine kinase (HSV TK), (M. Wigler et al., 1977, Cell, 11:223-32) and adenine phosphoribosyltransferase (I. Lowy et al., 1980, Cell, 22:817-23) genes which can be employed in tk⁻ or aprt⁻ cells, respectively. Also, anti-metabolite, antibiotic or herbicide resistance can be used as the basis for selection; for example, dhfr, which confers resistance to methotrexate (M. Wigler et al., 1980, Proc. Natl. Acad. Sci., 77:3567-70); npt, which confers resistance to the aminoglycosides neomycin and G-418 (F. Colbere-Garapin et al., 1981, J. Mol. Biol., 150:1-14); and als or pat, which confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine (S. C. Hartman and R. C. Mulligan, 1988, Proc. Natl. Acad. Sci., 85:8047-51). Recently, the use of visible markers has gained popularity with such markers as the anthocyanins, β-glucuronidase and its substrate GUS, and luciferase and its substrate luciferin, which are widely used not only to identify transformants, but also to quantify the amount of transient or stable protein expression that is attributable to a specific vector system (C. A. Rhodes et al., 1995, Methods Mol. Biol., 55:121-131).

Although the presence or absence of marker gene expression suggests that the gene of interest is also present, the presence and expression of the desired gene of interest may need to be confirmed. For example, if the nucleic acid sequence encoding a GPCR polypeptide is inserted within a marker gene sequence, recombinant cells containing polynucleotide sequence encoding the GPCR polypeptide can be identified by the absence of marker gene function. Alternatively, a marker gene can be placed in tandem with a sequence encoding a GPCR polypeptide under the control of a single promoter. Expression of the marker gene in response to induction or selection typically indicates co-expression of the tandem gene.

A wide variety of labels and conjugation techniques are known and employed by those skilled in the art and may be used in various nucleic acid and amino acid assays. Means for producing labeled hybridization or PCR probes for detecting sequences related to polynucleotides encoding a GPCR polypeptide include oligo-labeling, nick translation, end-labeling, or PCR amplification using a labeled nucleotide. Alternatively, the sequences encoding a GPCR polypeptide of this invention, or any portion or fragment thereof, can be cloned into a vector for the production of an mRNA probe. Such vectors are known in the art, are commercially available, and may be used to synthesize RNA probes in vitro by addition of an appropriate RNA polymerase, such as T7, T3, or SP(6) and labeled nucleotides. These procedures may be conducted using a variety of commercially available kits (e.g., Amersham Pharmacia Biotech, Promega and U.S. Biochemical Corp.). Suitable reporter molecules or labels which can be used include radionuclides, enzymes, fluorescent, chemiluminescent, or chromogenic agents, as well as substrates, cofactors, inhibitors, magnetic particles, and the like.

Alternatively, host cells which contain the nucleic acid sequence coding for a GPCR polypeptide of the invention and which express the GPCR polypeptide product may be identified by a variety of procedures known to those having skill in the art. These procedures include, but are not limited to, DNA-DNA or DNA-RNA hybridizations and protein bioassay or immunoassay techniques, including membrane, solution, or chip based technologies, for the detection and/or quantification of nucleic acid or protein.

The presence of polynucleotide sequences encoding GPCR polypeptides can be detected by DNA-DNA or DNA-RNA hybridization, or by amplification using probes, portions, or fragments of polynucleotides encoding a GPCR polypeptide. Nucleic acid amplification based assays involve the use of oligonucleotides or oligomers based on the nucleic acid sequences encoding a GPCR polypeptide to detect transformants containing DNA or RNA encoding GPCR polypeptide.

In addition to recombinant production, fragments of GPCR polypeptides may be produced by direct peptide synthesis using solid phase techniques (J. Merrifield, 1963, J. Am. Chem. Soc., 85:2149-2154). Protein synthesis may be performed using manual techniques or by automation. Automated synthesis may be achieved, for example, using ABI 431A Peptide Synthesizer (PE Biosystems). Various fragments of the GPCR polypeptides can be chemically synthesized separately and then combined using chemical methods to produce the full length molecule.

Diagnostic Assays

In another embodiment of the present invention, antibodies which specifically bind to a GPCR polypeptide may be used for the diagnosis of conditions or diseases characterized by expression (or overexpression) of the GPCR polynucleotide or polypeptide, or in assays to monitor patients being treated with one or more of the GPCR polypeptides, or agonists, antagonists, or inhibitors of the novel GPCRs. The antibodies useful for diagnostic purposes can be prepared in the same manner as those described herein for use in therapeutic methods. Diagnostic assays for the GPCR polypeptides include methods which utilize the antibody and a label to detect the protein in human body fluids or extracts of cells or tissues. The antibodies may be used with or without modification, and may be labeled by joining them, either covalently or non-covalently, with a reporter molecule. A wide variety of reporter molecules known to those in the art may be used, several of which are described herein.

Another embodiment of the present invention contemplates a method of detecting a GPCR homologue, or an antibody-reactive fragment thereof, in a sample. The method comprises a) contacting the sample with an antibody specific for a GPCR polypeptide of the present invention, or an antigenic fragment thereof, under conditions in which an antigen-antibody complex can form between the antibody and the polypeptide or antigenic fragment thereof in the sample; and b) detecting the antigen-antibody complex formed in step a), wherein detection of the complex indicates the presence of the GPCR polypeptide, or an antigenic fragment thereof, in the sample.

Several assay protocols including enzyme-linked immunosorbent assay (ELISA), radioimmunoassay (RIA), and fluorescence activated cell sorting (FACS) for measuring GPCR polypeptide are known in the art and provide a basis for diagnosing altered or abnormal levels of GPCR polypeptide expression. Normal or standard values for GPCR polypeptide expression are established by combining body fluids or cell extracts taken from normal mammalian subjects, preferably human, with antibody to the GPCR polypeptide under conditions suitable for complex formation. The amount of standard complex formation may be quantified by various methods; photometric means are preferred. Quantities of GPCR polypeptide expressed in a subject or test sample, control sample, and disease samples from biopsied tissues are compared with the standard values. Deviation between standard and subject values establishes the parameters for diagnosing disease.

A variety of protocols for detecting and measuring the expression of GPCR polypeptide using either polyclonal or monoclonal antibodies specific for the polypeptide, or epitopic portions thereof, are known and practiced in the art. Examples include enzyme-linked immunosorbent assay (ELISA), radioimmunoassay (RIA), and fluorescence activated cell sorting (FACS). A two-site, monoclonal-based immunoassay utilizing monoclonal antibodies reactive with two non-interfering epitopes on a GPCR polypeptide is preferred, but a competitive binding assay may also be employed. These and other assays are described in the art as represented by the publication of R. Hampton et al., 1990; Serological Methods, a Laboratory Manual, APS Press, St Paul, Minn. and D. E. Maddox et al., 1983; J. Exp. Med., 158:1211-1216).

Uses for Antibodies Directed Against Polypeptides of the Invention

The antibodies of the present invention have various utilities. For example, such antibodies may be used in diagnostic assays to detect the presence or quantification of the polypeptides of the invention in a sample. Such a diagnostic assay may be comprised of at least two steps. The first, subjecting a sample with the antibody, wherein the sample is a tissue (e.g., human, animal, etc.), biological fluid (e.g., blood, urine, sputum, semen, amniotic fluid, saliva, etc.), biological extract (e.g., tissue or cellular homogenate, etc.), a protein microchip (e.g., See Arenkov P, et al., Anal Biochem., 278(2):123-131 (2000)), or a chromatography column, etc. And a second step involving the quantification of antibody bound to the substrate. Alternatively, the method may additionally involve a first step of attaching the antibody, either covalently, electrostatically, or reversibly, to a solid support, and a second step of subjecting the bound antibody to the sample, as defined above and elsewhere herein.

Various diagnostic assay techniques are known in the art, such as competitive binding assays, direct or indirect sandwich assays and immunoprecipitation assays conducted in either heterogeneous or homogenous phases (Zola, Monoclonal Antibodies: A Manual of Techniques, CRC Press, Inc., (1987), pp147-158). The antibodies used in the diagnostic assays can be labeled with a detectable moiety. The detectable moiety should be capable of producing, either directly or indirectly, a detectable signal. For example, the detectable moiety may be a radioisotope, such as 2H, 14C, 32P, or 125I, a florescent or chemiluminescent compound, such as fluorescein isothiocyanate, rhodamine, or luciferin, or an enzyme, such as alkaline phosphatase, beta-galactosidase, green fluorescent protein, or horseradish peroxidase. Any method known in the art for conjugating the antibody to the detectable moiety may be employed, including those methods described by Hunter et al., Nature, 144:945 (1962); Dafvid et al., Biochem., 13:1014 (1974); Pain et al., J. Immunol. Metho., 40:219(1981); and Nygren, J. Histochem. And Cytochem., 30:407 (1982).

Antibodies directed against the polypeptides of the present invention are useful for the affinity purification of such polypeptides from recombinant cell culture or natural sources. In this process, the antibodies against a particular polypeptide are immobilized on a suitable support, such as a Sephadex resin or filter paper, using methods well known in the art. The immobilized antibody then is contacted with a sample containing the polypeptides to be purified, and thereafter the support is washed with a suitable solvent that will remove substantially all the material in the sample except for the desired polypeptides, which are bound to the immobilized antibody. Finally, the support is washed with another suitable solvent that will release the desired polypeptide from the antibody.

Immunophenotyping

The antibodies of the invention may be utilized for immunophenotyping of cell lines and biological samples. The translation product of the gene of the present invention may be useful as a cell specific marker, or more specifically as a cellular marker that is differentially expressed at various stages of differentiation and/or maturation of particular cell types. Monoclonal antibodies directed against a specific epitope, or combination of epitopes, will allow for the screening of cellular populations expressing the marker. Various techniques can be utilized using monoclonal antibodies to screen for cellular populations expressing the marker(s), and include magnetic separation using antibody-coated magnetic beads, “panning” with antibody attached to a solid matrix (i.e., plate), and flow cytometry (See, e.g., U.S. Pat. No. 5,985,660; and Morrison et al., Cell, 96:737-49 (1999)).

These techniques allow for the screening of particular populations of cells, such as might be found with hematological malignancies (i.e. minimal residual disease (MRD) in acute leukemic patients) and “non-self” cells in transplantations to prevent Graft-versus-Host Disease (GVHD). Alternatively, these techniques allow for the screening of hematopoietic stem and progenitor cells capable of undergoing proliferation and/or differentiation, as might be found in human umbilical cord blood.

Assays for Antibody Binding

The antibodies of the invention may be assayed for immunospecific binding by any method known in the art. The immunoassays which can be used include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al, eds, 1994, Current Protocols in Molecular Biology, Vol. 1, John Wiley & Sons, Inc., New York, which is incorporated by reference herein in its entirety). Exemplary immunoassays are described briefly below (but are not intended by way of limitation).

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody of interest to the cell lysate, incubating for a period of time (e.g., 1-4 hours) at 4° C., adding protein A and/or protein G sepharose beads to the cell lysate, incubating for about an hour or more at 4° C., washing the beads in lysis buffer and resuspending the beads in SDS/sample buffer. The ability of the antibody of interest to immunoprecipitate a particular antigen can be assessed by, e.g., western blot analysis. One of skill in the art would be knowledgeable as to the parameters that can be modified to increase the binding of the antibody to an antigen and decrease the background (e.g., pre-clearing the cell lysate with sepharose beads). For further discussion regarding immunoprecipitation protocols see, e.g., Ausubel et al, eds, 1994, Current Protocols in Molecular Biology, Vol. 1, John Wiley & Sons, Inc., New York at 10.16.1.

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with primary antibody (the antibody of interest) diluted in blocking buffer, washing the membrane in washing buffer, blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., 32P or 125I) diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols see, e.g., Ausubel et al, eds, 1994, Current Protocols in Molecular Biology, Vol. 1, John Wiley & Sons, Inc., New York at 10.8.1.

ELISAs comprise preparing antigen, coating the well of a 96 well microtiter plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art. For further discussion regarding ELISAs see, e.g., Ausubel et al, eds, 1994, Current Protocols in Molecular Biology, Vol. 1, John Wiley & Sons, Inc., New York at 11.2.1.

The binding affinity of an antibody to an antigen and the off-rate of an antibody-antigen interaction can be determined by competitive binding assays. One example of a competitive binding assay is a radioimmunoassay comprising the incubation of labeled antigen (e.g., 3H or 125I) with the antibody of interest in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest conjugated to a labeled compound (e.g., 3H or 125I) in the presence of increasing amounts of an unlabeled second antibody.

Therapeutic Uses Of Antibodies

The present invention is further directed to antibody-based therapies which involve administering antibodies of the invention to an animal, preferably a mammal, and most preferably a human, patient for treating one or more of the disclosed diseases, disorders, or conditions. Therapeutic compounds of the invention include, but are not limited to, antibodies of the invention (including fragments, analogs and derivatives thereof as described herein) and nucleic acids encoding antibodies of the invention (including fragments, analogs and derivatives thereof and anti-idiotypic antibodies as described herein). The antibodies of the invention can be used to treat, inhibit or prevent diseases, disorders or conditions associated with aberrant expression and/or activity of a polypeptide of the invention, including, but not limited to, any one or more of the diseases, disorders, or conditions described herein. The treatment and/or prevention of diseases, disorders, or conditions associated with aberrant expression and/or activity of a polypeptide of the invention includes, but is not limited to, alleviating symptoms associated with those diseases, disorders or conditions. Antibodies of the invention may be provided in pharmaceutically acceptable compositions as known in the art or as described herein.

A summary of the ways in which the antibodies of the present invention may be used therapeutically includes binding polynucleotides or polypeptides of the present invention locally or systemically in the body or by direct cytotoxicity of the antibody, e.g. as mediated by complement (CDC) or by effector cells (ADCC). Some of these approaches are described in more detail below. Armed with the teachings provided herein, one of ordinary skill in the art will know how to use the antibodies of the present invention for diagnostic, monitoring or therapeutic purposes without undue experimentation.

The antibodies of this invention may be advantageously utilized in combination with other monoclonal or chimeric antibodies, or with lymphokines or hematopoietic-growth factors (such as, e.g., IL-2, IL-3 and IL-7), for example, which serve to increase the number or activity of effector cells which interact with the antibodies.

The antibodies of the invention may be administered alone or in combination with other types of treatments (e.g., radiation therapy, chemotherapy, hormonal therapy, immunotherapy and anti-tumor agents). Generally, administration of products of a species origin or species reactivity (in the case of antibodies) that is the same species as that of the patient is preferred. Thus, in a preferred embodiment, human antibodies, fragments derivatives, analogs, or nucleic acids, are administered to a human patient for therapy or prophylaxis.

It is preferred to use high affinity and/or potent in vivo inhibiting and/or neutralizing antibodies against polypeptides or polynucleotides of the present invention, fragments or regions thereof, for both immunoassays directed to and therapy of disorders related to polynucleotides or polypeptides, including fragments thereof, of the present invention. Such antibodies, fragments, or regions, will preferably have an affinity for polynucleotides or polypeptides of the invention, including fragments thereof. Preferred binding affinities include those with a dissociation constant or Kd less than 5×10−2 M, 10−2 M, 5×10−3 M, 10−3 M, 5×10−4 M, 10−4 M, 5×10−5 M, 10−5 M, 5×10−6 M, 10−6 M, 5×10−7 M, 10−7 M, 5×10−8 M, 10−8 M, 5×10−9 M, 10−9 M, 5×10−10 M, 10−10 M, 5×10−11 M, 10-11 M, 5×10−12 M, 10−12 M, 5×10−13 M, 10−13 M, 5×10−14 M, 10−14 M, 5×10−15 M, and 10−15 M.

Antibodies directed against polypeptides of the present invention are useful for inhibiting allergic reactions in animals. For example, by administering a therapeutically acceptable dose of an antibody, or antibodies, of the present invention, or a cocktail of the present antibodies, or in combination with other antibodies of varying sources, the animal may not elicit an allergic response to antigens.

Likewise, one could envision cloning the gene encoding an antibody directed against a polypeptide of the present invention, said polypeptide having the potential to elicit an allergic and/or immune response in an organism, and transforming the organism with said antibody gene such that it is expressed (e.g., constitutively, inducibly, etc.) in the organism. Thus, the organism would effectively become resistant to an allergic response resulting from the ingestion or presence of such an immune/allergic reactive polypeptide. Moreover, such a use of the antibodies of the present invention may have particular utility in preventing and/or ameliorating autoimmune diseases and/or disorders, as such conditions are typically a result of antibodies being directed against endogenous proteins. For example, in the instance where the polypeptide of the present invention is responsible for modulating the immune response to auto-antigens, transforming the organism and/or individual with a construct comprising any of the promoters disclosed herein or otherwise known in the art, in addition, to a polynucleotide encoding the antibody directed against the polypeptide of the present invention could effective inhibit the organisms immune system from eliciting an immune response to the auto-antigen(s). Detailed descriptions of therapeutic and/or gene therapy applications of the present invention are provided elsewhere herein.

Alternatively, antibodies of the present invention could be produced in a plant (e.g., cloning the gene of the antibody directed against a polypeptide of the present invention, and transforming a plant with a suitable vector comprising said gene for constitutive expression of the antibody within the plant), and the plant subsequently ingested by an animal, thereby conferring temporary immunity to the animal for the specific antigen the antibody is directed towards (See, for example, U.S. Pat. Nos. 5,914,123 and 6,034,298).

In another embodiment, antibodies of the present invention, preferably polyclonal antibodies, more preferably monoclonal antibodies, and most preferably single-chain antibodies, can be used as a means of inhibiting gene expression of a particular gene, or genes, in a human, mammal, and/or other organism. See, for example, International Publication Number WO 00/05391, published Feb. 3, 2000, to Dow Agrosciences LLC. The application of such methods for the antibodies of the present invention are known in the art, and are more particularly described elsewhere herein.

In yet another embodiment, antibodies of the present invention may be useful for multimerizing the polypeptides of the present invention. For example, certain proteins may confer enhanced biological activity when present in a multimeric state (i.e., such enhanced activity may be due to the increased effective concentration of such proteins whereby more protein is available in a localized location).

Antibody-Based Gene Therapy

In a specific embodiment, nucleic acids comprising sequences encoding antibodies or functional derivatives thereof, are administered to treat, inhibit or prevent a disease or disorder associated with aberrant expression and/or activity of a polypeptide of the invention, by way of gene therapy. Gene therapy refers to therapy performed by the administration to a subject of an expressed or expressible nucleic acid. In this embodiment of the invention, the nucleic acids produce their encoded protein that mediates a therapeutic effect.

Any of the methods for gene therapy available in the art can be used according to the present invention. Exemplary methods are described below.

For general reviews of the methods of gene therapy, see Goldspiel et al., Clinical Pharmacy 12:488-505 (1993); Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); May, TIBTECH 11(5):155-215 (1993). Methods commonly known in the art of recombinant DNA technology which can be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

In a preferred aspect, the compound comprises nucleic acid sequences encoding an antibody, said nucleic acid sequences being part of expression vectors that express the antibody or fragments or chimeric proteins or heavy or light chains thereof in a suitable host. In particular, such nucleic acid sequences have promoters operably linked to the antibody coding region, said promoter being inducible or constitutive, and, optionally, tissue-specific. In another particular embodiment, nucleic acid molecules are used in which the antibody coding sequences and any other desired sequences are flanked by regions that promote homologous recombination at a desired site in the genome, thus providing for intrachromosomal expression of the antibody encoding nucleic acids (Koller and Smithies, Proc. Natl. Acad. Sci. USA 86:8932-8935 (1989); Zijlstra et al., Nature 342:435-438 (1989). In specific embodiments, the expressed antibody molecule is a single chain antibody; alternatively, the nucleic acid sequences include sequences encoding both the heavy and light chains, or fragments thereof, of the antibody.

Delivery of the nucleic acids into a patient may be either direct, in which case the patient is directly exposed to the nucleic acid or nucleic acid-carrying vectors, or indirect, in which case, cells are first transformed with the nucleic acids in vitro, then transplanted into the patient. These two approaches are known, respectively, as in vivo or ex vivo gene therapy.

In a specific embodiment, the nucleic acid sequences are directly administered in vivo, where it is expressed to produce the encoded product. This can be accomplished by any of numerous methods known in the art, e.g., by constructing them as part of an appropriate nucleic acid expression vector and administering it so that they become intracellular, e.g., by infection using defective or attenuated retrovirals or other viral vectors (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu, J. Biol. Chem. 262:4429-4432 (1987)) (which can be used to target cell types specifically expressing the receptors), etc. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180; WO 92/22635; WO92/20316; WO93/14188, WO 93/20221). Alternatively, the nucleic acid can be introduced intracellularly and incorporated within host cell DNA for expression, by homologous recombination (Koller and Smithies, Proc. Natl. Acad. Sci. USA 86:8932-8935 (1989); Zijlstra et al., Nature 342:435-438 (1989)).

In a specific embodiment, viral vectors that contains nucleic acid sequences encoding an antibody of the invention are used. For example, a retroviral vector can be used (see Miller et al., Meth. Enzymol. 217:581-599 (1993)). These retroviral vectors contain the components necessary for the correct packaging of the viral genome and integration into the host cell DNA. The-nucleic acid sequences encoding the antibody to be used in gene therapy are cloned into one or more vectors, which facilitates delivery of the gene into a patient. More detail about retroviral vectors can be found in Boesen et al., Biotherapy 6:291-302 (1994), which describes the use of a retroviral vector to deliver the mdr1 gene to hematopoietic stem cells in order to make the stem cells more resistant to chemotherapy. Other references illustrating the use of retroviral vectors in gene therapy are: Clowes et al., J. Clin. Invest. 93:644-651 (1994); Kiem et al., Blood 83:1467-1473 (1994); Salmons and Gunzberg, Human Gene Therapy 4:129-141 (1993); and Grossman and Wilson, Curr. Opin. in Genetics and Devel. 3:110-114 (1993).

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, Current Opinion in Genetics and Development 3:499-503 (1993) present a review of adenovirus-based gene therapy. Bout et al., Human Gene Therapy 5:3-10 (1994) demonstrated the use of adenovirus vectors to transfer genes to the respiratory epithelia of rhesus monkeys. Other instances of the use of adenoviruses in gene therapy can be found in Rosenfeld et al., Science 252:431-434 (1991); Rosenfeld et al., Cell 68:143-155 (1992); Mastrangeli et al., J. Clin. Invest. 91:225-234 (1993); PCT Publication WO94/12649; and Wang, et al., Gene Therapy 2:775-783 (1995). In a preferred embodiment, adenovirus vectors are used.

Adeno-associated virus (AAV) has also been proposed for use in gene therapy (Walsh et al., Proc. Soc. Exp. Biol. Med. 204:289-300 (1993); U.S. Pat. No. 5,436,146).

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see, e.g., Loeffler and Behr, Meth. Enzymol. 217:599-618 (1993); Cohen et al., Meth. Enzymol. 217:618-644 (1993); Cline, Pharmac. Ther. 29:69-92m (1985) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a patient by various methods known in the art. Recombinant blood cells (e.g., hematopoietic stem or progenitor cells) are preferably administered intravenously. The amount of cells envisioned for use depends on the desired effect, patient state, etc., and can be determined by one skilled in the art.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; blood cells such as Tlymphocytes, Blymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc.

In a preferred embodiment, the cell used for gene therapy is autologous to the patient.

In an embodiment in which recombinant cells are used in gene therapy, nucleic acid sequences encoding an antibody are introduced into the cells such that they are expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the present invention (see e.g. PCT Publication WO 94/08598; Stemple and Anderson, Cell 71:973-985 (1992); Rheinwald, Meth. Cell Bio. 21A:229 (1980); and Pittelkow and Scott, Mayo Clinic Proc. 61:771 (1986)).

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription. Demonstration of Therapeutic or Prophylactic Activity

The compounds or pharmaceutical compositions of the invention are preferably tested in vitro, and then in vivo for the desired therapeutic or prophylactic activity, prior to use in humans. For example, in vitro assays to demonstrate the therapeutic or prophylactic utility of a compound or pharmaceutical composition include, the effect of a compound on a cell line or a patient tissue sample. The effect of the compound or composition on the cell line and/or tissue sample can be determined utilizing techniques known to those of skill in the art including, but not limited to, rosette formation assays and cell lysis assays. In accordance with the invention, in vitro assays which can be used to determine whether administration of a specific compound is indicated, include in vitro cell culture assays in which a patient tissue sample is grown in culture, and exposed to or otherwise administered a compound, and the effect of such compound upon the tissue sample is observed.

Therapeutic/Prophylactic Administration and Compositions

The invention provides methods of treatment, inhibition and prophylaxis by administration to a subject of an effective amount of a compound or pharmaceutical composition of the invention, preferably an antibody of the invention. In a preferred aspect, the compound is substantially purified (e.g., substantially free from substances that limit its effect or produce undesired side-effects). The subject is preferably an animal, including but not limited to animals such as cows, pigs, horses, chickens, cats, dogs, etc., and is preferably a mammal, and most preferably human.

Formulations and methods of administration that can be employed when the compound comprises a nucleic acid or an immunoglobulin are described above; additional appropriate formulations and routes of administration can be selected from among those described herein below.

Various delivery systems are known and can be used to administer a compound of the invention, e.g., encapsulation in liposomes, microparticles, microcapsules, recombinant cells capable of expressing the compound, receptor-mediated endocytosis (see, e.g., Wu and Wu, J. Biol. Chem. 262:4429-4432 (1987)), construction of a nucleic acid as part of a retroviral or other vector, etc. Methods of introduction include but are not limited to intradermal, intramuscular, intraperitoneal, intravenous, subcutaneous, intranasal, epidural, and oral routes. The compounds or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compounds or compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compounds or compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a protein, including an antibody, of the invention, care must be taken to use materials to which the protein does not absorb.

In another embodiment, the compound or composition can be delivered in a vesicle, in particular a liposome (see Langer, Science 249:1527-1533 (1990); Treat et al., in Liposomes in the Therapy of-Infectious Disease and Cancer, Lopez-Berestein and Fidler (eds.), Liss, New York, pp. 353-365 (1989); Lopez-Berestein, ibid., pp. 317-327; see generally ibid.)

In yet another embodiment, the compound or composition can be delivered in a controlled release system. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J., Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target, i.e., the brain, thus requiring only a fraction of the systemic dose (see, e.g., Goodson, in Medical Applications of Controlled Release, supra, vol. 2, pp. 115-138 (1984)).

Other controlled release systems are discussed in the review by Langer (Science 249:1527-1533 (1990)).

In a specific embodiment where the compound of the invention is a nucleic acid encoding a protein, the nucleic acid can be administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., Proc. Natl. Acad. Sci. USA 88:1864-1868 (1991)), etc. Alternatively, a nucleic acid can be introduced intracellularly and incorporated within host cell DNA for expression, by homologous recombination.

The present invention also provides pharmaceutical compositions. Such compositions comprise a therapeutically effective amount of a compound, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the patient. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The amount of the compound of the invention which will be effective in the treatment, inhibition and prevention of a disease or disorder associated with aberrant expression and/or activity of a polypeptide of the invention can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses may be extrapolated from dose-response curves derived from in vitro or animal model test systems.

For antibodies, the dosage administered to a patient is typically 0.1 mg/kg to 100 mg/kg of the patient's body weight. Preferably, the dosage administered to a patient is between 0.1 mg/kg and 20 mg/kg of the patient's body weight, more preferably 1 mg/kg to 10 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the invention may be reduced by enhancing uptake and tissue penetration (e.g., into the brain) of the antibodies by modifications such as, for example, lipidation.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

Diagnosis and Imaging With Antibodies

Labeled antibodies, and derivatives and analogs thereof, which specifically bind to a polypeptide of interest can be used for diagnostic purposes to detect, diagnose, or monitor diseases, disorders, and/or conditions associated with the aberrant expression and/or activity of a polypeptide of the invention. The invention provides for the detection of aberrant expression of a polypeptide of interest, comprising (a) assaying the expression of the polypeptide of interest in cells or body fluid of an individual using one or more antibodies specific to the polypeptide interest and (b) comparing the level of gene expression with a standard gene expression level, whereby an increase or decrease in the assayed polypeptide gene expression level compared to the standard expression level is indicative of aberrant expression.

The invention provides a diagnostic assay for diagnosing a disorder, comprising (a) assaying the expression of the polypeptide of interest in cells or body fluid of an individual using one or more antibodies specific to the polypeptide interest and (b) comparing the level of gene expression with a standard gene expression level, whereby an increase or decrease in the assayed polypeptide gene expression level compared to the standard expression level is indicative of a particular disorder. With respect to cancer, the presence of a relatively high amount of transcript in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier thereby preventing the development or further progression of the cancer.

Antibodies of the invention can be used to assay protein levels in a biological sample using classical immunohistological methods known to those of skill in the art (e.g., see Jalkanen, et al., J. Cell. Biol. 101:976-985 (1985); Jalkanen, et al., J. Cell. Biol. 105:3087-3096 (1987)). Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase; radioisotopes, such as iodine (125I, 121I), carbon (14C), sulfur (35S), tritium (3H), indium (112In), and technetium (99Tc); luminescent labels, such as luminol; and fluorescent labels, such as fluorescein and rhodamine, and biotin.

One aspect of the invention is the detection and diagnosis of a disease or disorder associated with aberrant expression of a polypeptide of interest in an animal, preferably a mammal and most preferably a human. In one embodiment, diagnosis comprises: a) administering (for example, parenterally, subcutaneously, or intraperitoneally) to a subject an effective amount of a labeled molecule which specifically binds to the polypeptide of interest; b) waiting for a time interval following the administering for permitting the labeled molecule to preferentially concentrate at sites in the subject where the polypeptide is expressed (and for unbound labeled molecule to be cleared to background level); c) determining background level; and d) detecting the labeled molecule in the subject, such that detection of labeled molecule above the background level indicates that-the subject has a particular disease or disorder associated with aberrant expression of the polypeptide of interest. Background level can be determined by various methods including, comparing the amount of labeled molecule detected to a standard value previously determined for a particular system.

It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of 99 mTc. The labeled antibody or antibody fragment will then preferentially accumulate at the location of cells which contain the specific protein. In vivo tumor imaging is described in S. W. Burchiel et al., “Immunopharmacokinetics of Radiolabeled Antibodies and Their Fragments.” (Chapter 13 in Tumor Imaging: The Radiochemical Detection of Cancer, S. W. Burchiel and B. A. Rhodes, eds., Masson Publishing Inc. (1982).

Depending on several variables, including the type of label used and the mode of administration, the time interval following the administration for permitting the labeled molecule to preferentially concentrate at sites in the subject and for unbound labeled molecule to be cleared to background level is 6 to 48 hours or 6 to 24 hours or 6 to 12 hours. In another embodiment the time interval following administration is 5 to 20 days or 5 to 10 days.

In an embodiment, monitoring of the disease or disorder is carried out by repeating the method for diagnosing the disease or disease, for example, one month after initial diagnosis, six months after initial diagnosis, one year after initial diagnosis, etc.

Presence of the labeled molecule can be detected in the patient using methods known in the art for in vivo scanning. These methods depend upon the type of label used. Skilled artisans will be able to determine the appropriate method for detecting a particular label. Methods and devices that may be used in the diagnostic methods of the invention include, but are not limited to, computed tomography (CT), whole body scan such as position emission tomography (PET), magnetic resonance imaging (MRI), and sonography.

In a specific embodiment, the molecule is labeled with a radioisotope and is detected in the patient using a radiation responsive surgical instrument (Thurston et al., U.S. Pat. No. 5,441,050). In another embodiment, the molecule is labeled with a fluorescent compound and is detected in the patient using a fluorescence responsive scanning instrument. In another embodiment, the molecule is labeled with a positron emitting metal and is detected in the patent using positron emission-tomography. In yet another embodiment, the molecule is labeled with a paramagnetic label and is detected in a patient using magnetic resonance imaging (MRI).

Kits

The present invention provides kits that can be used in the above methods. In one embodiment, a kit comprises an antibody of the invention, preferably a purified antibody, in one or more containers. In a specific embodiment, the kits of the present invention contain a substantially isolated polypeptide comprising an epitope which is specifically immunoreactive with an antibody included in the kit. Preferably, the kits of the present invention further comprise a control antibody which does not react with the polypeptide of interest. In another specific embodiment, the kits of the present invention contain a means for detecting the binding of an antibody to a polypeptide of interest (e.g., the antibody may be conjugated to a detectable substrate such as a fluorescent compound, an enzymatic substrate, a radioactive compound or a luminescent compound, or a second antibody which recognizes the first antibody may be conjugated to a detectable substrate).

In another specific embodiment of the present invention, the kit is a diagnostic kit for use in screening serum containing antibodies specific against proliferative and/or cancerous polynucleotides and polypeptides. Such a kit may include a control antibody that does not react with the polypeptide of interest. Such a kit may include a substantially isolated polypeptide antigen comprising an epitope which is specifically immunoreactive with at least one anti-polypeptide antigen antibody. Further, such a kit includes means for detecting the binding of said antibody to the antigen (e.g., the antibody may be conjugated to a fluorescent compound such as fluorescein or rhodamine which can be detected by flow cytometry). In specific embodiments, the kit may include a recombinantly produced or chemically synthesized polypeptide antigen. The polypeptide antigen of the kit may also be attached to a solid support.

In a more specific embodiment the detecting means of the above-described kit includes a solid support to which said polypeptide antigen is attached. Such a kit may also include a non-attached reporter-labeled anti-human antibody. In this embodiment, binding of the antibody to the polypeptide antigen can be detected by binding of the said reporter-labeled antibody.

In an additional embodiment, the invention includes a diagnostic kit for use in screening serum containing antigens of the polypeptide of the invention. The diagnostic kit includes a substantially isolated antibody specifically immunoreactive with polypeptide or polynucleotide antigens, and means for detecting the binding of the polynucleotide or polypeptide antigen to the antibody. In one embodiment, the antibody is attached to a solid support. In a specific embodiment, the antibody may be a monoclonal antibody. The detecting means of the kit may include a second, labeled monoclonal antibody. Alternatively, or in addition, the detecting means may include a labeled, competing antigen.

In one diagnostic configuration, test serum is reacted with a solid phase reagent having a surface-bound antigen obtained by the methods of the present invention. After binding with specific antigen antibody to the reagent and removing unbound serum components by washing, the reagent is reacted with reporter-labeled anti-human antibody to bind reporter to the reagent in proportion to the amount of bound anti-antigen antibody on the solid support. The reagent is again washed to remove unbound labeled antibody, and the amount of reporter associated with the reagent is determined. Typically, the reporter is an enzyme which is detected by incubating the solid phase in the presence of a suitable fluorometric, luminescent or colorimetric substrate (Sigma, St. Louis, Mo.).

The solid surface reagent in the above assay is prepared by known techniques for attaching protein material to solid support material, such as polymeric beads, dip sticks, 96-well plate or filter material. These attachment methods generally include non-specific adsorption of the protein to the support or covalent attachment of the protein, typically through a free amine group, to a chemically reactive group on the solid support, such as an activated carboxyl, hydroxyl, or aldehyde group. Alternatively, streptavidin coated plates can be used in conjunction with biotinylated antigen(s).

Thus, the invention provides an assay system or kit for carrying out this diagnostic method. The kit generally includes a support with surface-bound recombinant antigens, and a reporter-labeled anti-human antibody for detecting surface-bound anti-antigen antibody.

In another embodiment of the present invention, a method of using a GPCR-encoding polynucleotide sequence to purify a molecule or compound in a sample, wherein the molecule or compound specifically binds to the polynucleotide, is contemplated. The method comprises: a) combining a GPCR-encoding polynucleotide of the invention with a sample undergoing testing to determine if the sample contains the molecule or compound, under conditions to allow specific binding; b) detecting specific binding between the GPCR-encoding polynucleotide and the molecule or compound, if present; c) recovering the bound polynucleotide; and d) separating the polynucleotide from the molecule or compound, thereby obtaining a purified molecule or compound.

This invention also relates to a method of using GPCR polynucleotides as diagnostic reagents. For example, the detection of a mutated form of the GPCR gene associated with a dysfunction can provide a diagnostic tool that can add to or define diagnosis of a disease or susceptibility to a disease which results from under-expression, over-expression, or altered expression of GPCRs. Individuals carrying mutations in the GPCR gene may be detected at the DNA level by a variety of techniques.

Nucleic acids for diagnosis may be obtained from various sources of a subject, for example, from cells, tissue, blood, urine, saliva, tissue biopsy or autopsy material. Genomic DNA may be used directly for detection or may be amplified by using PCR or other amplification techniques prior to analysis. RNA or cDNA may also be used in similar fashion. Deletions and insertions in GPCR-encoding polynucleotide can be detected by a change in size of the amplified product compared with that of the normal genotype. Hybridizing amplified DNA to labeled GPCR polynucleotide sequences can identify point mutations. Perfectly matched sequences can be distinguished from mismatched duplexes by RNase digestion or by differences in melting temperatures. DNA sequence differences may also be detected by alterations in electrophoretic mobility of DNA fragments in gels, with or without denaturing agents, or by direct DNA sequencing. See, for example, Myers et al., Science (1985) 230:1242. Sequence changes at specific locations may also be revealed by nuclease protection assays, such as RNase and S1 protection or the chemical cleavage method. (See Cotton et al., Proc. Natl. Acad. Sci., USA (1985) 85:43297-4401).

In another embodiment, an array of oligonucleotide probes comprising GPCR nucleotide sequence or fragments thereof can be constructed to conduct efficient screening of, for example, genetic mutations. Array technology methods are well known, have general applicability and can be used to address a variety of questions in molecular genetics, including gene expression, genetic linkage, and genetic variability (see for example: M. Chee et al., Science, 274:610-613, 1996).

Yet another aspect of the present invention involves a method of screening a library of molecules or compounds with a GPCR-encoding polynucleotide to identify at least one molecule or compound therein which specifically binds to the GPCR polynucleotide sequence. Such a method includes a) combining a GPCR-encoding polynucleotide of the present invention with a library of molecules or compounds under conditions to allow specific binding; and b) detecting specific binding, thereby identifying a molecule or compound, which specifically binds to a GPCR-encoding polynucleotide sequence, wherein the library is selected from DNA molecules, RNA molecules, artificial chromosome constructions, PNAs, peptides and proteins.

The present invention provides diagnostic assays for determining or monitoring through detection of a mutation in a GPCR gene (polynucleotide) described herein susceptibility to the following conditions, diseases, or disorders: cancers; anorexia; bulimia; asthma; Parkinson's disease; acute heart failure; hypotension; hypertension; urinary retention; osteoporosis; angina pectoris; myocardial infarction; ulcers; asthma; allergies; benign prostatic hypertrophy; and psychotic and neurological disorders, including anxiety, headache, migraine, schizophrenia, manic depression, delirium, dementia, severe mental retardation and dyskinesias, such as Huntington's disease or Gilles de la Tourette's syndrome.

In addition, such diseases, disorder, or conditions, can be diagnosed by methods of determining from a sample derived from a subject having an abnormally decreased or increased level of GPCR polypeptide or GPCR mRNA. Decreased or increased expression can be measured at the RNA level using any of the methods well known in the art for the quantification of polynucleotides, such as, for example, PCR, RT-PCR, RNase protection, Northern blotting and other hybridization methods. Assay techniques that can be used to determine levels of a protein, such as a GPCR in a sample derived from a host are well known to those of skill in the art. Such assay methods include, without limitation, radioimmunoassays, competitive-binding assays, Western Blot analysis and ELISA assays.

In another of its aspects, this invention relates to a kit for detecting and diagnosing a GPCR-associated disease or susceptibility to such a disease, which comprises a GPCR polynucleotide, preferably the nucleotide sequence of SEQ ID NOs:1-13, or a fragment thereof; or a nucleotide sequence complementary to the GPCR polynucleotide of SEQ ID NOs:1-13; or a GPCR polypeptide, preferably the polypeptide of SEQ ID NOs:14-26, or a fragment thereof; or an antibody to the GPCR polypeptide, preferably to the polypeptide of SEQ ID NOs:14-26, an epitope-containing portion thereof, or combinations of the foregoing. It will be appreciated that in any such kit, any of the previously mentioned components may comprise a substantial component. Also preferably included are instructions for use.

The GPCR polynucleotides which may be used in the diagnostic assays according to the present invention include oligonucleotide sequences, complementary RNA and DNA molecules, and PNAs. The polynucleotides may be used to detect and quantify GPCR-encoding nucleic acid expression in biopsied tissues in which expression (or under- or over-expression) of the GPCR polynucleotide may be determined, as well as correlated with disease. The diagnostic assays may be used to distinguish between the absence of GPCR, the presence of GPCR, or the excess expression of GPCR, and to monitor the regulation of GPCR polynucleotide levels during therapeutic treatment or intervention.

In a related aspect, hybridization with PCR probes which are capable of detecting polynucleotide sequences, including genomic sequences, encoding a GPCR polypeptide according to the present invention, or closely related molecules, may be used to identify nucleic acid sequences which encode a GPCR polypeptide. The specificity of the probe, whether it is made from a highly specific region, for example, about 8 to 10 contiguous nucleotides in the 5′ regulatory region, or a less specific region, for example, especially in the 3′ coding region, and the stringency of the hybridization or amplification (maximal, high, intermediate, or low) will determine whether the probe identifies only naturally occurring sequences encoding GPCR polypeptide, alleles thereof, or related sequences.

Probes may also be used for the detection of related sequences, and should preferably contain at least 50% of the nucleotides encoding the GPCR polypeptide. The hybridization probes or primers of this invention may be DNA or RNA and may be derived from the nucleotide sequences of SEQ ID NOs:1-13, or as listed in Tables 2 and 3, or may be derived from genomic sequences including promoter, enhancer elements, and introns of the naturally occurring GPCR protein, wherein the probes or primers comprise a polynucleotide sequence capable of hybridizing with a polynucleotide of SEQ ID NOs:1-13, under low, moderate, or high stringency conditions.

Methods for producing specific hybridization probes for DNA encoding the GPCR polypeptides include the cloning of a nucleic acid sequence that encodes the GPCR polypeptide, or GPCR derivatives, into vectors for the production of mRNA probes. Such vectors are known in the art, or are commercially available, and may be used to synthesize RNA probes in vitro by means of the addition of the appropriate RNA polymerases and the appropriate labeled nucleotides. Hybridization probes may be labeled by a variety of detector/reporter groups, including, but not limited to, radionuclides such as ³²P or ³⁵S, or enzymatic labels, such as alkaline phosphatase coupled to the probe via avidin/biotin coupling systems, and the like.

The polynucleotide sequences encoding the GPCR polypeptides of this invention, or fragments thereof, may be used for the diagnosis of disorders associated with expression of GPCRs. The polynucleotide sequence encoding the GPCR polypeptide may be used in Southern or Northern analysis, dot blot, or other membrane-based technologies; in PCR technologies; or in dipstick, pin, ELISA or chip assays utilizing fluids or tissues from patient biopsies to detect the status of, for example, levels of, or overexpression of, a GPCR, or to detect altered GPCR expression or levels. Such qualitative or quantitative methods are commonly practiced in the art.

In a particular aspect, a nucleotide sequence encoding a GPCR polypeptide as described herein may be useful in assays that detect activation or induction of various neoplasms, cancers, or other GPCR-related diseases, disorders, or conditions. The nucleotide sequence encoding a GPCR polypeptide may be labeled by standard methods, and added to a fluid or tissue sample from a patient, under conditions suitable for the formation of hybridization complexes. After a suitable incubation period, the sample is washed and the signal is quantified and compared with a standard value. If the amount of signal in the biopsied or extracted sample is significantly altered from that of a comparable control sample, the nucleotide sequence has hybridized with nucleotide sequence present in the sample, and the presence of altered levels of nucleotide sequence encoding the GPCR polypeptide in the sample indicates the presence of the associated disease. Such assays may also be used to evaluate the efficacy of a particular therapeutic treatment regimen in animal studies, in clinical trials, or in monitoring the treatment or responsiveness of an individual patient.

Once disease is established and a treatment protocol is initiated, hybridization assays may be repeated on a regular basis to evaluate whether the level of expression in the patient begins to approximate that which is observed in a normal individual. The results obtained from successive assays may be used to show the efficacy of treatment over a period ranging from several days to months.

With respect to tumors or cancer, the presence of an abnormal amount or level of a GPCR transcript in biopsied tissue from an individual may indicate a predisposition for the development of the disease, or may provide a means for detecting the disease prior to the appearance of actual clinical symptoms. A more definitive diagnosis of this type may allow health practitioners to employ preventative measures or aggressive treatment earlier, thereby preventing the development or further progression of the tumor or cancer.

Additional diagnostic uses for oligonucleotides designed from the nucleic acid sequences encoding the novel GPCR polypeptides of this invention can involve the use of PCR. Such oligomers may be chemically synthesized, generated enzymatically, or produced from a recombinant source. Oligomers will preferably comprise two nucleotide sequences: one with sense orientation (5′→3′) and another with antisense orientation (3′→5′), employed under optimized conditions for identification of a specific gene or condition. The same two oligomers, nested sets of oligomers, or even a degenerate pool of oligomers may be employed under less stringent conditions for detection and/or quantification of closely related DNA or RNA sequences.

Methods suitable for quantifying the expression of GPCR include radiolabeling or biotinylating nucleotides, co-amplification of a control nucleic acid, and standard curves onto which the experimental results are interpolated (P. C. Melby et al., 1993, J. Immunol. Methods, 159:235-244; and C. Duplaa et al., 1993, Anal. Biochem., 229-236). The speed of quantifying multiple samples may be accelerated by running the assay in an ELISA format where the oligomer of interest is presented in various dilutions and a spectrophotometric or calorimetric response gives rapid quantification.

In one embodiment of the invention, a compound to be tested can be radioactively, calorimetrically or fluorimetrically labeled using methods well known in the art and incubated with the GPCR for testing. After incubation, it is determined whether the test compound is bound to the GPCR polypeptide. If so, the compound is to be considered a potential agonist or antagonist. Functional assays are performed to determine whether the receptor activity is activated (or enhanced or increased) or inhibited (or decreased or reduced). These assays include, but are not limited to, cell cycle analysis and in vivo tumor formation assays. Responses can also be measured in cells expressing the receptor using signal transduction systems including, but not limited to, protein phosphorylation, adenylate cyclase activity, phosphoinositide hydrolysis, guanylate cyclase activity, ion fluxes (i.e. calcium) and pH changes. These types of responses can either be present in the host cell or introduced into the host cell along with the receptor.

The present invention further embraces a method of screening for candidate compounds capable of modulating the activity of a GPCR-encoding polypeptide. Such a method comprises a) contacting a test compound with a cell or tissue expressing a GPCR polypeptide of the invention (e.g., recombinant expression); and b) selecting as candidate modulating compounds those test compounds that modulate activity of the GPCR polypeptide. Those candidate compounds which modulate GPCR activity are preferably agonists or antagonists, more preferably antagonists of GPCR activity.

The present invention encompasses the identification of compounds and drugs which stimulate Gene 1, 2, 3, 4, 5, 6, 7, 8, 9, 10, 11, 12, or 13 on the one hand (i.e., agonists) and which inhibit the function of Gene 1, 2, 3, 4, 5, 6, 7, 8, 9, 10, 11, 12, or 13 on the other hand (i.e., antagonists). In general, such screening procedures involve providing appropriate cells which express the receptor polypeptide of the present invention on the surface thereof. Such cells may include, for example, cells from mammals, yeast, Drosophila or E. coli. In a preferred embodimenta, a polynucleotide encoding the receptor of the present invention may be employed to transfect cells to thereby express the Gene 1, 2, 3, 4, 5, 6, 7, 8, 9, 10, 11, 12, or 13 polypeptide. The expressed receptor may then be contacted with a test compound to observe binding, stimulation or inhibition of a functional response.

One such screening procedure involves the use of melanophores which are transfected to express the Gene 1, 2, 3, 4, 5, 6, 7, 8, 9, 10, 11, 12, or 13 polypeptide of the present invention. Such a screening technique is described in PCT WO 92/01810, published Feb. 6, 1992. Such an assay may be employed to screen for a compound which inhibits activation of the receptor polypeptide of the present invention by contacting the melanophore cells which encode the receptor with both the receptor ligand, such as LPA, and a compound to be screened. Inhibition of the signal generated by the ligand indicates that a compound is a potential antagonist for the receptor, i.e., inhibits activation of the receptor.

The technique may also be employed for screening of compounds which activate the receptor by contacting such cells with compounds to be screened and determining whether such compound generates a signal, i.e., activates the receptor. Other screening techniques include the use of cells which express the Gene 1, 2, 3, 4, 5, 6, 7, 8, 9, 10, 11, 12, or 13 polypeptide (for example, transfected CHO cells) in a system which measures extracellular pH changes caused by receptor activation. In this technique, compounds may be contacted with cells expressing the receptor polypeptide of the present invention. A second messenger response, e.g., signal transduction or pH changes, is then measured to determine whether the potential compound activates or inhibits the receptor.

Another screening technique involves expressing the Gene 1, 2, 3, 4, 5, 6, 7, 8, 9, 10, 11, 12, or 13 polypeptide in which the receptor is linked to phospholipase C or D. Representative examples of such cells include, but are not limited to, endothelial cells, smooth muscle cells, and embryonic kidney cells. The screening may be accomplished as hereinabove described by detecting activation of the receptor or inhibition of activation of the receptor from the phospholipase second signal.

Another method involves screening for compounds which are antagonists or agonists by determining inhibition of binding of labeled ligand, such as LPA, to cells which have the receptor on the surface thereof, or cell membranes containing the receptor. Such a method involves transfecting a cell (such as eukaryotic cell) with DNA encoding the Gene 1, 2, 3, 4, 5, 6, 7, 8, 9, 10, 11, 12, or 13 polypeptide such that the cell expresses the receptor on its surface. The cell is then contacted with a potential antagonist or agonist in the presence of a labeled form of a ligand, such as LPA. The ligand can be labeled, e.g., by radioactivity. The amount of labeled ligand bound to the receptors is measured, e.g., by measuring radioactivity associated with transfected cells or membrane from these cells. If the compound binds to the receptor, the binding of labeled ligand to the receptor is inhibited as determined by a reduction of labeled ligand which binds to the receptors. This method is called binding assay.

Another screening procedure involves the use of mammalian cells (CHO, HEK 293, Xenopus Oocytes, RBL-2H3, etc) which are transfected to express the receptor of interest. The cells are loaded with an indicator dye that produces a fluorescent signal when bound to calcium, and the cells are contacted with a test substance and a receptor agonist, such as LPA. Any change in fluorescent signal is measured over a defined period of time using, for example, a fluorescence spectrophotometer or a fluorescence imaging plate reader. A change in the fluorescence signal pattern generated by the ligand indicates that a compound is a potential antagonist or agonist for the receptor.

Another screening procedure involves use of mammalian cells (CHO, HEK293, Xenopus Oocytes, RBL-2H3, etc.) which are transfected to express the receptor of interest, and which are also transfected with a reporter gene construct that is coupled to activation of the receptor (for example, luciferase or beta-galactosidase behind an appropriate promoter). The cells are contacted with a test substance and the receptor agonist (ligand), such as LPA, and the signal produced by the reporter gene is measured after a defined period of time. The signal can be measured using a luminometer, spectrophotometer, fluorimeter, or other such instrument appropriate for the specific reporter construct used. Change of the signal generated by the ligand indicates that a compound is a potential antagonist or agonist for the receptor.

Another screening technique for antagonists or agonits involves introducing RNA encoding-the Gene 1, 2, 3, 4, 5, 6, 7, 8, 9, 10, 11, 12, or 13 polypeptide into Xenopus oocytes (or CHO, HEK 293, RBL-2H3, etc.) to transiently or stably express the receptor. The receptor oocytes are then contacted with the receptor ligand, such as LPA, and a compound to be screened. Inhibition or activation of the receptor is then determined by detection of a signal, such as, cAMP, calcium, proton, or other ions.

Another method involves screening for Gene 1, 2, 3, 4, 5, 6, 7, 8, 9, 10, 11, 12, or 13 polypeptide inhibitors by determining inhibition or stimulation of Gene 1, 2, 3, 4, 5, 6, 7, 8, 9, 10, 11, 12, or 13 polypeptide-mediated cAMP and/or adenylate cyclase accumulation or dimunition. Such a method involves transiently or stably transfecting a eukaryotic cell with Gene 1, 2, 3, 4, 5, 6, 7, 8, 9, 10, 11, 12, or 13 polypeptide receptor to express the receptor on the cell surface.

The cell is then exposed to potential antagonists or agonists in the presence of Gene 1, 2, 3, 4, 5, 6, 7, 8, 9, 10, 11, 12, or 13 polypeptide ligand, such as LPA. The changes in levels of cAMP is then measured over a defined period of time, for example, by radio-immuno or protein binding assays (for example using Flashplates or a scintillation proximity assay). Changes in cAMP levels can also be determined by directly measuring the activity of the enzyme, adenylyl cyclase, in broken cell preparations. If the potential antagonist or agonist binds the receptor, and thus inhibits Gene 1, 2, 3, 4, 5, 6, 7, 8, 9, 10, 11, 12, or 13 polypeptide-ligand binding, the levels of Gene 1, 2, 3, 4, 5, 6, 7, 8, 9, 10, 11, 12, or 13 polypeptide-mediated cAMP, or adenylate cyclase activity, will be reduced or increased.

One preferred screening method involves co-transfecting HEK-293 cells with a mammalian expression plasmid encoding a G-protein coupled receptor (GPCR), such as Gene 1, 2, 3, 4, 5, 6, 7, 8, 9, 10, 11, 12, or 13, along with a mixture comprised of mammalian expression plasmids cDNAs encoding GU15 (Wilkie T. M. et al Proc Natl Acad Sci USA 1991 88: 10049-10053), GU16 (Amatruda T. T. et al Proc Natl Acad Sci USA 1991 8: 5587-5591, and three chimeric G-proteins refered to as Gqi5, Gqs5, and Gqo5 (Conklin B R et al Nature 1993 363: 274-276, Conklin B. R. et al Mol Pharmacol 1996 50: 885-890). Following a 24 h incubation the trasfected HEK-293 cells are plated into poly-D-lysine coated 96 well black/clear plates (Becton Dickinson, Bedford, Mass.).

The cells are assayed on FLIPR (Fluorescent Imaging Plate Reader, Molecular Devices, Sunnyvale, Calif.) for a calcium mobilization response following addition of test ligands. Upon identification of a ligand which stimulates calcium mobilization in HEK-293 cells expressing a given GPCR and the G-protein mixtures, subsequent experiments are performed to determine which, if any, G-protein is required for the functional response. HEK-293 cells are then transfected with the test GPCR, or co-transfected with the test GPCR and GO15, GD16, GqiS, Gqs5, or Gqo5. If the GPCR requires the presence of one of the G-proteins for functional expression in HEK-293 cells, all subsequent experiments are performed with HEK-293 cell cotransfected with the GPCR and the G-protein which gives the best response. Alternatively, the receptor can be expressed in a different cell line, for example RBL-2H3, without additional Gproteins.

Another screening method for agonists and antagonists relies on the endogenous pheromone response pathway in the yeast, Saccharomyces cerevisiae. Heterothallic strains of yeast can exist in two mitotically stable haploid mating types, MATa and MATa. Each cell type secretes a small peptide hormone that binds to a G-protein coupled receptor on opposite mating type cells which triggers a MAP kinase cascade leading to G1 arrest as a prelude to cell fusion.

Genetic alteration of certain genes in the pheromone response pathway can alter the normal response to pheromone, and heterologous expression and coupling of human G-protein coupled receptors and humanized G-protein subunits in yeast cells devoid of endogenous pheromone receptors can be linked to downstream signaling pathways and reporter genes (e.g., U.S. Pat. Nos. 5,063,154; 5,482,835; 5,691,188). Such genetic alterations include, but are not limited to, (i) deletion of the STE2 or STE3 gene encoding the endogenous G-protein coupled pheromone receptors; (ii) deletion of the FAR1 gene encoding a protein that normally associates with cyclindependent kinases leading to cell cycle arrest; and (iii) construction of reporter genes fused to the FUS 1 gene promoter (where FUS 1 encodes a membrane-anchored glycoprotein required for cell fusion). Downstream reporter genes can permit either a positive growth selection (e.g., histidine prototrophy using the FUS1-HIS3 reporter), or a calorimetric, fluorimetric or spectrophotometric readout, depending on the specific reporter construct used (e.g., b-galactosidase induction using a FUS1-LacZ reporter).

The yeast cells can be further engineered to express and secrete small peptides from random peptide libraries, some of which can permit autocrine activation of heterologously expressed human (or mammalian) G-protein coupled receptors (Broach, J. R. and Thorner, J., Nature 384: 14-16, 1996; Manfredi et al., Mol. Cell. Biol. 16: 4700-4709,1996). This provides a rapid direct growth selection (e.g, using the FUS 1-HIS3 reporter) for surrogate peptide agonists that activate characterized or orphan receptors. Alternatively, yeast cells that functionally express human (or mammalian) G-protein coupled receptors linked to a reporter gene readout (e.g., FUS1-LacZ) can be used as a platform for high-throughput screening of known ligands, fractions of biological extracts and libraries of chemical compounds for either natural or surrogate ligands.

Functional agonists of sufficient potency (whether natural or surrogate) can be used as screening tools in yeast cell-based assays for identifying G-protein coupled receptor antagonists. For example, agonists will promote growth of a cell with FUS-HIS3 reporter or give positive readout for a cell with FUSI-LacZ. However, a candidate compound which inhibits growth or negates the positive readout induced by an agonist is an antagonist. For this purpose, the yeast system offers advantages over mammalian expression systems due to its ease of utility and null receptor background (lack of endogenous G-protein coupled receptors) which often interferes with the ability to identify agonists or antagonists.

Therapeutic Assays

The GPCR proteins according to this invention may play a role in cell signaling, in cell cycle regulation, and/or in neurological disorders. The GPCR proteins may further be involved in neoplastic, cardiovascular, and immunological disorders.

In one embodiment in accordance with the present invention, the novel GPCR protein may play a role in neoplastic disorders. An antagonist or inhibitor of the GPCR protein may be administered to an individual to prevent or treat a neoplastic disorder. Such disorders may include, but are not limited to, adenocarcinoma, leukemia, lymphoma, melanoma, myeloma, sarcoma, and teratocarcinoma, and particularly, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus. In a related aspect, an antibody which specifically binds to GPCR may be used directly as an antagonist or indirectly as a targeting or delivery mechanism for bringing a pharmaceutical agent to cells or tissue which express the GPCR polypeptide.

In yet another embodiment of the present invention, an antagonist or inhibitory agent of the GPCR polypeptide may be administered therapeutically to an individual to prevent or treat an immunological disorder. Such disorders may include, but are not limited to, AIDS, HIV infection, Addison's disease, adult respiratory distress syndrome, allergies, anemia, asthma, atherosclerosis, bronchitis, cholecystitis, Crohn's disease, ulcerative colitis, atopic dermatitis, dermatomyositis, diabetes mellitus, emphysema, erythema nodosum, atrophic gastritis, glomerulonephritis, gout, Graves' disease, hypereosinophilia, irritable bowel syndrome, lupus erythematosus, multiple sclerosis, myasthenia gravis, myocardial or pericardial inflammation, osteoarthritis, osteoporosis, pancreatitis, polymyositis, rheumatoid arthritis, scleroderma, Sjogren's syndrome, and autoimmune thyroiditis; complications of cancer, hemodialysis, extracorporeal circulation; viral, bacterial, fungal, parasitic, protozoal, and helminthic infections, trauma, and neurological disorders including, but not limited to, akathesia, Alzheimer's disease, amnesia, amyotrophic lateral sclerosis, bipolar disorder, catatonia, cerebral neoplasms, dementia, depression, Down's syndrome, tardive dyskinesia, dystonias, epilepsy, Huntington's disease, multiple sclerosis, Parkinson's disease, paranoid psychoses, schizophrenia, and Tourette's disorder.

A preferred method of treating a GPCR associated disease, disorder, syndrome, or condition in a mammal comprises administration of a modulator, preferably an inhibitor or antagonist, of a GPCR polypeptide or homologue of the invention, in an amount effective to treat, reduce, and/or ameliorate the symptoms incurred by the GPCR-associated disease, disorder, syndrome, or condition. In some instances, an agonist or enhancer of a GPCR polypeptide or homologue of the invention is administered in an amount effective to treat and/or ameliorate the symptoms incurred by a GPCR-related disease, disorder, syndrome, or condition. In other instances, the administration of a novel GPCR polypeptide or homologue thereof pursuant to the present invention is envisioned for administration to treat a GPCR associated disease.

In yet another embodiment of the present invention, an expression vector containing the complement of the polynucleotide encoding a GPCR polypeptide is administered to an individual to treat or prevent any one of the types of diseases, disorders, or conditions previously described, in an antisense therapy method.

The GPCR proteins; modulators, including antagonists, antibodies, and agonists; complementary sequences; or vectors of the present invention can also be administered in combination with other appropriate therapeutic agents as necessary or desired. Selection of the appropriate agents for use in combination therapy may be made by the skilled practitioner in the art, according to conventional pharmaceutical and clinical principles. The combination of therapeutic agents may act synergistically to effect the treatment or prevention of the various disorders described above. Using this approach, one may be able to achieve therapeutic efficacy with lower dosages of each agent, thus reducing the potential for adverse side effects or adverse events.

Antagonists or inhibitors of the GPCR polypeptide of this invention can be produced using methods which are generally known in the art. In particular, purified GPCR protein, or fragments thereof, can be used to produce antibodies, or to screen libraries of pharmaceutical agents, to identify those which specifically bind to the novel GPCR polypeptides as described herein.

Antibodies specific for GPCR polypeptide, or immunogenic peptide fragments thereof, can be generated using methods that have long been known and conventionally practiced in the art. Such antibodies may include, but are not limited to, polyclonal, monoclonal, neutralizing antibodies, (i.e., those which inhibit dimer formation), chimeric, single chain, Fab fragments, and fragments produced by an Fab expression library. Non-limiting examples of GPCR polypeptides or immunogenic fragments thereof that may be used to generate antibodies are provided in SEQ ID NOs:14-26.

For the production of antibodies, various hosts, including goats, rabbits, sheep, rats, mice, humans, and others, can be immunized by injection with one or more of the GPCR polypeptides, or any immunogenic and/or epitope-containing fragment or oligopeptide thereof, which have immunogenic properties. Depending on the host species, various adjuvants may be used to increase the immunological response. Non-limiting examples of suitable adjuvants include Freund's (incomplete), mineral gels such as aluminum hydroxide or silica, and surface active substances such as lysolecithin, pluronic polyols, polyanions, peptides, oil emulsions, KLH, and dinitrophenol. Adjuvants typically used in humans include BCG (bacilli Calmette Guérin) and Corynebacterium parvumn.

Preferably, the GPCR polypeptides, peptides, fragments, or oligopeptides used to induce antibodies to the GPCR polypeptide immunogens have an amino acid sequence of at least five amino acids in length, and more preferably, at least 7-10, or more, amino acids. It is also preferable that the immunogens are identical to a portion of the amino acid sequence of the natural protein; they may also contain the entire amino acid sequence of a small, naturally occurring molecule. The peptides, fragments or oligopeptides may comprise a single epitope or antigenic determinant or multiple epitopes. Short stretches of GPCR amino acids may be fused with another protein as carrier, such as KLH, such that antibodies are produced against the chimeric molecule.

Monoclonal antibodies to the GPCR polypeptides, or immunogenic fragments thereof, may be prepared using any technique which provides for the production of antibody molecules by continuous cell lines in culture. Such techniques are conventionally used in the art. These include, but are not limited to, the hybridoma technique, the human B-cell hybridoma technique, and the EBV-hybridoma technique (G. Kohler et al., 1975, Nature, 256:495-497; D. Kozbor et al., 1985, J. Immunol. Methods, 81:31-42; R. J. Cote et al., 1983, Proc. Natl. Acad. Sci. USA, 80:2026-2030; and S. P. Cole et al., 1984, Mol. Cell Biol., 62:109-120). The production of monoclonal antibodies to immunogenic proteins and peptides is well known and routinely used in the art.

In addition, techniques developed for the production of “chimeric antibodies,” the splicing of mouse antibody genes to human antibody genes to obtain a molecule with appropriate antigen specificity and biological activity can be used (S. L. Morrison et al., 1984, Proc. Natl. Acad. Sci. USA, 81:6851-6855; M. S. Neuberger et al., 1984, Nature, 312:604-608; and S. Takeda et al., 1985, Nature, 314:452-454). Alternatively, techniques described for the production of single chain antibodies may be adapted, using methods known in the art, to produce GPCR polypeptide-specific single chain antibodies. Antibodies with related specificity, but of distinct idiotypic composition, may be generated by chain shuffling from random combinatorial immunoglobulin libraries (D. R. Burton, 1991, Proc. Natl. Acad. Sci. USA, 88:11120-3). Antibodies may also be produced by inducing in vivo production in the lymphocyte population or by screening recombinant immunoglobulin libraries or panels of highly specific binding reagents as disclosed in the literature (R. Orlandi et al., 1989, Proc. Natl. Acad. Sci. USA, 86:3833-3837 and G. Winter et al., 1991, Nature, 349:293-299).

Antibody fragments, which contain specific binding sites for a GPCR polypeptide, may also be generated. For example, such fragments include, but are not limited to, F(ab′)₂ fragments which can be produced by pepsin digestion of the antibody molecule and Fab fragments which can be generated by reducing the disulfide bridges of the F(ab′)₂ fragments. Alternatively, Fab expression libraries may be constructed to allow rapid and easy identification of monoclonal Fab fragments with the desired specificity (e.g., W. D. Huse et al., 1989, Science, 254.1275-1281).

Various immunoassays can be used for screening to identify antibodies having the desired specificity. Numerous protocols for competitive binding or immunoradiometric assays using either polyclonal or monoclonal antibodies with established specificities are well known in the art. Such immunoassays typically involve measuring the formation of complexes between a GPCR polypeptide and its specific antibody. A two-site, monoclonal-based immunoassay utilizing monoclonal antibodies reactive with two non-interfering GPCR polypeptide epitopes is suitable, but a competitive binding assay may also be employed (Maddox, supra).

To induce an immunological response in a mammal, a host animal is inoculated with a GPCR polypeptide, or a fragment thereof, of this invention in an amount adequate to produce an antibody and/or a T cell immune response to protect the animal from a disease or disorder associated with the expression or production of a GPCR polypeptide. Yet another aspect of the invention relates to a method of inducing immunological response in a mammal, if applicable or required. Such a method comprises delivering GPCR polypeptide via a vector directing expression of GPCR polynucleotide in vivo in order to induce such an immunological response to produce antibody to protect said animal from GPCR-related diseases.

A further aspect of the invention relates to an immunological vaccine or immunogen formulation or composition which, when introduced into a mammalian host, induces an immunological response in that mammal to a GPCR polypeptide wherein the composition comprises a GPCR polypeptide or GPCR gene. The vaccine or immunogen formulation may further comprise a suitable carrier. Since the GPCR polypeptide may be broken down in the stomach, it is preferably administered parenterally (including subcutaneous, intramuscular, intravenous, intradermal, etc., injection). Formulations suitable for parenteral administration include aqueous and non-aqueous sterile injection solutions which may contain anti-oxidants, buffers, bacteriostats and solutes which render the formulation isotonic with the blood of the recipient; and aqueous and non-aqueous sterile suspensions which may include suspending agents or thickening agents.

The formulations may be presented in unit-dose or multi-dose containers, for example, sealed ampoules and vials, and may be stored in a freeze-dried condition requiring only the addition of the sterile liquid carrier immediately prior to use. A vaccine formulation may also include adjuvant systems for enhancing the immunogenicity of the formulation, such as oil-in-water systems and other systems known in the art. The dosage will depend on the specific activity of the vaccine and can be readily determined by routine experimentation.

In a specific embodiment, formulations of the present invention may further comprise antagonists of P-glycoprotein (also referred to as the multiresistence protein, or PGP), including antagonists of its encoding polynucleotides (e.g., antisense oligonucleotides, ribozymes, zinc-finger proteins, etc.). P-glycoprotein is well known for decreasing the efficacy of various drug administrations due to its ability to export intracellular levels of absorbed drug to the cell exterior. While this activity has been particularly pronounced in cancer cells in response to the administration of chemotherapy regimens, a variety of other cell types and the administration of other drug classes have been noted (e.g., T-cells and anti-HIV drugs). In fact, certain mutations in the PGP gene significantly reduces PGP function, making it less able to force drugs out of cells. People who have two versions of the mutated gene—one inherited from each parent—have more than four times less PGP than those with two normal versions of the gene. People may also have one normal gene and one mutated one. Certain ethnic populations have increased incidence of such PGP mutations. Among individuals from Ghana, Kenya, the Sudan, as well as African Americans, frequency of the normal gene ranged from 73% to 84%. In contrast, the frequency was 34% to 59% among British whites, Portuguese, Southwest Asian, Chinese, Filipino and Saudi populations. As a result, certain ethnic populations may require increased administration of PGP antagonist in the formulation of the present invention to arrive at the an efficacious dose of the therapeutic (e.g., those from African descent). Conversely, certain ethnic populations, particularly those having increased frequency of the mutated PGP (e.g., of Caucasian descent, or non-African descent) may require less pharmaceutical compositions in the formulation due to an effective increase in efficacy of such compositions as a result of the increased effective absorption (e.g., less PGP activity) of said composition.

Moreover, in another specific embodiment, formulations of the present invention may further comprise antagonists of OATP2 (also referred to as the multiresistance protein, or MRP2), including antagonists of its encoding polynucleotides (e.g., antisense oligonucleotides, ribozymes, zinc-finger proteins, etc.). The invention also further comprises any additional antagonists known to inhibit proteins thought to be attributable to a multidrug resistant phenotype in proliferating cells.

Preferred antagonists that formulations of the present may comprise include the potent P-glycoprotein inhibitor elacridar, and/or LY-335979. Other P-glycoprotein inhibitors known in the art are also encompassed by the present invention.

Polypeptide or polynucleotides and/or agonist or antagonists of the present invention may also be used to increase the efficacy of a pharmaceutical composition, either directly or indirectly. Such a use may be administered in simultaneous conjunction with said pharmaceutical, or separately through either the same or different route of administration (e.g., intravenous for the polynucleotide or polypeptide of the present invention, and orally for the pharmaceutical, among others described herein.).

In an aspect of the present invention, the polynucleotide encoding a GPCR polypeptide, or any fragment or complement thereof, as described herein may be used for therapeutic purposes. For instance, antisense to a GPCR polynucleotide encoding a GPCR polypeptide, may be used in situations in which it would be desirable to block the transcription of GPCR mRNA. In particular, cells may be transformed, transfected, or injected with sequences complementary to polynucleotides encoding GPCR polypeptide. Thus, complementary molecules may be used to modulate GPCR polynucleotide and polypeptide activity, or to achieve regulation of gene function. Such technology is well known in the art, and sense or antisense oligomers or oligonucleotides, or larger fragments, can be designed from various locations along the coding or control regions of the GPCR polynucleotide sequences encoding the novel GPCR polypeptides.

Polypeptides used in treatment can also be generated endogenously in the subject, in treatment modalities often referred to as “gene therapy”. Thus for example, cells from a subject may be engineered with a polynucleotide, such as DNA or RNA, to encode a polypeptide ex vivo, for example, by the use of a retroviral plasmid vector. The cells can then be introduced into the subject's body in which the desired polypeptide is expressed.

A gene encoding a GPCR polypeptide can be turned off by transforming a cell or tissue with an expression vector that expresses high levels of a GPCR polypeptide-encoding polynucleotide, or a fragment thereof. Such constructs may be used to introduce untranslatable sense or antisense sequences into a cell. Even in the absence of integration into the DNA, such vectors may continue to transcribe RNA molecules until they are disabled by endogenous nucleases. Transient expression may last for a month or more with a non-replicating vector, and even longer if appropriate replication elements are designed to be part of the vector system.

Modifications of gene expression can be obtained by designing antisense molecules or complementary nucleic acid sequences (DNA, RNA, or PNA), to the control, 5′, or regulatory regions of a GPCR polynucleotide sequence encoding a GPCR polypeptide, (e.g., a signal sequence, promoters, enhancers, and introns). Oligonucleotides may be derived from the transcription initiation site, for example, between positions −10 and +10 from the start site.

Similarly, inhibition can be achieved using “triple helix” base-pairing methodology. Triple helix pairing is useful because it causes inhibition of the ability of the double helix to open sufficiently for the binding of polymerases, transcription factors, or regulatory molecules. Recent therapeutic advances using triplex DNA have been described (see, for example, J. E. Gee et al., 1994, In: B. E. Huber and B. I. Carr, Molecular and Immunologic Approaches, Futura Publishing Co., Mt. Kisco, N.Y.). The antisense molecule or complementary sequence may also be designed to block translation of mRNA by preventing the transcript from binding to ribosomes.

Many methods for introducing vectors into cells or tissues are available and are equally suitable for use in vivo, in vitro, and ex vivo. For ex vivo therapy, vectors may be introduced into stem cells or bone marrow cells obtained from the patient and clonally propagated for autologous transplant back into that same patient. Delivery by transfection, direct injection (e.g., microparticle bombardment) and by liposome injections may be achieved using methods which are well known in the art.

Any of the therapeutic methods described above can be applied to any individual in need of such therapy, including, for example, mammals such as dogs, cats, cows, horses, rabbits, monkeys, and most preferably, humans.

Administration

A further embodiment of the present invention embraces the administration of a pharmaceutical composition, in conjunction with a pharmaceutically acceptable carrier, diluent, or excipient, to achieve any of the above-described therapeutic uses and effects. Such pharmaceutical compositions can comprise GPCR nucleic acid, polypeptide, or peptides, antibodies to GPCR polypeptide, mimetics, GPCR modulators, such as agonists, antagonists, or inhibitors of a GPCR polypeptide or polynucleotide. The compositions can be administered alone, or in combination with at least one other agent or reagent, such as a stabilizing compound, which may be administered in any sterile, biocompatible pharmaceutical carrier, including, but not limited to, saline, buffered saline, dextrose, and water. The compositions may be administered to a patient alone, or in combination with other agents, drugs, hormones, or biological response modifiers.

The pharmaceutical compositions for use in the present invention can be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, vaginal, or rectal means.

In addition to the active ingredients (e.g., GPCR nucleic acid or polypeptide, or functional fragments thereof, or a GPCR agonist or antagonist), the pharmaceutical compositions may contain pharmaceutically acceptable/physiologically suitable carriers or excipients comprising auxiliaries which facilitate processing of the active compounds into preparations that can be used pharmaceutically. Further details on techniques for formulation and administration are provided in the latest edition of Remington's Pharmaceutical Sciences (Mack Publishing Co., Easton, Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

In addition, pharmaceutical preparations for oral use can be obtained by the combination of active compounds with a solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropyl-methylcellulose, or sodium carboxymethylcellulose; gums, including arabic and tragacanth, and proteins such as gelatin and collagen. If desired, disintegrating or solubilizing agents may be added, such as cross-linked polyvinyl pyrrolidone, agar, alginic acid, or a physiologically acceptable salt thereof, such as sodium alginate.

Dragee cores may be used in conjunction with physiologically suitable coatings, such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification, or to characterize the quantity of active compound, i.e., dosage.

Pharmaceutical preparations, which can be used orally, further include push-fit capsules made of gelatin, as well as soft, scaled capsules made of gelatin and a coating, such as glycerol or sorbitol. Push-fit capsules can contain active ingredients mixed with fillers or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers.

Pharmaceutical formulations suitable for parenteral administration may be formulated in aqueous solutions, preferably in physiologically compatible buffers such as Hanks' solution, Ringer's solution, or physiologically buffered saline. Aqueous injection suspensions may contain substances, which increase the viscosity of the suspension, such as sodium carboxymethyl cellulose, sorbitol, or dextran. In addition, suspensions of the active compounds may be prepared as appropriate oily injection suspensions. Suitable lipophilic solvents or vehicles include fatty oils such as sesame oil, or synthetic fatty acid esters, such as ethyloleate or triglycerides, or liposomes. Optionally, the suspension may also contain suitable stabilizers or agents which increase the solubility of the compounds to allow for the preparation of highly concentrated solutions.

For topical or nasal administration, penetrants or permeation agents (enhancers) that are appropriate to the particular barrier to be permeated are used in the formulation. Such penetrants are generally known in the art.

The pharmaceutical compositions of the present invention may be manufactured in a manner that is known in the art, e.g., by means of conventional mixing, dissolving, granulating, dragee-making, levigating, emulsifying, encapsulating, entrapping, or lyophilizing processes.

A pharmaceutical composition may be provided as a salt and can be formed with many acids, including but not limited to, hydrochloric, sulfuric, acetic, lactic, tartaric, malic, succinic, and the like. Salts tend to be more soluble in aqueous solvents, or other protonic solvents, than are the corresponding free base forms. In other cases, the preferred preparation may be a lyophilized powder which may contain any or all of the following: 1-50 mM histidine, 0.1%-2% sucrose, and 2-7% mannitol, at a pH range of 4.5 to 5.5, combined with a buffer prior to use. After the pharmaceutical compositions have been prepared, they can be placed in an appropriate container and labeled for treatment of an indicated condition. For administration of a GPCR product, such labeling would include amount, frequency, and method of administration.

Pharmaceutical compositions suitable for use in the present invention include compositions wherein the active ingredients are contained in an effective amount to achieve the intended purpose. The determination of an effective dose or amount is well within the capability of those skilled in the art. For any compound, the therapeutically effective dose can be estimated initially either in cell culture assays, for example, using neoplastic cells, or in animal models, usually mice, rabbits, dogs, or pigs. The animal model may also be used to determine the appropriate concentration range and route of administration. Such information can then be used and extrapolated to determine useful doses and routes for administration in humans.

A therapeutically effective dose refers to that amount of active ingredient, for example, GPCR polynucleotide, GPCR polypeptide, or fragments thereof, antibodies to GPCR polypeptide, agonists, antagonists or inhibitors of GPCR polypeptide, which ameliorates, reduces, diminishes, or eliminates the symptoms or condition. Therapeutic efficacy and toxicity can be determined by standard pharmaceutical procedures in cell cultures or in experimental animals, e.g., ED₅₀ (the dose therapeutically effective in 50% of the population) and LD₅₀ (the dose lethal to 50% of the population). The dose ratio of toxic to therapeutic effects is the therapeutic index, which can be expressed as the ratio, LD₅₀/ED₅₀. Pharmaceutical compositions which exhibit large therapeutic indices are preferred. The data obtained from cell culture assays and animal studies are used in determining a range of dosages for human use. Preferred dosage contained in a pharmaceutical composition is within a range of circulating concentrations that include the ED₅₀ with little or no toxicity. The dosage varies within this range depending upon the dosage form employed, the sensitivity of the patient, and the route of administration.

The practitioner, who will consider the factors related to an individual requiring treatment, will determine the exact dosage. Dosage and administration are adjusted to provide sufficient levels of the active component, or to maintain the desired effect. Factors which may be taken into account include the severity of the individual's disease state; the general health of the patient; the age, weight, and gender of the patient; diet; time and frequency of administration; drug combination(s); reaction sensitivities; and tolerance/response to therapy. As a general guide, long-acting pharmaceutical compositions may be administered every 3 to 4 days, every week, or once every two weeks, depending on half-life and clearance rate of the particular formulation. Variations in these dosage levels can be adjusted using standard empirical routines for optimization, as is well understood in the art.

As a guide, normal dosage amounts may vary from 0.1 to 100,000 micrograms (μg), up to a total dose of about 1 gram (g), depending upon the route of administration. Guidance as to particular dosages and methods of delivery is provided in the literature and is generally available to practitioners in the art. Those skilled in the art will employ different formulations for nucleotides than for proteins or their inhibitors or activators. Similarly, the delivery of polynucleotides or polypeptides will be specific to particular cells, conditions, locations, and the like.

Microarrays and Screening Assays

In another embodiment of the present invention, oligonucleotides, or longer fragments derived from the GPCR polynucleotide sequences described herein can be used as targets in a microarray. The microarray can be used to monitor the expression levels of large numbers of genes simultaneously (to produce a transcript image), and to identify genetic variants, mutations and polymorphisms. This information may be used to determine gene function, to understand the genetic basis of a disease, to diagnose disease, and to develop and monitor the activities of therapeutic agents. In a particular aspect, the microarray is prepared and used according to the methods described in WO 95/11995 (Chee et al.); D. J. Lockhart et al., 1996, Nature Biotechnology, 14:1675-1680; and M. Schena et al., 1996, Proc. Natl. Acad. Sci. USA, 93:10614-10619). Microarrays are further described in U.S. Pat. No. 6,015,702 to P. Lal et al.

In another embodiment of this invention, a nucleic acid sequence which encodes a novel GPCR polypeptide, may also be used to generate hybridization probes, which are useful for mapping the naturally occurring genomic sequence. The sequences may be mapped to a particular chromosome, to a specific region of a chromosome, or to artificial chromosome constructions (HACs), yeast artificial chromosomes (YACs), bacterial artificial chromosomes (BACs), bacterial PI constructions, or single chromosome cDNA libraries, as reviewed by C. M. Price, 1993, Blood Rev., 7:127-134 and by B. J. Trask, 1991, Trends Genet., 7:149-154.

In another embodiment of the present invention, a GPCR polypeptide of this invention, its catalytic or immunogenic fragments, or oligopeptides thereof, can be used for screening libraries of compounds in any of a variety of drug screening techniques. The fragment employed in such screening may be free in solution, affixed to a solid support, borne on a cell surface, or located intracellularly. The formation of binding complexes, between the GPCR polypeptide, or a portion thereof, and the agent being tested, may be measured utilizing techniques commonly practiced in the art.

Another technique for drug screening, which may be employed, provides for high throughput screening of compounds having suitable binding affinity to the protein of interest as described in WO 84/03564 (Venton, et al.). In this method, as applied to the GPCR protein, large numbers of different small test compounds are synthesized on a solid substrate, such as plastic pins or some other surface. The test compounds are reacted with the GPCR polypeptide, or fragments thereof, and washed. Bound GPCR polypeptide is then detected by methods well known in the art. Purified GPCR polypeptide can also be coated directly onto plates for use in the aforementioned drug screening techniques. Alternatively, non-neutralizing antibodies can be used to capture the peptide and immobilize it on a solid support.

In a further embodiment, competitive drug screening assays can be used in which neutralizing antibodies, capable of binding a GPCR polypeptide according to this invention, specifically compete with a test compound for binding to the GPCR polypeptide. In this manner, the antibodies can be used to detect the presence of any peptide that shares one or more antigenic determinants with the GPCR polypeptide.

The human Gene 1, 2, 3, 4, 5, 6, 7, 8, 9, 10, 11, 12, or 13 polypeptides and/or peptides of the present invention, or immunogenic fragments or oligopeptides thereof, can be used for screening therapeutic drugs or compounds in a variety of drug screening techniques. The fragment employed in such a screening assay may be free in solution, affixed to a solid support, borne on a cell surface, or located intracellularly. The reduction or abolition of activity of the formation of binding complexes between the ion channel protein and the agent being tested can be measured. Thus, the present invention provides a method for screening or assessing a plurality of compounds for their specific binding affinity with a Gene 1, 2, 3, 4, 5, 6, 7, 8, 9, 10, 11, 12, or 13 polypeptide, or a bindable peptide fragment, of this invention, comprising providing a plurality of compounds, combining the Gene 1, 2, 3, 4, 5, 6, 7, 8, 9, 10, 11, 12, or 13 polypeptide, or a bindable peptide fragment, with each of a plurality of compounds for a time sufficient to allow binding under suitable conditions and detecting binding of the Gene 1, 2, 3, 4, 5, 6, 7, 8, 9, 10, 11, 12, or 13 polypeptide or peptide to each of the plurality of test compounds, thereby identifying the compounds that specifically bind to the Gene 1, 2, 3, 4, 5, 6, 7, 8, 9, 10, 11, 12, or 13 polypeptide or peptide.

Methods of identifying compounds that modulate the activity of the novel human Gene 1, 2, 3, 4, 5, 6, 7, 8, 9, 10, 11, 12, or 13 polypeptides and/or peptides are provided by the present invention and comprise combining a potential or candidate compound or drug modulator of GPCR biological activity with an Gene 1, 2, 3, 4, 5, 6, 7, 8, 9, 10, 11, 12, or 13 polypeptide or peptide, for example, the Gene 1, 2, 3, 4, 5, 6, 7, 8, 9, 10, 11, 12, or 13 amino acid sequence as set forth in SEQ ID NO:2, and measuring an effect of the candidate compound or drug modulator on the biological activity of the Gene 1, 2, 3, 4, 5, 6, 7, 8, 9, 10, 11, 12, or 13 polypeptide or peptide.

Such measurable effects include, for example, physical binding interaction; the ability to cleave a suitable GPCR substrate; effects on native and cloned Gene 1, 2, 3, 4, 5, 6, 7, 8, 9, 10, 11, 12, or 13-expressing cell line; and effects of modulators or other GPCR-mediated physiological measures.

Another method of identifying compounds that modulate the biological activity of the novel Gene 1, 2, 3, 4, 5, 6, 7, 8, 9, 10, 11, 12, or 13 polypeptides of the present invention comprises combining a potential or candidate compound or drug modulator of a GPCR biological activity with a host cell that expresses the Gene 1, 2, 3, 4, 5, 6, 7, 8, 9, 10, 11, 12, or 13 polypeptide and measuring an effect of the candidate compound or drug modulator on the biological activity of the Gene 1, 2, 3, 4, 5, 6, 7, 8, 9, 10, 11, 12, or 13 polypeptide. The host cell can also be capable of being induced to express the Gene 1, 2, 3, 4, 5, 6, 7, 8, 9, 10, 11, 12, or 13 polypeptide, e.g., via inducible expression. Physiological effects of a given modulator candidate on the Gene 1, 2, 3, 4, 5, 6, 7, 8, 9, 10, 11, 12, or 13 polypeptide can also be measured. Thus, cellular assays for particular GPCR modulators may be either direct measurement or quantification of the physical biological activity of the Gene 1, 2, 3, 4, 5, 6, 7, 8, 9, 10, 11, 12, or 13 polypeptide, or they may be measurement or quantification of a physiological effect. Such methods preferably employ a Gene 1, 2, 3, 4, 5, 6, 7, 8, 9, 10, 11, 12, or 13 polypeptide as described herein, or an overexpressed recombinant Gene 1, 2, 3, 4, 5, 6, 7, 8, 9, 10, 11, 12, or 13 polypeptide in suitable host cells containing an expression vector as described herein, wherein the Gene 1, 2, 3, 4, 5, 6, 7, 8, 9, 10, 11, 12, or 13 polypeptide is expressed, overexpressed, or undergoes upregulated expression.

Another aspect of the present invention embraces a method of screening for a compound that is capable of modulating the biological activity of a Gene 1, 2, 3, 4, 5, 6, 7, 8, 9, 10, 11, 12, or 13 polypeptide, comprising providing a host cell containing an expression vector harboring a nucleic acid sequence encoding a Gene 1, 2, 3, 4, 5, 6, 7, 8, 9, 10, 11, 12, or 13 polypeptide, or a functional peptide or portion thereof (e.g., SEQ ID NOS:2); determining the biological activity of the expressed Gene 1, 2, 3, 4, 5, 6, 7, 8, 9, 10, 11, 12, or 13 polypeptide in the absence of a modulator compound; contacting the cell with the modulator compound and determining the biological activity of the expressed Gene 1, 2, 3, 4, 5, 6, 7, 8, 9, 10, 11, 12, or 13 polypeptide in the presence of the modulator compound. In such a method, a difference between the activity of the Gene 1, 2, 3, 4, 5, 6, 7, 8, 9, 10, 11, 12, or 13 polypeptide in the presence of the modulator compound and in the absence of the modulator compound indicates a modulating effect of the compound.

Essentially any chemical compound can be employed as a potential modulator or ligand in the assays according to the present invention. Compounds tested as GPCR modulators can be any small chemical compound, or biological entity (e.g., protein, sugar, nucleic acid, lipid). Test compounds will typically be small chemical molecules and peptides. Generally, the compounds used as potential modulators can be dissolved in aqueous or organic (e.g., DMSO-based) solutions. The assays are designed to screen large chemical libraries by automating the assay steps and providing compounds from any convenient source. Assays are typically run in parallel, for example, in microtiter formats on microtiter plates in robotic assays. There are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs, Switzerland), for example. Also, compounds may be synthesized by methods known in the art.

High throughput screening methodologies are particularly envisioned for the detection of modulators of the novel Gene 1, 2, 3, 4, 5, 6, 7, 8, 9, 10, 11, 12, or 13 polynucleotides and polypeptides described herein. Such high throughput screening methods typically involve providing a combinatorial chemical or peptide library containing a large number of potential therapeutic compounds (e.g., ligand or modulator compounds). Such combinatorial chemical libraries or ligand libraries are then screened in one or more assays to identify those library members (e.g., particular chemical species or subclasses) that display a desired characteristic activity. The compounds so identified can serve as conventional lead compounds, or can themselves be used as potential or actual therapeutics.

A combinatorial chemical library is a collection of diverse chemical compounds generated either by chemical synthesis or biological synthesis, by combining a number of chemical building blocks (i.e., reagents such as amino acids). As an example, a linear combinatorial library, e.g., a polypeptide or peptide library, is formed by combining a set of chemical building blocks in every possible way for a given compound length (i.e., the number of amino acids in a polypeptide or peptide compound). Millions of chemical compounds can be synthesized through such combinatorial mixing of chemical building blocks.

The preparation and screening of combinatorial chemical libraries is well known to those having skill in the pertinent art. Combinatorial libraries include, without limitation, peptide libraries (e.g. U.S. Pat. No. 5,010,175; Furka, 1991, Int. J. Pept. Prot. Res., 37:487-493; and Houghton et al., 1991, Nature, 354:84-88). Other chemistries for generating chemical diversity libraries can also be used. Nonlimiting examples of chemical diversity library chemistries include, peptoids (PCT Publication No. WO 91/019735), encoded peptides (PCT Publication No. WO 93/20242), random bio-oligomers (PCT Publication No. WO 92/00091), benzodiazepines (U.S. Pat. No. 5,288,514), diversomers such as hydantoins, benzodiazepines and dipeptides (Hobbs et al., 1993, Proc. Natl. Acad. Sci. USA, 90:6909-6913), vinylogous polypeptides (Hagihara et al., 1992, J. Amer. Chem. Soc., 114:6568), nonpeptidal peptidomimetics with glucose scaffolding (Hirschmann et al., 1992, J. Amer. Chem. Soc., 114:9217-9218), analogous organic synthesis of small compound libraries (Chen et al., 1994, J. Amer. Chem. Soc., 116:2661), oligocarbamates (Cho et al., 1993, Science, 261:1303), and/or peptidyl phosphonates (Campbell et al., 1994, J. Org. Chem., 59:658), nucleic acid libraries (see Ausubel, Berger and Sambrook, all supra), peptide nucleic acid libraries (U.S. Pat. No. 5,539,083), antibody libraries (e.g., Vaughn et al., 1996, Nature Biotechnology, 14(3):309-314) and PCT/US96/10287), carbohydrate libraries (e.g., Liang et al., 1996, Science, 274-1520-1522) and U.S. Pat. No. 5,593,853), small organic molecule libraries (e.g., benzodiazepines, Baum C&EN, Jan. 18, 1993, page 33; and U.S. Pat. No. 5,288,514; isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; and the like).

Devices for the preparation of combinatorial libraries are commercially available (e.g., 357 MPS, 390 MPS, Advanced Chem Tech, Louisville Ky.; Symphony, Rainin, Woburn, Mass.; 433A Applied Biosystems, Foster City, Calif.; 9050 Plus, Millipore, Bedford, Mass.). In addition, a large number of combinatorial libraries are commercially available (e.g., ComGenex, Princeton, N.J.; Asinex, Moscow, Russia; Tripos, Inc., St. Louis, Mo.; ChemStar, Ltd., Moscow, Russia; 3D Pharmaceuticals, Exton, Pa.; Martek Biosciences, Columbia, Md., and the like).

In one embodiment, the invention provides solid phase based in vitro assays in a high throughput format, where the cell or tissue expressing an ion channel is attached to a solid phase substrate. In such high throughput assays, it is possible to screen up to several thousand different modulators or ligands in a single day. In particular, each well of a microtiter plate can be used to perform a separate assay against a selected potential modulator, or, if concentration or incubation time effects are to be observed, every 5-10 wells can test a single modulator. Thus, a single standard microtiter plate can assay about 96 modulators. If 1536 well plates are used, then a single plate can easily assay from about 100 to about 1500 different compounds. It is possible to assay several different plates per day; thus, for example, assay screens for up to about 6,000-20,000 different compounds are possible using the described integrated systems.

In another of its aspects, the present invention encompasses screening and small molecule (e.g., drug) detection assays which involve the detection or identification of small molecules that can bind to a given protein, i.e., a Gene 1, 2, 3, 4, 5, 6, 7, 8, 9, 10, 11, 12, or 13 polypeptide or peptide. Particularly preferred are assays suitable for high throughput screening methodologies.

In such binding-based detection, identification, or screening assays, a functional assay is not typically required. All that is needed is a target protein, preferably substantially purified, and a library or panel of compounds (e.g., ligands, drugs, small molecules) or biological entities to be screened or assayed for binding to the protein target. Preferably, most small molecules that bind to the target protein will modulate activity in some manner, due to preferential, higher affinity binding to functional areas or sites on the protein.

An example of such an assay is the fluorescence based thermal shift assay (3-Dimensional Pharmaceuticals, Inc., 3DP, Exton, Pa.) as described in U.S. Pat. Nos. 6,020,141 and 6,036,920 to Pantoliano et al.; see also, J. Zimmerman, 2000, Gen. Eng. News, 20(8)). The assay allows the detection of small molecules (e.g., drugs, ligands) that bind to expressed, and preferably purified, ion channel polypeptide based on affinity of binding determinations by analyzing thermal unfolding curves of protein-drug or ligand complexes. The drugs or binding molecules determined by this technique can be further assayed, if desired, by methods, such as those described herein, to determine if the molecules affect or modulate function or activity of the target protein.

To purify a Gene 1, 2, 3, 4, 5, 6, 7, 8, 9, 10, 11, 12, or 13 polypeptide or peptide to measure a biological binding or ligand binding activity, the source may be a whole cell lysate that can be prepared by successive freeze-thaw cycles (e.g., one to three) in the presence of standard protease inhibitors. The Gene 1, 2, 3, 4, 5, 6, 7, 8, 9, 10, 11, 12, or 13 polypeptide may be partially or completely purified by standard protein purification methods, e.g., affinity chromatography using specific antibody described infra, or by ligands specific for an epitope tag engineered into the recombinant Gene 1, 2, 3, 4, 5, 6, 7, 8, 9, 10, 11, 12, or 13 polypeptide molecule, also as described herein. Binding activity can then be measured as described.

Compounds which are identified according to the methods provided herein, and which modulate or regulate the biological activity or physiology of the Gene 1, 2, 3, 4, 5, 6, 7, 8, 9, 10, 11, 12, or 13 polypeptides according to the present invention are a preferred embodiment of this invention. It is contemplated that such modulatory compounds may be employed in treatment and therapeutic methods for treating a condition that is mediated by the novel Gene 1, 2, 3, 4, 5, 6, 7, 8, 9, 10, 11, 12, or 13 polypeptides by administering to an individual in need of such treatment a therapeutically effective amount of the compound identified by the methods described herein.

In addition, the present invention provides methods for treating an individual in need of such treatment for a disease, disorder, or condition that is mediated by the Gene 1, 2, 3, 4, 5, 6, 7, 8, 9, 10, 11, 12, or 13 polypeptides of the invention, comprising administering to the individual a therapeutically effective amount of the Gene 1, 2, 3, 4, 5, 6, 7, 8, 9, 10, 11, 12, or 13-modulating compound identified by a method provided herein.

### EXAMPLES

The Examples herein are meant to exemplify the various aspects of carrying out the invention and are not intended to limit the scope of the invention in any way.

The Examples do not include detailed descriptions for conventional methods employed, such as in the construction of vectors, the insertion of cDNA into such vectors, or the introduction of the resulting vectors into the appropriate host. Such methods are well known to those skilled in the art and are described in numerous publications, for example, Sambrook, Fritsch, and Maniatis, Molecular Cloning: A Laboratory Manual, 2^{nd} Edition, Cold Spring Harbor Laboratory Press, USA, (1989).

### Example 1

### Bioinformatics Analysis

Currently, one approach used for identifying and characterizing the genes distributed along the human genome includes utilizing large fragments of genomic DNA which are isolated, cloned, and sequenced. Potential open reading frames in these genomic sequences were identified using bioinformatics software.

GPCR sequences were obtained from the GPCR database at European Molecular Biology Laboratory (EMBL) (http://www.7tm.org/gpcr/). These sequences (more than 1300 protein sequences) were used as probes to search the human genomic, public and private EST databases. The search program used was BLAST2. The alignment was performed using the BLAST 2 algorithm according to the default parameters (S. F. Altschul, et al., Nucleic Acids Res. 25:3389-3402, 1997). The BLAST results were analyzed for potential novel GPCR candidates. The candidate sequences, from genomic or EST data, were then characterized. The characterization methods include sequence and profile-based analyses. The functional prediction is based on sequence identity and homology and/or domain information. The “query” sequence represents the novel GPCR amino acid sequence of the invention; the subject (“sbjct”) sequence represents the local matching sequence of the protein found in the database. The amino acids between the query and target sequences represent matching identical amino acids between the two sequences. Plus signs (“+”) between the query and target sequences represent similar amino acids between the two sequences. Spaces between the query and the target sequences indicate regions of non-identity for the aligned polypeptides.

FIG. 27A shows the regions of local identity (50%) and similarity (66%) between the novel human GPCR Gene 1 encoded amino acid sequence (SEQ ID NO:14, FIG. 2) of the present invention and the human olfactory receptor 5U1, i.e., the “sbjt” sequence (SEQ ID NO:72). For Gene 1, a domain prediction was also determined (FIG. 27B). The results suggest that the Gene 1 GPCR polypeptide of this invention represents a novel member of the rhodopsin protein family. Based upon this prediction, it is expected that the Gene 1 GPCR polypeptide may share at least some biological activity with members of the rhodopsin family of transmembrane receptors, in addition to specific members known in the art, or as otherwise described herein.

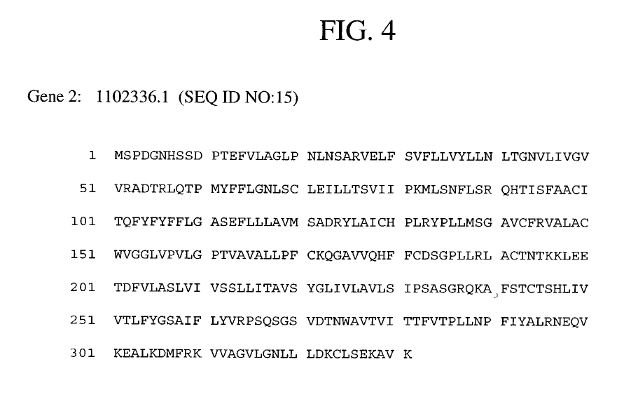

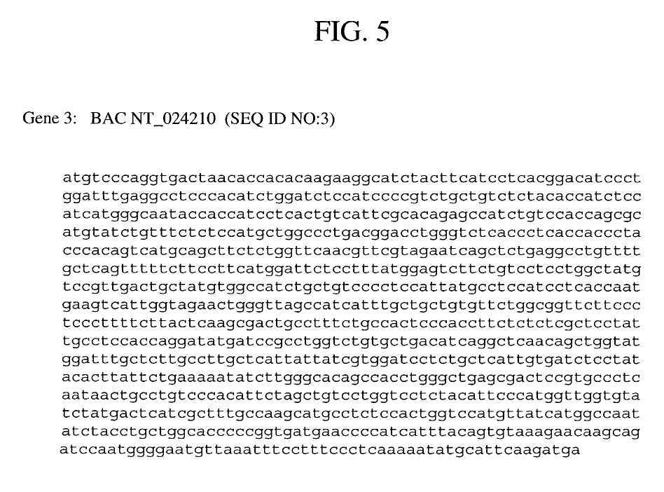

FIG. 28A shows the regions of local identity (98%) and similarity (98%) between the novel human GPCR Gene 2 encoded amino acid sequence (SEQ ID NO:15, FIG. 4) of the present invention and the human G protein-coupled receptor 61, i.e., the “sbjt” sequence (SEQ ID NO:73). FIG. 28B shows the regions of local identity (98%) and similarity (98%) between the novel human GPCR Gene 2 encoded amino acid sequence (SEQ ID NO:15, FIG. 4) of the present invention and a portion of rabbit G protein-coupled receptor protein, i.e., the “sbjt” sequence (SEQ ID NO:74). For Gene 2, a domain prediction was also determined (FIG. 28C). The results suggest that the Gene 2 GPCR polypeptide of this invention represents a novel member of the rhodopsin protein family. Based upon this prediction, it is expected that the Gene 2 GPCR polypeptide may share at least some biological activity with members of the rhodopsin family of transmembrane receptors, in addition to specific members known in the art, or as otherwise described herein.

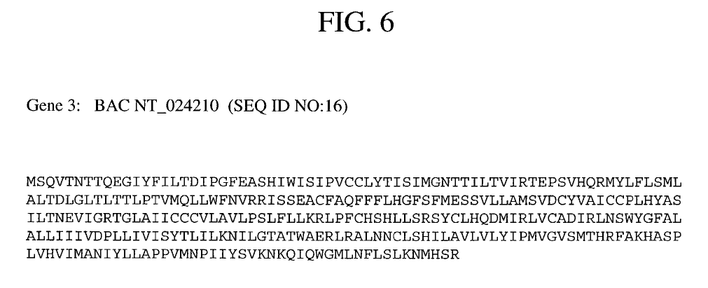

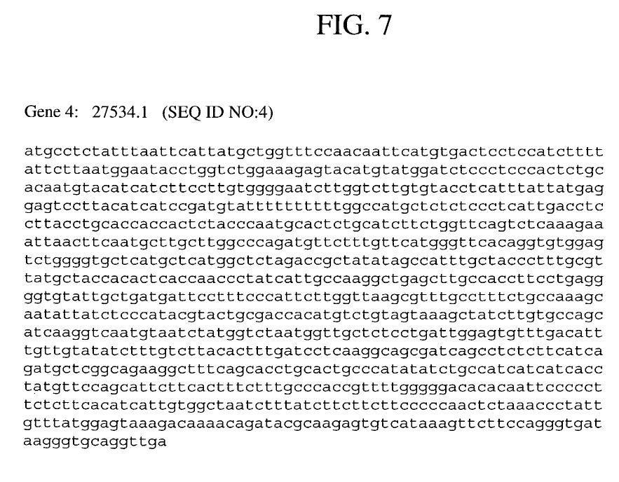

FIG. 29A shows the regions of local identity (48%) and similarity (61%) between the novel human GPCR Gene 3 encoded amino acid sequence (SEQ ID NO:16, FIG. 6) of the present invention and mouse olfactory receptor protein 3′Beta4, i.e., the “sbjt” sequence (SEQ ID NO:75). For Gene 3, a domain prediction was also determined (FIG. 29B). The results suggest that the Gene 3 GPCR polypeptide of this invention represents a novel member of the rhodopsin protein family. Based upon this prediction, it is expected that the Gene 3 GPCR polypeptide may share at least some biological activity with members of the rhodopsin family of transmembrane receptors, in addition to specific members known in the art, or as otherwise described herein.

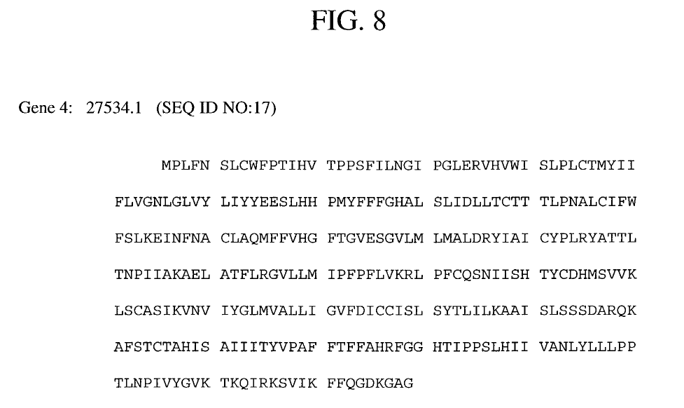

FIG. 30A shows the regions of local identity (48%) and similarity (64%) between the novel human GPCR Gene 4 encoded amino acid sequence (SEQ ID NO:17, FIG. 8) of the present invention and mouse olfactory receptor protein 3′Beta1, i.e., the “sbjt” sequence (SEQ ID NO:76). For Gene 4, a domain prediction was also determined (FIG. 30B). The results suggest that the Gene 4 GPCR polypeptide of this invention represents a novel member of the rhodopsin protein family. Based upon this prediction, it is expected that the Gene 4 GPCR polypeptide may share at least some biological activity with members of the rhodopsin family of transmembrane receptors, in addition to specific members known in the art, or as otherwise described herein.

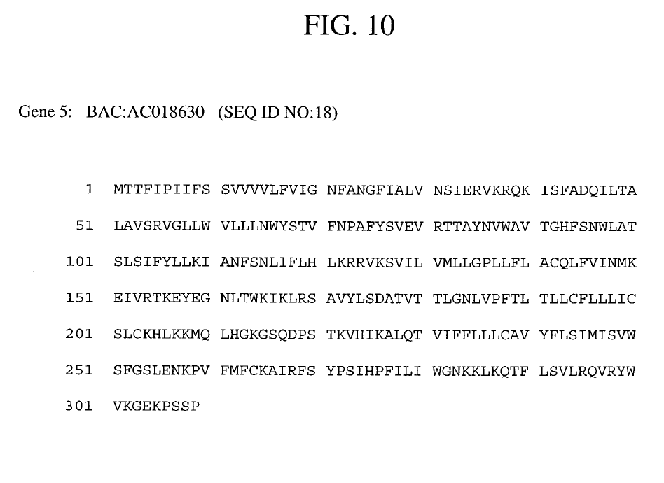

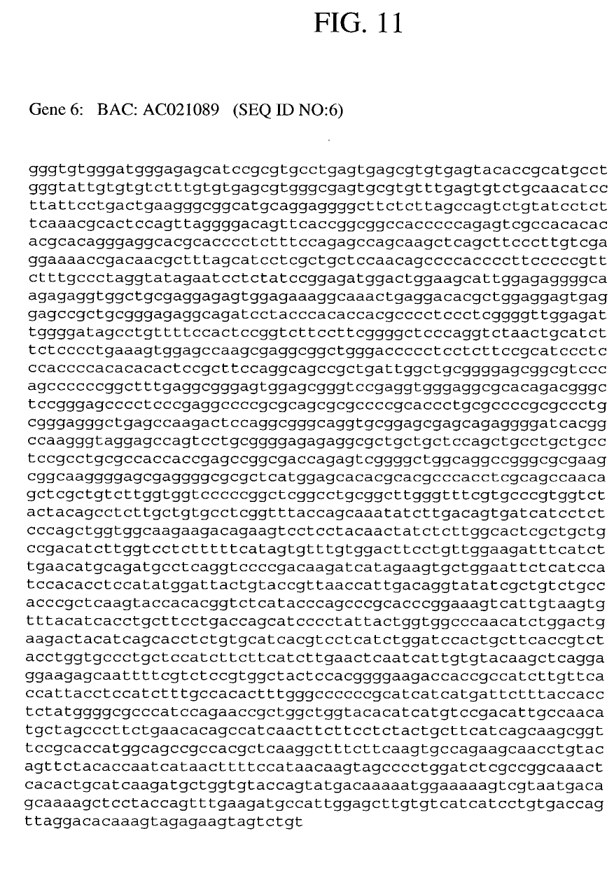

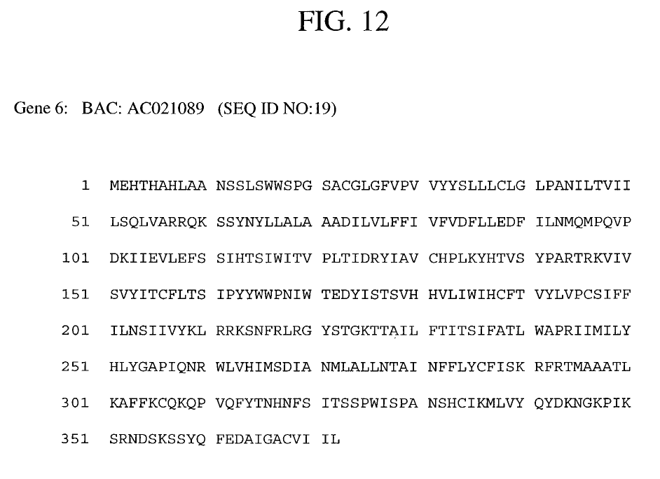

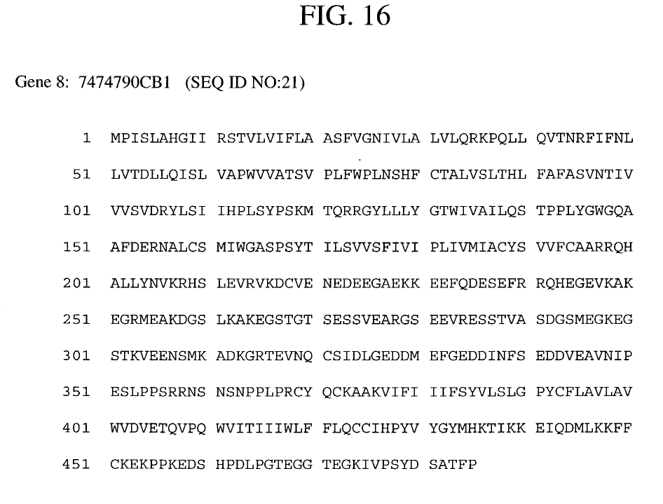

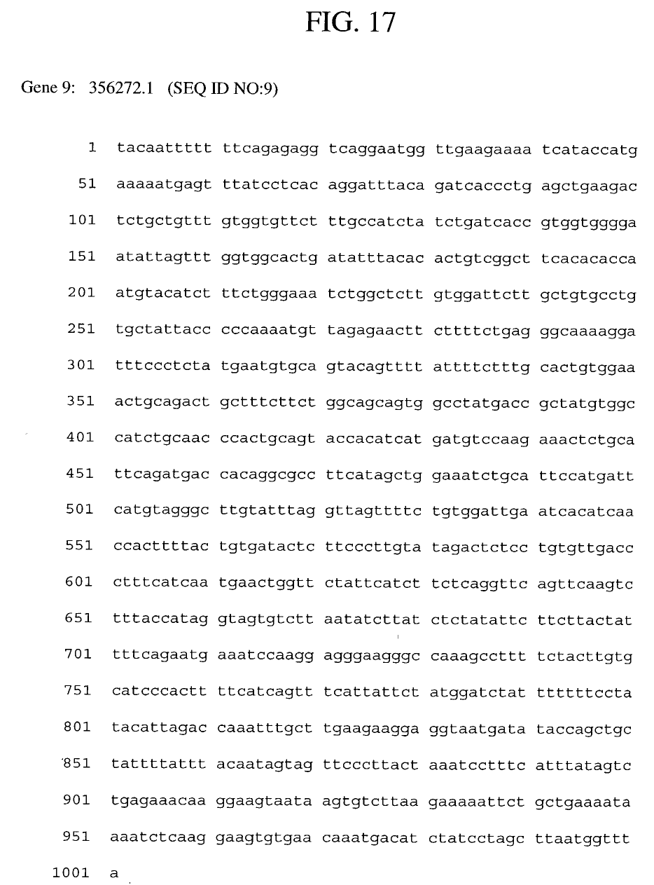

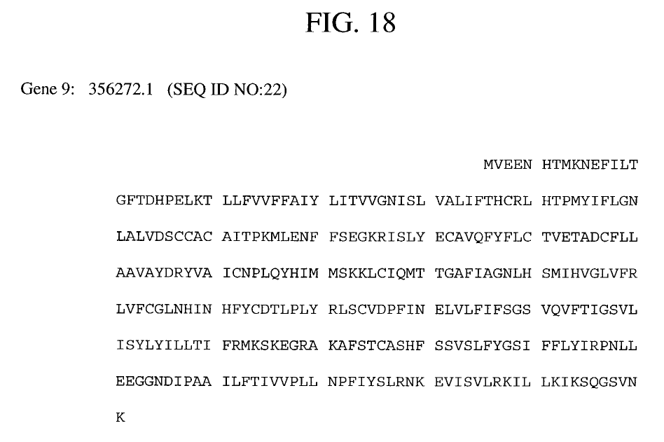

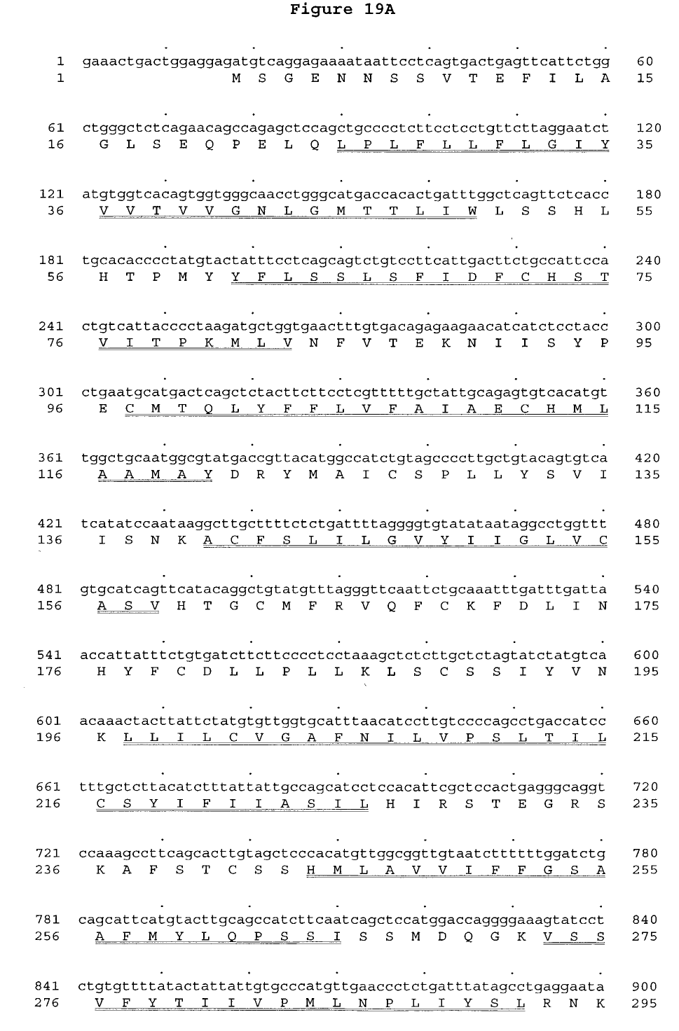

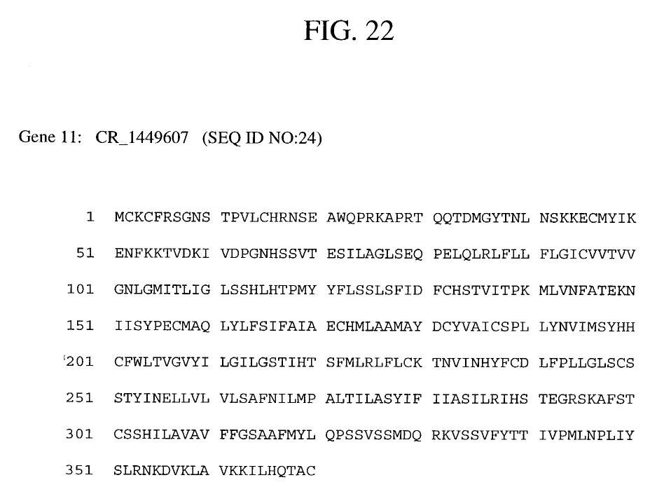

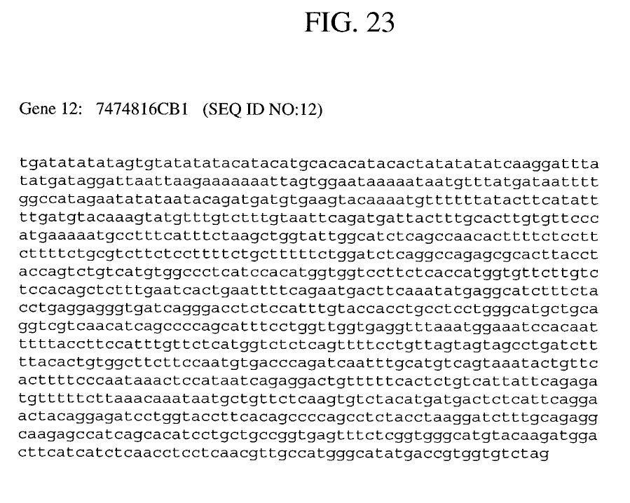

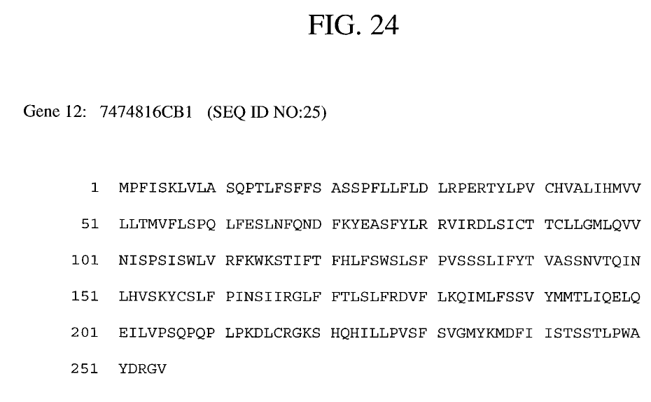

FIG. 31 shows the regions of local identity (44%) and similarity (61%) between the novel human GPCR Gene 5 encoded amino acid sequence (SEQ ID NO: 18, FIG. 10) of the present invention and human taste receptor T2R13, i.e., the “sbjt” sequence (SEQ ID NO:77). FIG. 32 shows the regions of local identity (26%) and similarity (47%) between the novel human GPCR Gene 6 encoded amino acid sequence (SEQ ID NO:19, FIG. 12) of the present invention and human chemokine receptor 1, i.e., the “sbjt” sequence (SEQ ID NO:78). FIG. 33 shows the regions of local identity (77%) and similarity (82%) between the novel human GPCR Gene 7 encoded amino acid sequence (SEQ ID NO:20, FIG. 14) of the present invention and human G protein-coupled receptor hHI7T213, i.e., the “sbjt” sequence (SEQ ID NO:79). FIG. 34 shows the regions of local identity (32%) and similarity (54%) between the novel human GPCR Gene 8 encoded amino acid sequence (SEQ ID NO:21, FIG. 16) of the present invention and human G protein-coupled receptor RE2, i.e., the “sbjt” sequence (SEQ ID NO:80). FIG. 35 shows the regions of local identity (47%) and similarity (67%) between the novel human GPCR Gene 9 encoded amino acid sequence (SEQ ID NO:22, FIG. 18) of the present invention and olfactory receptor OR93Gib, i.e., the “sbjt” sequence (SEQ ID NO:81). FIG. 36 shows the regions of local identity (76%) and similarity (87%) between the novel human GPCR Gene 10 encoded amino acid sequence (SEQ ID NO:23, FIG. 20) of the present invention and mouse odorant receptor K11, i.e., the “sbjt” sequence (SEQ ID NO:82). FIG. 37 shows the regions of local identity (78%) and similarity (85%) between the novel human GPCR Gene 11 encoded amino acid sequence (SEQ ID NO:24, FIG. 22) of the present invention and mouse odorant receptor K4h11, i.e., the “sbjt” sequence (SEQ ID NO:83). FIG. 38 shows the regions of local identity (43%) and similarity (59%) between the novel human GPCR Gene 12 encoded amino acid sequence (SEQ ID NO:25, FIG. 24) of the present invention and mouse vomeronasal receptor V1RC3, i.e., the “sbjt” sequence (SEQ ID NO:84). FIG. 39 shows the regions of local identity (30%) and similarity (53%) between the novel human GPCR Gene 13 encoded amino acid sequence (SEQ ID NO:26, FIG. 26) of the present invention and human very large G protein-coupled receptor-1, i.e., the “sbjt” sequence (SEQ ID NO:85).

In the case of Gene 13, the top genomic exon hits from the BLAST results were searched back against the non-redundant protein and patent sequence databases. From this analysis, exons encoding novel GPCR's were identified based on sequence homology. Also, the genomic region surrounding the matching exons was analyzed. Based on this analysis, the potential full length nucleotide sequence of Gene 13 (SEQ ID NO:13, FIGS. 25A-B) of the novel human GPCR, Gene 13, also referred to as GPCR-P20 and GPCR-P151, was identified directly from the genomic sequence (Genbank Acc ID:AC068323).

The amino acid sequence of the Gene 13 polypeptide (SEQ ID NO:26) encoded by the Gene 13 polynucleotide sequence (SEQ ID NO:13) was searched against the non-redundant protein and patent sequence databases. The alignment of Gene 13 polypeptide sequence (SEQ ID NO:26) with the top matching hits was performed using the GCG pileup program. The GAP global alignment program in GCG was used to calculate the percent identity and similarity values. The GAP program uses an algorithm based on (S. B. Needleman, C. D. Wunsch, J. Mol. Biol. 48(3):443-53, 1970), and the following parameters in the program was used: gap creation penalty:6 and gap extension penalty:2. In the alignment results, the blackened areas represent identical amino acids, the grey highlighted areas represent similar amino acids and dotted areas represent gaps in more than half of the listed sequences.

FIGS. 40A-40E show the regions of local identity (29.7%) and similarity (41.6%) between the novel human Gene 13 encoded amino acid sequence (SEQ ID NO:26, FIGS. 25A-B) of the present invention and the human GPCR receptor human_hypothetical 1, (Genbank Acc ID: 12044471; SEQ ID NO:149). Also shown in FIGS. 40A-40E are the regions of local identity (29.7%) and similarity (41.6%) between the novel human Gene 13 encoded amino acid sequence (SEQ ID NO:26, FIGS. 25A-B) of the present invention and the human GPCR receptor, human_hypothetical 2 (Genbank Acc ID: 14729798; SEQ ID NO:150)). These results indicate that the Gene 13 polypeptide of this invention represents a novel member of the GPCR protein family. It is thus expected that the Gene 13 polypeptide shares at least some biological activity with members of the GPCR family of transmembrane receptors, in addition to specific members known in the art, or as otherwise described herein.

The sequence information from the novel gene candidates was used for full-length cloning and expression profiling. Primer sequences were obtained using the primer3 program (Steve Rozen, Helen J. Skaletsky (1996, 1997) Primer3. Code available at http://www-genome.wi.mit.edu/genome_software/other/primer3.html). The right and left primers as presented in Table 2 were used in the cloning process and the “internal oligo” as presented in Table 3 was used as a hybridization probe to detect the PCR product after amplification.

### Example 2

### Cloning of the Novel Human GPCRs

Using the EST sequence, an antisense oligonucleotide with biotin on the 5′ end complementary to the putative coding region of GPCR was designed. This biotinylated oligo can be incubated with a mixture of single-stranded covalently closed circular cDNA libraries, which contain DNA corresponding to the sense strand. Hybrids between the biotinylated oligo and the circular cDNA are captured on streptavidin magnetic beads. Upon thermal release of the cDNA from the biotinylated oligo, the single stranded cDNA is converted into double strands using a primer homologous to a sequence on the cDNA cloning vector. The double stranded cDNA is introduced into E. coli by electroporation and the resulting colonies are screened by PCR, using a primer pair designed from the EST sequence to identify the proper cDNA. Oligos used to identify the cDNA of a GPCR gene of this invention by PCR can be selected from any one of GPCR sequences as represented in SEQ ID NOs:1-13.

### Example 3

### Multiplex Cloning

Construction of Size Fractionated cDNA Libraries

PolyA+ RNA was purchased from Clontech, treated with DNase I to remove traces of genomic DNA contamination and converted into double stranded cDNA using the SuperScript™ Plasmid System for cDNA Synthesis and Plasmid Cloning (Life Technologies). No radioisotope was incorporated in either of the cDNA synthesis steps. The cDNA was then size fractionated on a TransGenomics HPLC system equipped with a size exclusion column (TosoHass) with dimensions of 7.8 mm×30 cm and a particle size of 10 μm. Tris buffered saline (TBS) was used as the mobile phase, and the column was run at a flow rate of 0.5 mL/min. The system was calibrated by running a 1 kb ladder through the column and analyzing the fractions by agarose gel electrophoresis. Using these data, it can be determine which fractions are to be pooled to obtain the largest cDNA library. Generally, fractions that eluted in the range of 12 to 15 minutes were used.

The cDNA was precipitated, concentrated and then ligated into the SalI/NotI sites in pSPORT. After electroporation into E. coli DH12S, colonies were subjected to a miniprep procedure and the resulting cDNA was digested using SalI/NotI restriction enzymes. Generally, the average insert size of libraries made in this fashion was greater the 3.5 Kb; the overall complexity of the library is optimally greater than 10⁷.independent clones. The library was amplified in semi-solid agar for 2 days at 30° C. An aliquot (200 microliters) of the amplified library was inoculated into a 200 mL culture for single-stranded DNA isolation by super-infection with an f1 helper phage. The single stranded circular DNA was concentrated by ethanol precipitation, resuspended at a concentration of one microgram per microliter and used for the cDNA capture experiments.

Conversion of Double-Stranded cDNA Libraries into Single-Stranded Circular Form

To prepare cultures, 200 mL LB with 400 μL carbenicillin (100 mg/mL stock solution) was inoculated with from 200 μL to 1 mL of thawed cDNA library and incubated at 37° C. while shaking at 250 rpm for approximately 45 minutes, or until an OD600 of 0.025-0.040 was attained. M13K07 helper phage (1 mL) was added to the culture and grown for 2 hours, after which Kanamycin (500 μl; 30 mg/mL) was added and the culture was grown for an additional 15-18 hours.

The culture was then poured into 6 screw-cap tubes (50 mL autoclaved tubes) and cells subjected to centrifugation at 10K in an HB-6 rotor for 15 minutes at 4° C. to pellet the cells. The supernatant was filtered through a 0.2 μm filter and 12,000 units of Gibco DNase I was added. The mixture was incubated for 90 minutes at room temperature.

For PEG precipitation, 50 mL of ice-cold 40% PEG 8000, 2.5 M NaCl, and 10 mM MgSO₄ were added to the supernatant, mixed, and aliquotted into 6 centrifuge tubes (covered with parafilm). The tubes and contents were incubated for 1 hour on wet ice or at 4° C. overnight. The tubes were then centrifuged at 10K in a HB-6 rotor for 20 minutes at 4° C. to pellet the helper phage.

Following centrifugation, the supernatant was discarded and the sides of the tubes were dried. Each pellet was resuspended in 1 mL TE, pH 8. The resuspended pellets were pooled into a 14 mL tube (Sarstadt), (6 mL total). SDS was added to 0.1% (60 μl of stock 10% SDS). Freshly made proteinase K (20 mg/mL) was added (60 μl) and the suspension was incubated for 1 hour at 42° C.

For phenol/chloroform extractions, 1 mL of NaCl (5M) was added to the suspension in the tube. An equal volume of phenol/chloroform (6 mL) was added and the contents were vortexed or shaken. The suspension was then centrifuged at 5K in an HB-6 rotor for 5 minutes at 4° C. The aqueous (top) phase was transferred to a new tube (Sarstadt) and extractions were repeated until no interface was visible.

Ethanol precipitation was then performed on the aqueous phase whose volume was divided into 2 tubes (3 mL each). To each tube, 2 volumes of 100% ethanol was added and precipitation was carried out overnight at −20° C. The precipitated DNA was pelleted at 10K in an HB-6 rotor for 20 minutes at 4° C. The ethanol was discarded. Each pellet was resuspended in 700 μl of 70% ethanol. The contents of each tube were combined into one micro centrifuge tube and centrifuged in a micro centrifuge (Eppendorf) at 14K for 10 minutes at 4° C. After discarding the ethanol, the DNA pellet was dried in a speed vacuum. In order to remove oligosaccharides, the pellet was resuspended in 50 μl TE buffer, pH 8. The resuspension was incubated on dry ice for 10 minutes and centrifuged at 14K in an Eppendorf microfuge for 15 minutes at 4° C. The supernatant was then transferred to a new tube and the final volume was recorded.

To check purity, DNA was diluted 1:100 and added to a micro quartz cuvette, where DNA was analyzed by spectrometry at an OD260/OD280. The preferred purity ratio was between 1.7 and 2.0. The DNA was diluted to 1 μg/μL in TE, pH 8 and stored at 4° C. The concentration of DNA was calculated using the formula: (32 μg/mL*OD)(mL/1000 μL)(100)(OD260). The quality of single-stranded DNA was determined by first mixing 1 μL of 5 ng/μl ssDNA; 11 μL deionized water; 1.5 μL 10 μM T7 sport primer (fresh dilution of stock); 1.5 μl 10×Precision-Taq buffer per reaction. In the repair mix, a cocktail of 4 μl of 5 mM dNTPs (1.25 mM each); 1.5 μL 10×Precision-Taq buffer; 9.25μL deionized water; and 0.25 μL Precision-Taq polymerase was mixed per reaction and preheated at 70° C. until the middle of the thermal cycle.

The DNA mixes were aliquotted into PCR tubes and the thermal cycle was started. The PCR thermal cycle consisted of 1 cycle at 95° C. for 20 sec.; 59° C. for 1 min. (15 μL repair mix added); and 73° C. for 23 minutes. For ethanol precipitation, 15 μg glycogen, 16 μl ammonium acetate (7.5M), and 125 μL 100% ethanol were added and the contents were centrifuged at 14K in an Eppendorf microfuge for 30 minutes at 4° C. The resulting pellet was washed 1 time with 125 μL 70% ethanol and then the ethanol was discarded. The pellet was dried in a speed vacuum and resuspended in 10 μL TE buffer, pH 8.

Single-stranded DNA was electroporated into E. coli DH10B or DH12S cells by pre-chilling the cuvettes and sliding holder and thawing the cells on ice-water. DNA was aliquotted into micro centrifuge tubes (Eppendorf) as follows: 2 μL repaired library, (=1×10⁻³ μg); 1 μL unrepaired library (1 ng/μL), (=1×10⁻³ μg); and 1 μL pUC19 positive control DNA (0.01 μg/μL), (=1×10⁻⁵ μg). The mixtures were stored on ice until use.

One at a time, 40 μL of cells were added to a DNA aliquot. The cell/DNA mixture was not pipetted up and down more than one time. The mixture was then transferred via pipette into a cuvette between the metal plates and electroporation was performed at 1.8 kV. Immediately afterward, 1 mL SOC medium (i.e., SOB (bacto-tryptone; bacto-yeast extract; NaCl)+glucose (20 mM)+Mg²⁺) (See, J. Sambrook et al., Molecular Cloning: A Laboratory Manual, 2nd Ed., A.2, 1989) was added to the cuvette and the contents were transferred 15 mL media as commonly known in the art. The cells were allowed to recover for 1 hour at 37° C. with shaking (225 rpm).

Serial dilutions of the culture were made in 1:10 increments (20 μL into 180 μL LB) for plating the electroporated cells. For the repaired library, dilutions of 1:100, 1:1000, 1:10,000 were made. For the unrepaired library, dilutions of 1:10 and 1:100 were made. Positive control dilutions of 1:10 and 1:100 were made. Each dilution (100 μL) was plated onto small plates containing LB+carbenicillin and incubated at 37° C. overnight. The titer and background were calculated by methods well known in the art. Specifically, the colonies on each plate were counted using the lowest dilution countable. The titer was calculated using the formula: (# of colonies)(dilution factor)(200 μL/100 μL)(1000 μL/20 μL)=CFUs, where CFUs/μg DNA used=CFU/μg. The % background=((unrepaired CFU/μg)/(repaired CFU/μg))×100%.

Solution Hybridization and DNA Capture

One microliter of anti-sense biotinylated oligonucleotides (or sense oligonucleotides when annealing to single-stranded DNA from pSPORT2 vector) containing 150 ng of up to 50 different 80-mer oligonucleotide probes was added to 6 μL (6 μg) of a mixture of up to 15 single-stranded, covalently-closed, circular cDNA libraries and 7 μL of 100% formamide in a 0.5 mL PCR tube.

In the case of Gene 13, the following 80′mer oligonucleotides were used to clone the full-length Gene 13 polynucleotide:

| | |
|---|---|
| TCATGGAACTCTGTCTCCAGTGACTTTGCATTGG | (SEQ ID NO:155) |
| AACATAGACTCTGATCCTGATGGTGATCTCGCCT | |
| TCACCTCTGGCA-3′ and | |
| 5′-TTGGGCAGACGAGCGCCAATATCACTGTGGA | (SEQ ID NO:156) |
| GATATTGCCTGACGAAGACCCAGAACTGGATAAG | |
| GCATTCTCTGTGTCA-3′. | |

The mixture was heated in a thermal cycler to 95° C. for 2 minutes. Fourteen microliters of 2×hybridization buffer (50% formamide, 1.5 M NaCl, 0.04 M NaPO₄, pH 7.2, 5 mM EDTA, 0.2% SDS) were added to the heated probe/cDNA library mixture and incubated at 42° C. for 26 hours. Hybrids between the biotinylated oligo and the circular cDNA were isolated by diluting the hybridization mixture to 220 microliters in a solution containing 1 M NaCl, 10 mM Tris-HCl pH 7.5, 1 mM EDTA, pH 8.0 and adding 125 microliters of streptavidin magnetic beads. This solution was incubated at 42° C. for 60 minutes, and mixed every 5 minutes to resuspend the beads. The beads were separated from the solution with a magnet and washed three times in 200 microliters of 0.1×SSPE, 0.1% SDS at 45° C.

The single stranded cDNAs was release from the biotinylated oligo/streptavidin magnetic bead complex by adding 50 microliters of 0.1 N NaOH and incubating at room temperature for 10 minutes. Six microliters of 3 M sodium acetate was added along with 15 μg of glycogen and the solution was ethanol precipitated with 120 microliters of 100% ethanol. The precipitated DNA was resuspend in 12 μL of TE (10 mM Tris-HCl, pH 8.0, 1 mM EDTA, pH 8.0). The single stranded cDNA was converted into double strands in a thermal cycler by mixing 5 μL of the captured DNA with 1.5 μL of 10 μM of standard SP6 primer for libraries (ATTTAGGTGACACTATAG-3′(SEQ ID NO:157)) in pSPORT 1 and 2, and T7 Sport primer (5′-TAATACGACTCACTATAGGG-3′ (SEQ ID NO:158)) for libraries in pCMVSPORT, and 1.5 μL of 10×PCR buffer. The mixture was heated to 95° C. for 20 seconds, and then ramped down to 59° C. At this time 15 μL of a repair mix, preheated to 70° C., was added to the DNA (Repair mix contains 4 μL of 5 mM dNTPs (1.25 mM each), 1.5 μL of 10×PCR buffer, 9.25 μL of water, and 0.25 μL of Taq polymerase). The solution was ramped back to 73° C. and incubated for 23 minutes.

The repaired DNA was ethanol precipitated and resuspended in 10 μL of TE. Two μL were electroporated per tube containing 40 μL of E. coli DH12S cells. Three hundred and thirty three μL were plated onto one 150 mm plate of LB agar plus 100 μg/mL of ampicillin. After overnight incubation at 37° C., the colonies from all plates were harvested by scraping into 10 mL of LB+50 μg/mL of ampicillin and 2 mL of sterile glycerol.

The second round of selection was initiated by making single-strand circular DNA from the primary selected library using the above-described method. The purified single-stranded circular DNA was then assayed with gene-specific primers for each of the targeted sequences using standard PCR conditions.

In the case of Gene 13, the following gene-specific primers (GSPs) were used:

Primer Set One: left primer 1: 5′-GTGACAATTGCAGCCTCTGA-3′ (SEQ ID NO:151), right primer 1: 5′-AGTGATATTGGCGCTCGTCT-3′ (SEQ ID NO:152);

Primer Set Two: left primer 2: 5′-CTTCACCTCTGGCAACATCA-3′ (SEQ ID NO:153), right primer 2: 5′-ACTTTTCCCATGAGGCCTTT-3′ (SEQ ID NO:154),

The hybridization was performed including only those 80 mer biotinylated probes whose targeted sequences had a positive result with the GSPs. In the case of Gene 13, SEQ ID NO:155 and 156 were used. The resulting single-stranded circular DNA was converted into double strands using the antisense oligo for each target sequence as the repair primer (the sense primer was used for material captured from pSPORT2 libraries). The resulting double stranded DNA was electroporated into DH10B cells and the resulting colonies were inoculated into 96 deep well blocks. After overnight growth, DNA was prepared and sequentially screened for each of the targeted sequences using the GSPs. The DNA was also digested with SalI and NotI restriction enzymes and the inserts were sized by agarose gel electrophoresis.

### Example 4

### RNA Ligase Protocol for Generating the 5′ or 3′ End Sequences to Obtain Full-Length GPCR Genes

Once a GPCR gene/polynucleotide sequence of interest is identified, several methods are available for the identification of the 5′ or 3′ portions of the gene which may not be present in the original cDNA plasmid. These methods include, but are not limited to, filter probing, clone enrichment using specific probes and protocols similar and identical to 5′ and 3′RACE. While the full-length gene may be present in the library and can be identified by probing, a useful method for generating the 5′ or 3′ end is to use the existing sequence information from the original cDNA to generate the missing information. A method similar to 5′RACE is available for generating the missing 5′ end of a desired full-length gene. (This method was published by Fromont-Racine et al., Nucleic Acids Res., 21(7): 1683-1684 (1993)).

Briefly, a specific RNA oligonucleotide is ligated to the 5′ ends of a population of RNA, preferably 30, containing full-length gene RNA transcripts and a primer set containing a primer specific to the ligated RNA oligonucleotide and a primer specific to a known sequence of the gene of interest, and is used to PCR amplify the 5′ portion of the desired full length gene which may then be sequenced and used to generate the full-length gene. This method starts with total RNA isolated from the desired source. PolyA RNA may be used, but is not a prerequisite for this procedure. The RNA preparation is then be treated with phosphatase if necessary to eliminate 5′ phosphate groups on degraded or damaged RNA which may interfere with the later RNA ligase step. The phosphatase, if used, is then inactivated and the RNA is treated with tobacco acid pyrophosphatase in order to remove the cap structure present at the 5′ ends of messenger RNAs. This reaction leaves a 5′ phosphate group at the 5′ end of the cap cleaved RNA which can then be ligated to an RNA oligonucleotide using T4 RNA ligase. This modified RNA preparation can then be used as a template for first strand cDNA synthesis using a gene specific oligonucleotide. The first strand synthesis reaction can then be used as a template for PCR amplification of the desired 5′ end using a primer specific to the ligated RNA oligonucleotide and a primer specific to the known sequence of the apoptosis related of interest. The resultant product is then sequenced and analyzed to confirm that the 5′ end sequence belongs to the relevant sequence of interest.

### Example 5

### Signal Transduction Assays

The activity of GPCRs or homologues thereof, can be measured using any assay suitable for the measurement of the activity of a G protein-coupled receptor, as commonly known in the art. Signal transduction activity of a G protein-coupled receptor can be determined by monitoring intracellular Ca²⁺, cAMP, inositol-1,4,5-triphosphate (IP₃), or 1,2-diacylglycerol (DAG). Assays for the measurement of intracellular Ca²⁺ are described, for example, in Sakurai et al. (EP 480 381). Intracellular IP₃ can be measured using a kit available from Amersham, Inc. (Arlington Heights, Ill.). A kit for measuring intracellular cAMP is available from Diagnostic Products, Inc. (Los Angeles, Calif.).

Activation of a G protein-coupled receptor triggers the release of Ca²⁺ ions sequestered in the mitochondria, endoplasmic reticulum, and other cytoplasmic vesicles into the cytoplasm. Fluorescent dyes, e.g., fura-2, can be used to measure the concentration of free cytoplasmic Ca²⁺. The ester of fura-2, which is lipophilic and can diffuse across the cell membrane, is added to the culture medium of the host cells which recombinantly express GPCRs. Once inside the cell, the fura-2 ester is hydrolyzed by cytosolic esterases to its non-lipophilic form, and then the dye cannot diffuse out of the cell. The non-lipophilic form of fura-2 fluoresces when it binds to free Ca²⁺. The fluorescence can be measured without lysing the cells at an excitation spectrum of 340 nm or 380 nm and at fluorescence spectrum of 500 nm (Sakurai et al., EP 480 381).

Upon activation of a GPCR, the rise of free cytosolic Ca²⁺ concentration is preceded by the hydrolysis of phosphatidylinositol 4,5-bisphosphate. Hydrolysis of this phospholipid by the phospholipase, phospholipase C, yields 1,2-diacylglycerol (DAG), which remains in the membrane, and water-soluble inositol 1,4,5-triphosphate (IP₃). Binding of ligands or agonists will increase the concentration of DAG and IP₃. Thus, signal transduction activity can be measured by monitoring the concentration of these hydrolysis products.

To measure IP₃ concentration, radioactively-labeled ([³H])-inositol is added to the culture medium of host cells expressing GPCRs. The ³H-inositol is taken up by the cells and incorporated into IP₃. The resulting inositol triphosphate is separated from the mono- and di-phosphate forms and measured (Sakurai et al., EP 480 381). Alternatively, an inositol 1,4,5-triphosphate assay system (Amersham) is commercially available for such use. With this system, the supplier (Amersham) provides tritium-labeled inositol 1,4,5-triphosphate and a receptor capable of distinguishing the radioactive inositol from other inositol phosphates. With these reagents, an effective and accurate competition assay can be performed to determine the inositol triphosphate levels.

Cyclic AMP levels can be measured according to the methods described in Gilman et al., Proc. Natl. Acad. Sci, 67:305-312 (1970). In addition, a kit for assaying levels of cAMP is available from Diagnostic Products Corp. (Los Angeles, Calif.).

### Example 6

### Expression Profiling of Novel Human GPCR Polypeptides

The same PCR primer pairs used to identify GPCR cDNA clones can be used to measure the steady state levels of mRNA by quantitative PCR. For example, the PCR primer pairs as set forth in Table 2 herein can be used to measure the steady state levels of the newly described GPCR mRNA by quantitative PCR. In the case of Gene 13, the SEQ ID NO:151 and 152, and/or SEQ ID NO:153 and 154 primers pairs were used for expression profiling.

Briefly, first strand cDNA is made from commercially available mRNA (Clontech) and subjected to real time quantitative PCR using a PE 5700 instrument (Applied Biosystems, Foster City, Calif.) which detects the amount of DNA amplified during each cycle by the fluorescent output of SYBR green, a DNA binding dye specific for double strands. The specificity of the primer pair for its target is verified by performing a thermal denaturation profile at the end of the run which provided an indication of the number of different DNA sequences present by determining melting Tm. The contribution of contaminating genomic DNA to the assessment of tissue abundance is controlled for by performing the PCR with first strand made with and without reverse transcriptase. In all cases, the contribution of material amplified in the no reverse transcriptase controls is expected to be negligible.

Small variations in the amount of cDNA used in each tube are determined by performing a parallel experiment using a primer pair for the cyclophilin gene, which is expressed in equal amounts in all tissues. These data are used to normalize the data obtained with the primer pairs. The PCR data are converted into a relative assessment of the differences in transcript abundance among the tissues tested.

As indicated in Table 1, transcripts corresponding to GPCR Gene 4 as described herein were found to be expressed in lung; transcripts corresponding to GPCR Gene 5 as described herein were found to be expressed in uterus; transcripts corresponding to GPCR Gene 7 as described herein were found to be expressed in skull tumor; transcripts corresponding to GPCR Gene 9 as described herein were found to be expressed in cartilage; and transcripts corresponding to GPCR Gene 13 as described herein were found to be expressed in brain.

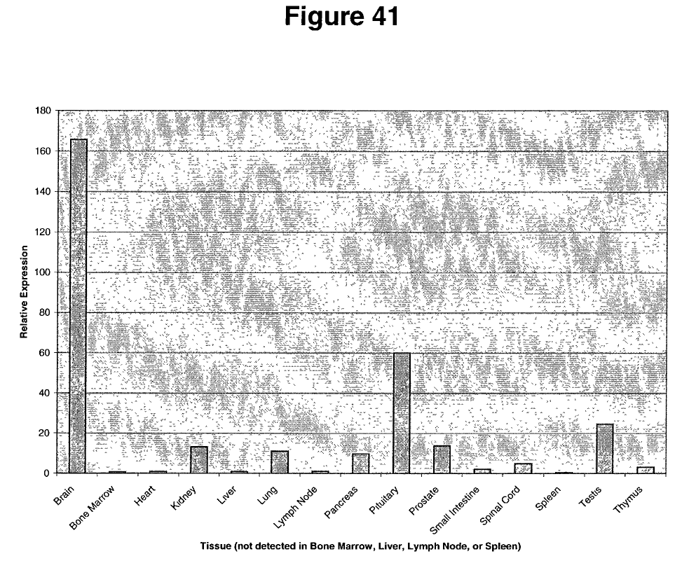

Moreover, as indicated in FIG. 41, transcripts corresponding to Gene 13 as described herein were found to be expressed to varying extents in brain, heart, kidney, lung, pancreas, pituitary, small intestine, spinal cord, testis and thymus.

As indicated in FIG. 42, transcripts corresponding to Gene 13 as described herein were found to be expressed in the following brain sub regions: amygdala, cerebellum, corpus callosum, caudate nucleus, hippocampus, subtantia nigra and thalamus.

### Example 7

### GPCR Activity

This example describes another method for screening compounds which are GPCR antagonists, and thus inhibit the activation or function of the GPCR polypeptides of the present invention. The method involves determining inhibition of binding of a labeled ligand, such as dATP, dAMP, or UTP, to cells expressing a novel GPCR on the cell surface, or to cell membranes containing the GPCR.

Such a method further involves transfecting a eukaryotic cell with DNA encoding a GPCR polypeptide such that the cell expresses the receptor on its surface. The cell is then contacted with a potential antagonist in the presence of a labeled form of a ligand, such as dATP, dAMP, or UTP. The ligand can be labeled, e.g., by radioactivity, fluorescence, chemiluminescence, or any other suitable detectable label commonly known in the art. The amount of labeled ligand bound to the expressed GPCR receptors is measured, e.g., by measuring radioactivity associated with transfected cells, or membranes from these cells. If the compound binds to the expressed GPCR, the binding of labeled ligand to the receptor is inhibited, as determined by a reduction of labeled ligand which also binds to the GPCR. This method is called a binding assay. The above-described technique can also be used to determine binding of GPCR agonists.

In a further screening procedure, mammalian cells, for example, but not limited to, CHO, HEK 293, Xenopus oocytes, RBL-2H3, etc., which are transfected with nucleic acid encoding a novel GPCR, are used to express the receptor of interest. The cells are loaded with an indicator dye that produces a fluorescent signal when bound to calcium, and the cells are contacted with a test substance and a receptor agonist, such as DATP, DAMP, or UTP. Any change in fluorescent signal is measured over a defined period of time using, for example, a fluorescence spectrophotometer or a fluorescence imaging plate reader. A change in the fluorescence signal pattern generated by the ligand relative to control indicates that a compound is a potential antagonist or agonist for the receptor.

In yet another screening procedure, mammalian cells are transfected with a GPCR-encoding polynucleotide sequence so as to express the GPCR of interest. The same cells are also transfected with a reporter gene construct that is coupled to/associated with activation of the receptor. Nonlimiting examples of suitable reporter gene systems include luciferase or beta-galactosidase regulated by an appropriate promoter. The engineered cells are contacted with a test substance or compound and a receptor ligand, such as dATP, dAMP, or UTP, and the signal produced by the reporter gene is measured after a defined period of time. The signal can be measured using a luminometer, spectrophotometer, fluorimeter, or other such instrument appropriate for the specific reporter construct used. Inhibition of the signal generated by the ligand indicates that a compound is a potential antagonist for the receptor.

Another screening technique for determining GPCR antagonists or agonists involves introducing RNA encoding the GPCR polypeptide into cells (e.g., CHO, HEK 293, RBL-2H3 cells, and the like) in which the receptor is transiently or stably expressed. The receptor cells are then contacted with a ligand for the GPCR, such as dATP, dAMP, or UTP, and a compound to be screened. Inhibition or activation of the receptor is then determined by detection of a signal, such as, cAMP, calcium, proton, or other ions.

### Example 8

### Functional Characterization of the Novel Human GPCR, Gene 13

The use of mammalian cell reporter assays to demonstrate functional coupling of known GPCRs has been well documented in the literature (Gilman, 1987 Ann. Rev. Biochem. 56: 615-649; Boss et al., 1996, J. Biol. Chem., 271: 10429-14032; Alam & Cook, 1990, Anal. Biochem., 188: 245-254; George et al., 1997, J. Neurochem., 69: 1278-1285; Selbie & Hill, 1998, TiPs, 19: 87-93; Rees et al., 1999, In Milligan G. (ed.): Signal Transduction: A practical approach, Oxford: Oxford Univ. Press, 171-221). In fact, reporter assays have been successfully used for identifying novel small molecule agonists or antagonists against GPCRs as a class of drug targets (Zlokarnik et al., 1998, Science, 279: 84-88; George et al; Boss et al.; Rees et al.). In such reporter assays, a promoter is regulated as a direct consequence of activation of specific signal transduction cascades following agonist binding to a GPCR (Alam & Cook 1990; Selbie & Hill, 1998; Boss et al.; George et al. 1997; Gilman, 1987).

A number of response element-based reporter systems have been developed that enable the study of GPCR function. These include cAMP response element (CRE)-based reporter genes for G alpha i/o, G alpha s-coupled GPCRs, Nuclear Factor Activator of Transcription (NFAT)-based reporters for G alpha q/11-coupled receptors and MAP kinase reporter genes for use in Galpha i/o coupled receptors (Selbie & Hill, 1998; Boss et al. 1996; George et al. 1997; Gilman 1987; Rees et al. 1999). Transcriptional response elements that regulate the expression of Beta-Lactamase within a CHO K1 cell line (Cho/NFAT-CRE: Aurora Biosciences™) (Zlokarnik et al., 1998) have been implemented to characterize the function of the Gene 13 polypeptide of the present invention. The system enables demonstration of constitutive G-protein coupling to endogenous cellular signaling components upon intracellular overexpression of GPCR receptors. Overexpression has been shown to represent a physiologically relevant event. For example, it has been shown that overexpression occurs in nature during metastatic carcinomas, wherein defective expression of the monocyte chemotactic protein 1 receptor, CCR2, in macrophages is associated with the incidence of human ovarian carcinoma (Sica, et al., 2000, J. Immunol., 164: 733-8; Salcedo et al., 2000, Blood, 96(1): 34-40). Indeed, it has been shown that overproduction of the Beta 2 Adrenergic Receptor in transgenic mice leads to constitutive activation of the receptor signaling pathway such that these mice exhibit increased cardiac output (Kypson et al., 1999, Gene Therapy, 6: 1298-1304; Dorn et al., 1999, PNAS, 96: 6400-5). These are only a few of the many examples demonstrating constitutive activation of GPCRs whereby many of these receptors are likely to be in the active, R*, conformation (Wess, 1997, FASEB J., 11(5): 346-354).

A. Materials and Methods:

DNA Constructs:

The putative GPCR Gene 13 cDNA may be PCR amplified using PFU™ (Stratagene). The primers used in the PCR reaction are specific to the Gene 13 polynucleotide and are ordered from Gibco BRL (5 prime primer: 5′-CCCAAGCTTATGCAGGCGCTTAACATTACCCCG-3′ (SEQ ID NO:159), 3 prime primer: 5′-CGGGATCCTTAATGCCACTGTCTAAAGGAAGA-3′ (SEQ ID NO: 160). The following 3 prime primer may be used to add a Flag-tag epitope to the Gene 13 polypeptide for immunocytochemistry: 5′-CGGGATCCTTACTTGTCGTCGTCGTCCTTGTAGTCCATATGCCCACTGTCT AAAGGAGAATTCTCAAC-3′(SEQ ID NO:161). The product from the PCR reaction may be isolated from a 0.8% Agarose gel (Invitrogen) and purified using a Gel Extraction Kit™ from Qiagen.

The purified product may be then digested overnight along with the pcDNA3.1 Hygro™ mammalian expression vector from Invitrogen using the HindIII and BamHI restriction enzymes (New England Biolabs). These digested products are then purified using the Gel Extraction Kit™ from Qiagen and subsequently ligated to the pcDNA3.1 Hygro™ expression vector using a DNA molar ratio of 4 parts insert: 1 vector. All DNA modification enzymes are purchased from NEB. The ligation may be incubated overnight at 16 degrees Celsius, after which time, one microliter of the mix may be used to transform DH5 alpha cloning efficiency competent E. coli™ (Gibco BRL). A detailed description of the pcDNA3.1 Hygro™ mammalian expression vector is available from Invitrogen (1600 Faraday Avenue, P.O. Box 6482, Carlsbad, Calif. 92008). The plasmid DNA from the ampicillin resistant clones are isolated using the Wizard DNA Miniprep System™ from Promega. Positive clones are then confirmed and scaled up for purification using the Qiagen Maxiprep™ plasmid DNA purification kit.

B. Cell Line Generation:

The pcDNA3.1 hygro vector containing the GPCR Gene 13 cDNA are used to transfect Cho/NFAT-CRE (Aurora Biosciences) cells using Lipofectamine 2000™ according to the manufacturers specifications (Gibco BRL). Two days later, the cells are split 1:3 into selective media (DMEM 11056, 600 ug/ml Hygromycin, 200 ug/ml Zeocin, 10% FBS). All cell culture reagents are purchased from Gibco BRL-Invitrogen.

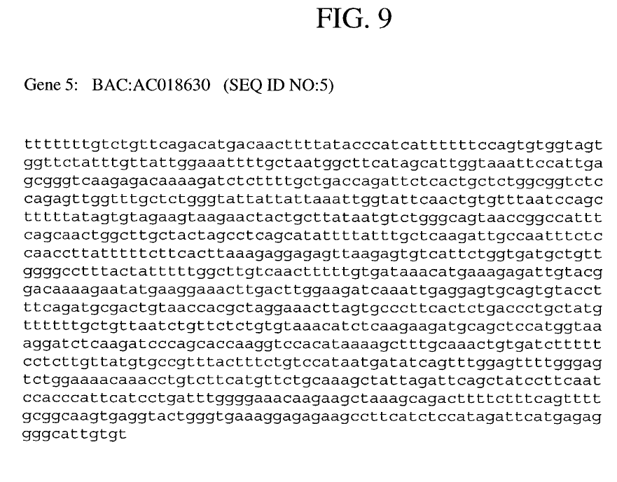

The Cho/NFAT-CRE cell lines, transiently or stably transfected with the Gene 13 GPCR, are analyzed using the FACS Vantage SE™ (BD), fluorescence microscopy (Nikon), and the LJL Analyst™ (Molecular Devices). In this system, changes in real-time gene expression, as a consequence of constitutive G-protein coupling of the Gene 13 GPCR, is examined by analyzing the fluorescence emission of the transformed cells at 447 nm and 518 nm. The changes in gene expression can be visualized using Beta-Lactamase as a reporter, that, when induced by the appropriate signaling cascade, hydrolyzes an intracellularly loaded, membrane-permeant ester, Cephalosporin-Coumarin-Fluorescein-2/Acetoxymethyl™ (CCF2/AM™ Aurora Biosciences; Zlokarnik, et al., 1998). The CCF2/AM™ substrate is a 7-hydroxycoumarin cephalosporin with a fluorescein attached through a stable thioether linkage. Induced expression of the Beta-Lactamase enzyme is readily apparent since each enzyme molecule produced is capable of changing the fluorescence of many CCF2/AM™ substrate molecules. A schematic of this cell based system is shown in FIG. 9.

In summary, CCF2/AM™ is a membrane permeant, intracellularly-trapped, fluorescent substrate with a cephalosporin core that links a 7-hydroxycoumarin to a fluorescein. For the intact molecule, excitation of the coumarin at 409 nm results in Fluorescence Resonance Energy Transfer (FRET) to the fluorescein which emits green light at 518 nm. Production of active Beta-Lactamase results in cleavage of the Beta-Lactam ring, leading to disruption of FRET, and excitation of the coumarin only-thus giving rise to blue fluorescent emission at 447 nm.

Fluorescent emissions are detected using a Nikon-TE300 microscope equipped with an excitation filter (D405/10X-25), dichroic reflector (430DCLP), and a barrier filter for dual DAPI/FITC (510 nM) to visually capture changes in Beta-Lactamase expression. The FACS Vantage SE is equipped with a Coherent Enterprise II Argon Laser and a Coherent 302C Krypton laser. In flow cytometry, UV excitation at 351-364 nm from the Argon Laser or violet excitation at 407 nm from the Krypton laser are used. The optical filters on the FACS Vantage SE are HQ460/50m and HQ535/40m bandpass separated by a 490 dichroic mirror.

Prior to analyzing the fluorescent emissions from the cell lines as described above, the cells are loaded with the CCF2/AM substrate. A 6×CCF2/AM loading buffer may be prepared whereby 1 mM CCF2/AM (Aurora Biosciences) may be dissolved in 100% DMSO (Sigma). 12 ul of this stock solution may be added to 60 ul of 100 mg/ml Pluronic F127 (Sigma) in DMSO containing 0.1% Acetic Acid (Sigma). This solution may be added while vortexing to 1 mL of Sort Buffer (PBS minus calcium and magnesium-Gibco-25 mM HEPES-Gibco-pH 7.4, 0.1% BSA). Cells are placed in serum-free media and the 6×CCF2/AM may be added to a final concentration of 1×. The cells are then loaded at room temperature for one to two hours, and then subjected to fluorescent emission analysis as described herein. Additional details relative to the cell loading methods and/or instrument settings may be found by reference to the following publications: see Zlokarnik, et al., 1998; Whitney et al., 1998, Nature Biotech. 16: 1329-1333; and BD Biosciences, 1999, FACS Vantage SE Training Manual.

C. Immunocytochemistry:

The cell lines transfected and selected for expression of Flag-epitope tagged GPCRs are analyzed by immunocytochemistry. The cells are plated at 1×10ˆ 3 in each well of a glass slide (VWR). The cells are rinsed with PBS followed by acid fixation for 30 minutes at room temperature using a mixture of 5% Glacial Acetic Acid/90% ETOH. The cells are then blocked in 2% BSA and 0.1% Triton in PBS, incubated for 2 h at room temperature or overnight at 4° C. A monoclonal anti-Flag FITC antibody may be diluted at 1:50 in blocking solution and incubated with the cells for 2 h at room temperature. Cells are then washed three times with 0.1% Triton in PBS for five minutes. The slides are overlayed with mounting media dropwise with Biomedia-Gel Mount™ (Biomedia; Containing Anti-Quenching Agent). Cells are examined at 10× magnification using the Nikon TE300 equiped with FITC filter (535 nm).

D. Demonstration of Cell Surface Expression:

Gene 13 may be tagged at the C-terminus using the Flag epitope and inserted into the pcDNA3.1 hygro™ expression vector, as described herein.

Immunocytochemistry of Cho Nfat-CRE cell lines transfected with the Flag-tagged Gene 13 construct with FITC conjugated Anti Flag monoclonal antibody demonstrated that Gene 13 is indeed a cell surface receptor. The immunocytochemistry also confirmed expression of the Gene 13 in the Cho Nfat-CRE cell lines. Briefly, Cho Nfat-CRE cell lines are transfected with pcDNA3.1 TM hygro™/Gene 13-Flag vector, fixed with 70% methanol, and permeablized with 0.1% TritonX100. The cells are then blocked with 1% Serum and incubated with a FITC conjugated Anti Flag monoclonal antibody at 1:50 dilution in PBS-Triton. The cells are then washed several times with PBS-Triton, overlayed with mounting solution, and fluorescent images are captured. The control cell line, non-transfected ChoNfat CRE cell line, exhibited no detectable background fluorescence. Plasma membrane localization would be consistent with Gene 13 representing a 7 transmembrane domain containing GPCR.

E. Screening Paradigm

The Aurora Beta-Lactamase technology provides a clear path for identifying agonists and antagonists of the Gene 13 polypeptide. Cell lines that exhibit a range of constitutive coupling activity may be identified by sorting through Gene 13 transfected cell lines using the FACS Vantage SE (see FIG. 10). For example, cell lines that exhibit an intermediate coupling response, using the LJL analyst, would provide the opportunity to screen, indirectly, for both agonists and antogonists of Gene 13 by looking for inhibitors that block the beta lactamase response, or agonists that increase the beta lactamase response. As described herein, modulating the expression level of beta lactamase directly correlates with the level of cleaved CCR2 substrate. For example, this screening paradigm has been shown to work for the identification of modulators of a known GPCR, 5HT6, that couples through Adenylate Cyclase, in addition to, the identification of modulators of the 5HT2c GPCR, that couples through changes in [Ca²⁺]i. Gene 13 modulator screens may be carried out using a variety of high throughput methods known in the art, though preferably using the fully automated Aurora UHTSS system.

In preferred embodiments, the Gene 13 transfected Cho Nfat-CRE cell lines are useful for the identification of agonists and antagonists of the Gene 13 polypeptide. Representative uses of these cell lines would be their inclusion in a method of identifying Gene 13 agonists and antagonists. Preferably, the cell lines are useful in a method for identifying a compound that modulates the biological activity of the Gene 13 polypeptide, comprising the steps of (a) combining a candidate modulator compound with a host cell expressing the Gene 13 polypeptide having the sequence as set forth in SEQ ID NO:2; and (b) measuring an effect of the candidate modulator compound on the activity of the expressed Gene 13 polypeptide. Representative vectors expressing the Gene 13 polypeptide are referenced herein (e.g., pcDNA3.1 hygro™) or otherwise known in the art.

The cell lines are also useful in a method of screening for a compound that is capable of modulating the biological activity of Gene 13 polypeptide, comprising the steps of: (a) determining the biological activity of the Gene 13 polypeptide in the absence of a modulator compound; (b) contacting a host cell expression the Gene 13 polypeptide with the modulator compound; and (c) determining the biological activity of the Gene 13 polypeptide in the presence of the modulator compound; wherein a difference between the activity of the Gene 13 polypeptide in the presence of the modulator compound and in the absence of the modulator compound indicates a modulating effect of the compound. Additional uses for these cell lines are described herein or otherwise known in the art.

The present invention is meant to encompass the application of the same coupling assay to the functional characterization of Gene 7, and 10, as well.

### Example 9

### Method of Creating N- and C-Terminal Deletion Mutants Corresponding to the Gene 13 Polypeptide of the Present Invention

As described elsewhere herein, the present invention encompasses the creation of N- and C-terminal deletion mutants, in addition to any combination of N- and C-terminal deletions thereof, corresponding to the Gene 13 polypeptide of the present invention. A number of methods are available to one skilled in the art for creating such mutants. Such methods may include a combination of PCR amplification and gene cloning methodology. Although one of skill in the art of molecular biology, through the use of the teachings provided or referenced herein, and/or otherwise known in the art as standard methods, could readily create each deletion mutant of the present invention, exemplary methods are described below.

Briefly, using the isolated cDNA clone encoding the full-length Gene 13 polypeptide sequence (as described in Examples 3 and 4, for example), appropriate primers of about 15-25 nucleotides derived from the desired 5′ and 3′ positions of SEQ ID NO:13 may be designed to PCR amplify, and subsequently clone, the intended N- and/or C-terminal deletion mutant. Such primers could comprise, for example, an inititation and stop codon for the 5′ and 3′ primer, respectively. Such primers may also comprise restriction sites to facilitate cloning of the deletion mutant post amplification. Moreover, the primers may comprise additional sequences, such as, for example, flag-tag sequences, kozak sequences, or other sequences discussed and/or referenced herein.

For example, in the case of the L4 to Y294 N-terminal deletion mutant, the following primers in Table 5 could be used to amplify a cDNA fragment corresponding to this deletion mutant:

TABLE 5

| | |
|---|---|
| 5′ Primer | 5′-gcagca gcggccgc ctcttttcaaaaagttgttccttgg-3′ |
| | (SEQ ID NO:162) |
| | NotI |
| 3′ Primer | 5′-gcagca gtcgac ataataataacacactaagggatac-3′ |
| | (SEQ ID NO:163) |
| | SalI |

For example, in the case of the M1 to P288 C-terminal deletion mutant, the following primers in Table 6 could be used to amplify a cDNA fragment corresponding to this deletion mutant:

TABLE 6

| | |
|---|---|
| 5′ Primer | 5′-gcagca gcggccgc atggaaggactcttttcaaaaag-3′ |
| | (SEQ ID NO:164) |
| | NotI |
| 3′ Primer | 5′-gcagca gtcgac gggatacttacttgaacgactctg-3′ |
| | (SEQ ID NO:165) |
| | SalI |

Representative PCR amplification conditions are provided below, although the skilled artisan would appreciate that other conditions may be required for efficient amplification. A 100 ul PCR reaction mixture may be prepared using 10 ng of the template DNA (cDNA clone of Gene 13), 200 uM 4dNTPs, 1 uM primers, 0.25U Taq DNA polymerase (PE), and standard Taq DNA polymerase buffer. Typical PCR cycling condition are as follows: 20-25 cycles of: (45 sec, 93 degrees; 2 min, 50 degrees; 2 min, 72 degrees) and 1 cycle of: (10 min, 72 degrees). After the final extension step of PCR, 5U Klenow Fragment may be added and incubated for 15 min at 30 degrees.

Upon digestion of the fragment with the NotI and SalI restriction enzymes, the fragment could be cloned into an appropriate expression and/or cloning vector which has been similarly digested (e.g., pSport1, among others). The skilled artisan would appreciate that other plasmids could be equally substituted, and may be desirable in certain circumstances. The digested fragment and vector are then ligated using a DNA ligase, and then used to transform competent E.coli cells using methods provided herein and/or otherwise known in the art.

The 5′ primer sequence for amplifying any additional N-terminal deletion mutants may be determined by reference to the following formula: (S+(X*3)) to ((S+(X*3))+25), wherein ‘S’ is equal to the nucleotide position of the initiating start codon of the Gene 13 gene (SEQ ID NO:13), and ‘X’ is equal to the most N-terminal amino acid of the intended N-terminal deletion mutant. The first term will provide the start 5′ nucleotide position of the 5′ primer, while the second term will provide the end 3′ nucleotide position of the 5′ primer corresponding to sense strand of SEQ ID NO:13. Once the corresponding nucleotide positions of the primer are determined, the final nucleotide sequence may be created by the addition of applicable restriction site sequences to the 5′ end of the sequence, for example. As referenced herein, the addition of other sequences to the 5′ primer may be desired in certain circumstances (e.g., kozak sequences, etc.).

The 3′ primer sequence for amplifying any additional N-terminal deletion mutants may be determined by reference to the following formula: (S+(X*3)) to ((S+(X*3))−25), wherein ‘S’ is equal to the nucleotide position of the initiating start codon of the Gene 13 gene (SEQ ID NO:13), and ‘X’ is equal to the most C-terminal amino acid of the intended N-terminal deletion mutant. The first term will provide the start 5′ nucleotide position of the 3′ primer, while the second term will provide the end 3′ nucleotide position of the 3′ primer corresponding to the anti-sense strand of SEQ ID NO:1. Once the corresponding nucleotide positions of the primer are determined, the final nucleotide sequence may be created by the addition of applicable restriction site sequences to the 5′ end of the sequence, for example. As referenced herein, the addition of other sequences to the 3′ primer may be desired in certain circumstances (e.g., stop codon sequences, etc.). The skilled artisan would appreciate that modifications of the above nucleotide positions may be necessary for optimizing PCR amplification.

The same general formulas provided above may be used in identifying the 5′ and 3′ primer sequences for amplifying any C-terminal deletion mutant of the present invention. Moreover, the same general formulas provided above may be used in identifying the 5′ and 3′ primer sequences for amplifying any combination of N-terminal and C-terminal deletion mutant of the present invention. The skilled artisan would appreciate that modifications of the above nucleotide positions may be necessary for optimizing PCR amplification.

Moreover, the invention encompasses the application of the same formulas and methods to the creation of any N—, or C-terminal deletion mutant, or any combination thereof, of Gene 7 and/or 10.

### Example 10

### Method of Assessing the Expression Profile of the Novel Gene 13 Polypeptides of the Present Invention Using Expanded MRNA Tissue and Cell Sources

Total RNA from tissues was isolated using the TriZol protocol (Invitrogen) and quantified by determining its absorbance at 260 nM. An assessment of the 18s and 28s ribosomal RNA bands was made by denaturing gel electrophoresis to determine RNA integrity.

The specific sequence to be measured was aligned with related genes found in GenBank to identity regions of significant sequence divergence to maximize primer and probe specificity. Gene-specific primers and probes were designed using the ABI primer express software to amplify small amplicons (150 base pairs or less) to maximize the likelihood that the primers function at 100% efficiency. All primer/probe sequences were searched against Public Genbank databases to ensure target specificity. Primers and probes were obtained from ABI.

For Gene 13, the primer probe sequences were as follows

Forward Primer 5′-GCAGACGAGCGCCAATATC-3′ (SEQ ID NO:166)

Reverse Primer 5′-GACACAGAGAATGCCTTATCCAGTT-3′ (SEQ ID NO: 167)

TaqMan Probe5′-TGGGTCTTCGTCAGGCAATATCTCCACA-3′ (SEQ ID NO: 168)

I. DNA Contamination

To access the level of contaminating genomic DNA in the RNA, the RNA was divided into 2 aliquots and one half was treated with Rnase-free Dnase (Invitrogen). Samples from both the Dnase-treated and non-treated were then subjected to reverse transcription reactions with (RT+) and without (RT−) the presence of reverse transcriptase. TaqMan assays were carried out with gene-specific primers (see above) and the contribution of genomic DNA to the signal detected was evaluated by comparing the threshold cycles obtained with the RT+/RT− non-Dnase treated RNA to that on the RT+/RT− Dnase treated RNA. The amount of signal contributed by genomic DNA in the Dnased RT− RNA must be less that 10% of that obtained with Dnased RT+ RNA. If not the RNA was not used in actual experiments.

II. Reverse Transcription Reaction and Sequence Detection

100 ng of Dnase-treated total RNA was annealed to 2.5 μM of the respective gene-specific reverse primer in the presence of 5.5 mM Magnesium Chloride by heating the sample to 72° C. for 2 min and then cooling to 55° C. for 30 min. 1.25 U/μl of MuLv reverse transcriptase and 500 μM of each dNTP was added to the reaction and the tube was incubated at 37° C. for 30 min. The sample was then heated to 90° C. for 5 min to denature enzyme.

Quantitative sequence detection was carried out on an ABI PRISM 7700 by adding to the reverse transcribed reaction 2.5 μM forward and reverse primers, 2.0 μM of the TaqMan probe, 500 μM of each dNTP, buffer and 5U AmpliTaq Gold™. The PCR reaction was then held at 94° C. for 12 min, followed by 40 cycles of 94° C. for 15 sec and 60° C. for 30 sec.

III. Data Handling

The threshold cycle (Ct) of the lowest expressing tissue (the highest Ct value) was used as the baseline of expression and all other tissues were expressed as the relative abundance to that tissue by calculating the difference in Ct value between the baseline and the other tissues and using it as the exponent in 2^{(ΔCt)}

The expanded expression profile of the Gene 13 polypeptide is provided in FIG. 43 and described elsewhere herein.

### Example 11

### Method of Enhancing the Biological Activity/Functional Characteristics of Invention Through Molecular Evolution.

Although many of the most biologically active proteins known are highly effective for their specified function in an organism, they often possess characteristics that make them undesirable for transgenic, therapeutic, pharmaceutical, and/or industrial applications. Among these traits, a short physiological half-life is the most prominent problem, and is present either at the level of the protein, or the level of the proteins mRNA. The ability to extend the half-life, for example, would be particularly important for a proteins use in gene therapy, transgenic animal production, the bioprocess production and purification of the protein, and use of the protein as a chemical modulator among others. Therefore, there is a need to identify novel variants of isolated proteins possessing characteristics which enhance their application as a therapeutic for treating diseases of animal origin, in addition to the proteins applicability to common industrial and pharmaceutical applications.

Thus, one aspect of the present invention relates to the ability to enhance specific characteristics of invention through directed molecular evolution. Such an enhancement may, in a non-limiting example, benefit the inventions utility as an essential component in a kit, the inventions physical attributes such as its solubility, structure, or codon optimization, the inventions specific biological activity, including any associated enzymatic activity, the proteins enzyme kinetics, the proteins Ki, Kcat, Km, Vmax, Kd, protein-protein activity, protein-DNA binding activity, antagonist/inhibitory activity (including direct or indirect interaction), agonist activity (including direct or indirect interaction), the proteins antigenicity (e.g., where it would be desirable to either increase or decrease the antigenic potential of the protein), the immunogenicity of the protein, the ability of the protein to form dimers, trimers, or multimers with either itself or other proteins, the antigenic efficacy of the invention, including its subsequent use a preventative treatment for disease or disease states, or as an effector for targeting diseased genes. Moreover, the ability to enhance specific characteristics of a protein may also be applicable to changing the characterized activity of an enzyme to an activity completely unrelated to its initially characterized activity. Other desirable enhancements of the invention would be specific to each individual protein, and would thus be well known in the art and contemplated by the present invention.

For example, an engineered G-protein coupled receptor may be constitutively active upon binding of its cognate ligand. Alternatively, an engineered G-protein coupled receptor may be constitutively active in the absence of ligand binding. In yet another example, an engineered GPCR may be capable of being activated with less than all of the regulatory factors and/or conditions typically required for GPCR activation (e.g., ligand binding, phosphorylation, conformational changes, etc.). Such GPCRs would be useful in screens to identify GPCR modulators, among other uses described herein.

Directed evolution is comprised of several steps. The first step is to establish a library of variants for the gene or protein of interest. The most important step is to then select for those variants that entail the activity you wish to identify. The design of the screen is essential since your screen should be selective enough to eliminate non-useful variants, but not so stringent as to eliminate all variants. The last step is then to repeat the above steps using the best variant from the previous screen. Each successive cycle, can then be tailored as necessary, such as increasing the stringency of the screen, for example.

Over the years, there have been a number of methods developed to introduce mutations into macromolecules. Some of these methods include, random mutagenesis, “error-prone” PCR, chemical mutagenesis, site-directed mutagenesis, and other methods well known in the art (for a comprehensive listing of current mutagenesis methods, see Maniatis, Molecular Cloning: A Laboratory Manual, Cold Spring Harbor Press, Cold Spring, N.Y. (1982)). Typically, such methods have been used, for example, as tools for identifying the core functional region(s) of a protein or the function of specific domains of a protein (if a multi-domain protein). However, such methods have more recently been applied to the identification of macromolecule variants with specific or enhanced characteristics.

Random mutagenesis has been the most widely recognized method to date.

Typically, this has been carried out either through the use of “error-prone” PCR (as described in Moore, J., et al, Nature Biotechnology 14:458, (1996), or through the application of randomized synthetic oligonucleotides corresponding to specific regions of interest (as descibed by Derbyshire, K. M. et al, Gene, 46:145-152, (1986), and Hill, D E, et al, Methods Enzymol., 55:559-568, (1987). Both approaches have limits to the level of mutagenesis that can be obtained. However, either approach enables the investigator to effectively control the rate of mutagenesis. This is particularly important considering the fact that mutations beneficial to the activity of the enzyme are fairly rare. In fact, using too high a level of mutagenesis may counter or inhibit the desired benefit of a useful mutation.

While both of the aforementioned methods are effective for creating randomized pools of macromolecule variants, a third method, termed “DNA Shuffling”, or “sexual PCR” (WPC, Stemmer, PNAS, 91:10747, (1994)) has recently been elucidated. DNA shuffling has also been referred to as “directed molecular evolution”, “exon-shuffling”, “directed enzyme evolution”, “in vitro evolution”, and “artificial evolution”. Such reference terms are known in the art and are encompassed by the invention. This new, preferred, method apparently overcomes the limitations of the previous methods in that it not only propagates positive traits, but simultaneously eliminates negative traits in the resulting progeny.

DNA shuffling accomplishes this task by combining the principal of in vitro recombination, along with the method of “error-prone” PCR. In effect, you begin with a randomly digested pool of small fragments of your gene, created by Dnase I digestion, and then introduce said random fragments into an “error-prone” PCR assembly reaction. During the PCR reaction, the randomly sized DNA fragments not only hybridize to their cognate strand, but also may hybridize to other DNA fragments corresponding to different regions of the polynucleotide of interest—regions not typically accessible via hybridization of the entire polynucleotide. Moreover, since the PCR assembly reaction utilizes “error-prone” PCR reaction conditions, random mutations are introduced during the DNA synthesis step of the PCR reaction for all of the fragments -further diversifying the potential hybridation sites during the annealing step of the reaction.

A variety of reaction conditions could be utilized to carry-out the DNA shuffling reaction. However, specific reaction conditions for DNA shuffling are provided, for example, in PNAS, 91:10747, (1994). Briefly:

Prepare the DNA substrate to be subjected to the DNA shuffling reaction.

Preparation may be in the form of simply purifying the DNA from contaminating cellular material, chemicals, buffers, oligonucleotide primers, deoxynucleotides, RNAs, etc., and may entail the use of DNA purification kits as those provided by Qiagen, Inc., or by the Promega, Corp., for example.

Once the DNA substrate has been purified, it would be subjected to Dnase I digestion. About 2-4 ug of the DNA substrate(s) would be digested with 0.0015 units of Dnase I (Sigma) per ul in 100 ul of 50 mM Tris-HCL, pH 7.4/1 mM MgCl2 for 10-20 min. at room temperature. The resulting fragments of 10-50 bp could then be purified by running them through a 2% low-melting point agarose gel by electrophoresis onto DE81 ion-exchange paper (Whatman) or could be purified using Microcon concentrators (Amicon) of the appropriate molecular weight cuttoff, or could use oligonucleotide purification columns (Qiagen), in addition to other methods known in the art. If using DE81 ion-exchange paper, the 10-50 bp fragments could be eluted from said paper using 1M NaCL, followed by ethanol precipitation.

The resulting purified fragments would then be subjected to a PCR assembly reaction by re-suspension in a PCR mixture containing: 2 mM of each dNTP, 2.2 mM MgCl2, 50 mM KCl, 10 mM Tris.HCL, pH 9.0, and 0.1% Triton X-100, at a final fragment concentration of 10-30 ng/ul. No primers are added at this point. Taq DNA polymerase (Promega) would be used at 2.5 units per 100 ul of reaction mixture. A PCR program of 94 C for 60s; 94 C for 30s, 50-55 C for 30s, and 72 C for 30s using 30-45 cycles, followed by 72 C for 5 min using an MJ Research (Cambridge, Mass.) PTC-150 thermocycler. After the assembly reaction is completed, a 1:40 dilution of the resulting primerless product would then be introduced into a PCR mixture (using the same buffer mixture used for the assembly reaction) containing 0.8 um of each primer and subjecting this mixture to 15 cycles of PCR (using 94 C for 30s, 50 C for 30s, and 72 C for 30s). The referred primers would be primers corresponding to the nucleic acid sequences of the polynucleotide(s) utilized in the shuffling reaction. Said primers could consist of modified nucleic acid base pairs using methods known in the art and referred to else where herein, or could contain additional sequences (i.e., for adding restriction sites, mutating specific base-pairs, etc.).

The resulting shuffled, assembled, and amplified product can be purified using methods well known in the art (e.g., Qiagen PCR purification kits) and then subsequently cloned using appropriate restriction enzymes.

Although a number of variations of DNA shuffling have been published to date, such variations would be obvious to the skilled artisan and are encompassed by the invention. The DNA shuffling method can also be tailered to the desired level of mutagenesis using the methods described by Zhao, et al. (Nucl Acid Res., 25(6):1307-1308, (1997).

As described above, once the randomized pool has been created, it can then be subjected to a specific screen to identify the variant possessing the desired characteristic(s). Once the variant has been identified, DNA corresponding to the variant could then be used as the DNA substrate for initiating another round of DNA shuffling. This cycle of shuffling, selecting the optimized variant of interest, and then re-shuffling, can be repeated until the ultimate variant is obtained. Examples of model screens applied to identify variants created using DNA shuffling technology may be found in the following publications: J. C., Moore, et al., J. Mol. Biol., 272:336-347, (1997), F. R., Cross, et al., Mol. Cell. Biol., 18:2923-2931, (1998), and A. Crameri., et al., Nat. Biotech., 15:436-438, (1997).

DNA shuffling has several advantages. First, it makes use of beneficial mutations. When combined with screening, DNA shuffling allows the discovery of the best mutational combinations and does not assume that the best combination contains all the mutations in a population. Secondly, recombination occurs simultaneously with point mutagenesis. An effect of forcing DNA polymerase to synthesize full-length genes from the small fragment DNA pool is a background mutagenesis rate. In combination with a stringent selection method, enzymatic activity has been evolved up to 16000 fold increase over the wild-type form of the enzyme. In essence, the background mutagenesis yielded the genetic variability on which recombination acted to enhance the activity.

A third feature of recombination is that it can be used to remove deleterious mutations. As discussed above, during the process of the randomization, for every one beneficial mutation, there may be at least one or more neutral or inhibitory mutations. Such mutations can be removed by including in the assembly reaction an excess of the wild-type random-size fragments, in addition to the random-size fragments of the selected mutant from the previous selection. During the next selection, some of the most active variants of the polynucleotide/polypeptide/enzyme, should have lost the inhibitory mutations.

Finally, recombination enables parallel processing. This represents a significant advantage since there are likely multiple characteristics that would make a protein more desirable (e.g. solubility, activity, etc.). Since it is increasingly difficult to screen for more than one desirable trait at a time, other methods of molecular evolution tend to be inhibitory. However, using recombination, it would be possible to combine the randomized fragments of the best representative variants for the various traits, and then select for multiple properties at once.

DNA shuffling can also be applied to the polynucleotides and polypeptides of the present invention to decrease their immunogenicity in a specified host, particularly if the polynucleotides and polypeptides provide a therapeutic use. For example, a particular variant of the present invention may be created and isolated using DNA shuffling technology. Such a variant may have all of the desired characteristics, though may be highly immunogenic in a host due to its novel intrinsic structure. Specifically, the desired characteristic may cause the polypeptide to have a non-native structure which could no longer be recognized as a “self” molecule, but rather as a “foreign”, and thus activate a host immune response directed against the novel variant. Such a limitation can be overcome, for example, by including a copy of the gene sequence for a xenobiotic ortholog of the native protein in with the gene sequence of the novel variant gene in one or more cycles of DNA shuffling. The molar ratio of the ortholog and novel variant DNAs could be varied accordingly. Ideally, the resulting hybrid variant identified would contain at least some of the coding sequence which enabled the xenobiotic protein to evade the host immune system, and additionally, the coding sequence of the original novel varient that provided the desired characteristics.

Likewise, the invention encompasses the application of DNA shuffling technology to the evolution of polynucletotides and polypeptides of the invention, wherein one or more cycles of DNA shuffling include, in addition to the gene template DNA, oligonucleotides coding for known allelic sequences, optimized codon sequences, known variant sequences, known polynucleotide polymorphism sequences, known ortholog sequences, known homolog sequences, additional homologous sequences, additional non-homologous sequences, sequences from another species, and any number and combination of the above.

In addition to the described methods above, there are a number of related methods that may also be applicable, or desirable in certain cases. Representative among these are the methods discussed in PCT applications WO 98/31700, and WO 98/32845, which are hereby incorporated by reference. Furthermore, related methods can also be applied to the polynucleotide sequences of the present invention in order to evolve invention for creating ideal variants for use in gene therapy, protein engineering, evolution of whole cells containing the variant, or in the evolution of entire enzyme pathways containing polynucleotides of the invention as described in PCT applications WO 98/13485, WO 98/13487, WO 98/27230, WO 98/31837, and Crameri, A., et al., Nat. Biotech., 15:436-438, (1997), respectively.

Additional methods of applying “DNA Shuffling” technology to the polynucleotides and polypeptides of the present invention, including their proposed applications, may be found in U.S. Pat. No. 5,605,793; PCT Application No. WO 95/22625; PCT Application No. WO 97/20078; PCT Application No. WO 97/35966; and PCT Application No. WO 98/42832; PCT Application No. The forgoing are hereby incorporated in their entirety herein for all purposes.

### Example 12

### Method Of Isolating Antibody Fragments Directed Against Genes 1-13 from a Library of scFvs.

Naturally occurring V-genes isolated from human PBLs are constructed into a library of antibody fragments which contain reactivities against Genes 1-13 to which the donor may or may not have been exposed (see e.g., U.S. Pat. No. 5,885,793 incorporated herein by reference in its entirety).

Rescue of the Library. A library of scFvs is constructed from the RNA of human PBLs as described in PCT publication WO 92/01047. To rescue phage displaying antibody fragments, approximately 109 E. coli harboring the phagemid are used to inoculate 50 ml of 2×TY containing 1% glucose and 100 μg/ml of ampicillin (2×TY-AMP-GLU) and grown to an O.D. of 0.8 with shaking. Five ml of this culture is used to inoculate 50 ml of 2×TY-AMP-GLU, 2×108 TU of delta gene 3 helper (M13 delta gene III, see PCT publication WO 92/01047) are added and the culture incubated at 37° C. for 45 minutes without shaking and then at 37° C. for 45 minutes with shaking. The culture is centrifuged at 4000 r.p.m. for 10 min. and the pellet resuspended in 2 liters of 2×TY containing 100 μg/ml ampicillin and 50 ug/ml kanamycin and grown overnight. Phage are prepared as described in PCT publication WO 92/01047.

M13 delta gene III is prepared as follows: M13 delta gene III helper phage does not encode gene III protein, hence the phage(mid) displaying antibody fragments have a greater avidity of binding to antigen. Infectious M13 delta gene III particles are made by growing the helper phage in cells harboring a pUC19 derivative supplying the wild type gene III protein during phage morphogenesis. The culture is incubated for 1 hour at 37° C. without shaking and then for a further hour at 37° C. with shaking. Cells are spun down (IEC-Centra 8,400 r.p.m. for 10 min), resuspended in 300 ml 2×TY broth containing 100 μg ampicillin/ml and 25 μg kanamycin/ml (2×TY-AMP-KAN) and grown overnight, shaking at 37° C. Phage particles are purified and concentrated from the culture medium by two PEG-precipitations (Sambrook et al., 1990), resuspended in 2 ml PBS and passed through a 0.45 μm filter (Minisart NML; Sartorius) to give a final concentration of approximately 1013 transducing units/ml (ampicillin-resistant clones).

Panning of the Library. Immunotubes (Nunc) are coated overnight in PBS with 4 ml of either 100 μg/ml or 10 μg/ml of a polypeptide of the present invention. Tubes are blocked with 2% Marvel-PBS for 2 hours at 37° C. and then washed 3 times in PBS. Approximately 1013 TU of phage is applied to the tube and incubated for 30 minutes at room temperature tumbling on an over and under turntable and then left to stand for another 1.5 hours. Tubes are washed 10 times with PBS 0.1% Tween-20 and 10 times with PBS. Phage are eluted by adding 1 ml of 100 mM triethylamine and rotating 15 minutes on an under and over turntable after which the solution is immediately neutralized with 0.5 ml of 1.0M Tris-HCl, pH 7.4. Phage are then used to infect 10 ml of mid-log E. coli TG1 by incubating eluted phage with bacteria for 30 minutes at 37° C. The E. coli are then plated on TYE plates containing 1% glucose and 100 μg/ml ampicillin. The resulting bacterial library is then rescued with delta gene 3 helper phage as described above to prepare phage for a subsequent round of selection. This process is then repeated for a total of 4 rounds of affinity purification with tube-washing increased to 20 times with PBS, 0.1% Tween-20 and 20 times with PBS for rounds 3 and 4.

Characterization of Binders. Eluted phage from the 3rd and 4th rounds of selection are used to infect E. coli HB 2151 and soluble scFv is produced (Marks, et al., 1991) from single colonies for assay. ELISAs are performed with microtitre plates coated with either 10 pg/ml of the polypeptide of the present invention in 50 mM bicarbonate pH 9.6. Clones positive in ELISA are further characterized by PCR fingerprinting (see, e.g., PCT publication WO 92/01047) and then by sequencing. These ELISA positive clones may also be further characterized by techniques known in the art, such as, for example, epitope mapping, binding affinity, receptor signal transduction, ability to block or competitively inhibit antibody/antigen binding, and competitive agonistic or antagonistic activity.

Moreover, in another preferred method, the antibodies directed against the polypeptides of the present invention may be produced in plants. Specific methods are disclosed in U.S. Pat. Nos. 5,959,177, and 6,080,560, which are hereby incorporated in their entirety herein. The methods not only describe methods of expressing antibodies, but also the means of assembling foreign multimeric proteins in plants (i.e., antibodies, etc,), and the subsequent secretion of such antibodies from the plant.

The contents of all patents, patent applications, published PCT applications and articles, books, references, reference manuals, abstracts and internet websites cited herein are hereby incorporated by reference in their entirety to more fully describe the state of the art to which the invention pertains.

As various changes can be made in the above-described subject matter without departing from the scope and spirit of the present invention, it is intended that all subject matter contained in the above description, or defined in the appended claims, be interpreted as descriptive and illustrative of the present invention. Many modifications and variations of the present invention are possible in light of the above teachings.

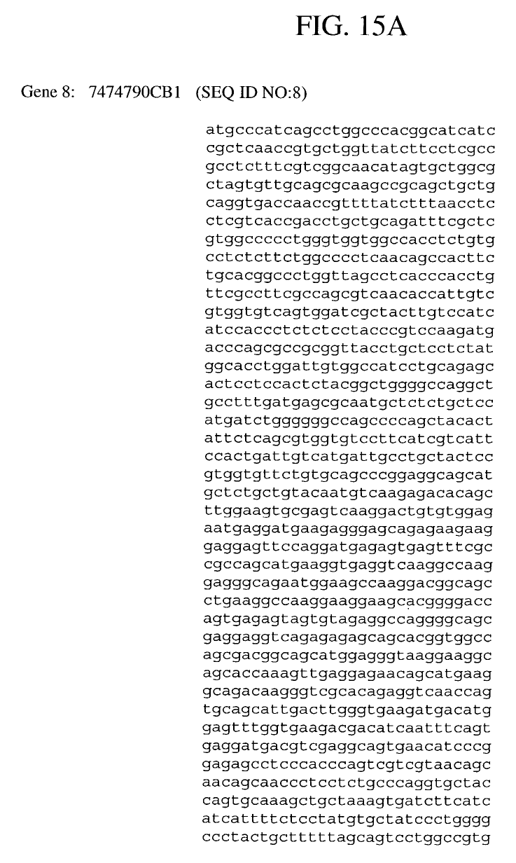

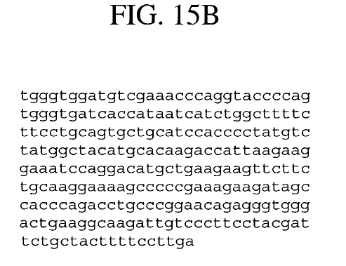

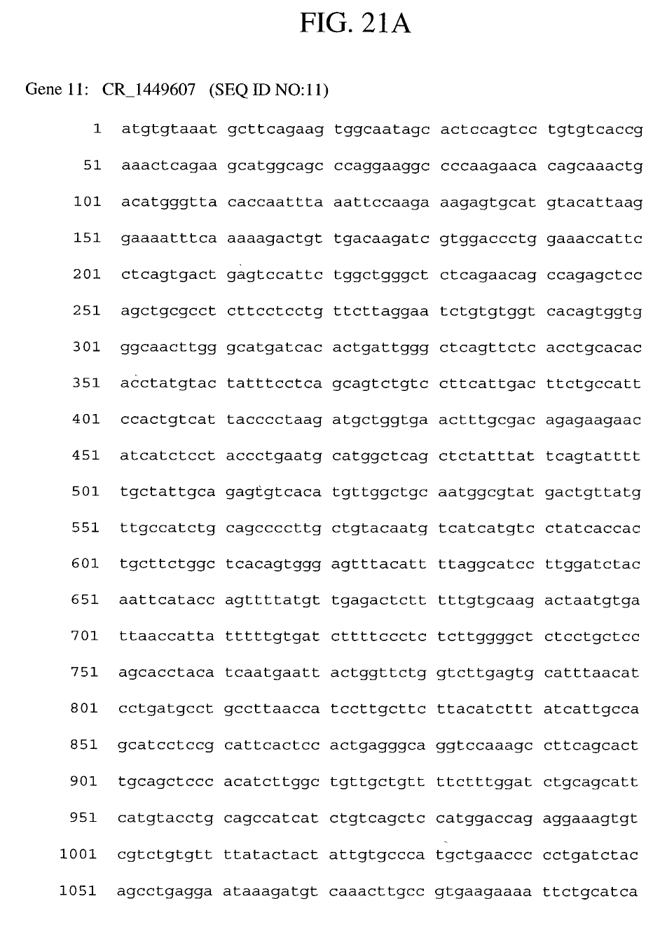

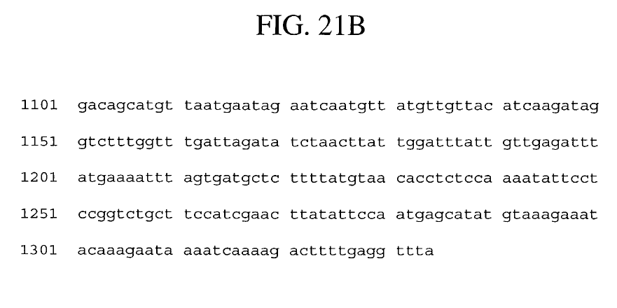

TABLE 1
Novel G-Protein Coupled Receptors of the Present Invention

| | | | | NUCLEIC ACID | |
|---|---|---|---|---|---|
| GPCR | CLONE ID/ | FUNCTIONAL | TISSUE | SEQ ID NO./FIG. | ENCODED AMINO ACID |
| GENENO. | BAC ID | ANNOTATION | EXPRESSION | NO. | SEQ ID NO./FIG. NO. |
| 1 | 1462859.1 | Sensory GPCR | Fetal Lung; | SEQ ID NO:1; | SEQ ID NO:14; |
| | | | Testis; B cells | | |
| 2 | 1102336.1 | Sensory GPCR | ND | SEQ ID NO:2; | SEQ ID NO:15; |
| 3 | BAC:NT_024210 | Sensory GPCR | ND | SEQ ID NO:3; | SEQ ID NO:16; |
| 4 | 27534.1 | Sensory GPCR | Lung | SEQ ID NO:4; | SEQ ID NO:17; |
| 5 | BAC:AC018630 | Sensory GPCR | Uterus | SEQ ID NO:5; | SEQ ID NO:18; |
| 6 | BAC:AC021089 | Chemokine | ND | SEQ ID NO:6; | SEQ ID NO:19; |
| | | GPCR | | | |
| 7 | 338589.1 | Human orphan | Skull tumor | SEQ ID NO:7; | SEQ ID NO:20; |
| | | GPCR | | FIGS. 13A/13B | |
| | | AAY90761 | | | |
| 8 | 7474790CB1 | Human orphan | ND | SEQ ID NO:8; | SEQ ID NO:21; |
| | | GPCR P1_312546 | | FIGS. 15A/15B | |
| 9 | 356272.1 | Sensory GPCR, | Cartilage | SEQ ID NO:9; | SEQ ID NO:22; |
| | | P1_314986 | | | |
| 10 | BAC:AC084434 | Sensory GPCR, | ND | SEQ ID NO:10; | SEQ ID NO:23; |
| | | P2_94452 | | | |
| 11 | CR_1449607 | Sensory GPCR, | ND | SEQ ID NO:11; | SEQ ID NO:24; |
| | | P2_94474 | | FIGS. 21A/21B | |
| 12 | 7474816CB1 | Sensory GPCR, | ND | SEQ ID NO:12; | SEQ ID NO:25; |
| | | P2_96621 | | | |
| 13 | 1137487.1 | Very Large | Brain | SEQ ID NO:13; | SEQ ID NO:26; |
| | | GPCR, | | | |
| | | P1_404650 | | | |

TABLE 2
Predicted Primers for Novel G-Protein Coupled Receptors

| GPCR | LEFT PRIMER | RIGHT PRIMER |
|---|---|---|
| 1 | tcacggctcccaaatctatc | tgtgcatcacagcaatcaga |
| | (SEQ ID NO:27) | (SEQ ID NO:28) |
| 2 | tctgtaagcagggtgctgtg | acaatgaggccgtaggacac |
| | (SEQ ID NO:29) | (SEQ ID NO:30) |
| 3 | tctcaccctcaccaccctac | cagcagcaaatgatggctaa |
| | (SEQ ID NO:31) | (SEQ ID NO:32) |
| 4 | acctgcaccaccactctacc | gggaaaggaatcatcagcaa |
| | (SEQ ID NO:33) | (SEQ ID NO:34) |
| 5 | taaattccattgagcgggtc | agcaagccagttgctgaaat |
| | (SEQ ID NO:35) | (SEQ ID NO:36) |
| 6a | gctggtacacatcatgtccg | acaggttgcttctggcactt |
| 1st | (SEQ ID NO:37) | (SEQ ID NO:38) |
| primer | | |
| 6b | cctccatctttgccacactt | ggcacttgaagaaagccttg |
| 2nd | (SEQ ID NO:39) | (SEQ ID NO:40) |
| primer | | |
| 7 | cagctcctgtaggcatctcc | caccagtctgatgacccctt |
| | (SEQ ID NO:41) | (SEQ ID NO:42) |
| 8 | tcagtgaggatgacgtcgag | gcaggaagaaaagccagatg |
| | (SEQ ID NO:43) | (SEQ ID NO:44) |
| 9 | gtggcctatgaccgctatgt | atggaatgcagatttccagc |
| | (SEQ ID NO:45) | (SEQ ID NO:46) |
| 10 | ataggcctggtttgtgcatc | tgtgggagctacaagtgctg |
| | (SEQ ID NO:47) | (SEQ ID NO:48) |
| 11 | catgatcacactgattgggc | ctctgtcgcaaagttcacca |
| | (SEQ ID NO:49) | (SEQ ID NO:50) |
| 12 | gccagagcgcacttacctac | ctcaccaaccaggaaatgct |
| | (SEQ ID NO:51) | (SEQ ID NO:52) |
| 13a | gtgacaattgcagcctctga | agtgatattggcgctcgtct |
| 1st | (SEQ ID NO:53) | (SEQ ID NO:54) |
| primer | | |
| 13b | cttcacctctggcaacatca | acttttcccatgaggccttt |
| 2nd | (SEQ ID NO:55) | (SEQ ID NO:56) |
| primer | | |

TABLE 3
Predicted Internal Primers for
Novel G-Protein Coupled Receptors

| GPCR | |
|---|---|
| GENE | INTERNAL OLIGONUCLEOTIDE |
| 1 | tgtcaccctctgcactatgatgtcatcatggacaggagcacctgtgtccaaagagccactgt |
| | gtcttggctgtatggggg |
| | (SEQ ID NO:57) |
| 2 | tccgcctggcttgcaccaacaccaagaagctggaggagactgactttgtcctggcctccct |
| | cgtcattgtatcttccttg |
| | (SEQ ID NO:58) |
| 3 | tctgtcctcctggctatgtccgttgactgctatgtggccatctgctgtcccctccattatgcctc |
| | catcctcaccaatga |
| | (SEQ ID NO:59) |
| 4 | ttgcttggcccagatgttctttgttcatgggttcacaggtgtggagtctggggtgctcatgctc |
| | atggctctagaccgct |
| | (SEQ ID NO:60) |
| 5 | aagagacaaaagatctcttttgctgaccagattctcactgctctggcggtctccagagttggtt |
| | tgctctgggtattatt |
| | (SEQ ID NO:61) |
| 6a | ttctgaacacagccatcaacttcttcctctactgcttcatcagcaagcggttccgcaccatgg |
| | cagccgccacgctcaag |
| | (SEQ ID NO:62) |
| 6b | gcgcccatccagaaccgctggctggtacacatcatgtccgacattgccaacatgctagccc |
| | ttctgaacacagccatcaa |
| | (SEQ ID NO:63) |
| 7 | cctctgaagtcactgaatcccagaaaggctctctacctttagcacaagggaggtcttcacca |
| | ctggacaaagaaggaacg |
| | (SEQ ID NO:64) |
| 8 | cccacccagtcgtcgtaacagcaacagcaaccctcctctgcccaggtgctaccagtgcaa |
| | agctgctaaagtgatcttca |
| | (SEQ ID NO:65) |
| 9 | ggccatctgcaacccactgcagtaccacatcatgatgtccaagaaactctgcattcagatga |
| | ccacaggcgccttcata |
| | (SEQ ID NO:66) |
| 10 | ccccagcctgaccatcctttgctcttacatctttattattgccagcatcctccacattcgctcca |
| | ctgagggcaggtcca |
| | (SEQ ID NO:67) |
| 11 | tctcacctgcacacacctatgtactatttcctcagcagtctgtccttcattgacttctgccattcc |
| | actgtcattacccc |
| | (SEQ ID NO:68) |
| 12 | cagtctgtcatgtggccctcatccacatggtggtccttctcaccatggtgttcttgtctccaca |
| | gctctttgaatcactg |
| | (SEQ ID NO:69) |
| 13a | tcatggaactctgtctccagtgactttgcattggaacatagactctgatcctgatggtgatctc |
| | gccttcacctctggca |
| | (SEQ ID NO:70) |
| 13b | ttgggcagacgagcgccaatatcactgtggagatattgcctgacgaagacccagaactgg |
| | ataaggcattctctgtgtca |
| | (SEQ ID NO:71) |

What is claimed is:

1. An isolated nucleic acid molecule comprising a polynucleotide having a nucleotide sequence selected from the group consisting of:
(a) a polynucleotide fragment of SEQ ID NO:7 or a polynucleotide fragment of the cDNA sequence included in ATCC Deposit No: ______, which is hybridizable to SEQ ID NO:7;
(b) a polynucleotide encoding a polypeptide fragment of SEQ ID NO:20 or a polypeptide fragment encoded by the cDNA sequence included in ATCC Deposit No: ______, which is hybridizable to SEQ ID NO:7;
(c) a polynucleotide encoding a polypeptide domain of SEQ ID NO:20 or a polypeptide domain encoded by the cDNA sequence included in ATCC Deposit No: ______, which is hybridizable to SEQ ID NO:7;
(d) a polynucleotide encoding a polypeptide epitope of SEQ ID NO:20 or a polypeptide epitope encoded by the cDNA sequence included in ATCC Deposit No: ______, which is hybridizable to SEQ ID NO:7;
(e) a polynucleotide encoding a polypeptide of SEQ ID NO:20 or the cDNA sequence included in ATCC Deposit No: ______, which is hybridizable to SEQ ID NO:7, having GPCR activity;
(f) an isolated polynucleotide comprising nucleotides 172 to 1152 of SEQ ID NO:7, wherein said nucleotides encode a polypeptide corresponding to amino acids 2 to 328 of SEQ ID NO:20 minus the start codon;
(g) an isolated polynucleotide comprising nucleotides 1 to 1152 of SEQ ID NO:7, wherein said nucleotides encode a polypeptide corresponding to amino acids 1 to 328 of SEQ ID NO:20 including the start codon;
(h) a polynucleotide which represents the complimentary sequence (antisense) of SEQ ID NO:7;
(i) (a) a polynucleotide fragment of SEQ ID NO:10 or a polynucleotide fragment of the cDNA sequence included in ATCC Deposit No: ______, which is hybridizable to SEQ ID NO:10;
(j) a polynucleotide encoding a polypeptide fragment of SEQ ID NO:23 or a polypeptide fragment encoded by the cDNA sequence included in ATCC Deposit No: ______, which is hybridizable to SEQ ID NO:10;
(k) a polynucleotide encoding a polypeptide domain of SEQ ID NO:23 or a polypeptide domain encoded by the cDNA sequence included in ATCC Deposit No: ______, which is hybridizable to SEQ ID NO:10;
(l) a polynucleotide encoding a polypeptide epitope of SEQ ID NO:23 or a polypeptide epitope encoded by the cDNA sequence included in ATCC Deposit No: ______, which is hybridizable to SEQ ID NO:10;
(m)a polynucleotide encoding a polypeptide of SEQ ID NO:23 or the cDNA sequence included in ATCC Deposit No: ______, which is hybridizable to SEQ ID NO:10, having GPCR activity;
(n) an isolated polynucleotide comprising nucleotides 21 to 950 of SEQ ID NO:10, wherein said nucleotides encode a polypeptide corresponding to amino acids 2 to 311 of SEQ ID NO:23 minus the start codon;
(o) an isolated polynucleotide comprising nucleotides 18 to 950 of SEQ ID NO:10, wherein said nucleotides encode a polypeptide corresponding to amino acids 1 to 311 of SEQ ID NO:23 including the start codon;
(p) a polynucleotide which represents the complimentary sequence (antisense) of SEQ ID NO:10;
(q) (a) a polynucleotide fragment of SEQ ID NO:13 or a polynucleotide fragment of the cDNA sequence included in ATCC Deposit No: PTA-3949, which is hybridizable to SEQ ID NO:13;
(r) a polynucleotide encoding a polypeptide fragment of SEQ ID NO:26 or a polypeptide fragment encoded by the cDNA sequence included in ATCC Deposit No: PTA-3949, which is hybridizable to SEQ ID NO:13;
(s) a polynucleotide encoding a polypeptide domain of SEQ ID NO:26 or a polypeptide domain encoded by the cDNA sequence included in ATCC Deposit No: PTA-3949, which is hybridizable to SEQ ID NO:13;
(t) a polynucleotide encoding a polypeptide epitope of SEQ ID NO:26 or a polypeptide epitope encoded by the cDNA sequence included in ATCC Deposit No: PTA-3949, which is hybridizable to SEQ ID NO:13;
(u) a polynucleotide encoding a polypeptide of SEQ ID NO:26 or the cDNA sequence included in ATCC Deposit No: PTA-3949, which is hybridizable to SEQ ID NO:13, having GPCR activity;
(v) an isolated polynucleotide comprising nucleotides 26 to 906 of SEQ ID NO:13, wherein said nucleotides encode a polypeptide corresponding to amino acids 2 to 295 of SEQ ID NO:26 minus the start codon;
(w) an isolated polynucleotide comprising nucleotides 23 to 950 of SEQ ID NO:13, wherein said nucleotides encode a polypeptide corresponding to amino acids 1 to 295 of SEQ ID NO:26 including the start codon;
(x) a polynucleotide which represents the complimentary sequence (antisense) of SEQ ID NO:13; and
(y) a polynucleotide capable of hybridizing under stringent conditions to any one of the polynucleotides specified in (a)-(x), wherein said polynucleotide does not hybridize under stringent conditions to a nucleic acid molecule having a nucleotide sequence of only A residues or of only T residues.

2. The isolated nucleic acid molecule of claim 1, wherein the polynucleotide fragment consists of a nucleotide sequence encoding a human G-protein coupled receptor.

3. A recombinant vector comprising the isolated nucleic acid molecule of claim 1.

4. A recombinant host cell comprising the vector sequences of claim 3.

5. An isolated polypeptide comprising an amino acid sequence selected from the group consisting of:
(a) a polypeptide fragment of SEQ ID NO:20 or the encoded sequence included in ATCC Deposit No: ______;
(b) a polypeptide fragment of SEQ ID NO:20 or the encoded sequence included in ATCC Deposit No: ______, having GPCR activity;
(c) a polypeptide domain of SEQ ID NO:20 or the encoded sequence included in ATCC Deposit No: ______;
(d) a polypeptide epitope of SEQ ID NO:20 or the encoded sequence included in ATCC Deposit No: ______;
(e) a full length protein of SEQ ID NO:20 or the encoded sequence included in ATCC Deposit No: ______;
(f) a polypeptide comprising amino acids 2 to 328 of SEQ ID NO:20, wherein said amino acids 2 to 328 comprising a polypeptide of SEQ ID NO:20 minus the start methionine;
(g) a polypeptide comprising amino acids 1 to 328 of SEQ ID NO:20;
(h) a polypeptide fragment of SEQ ID NO:23 or the encoded sequence included in ATCC Deposit No: ______;
(i) a polypeptide fragment of SEQ ID NO:23 or the encoded sequence included in ATCC Deposit No: ______, having GPCR activity;
(j) a polypeptide domain of SEQ ID NO:23 or the encoded sequence included in ATCC Deposit No: ______;
(k) a polypeptide epitope of SEQ ID NO:23 or the encoded sequence included in ATCC Deposit No: ______;
(l) a full length protein of SEQ ID NO:23 or the encoded sequence included in ATCC Deposit No: ______;
(m) a polypeptide comprising amino acids 2 to 311 of SEQ ID NO:23, wherein said amino acids 2 to 311 comprising a polypeptide of SEQ ID NO:23 minus the start methionine;
(n) a polypeptide comprising amino acids 1 to 311 of SEQ ID NO:23;
(o) a polypeptide fragment of SEQ ID NO:26 or the encoded sequence included in ATCC Deposit No: PTA-3949;
(p) a polypeptide fragment of SEQ ID NO:26 or the encoded sequence included in ATCC Deposit No: PTA-3949, having GPCR activity;
(q) a polypeptide domain of SEQ ID NO:26 or the encoded sequence included in ATCC Deposit No: PTA-3949;
(r) a polypeptide epitope of SEQ ID NO:26 or the encoded sequence included in ATCC Deposit No: PTA-3949;
(s) a full length protein of SEQ ID NO:26 or the encoded sequence included in ATCC Deposit No: PTA-3949;
(t) a polypeptide comprising amino acids 2 to 295 of SEQ ID NO:26, wherein said amino acids 2 to 295 comprising a polypeptide of SEQ ID NO:26 minus the start methionine; and
(u) a polypeptide comprising amino acids 1 to 295 of SEQ ID NO:26;

6. The isolated polypeptide of claim 5, wherein the full length protein comprises sequential amino acid deletions from either the C-terminus or the N-terminus.

7. An isolated antibody that binds specifically to the isolated polypeptide of claim 5.

8. A recombinant host cell that expresses the isolated polypeptide of claim 5.

9. A method of making an isolated polypeptide comprising:
(a) culturing the recombinant host cell of claim 8 under conditions such that said polypeptide is expressed; and
(b) recovering said polypeptide.

10. The polypeptide produced by claim 9.

11. A method for preventing, treating, or ameliorating a medical condition, comprising the step of administering to a mammalian subject a therapeutically effective amount of the polypeptide of claim 5, or a modulator thereof.

12. A method of diagnosing a pathological condition or a susceptibility to a pathological condition in a subject comprising:
(a) determining the presence or absence of a mutation in the polynucleotide of claim 1; and
(b) diagnosing a pathological condition or a susceptibility to a pathological condition based on the presence or absence of said mutation.

13. A method of diagnosing a pathological condition or a susceptibility to a pathological condition in a subject comprising:
(a) determining the presence or amount of expression of the polypeptide of claim 5 in a biological sample; and
(b) diagnosing a pathological condition or a susceptibility to a pathological condition based on the presence or amount of expression of the polypeptide.

14. An isolated nucleic acid molecule consisting of a polynucleotide having a nucleotide sequence selected from the group consisting of:
(a) a polynucleotide encoding a polypeptide of SEQ ID NO:20;
(b) an isolated polynucleotide consisting of nucleotides 172 to 1152 of SEQ ID NO:7, wherein said nucleotides encode a polypeptide corresponding to amino acids 2 to 328 of SEQ ID NO:20 minus the start codon;
(c) an isolated polynucleotide consisting of nucleotides 169 to 1152 of SEQ ID NO:7, wherein said nucleotides encode a polypeptide corresponding to amino acids 1 to 328 of SEQ ID NO:20 including the start codon;
(d) a polynucleotide encoding the Gene 7 polypeptide encoded by the cDNA clone contained in ATCC Deposit No. ______;
(e) a polynucleotide which represents the complimentary sequence (antisense) of SEQ ID NO:7;
(f) a polynucleotide encoding a polypeptide of SEQ ID NO:23;
(g) an isolated polynucleotide consisting of nucleotides 21 to 950 of SEQ ID NO: 10, wherein said nucleotides encode a polypeptide corresponding to amino acids 2 to 311 of SEQ ID NO:23 minus the start codon;
(h) an isolated polynucleotide consisting of nucleotides 18 to 950 of SEQ ID NO: 10, wherein said nucleotides encode a polypeptide corresponding to amino acids 1 to 311 of SEQ ID NO:23 including the start codon;
(i) a polynucleotide encoding the Gene 10 polypeptide encoded by the cDNA clone contained in ATCC Deposit No. ______;
(j) a polynucleotide which represents the complimentary sequence (antisense) of SEQ ID NO:10;
(k) a polynucleotide encoding a polypeptide of SEQ ID NO:26;
(l) an isolated polynucleotide consisting of nucleotides 26 to 906 of SEQ ID NO: 13, wherein said nucleotides encode a polypeptide corresponding to amino acids 2 to 295 of SEQ ID NO:26 minus the start codon;
(m) an isolated polynucleotide consisting of nucleotides 23 to 906 of SEQ ID NO: 13, wherein said nucleotides encode a polypeptide corresponding to amino acids 1 to 295 of SEQ ID NO:26 including the start codon;
(n) a polynucleotide encoding the Gene 13 polypeptide encoded by the cDNA clone contained in ATCC Deposit No. PTA-3949;
(o) a polynucleotide which represents the complimentary sequence (antisense) of SEQ ID NO:13;

15. The isolated nucleic acid molecule of claim 14, wherein the polynucleotide comprises a nucleotide sequence encoding a human G-protein coupled receptor.

16. A recombinant vector comprising the isolated nucleic acid molecule of claim 15.

17. A recombinant host cell comprising the recombinant vector of claim 16.

18. An isolated polypeptide consisting of an amino acid sequence selected from the group consisting of:
(a) a polypeptide fragment of SEQ ID NO:20 having GPCR activity;
(b) a polypeptide domain of SEQ ID NO:20 having GPCR activity;
(c) a full length protein of SEQ ID NO:20;
(d) a polypeptide corresponding to amino acids 2 to 328 of SEQ ID NO:20, wherein said amino acids 2 to 328 consisting of a polypeptide of SEQ ID NO:20 minus the start methionine;
(e) a polypeptide corresponding to amino acids 1 to 328 of SEQ ID NO:20;
(f) a polypeptide encoded by the cDNA contained in ATCC Deposit No. ______;
(g) a polypeptide fragment of SEQ ID NO:23 having GPCR activity;
(h) a polypeptide domain of SEQ ID NO:23 having GPCR activity;
(i) a full length protein of SEQ ID NO:23;
(j) a polypeptide corresponding to amino acids 2 to 311 of SEQ ID NO:23, wherein said amino acids 2 to 311 consisting of a polypeptide of SEQ ID NO:23 minus the start methionine;
(k) a polypeptide corresponding to amino acids 1 to 311 of SEQ ID NO:23;
(l) a polypeptide encoded by the cDNA contained in ATCC Deposit No. ______;
(m)a polypeptide fragment of SEQ ID NO:26 having GPCR activity;
(n) a polypeptide domain of SEQ ID NO:26 having GPCR activity;
(o) a full length protein of SEQ ID NO:26;
(p) a polypeptide corresponding to amino acids 2 to 295 of SEQ ID NO:26, wherein said amino acids 2 to 295 consisting of a polypeptide of SEQ ID NO:26 minus the start methionine;
(q) a polypeptide corresponding to amino acids 1 to 295 of SEQ ID NO:26;
(r) a polypeptide encoded by the cDNA contained in ATCC Deposit No. PTA-3949;

19. The method for preventing, treating, or ameliorating a medical condition of claim 11, wherein the medical condition is selected from the group consisting of a neural disorder; an endocrine disorder; a sleep disorder; disorders that affect the nucleus accumbens, disorders that affect the brains ‘reward center’ function, neurotransmitter release disorders, disorders affecting the release of dopamine, disorders affecting the release of opioid peptides, disorders affecting the release of serotonin, disorders affecting the release of GABA, pineal gland disorders, disorders affecting the establishment of circadian rhythms, disorders affecting the maintenance of circadian rhythms, disorders affecting the control of the sleep/wake cycle; melatonin secretion disorders, pituitary hormone secretion disorders, oxytocin secretion disorders, disorders affecting neuroendocrine response to stressful stimuli, disorders affecting oxytocin secretion during neuroendocrine response to stressful stimuli, disorders affecting nocturnal patterns of hormone secretion, disorders affecting the nocturnal hormone secretion of prolactin, disorders affecting the nocturnal hormone secretion of cortisol, and/or disorders affecting the nocturnal hormone secretion of growth hormone; neuro-pathologies, including responses to stress, and propensity to develop addictive behaviors, as well as a vast number of neuroendocrine abnormalities including sleep disorders; and brain tumors.
